SEQUENCE LISTING (134 sequences)

SEQ ID NO: 1 - protein, 106 aa, Artificial Sequence
  Description of Artificial Sequence Synthetic polypeptide
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIK

SEQ ID NO: 2 - protein, 23 aa, Artificial Sequence
  Description of Artificial Sequence Synthetic peptide
DIQLTQSPSSLSASVGDRVTITC

SEQ ID NO: 3 - protein, 11 aa, Artificial Sequence
  Description of Artificial Sequence Synthetic peptide
KASQDVGTSVA

SEQ ID NO: 4 - protein, 15 aa, Artificial Sequence
  Description of Artificial Sequence Synthetic peptide
WYQQKPGKAPKLLIY

SEQ ID NO: 5 - protein, 7 aa, Artificial Sequence
  Description of Artificial Sequence Synthetic peptide
WTSTRHT

SEQ ID NO: 6 - protein, 32 aa, Artificial Sequence
  Description of Artificial Sequence Synthetic polypeptide
GVPSRFSGSGSGTDFTFTISSLQPEDIATYYC

SEQ ID NO: 7 - protein, 8 aa, Artificial Sequence
  Description of Artificial Sequence Synthetic peptide
QQYSLYRS

SEQ ID NO: 8 - protein, 10 aa, Artificial Sequence
  Description of Artificial Sequence Synthetic peptide
FGQGTKVEIK

SEQ ID NO: 9 - protein, 119 aa, Artificial Sequence
  Description of Artificial Sequence Synthetic polypeptide
EVQLVESGGGVVQPGRSLRLSCSSSGFDFTTYWMSWVRQAPGKGLEWVAEIHPDSSTINYAPSLKDRFTISRDNSKNTLFLQMDSLRPEDTGVYFCASLYFGFPWFAYWGQGTPVTVSS

SEQ ID NO: 10 - protein, 30 aa, Artificial Sequence
  Description of Artificial Sequence Synthetic polypeptide
EVQLVESGGGVVQPGRSLRLSCSSSGFDFT

SEQ ID NO: 11 - protein, 5 aa, Artificial Sequence
  Description of Artificial Sequence Synthetic peptide
TYWMS

SEQ ID NO: 12 - protein, 14 aa, Artificial Sequence
  Description of Artificial Sequence Synthetic peptide
WVRQAPGKGLEWVA

SEQ ID NO: 13 - protein, 17 aa, Artificial Sequence
  Description of Artificial Sequence Synthetic peptide
EIHPDSSTINYAPSLKD

SEQ ID NO: 14 - protein, 32 aa, Artificial Sequence
  Description of Artificial Sequence Synthetic polypeptide
RFTISRDNSKNTLFLQMDSLRPEDTGVYFCAS

SEQ ID NO: 15 - protein, 10 aa, Artificial Sequence
  Description of Artificial Sequence Synthetic peptide
LYFGFPWFAY

SEQ ID NO: 16 - protein, 11 aa, Artificial Sequence
  Description of Artificial Sequence Synthetic peptide
WGQGTPVTVSS

SEQ ID NO: 17 - protein, 108 aa, Artificial Sequence
  Description of Artificial Sequence Synthetic polypeptide
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 18 - protein, 23 aa, Artificial Sequence
  Description of Artificial Sequence Synthetic peptide
DIQMTQSPSSLSASVGDRVTITC

SEQ ID NO: 19 - protein, 11 aa, Artificial Sequence
  Description of Artificial Sequence Synthetic peptide
KASAAVGTYVA

SEQ ID NO: 20 - protein, 15 aa, Artificial Sequence
  Description of Artificial Sequence Synthetic peptide
WYQQKPGKAPKLLIY

SEQ ID NO: 21 - protein, 7 aa, Artificial Sequence
  Description of Artificial Sequence Synthetic peptide
SASYRKR

SEQ ID NO: 22 - protein, 32 aa, Artificial Sequence
  Description of Artificial Sequence Synthetic polypeptide
GVPSRFSGSGSGTDFTLTISSLQPEDFATYYC

SEQ ID NO: 23 - protein, 10 aa, Artificial Sequence
  Description of Artificial Sequence Synthetic peptide
HQYYTYPLFT

SEQ ID NO: 24 - protein, 10 aa, Artificial Sequence
  Description of Artificial Sequence Synthetic peptide
FGQGTKLEIK

SEQ ID NO: 25 - protein, 30 aa, Artificial Sequence
  Description of Artificial Sequence Synthetic polypeptide
QVQLVQSGAEVKKPGASVKVSCKASGYTFT

SEQ ID NO: 26 - protein, 5 aa, Artificial Sequence
  Description of Artificial Sequence Synthetic peptide
EFGMN

SEQ ID NO: 27 - protein, 14 aa, Artificial Sequence
  Description of Artificial Sequence Synthetic peptide
WVRQAPGQGLEWMG

SEQ ID NO: 28 - protein, 17 aa, Artificial Sequence
  Description of Artificial Sequence Synthetic peptide
WINTKTGEATYVEEFKG

SEQ ID NO: 29 - protein, 32 aa, Artificial Sequence
  Description of Artificial Sequence Synthetic polypeptide
RVTFTTDTSTSTAYMELRSLRSDDTAVYYCAR

SEQ ID NO: 30 - protein, 12 aa, Artificial Sequence
  Description of Artificial Sequence Synthetic peptide
WDFAYYVEAMDY

SEQ ID NO: 31 - protein, 11 aa, Artificial Sequence
  Description of Artificial Sequence Synthetic peptide
WGQGTTVTVSS

SEQ ID NO: 32 - protein, 106 aa, Artificial Sequence
  Description of Artificial Sequence Synthetic polypeptide
ENVLTQSPSSMSASVGDRVNIACSASSSVSYMHWFQQKPGKSPKLWIYSTSNLASGVPSRFSGSGSGTDYSLTISSMQPEDAATYYCQQRSSYPLTFGGGTKLEIK

SEQ ID NO: 33 - protein, 23 aa, Artificial Sequence
  Description of Artificial Sequence Synthetic peptide
ENVLTQSPSSMSASVGDRVNIAC

SEQ ID NO: 34 - protein, 10 aa, Artificial Sequence
  Description of Artificial Sequence Synthetic peptide
SASSSVSYMH

SEQ ID NO: 35 - protein, 15 aa, Artificial Sequence
  Description of Artificial Sequence Synthetic peptide
WFQQKPGKSPKLWIY

SEQ ID NO: 36 - protein, 7 aa, Artificial Sequence
  Description of Artificial Sequence Synthetic peptide
STSNLAS

SEQ ID NO: 37 - protein, 32 aa, Artificial Sequence
  Description of Artificial Sequence Synthetic polypeptide
GVPSRFSGSGSGTDYSLTISSMQPEDAATYYC

SEQ ID NO: 38 - protein, 9 aa, Artificial Sequence
  Description of Artificial Sequence Synthetic peptide
QQRSSYPLT

SEQ ID NO: 39 - protein, 10 aa, Artificial Sequence
  Description of Artificial Sequence Synthetic peptide
FGGGTKLEIK

SEQ ID NO: 40 - protein, 120 aa, Artificial Sequence
  Description of Artificial Sequence Synthetic polypeptide
QVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSS

SEQ ID NO: 41 - protein, 30 aa, Artificial Sequence
  Description of Artificial Sequence Synthetic polypeptide
QVKLEQSGAEVVKPGASVKLSCKASGFNIK

SEQ ID NO: 42 - protein, 5 aa, Artificial Sequence
  Description of Artificial Sequence Synthetic peptide
DSYMH

SEQ ID NO: 43 - protein, 14 aa, Artificial Sequence
  Description of Artificial Sequence Synthetic peptide
WLRQGPGQRLEWIG

SEQ ID NO: 44 - protein, 17 aa, Artificial Sequence
  Description of Artificial Sequence Synthetic peptide
WIDPENGDTEYAPKFQG

SEQ ID NO: 45 - protein, 32 aa, Artificial Sequence
  Description of Artificial Sequence Synthetic polypeptide
KATFTTDTSANTAYLGLSSLRPEDTAVYYCNE

SEQ ID NO: 46 - protein, 11 aa, Artificial Sequence
  Description of Artificial Sequence Synthetic peptide
GTPTGPYYFDY

SEQ ID NO: 47 - protein, 11 aa, Artificial Sequence
  Description of Artificial Sequence Synthetic peptide
WGQGTLVTVSS

SEQ ID NO: 48 - protein, 106 aa, Artificial Sequence
  Description of Artificial Sequence Synthetic polypeptide
ENVLTQSPSSMSVSVGDRVTIACSASSSVPYMHWLQQKPGKSPKLLIYLTSNLASGVPSRFSGSGSGTDYSLTISSVQPEDAATYYCQQRSSYPLTFGGGTKLEIK

SEQ ID NO: 49 - protein, 23 aa, Artificial Sequence
  Description of Artificial Sequence Synthetic peptide
ENVLTQSPSSMSVSVGDRVTIAC

SEQ ID NO: 50 - protein, 10 aa, Artificial Sequence
  Description of Artificial Sequence Synthetic peptide
SASSSVPYMH

SEQ ID NO: 51 - protein, 15 aa, Artificial Sequence
  Description of Artificial Sequence Synthetic peptide
WLQQKPGKSPKLLIY

SEQ ID NO: 52 - protein, 7 aa, Artificial Sequence
  Description of Artificial Sequence Synthetic peptide
LTSNLAS

SEQ ID NO: 53 - protein, 32 aa, Artificial Sequence
  Description of Artificial Sequence Synthetic polypeptide
GVPSRFSGSGSGTDYSLTISSVQPEDAATYYC

SEQ ID NO: 54 - protein, 9 aa, Artificial Sequence
  Description of Artificial Sequence Synthetic peptide
QQRSSYPLT

SEQ ID NO: 55 - protein, 10 aa, Artificial Sequence
  Description of Artificial Sequence Synthetic peptide
FGGGTKLEIK

SEQ ID NO: 56 - protein, 120 aa, Artificial Sequence; identical to SEQ ID NO: 40
  Description of Artificial Sequence Synthetic polypeptide

SEQ ID NO: 57 - protein, 30 aa, Artificial Sequence; identical to SEQ ID NO: 41
  Description of Artificial Sequence Synthetic polypeptide

SEQ ID NO: 58 - protein, 5 aa, Artificial Sequence
  Description of Artificial Sequence Synthetic peptide
DSYMH

SEQ ID NO: 59 - protein, 14 aa, Artificial Sequence
  Description of Artificial Sequence Synthetic peptide
WLRQGPGQRLEWIG

SEQ ID NO: 60 - protein, 17 aa, Artificial Sequence
  Description of Artificial Sequence Synthetic peptide
WIDPENGDTEYAPKFQG

SEQ ID NO: 61 - protein, 32 aa, Artificial Sequence; identical to SEQ ID NO: 45
  Description of Artificial Sequence Synthetic polypeptide

SEQ ID NO: 62 - protein, 11 aa, Artificial Sequence
  Description of Artificial Sequence Synthetic peptide
GTPTGPYYFDY

SEQ ID NO: 63 - protein, 11 aa, Artificial Sequence
  Description of Artificial Sequence Synthetic peptide
WGQGTLVTVSS

SEQ ID NO: 64 - protein, 23 aa, Artificial Sequence
  Description of Artificial Sequence Synthetic peptide
QTVLSQSPAILSASPGEKVTMTC

SEQ ID NO: 65 - protein, 10 aa, Artificial Sequence
  Description of Artificial Sequence Synthetic peptide
RASSSVTYIH

SEQ ID NO: 66 - protein, 15 aa, Artificial Sequence
  Description of Artificial Sequence Synthetic peptide
WYQQKPGSSPKSWIY

SEQ ID NO: 67 - protein, 7 aa, Artificial Sequence
  Description of Artificial Sequence Synthetic peptide
ATSNLAS

SEQ ID NO: 68 - protein, 32 aa, Artificial Sequence
  Description of Artificial Sequence Synthetic polypeptide
GVPARFSGSGSGTSYSLTISRVEAEDAATYYC

SEQ ID NO: 69 - protein, 9 aa, Artificial Sequence
  Description of Artificial Sequence Synthetic peptide
QHWSSKPPT

SEQ ID NO: 70 - protein, 10 aa, Artificial Sequence
  Description of Artificial Sequence Synthetic peptide
FGGGTKLEIK

SEQ ID NO: 71 - protein, 121 aa, Artificial Sequence
  Description of Artificial Sequence Synthetic polypeptide
EVKLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTLTVSS

SEQ ID NO: 72 - protein, 30 aa, Artificial Sequence
  Description of Artificial Sequence Synthetic polypeptide
EVKLVESGGGLVQPGGSLRLSCATSGFTFT

SEQ ID NO: 73 - protein, 5 aa, Artificial Sequence
  Description of Artificial Sequence Synthetic peptide
DYYMN

SEQ ID NO: 74 - protein, 14 aa, Artificial Sequence
  Description of Artificial Sequence Synthetic peptide
WVRQPPGKALEWLG

SEQ ID NO: 75 - protein, 19 aa, Artificial Sequence
  Description of Artificial Sequence Synthetic peptide
FIGNKANGYTTEYSASVKG

SEQ ID NO: 76 - protein, 32 aa, Artificial Sequence
  Description of Artificial Sequence Synthetic polypeptide
RFTISRDKSQSILYLQMNTLRAEDSATYYCTR

SEQ ID NO: 77 - protein, 10 aa, Artificial Sequence
  Description of Artificial Sequence Synthetic peptide
DRGLRFYFDY

SEQ ID NO: 78 - protein, 11 aa, Artificial Sequence
  Description of Artificial Sequence Synthetic peptide
WGQGTTLTVSS

SEQ ID NO: 79 - protein, 111 aa, Artificial Sequence
  Description of Artificial Sequence Synthetic polypeptide
DIQLTQSPSSLSASVGDRVTITCRAGESVDIFGVGFLHWYQQKPGKAPKLLIYRASNLESGVPSRFSGSGSRTDFTLTISSLQPEDFATYYCQQTNEDPYTFGQGTKVEIK

SEQ ID NO: 80 - protein, 23 aa, Artificial Sequence
  Description of Artificial Sequence Synthetic peptide
DIQLTQSPSSLSASVGDRVTITC

SEQ ID NO: 81 - protein, 15 aa, Artificial Sequence
  Description of Artificial Sequence Synthetic peptide
RAGESVDIFGVGFLH

SEQ ID NO: 82 - protein, 15 aa, Artificial Sequence
  Description of Artificial Sequence Synthetic peptide
WYQQKPGKAPKLLIY

SEQ ID NO: 83 - protein, 7 aa, Artificial Sequence
  Description of Artificial Sequence Synthetic peptide
RASNLES

SEQ ID NO: 84 - protein, 32 aa, Artificial Sequence
  Description of Artificial Sequence Synthetic polypeptide
GVPSRFSGSGSRTDFTLTISSLQPEDFATYYC

SEQ ID NO: 85 - protein, 9 aa, Artificial Sequence
  Description of Artificial Sequence Synthetic peptide
QQTNEDPYT

SEQ ID NO: 86 - protein, 10 aa, Artificial Sequence
  Description of Artificial Sequence Synthetic peptide
FGQGTKVEIK

SEQ ID NO: 87 - protein, 121 aa, Artificial Sequence
  Description of Artificial Sequence Synthetic polypeptide
EVQLVESGGGLVQPGGSLRLSCAASGFNIKDTYMHWVRQAPGKGLEWVARIDPANGNSKYADSVKGRFTISADTSKNTAYLQMNSLRAEDTAVYYCAPFGYYVSDYAMAYWGQGTLVTVSS

SEQ ID NO: 88 - protein, 30 aa, Artificial Sequence
  Description of Artificial Sequence Synthetic polypeptide
EVQLVESGGGLVQPGGSLRLSCAASGFNIK

SEQ ID NO: 89 - protein, 5 aa, Artificial Sequence
  Description of Artificial Sequence Synthetic peptide
DTYMH

SEQ ID NO: 90 - protein, 14 aa, Artificial Sequence
  Description of Artificial Sequence Synthetic peptide
WVRQAPGKGLEWVA

SEQ ID NO: 91 - protein, 17 aa, Artificial Sequence
  Description of Artificial Sequence Synthetic peptide
RIDPANGNSKYADSVKG

SEQ ID NO: 92 - protein, 32 aa, Artificial Sequence
  Description of Artificial Sequence Synthetic polypeptide
RFTISADTSKNTAYLQMNSLRAEDTAVYYCAP

SEQ ID NO: 93 - protein, 12 aa, Artificial Sequence
  Description of Artificial Sequence Synthetic peptide
FGYYVSDYAMAY

SEQ ID NO: 94 - protein, 11 aa, Artificial Sequence
  Description of Artificial Sequence Synthetic peptide
WGQGTLVTVSS

SEQ ID NO: 95 - protein, 107 aa, Artificial Sequence [sequence omitted]

SEQ ID NO: 96 - protein, 23 aa, Artificial Sequence
  Description of Artificial Sequence Synthetic peptide
DIQMTQSPASLSASVGDRVTITC

SEQ ID NO: 97 - protein, 11 aa, Artificial Sequence
  Description of Artificial Sequence Synthetic peptide
RASENIFSYLA

SEQ ID NO: 98 - protein, 15 aa, Artificial Sequence
  Description of Artificial Sequence Synthetic peptide
WYQQKPGKSPKLLVY

SEQ ID NO: 99 - protein, 7 aa, Artificial Sequence
  Description of Artificial Sequence Synthetic peptide
NTRTLAE

SEQ ID NO: 100 - protein, 32 aa, Artificial Sequence
  Description of Artificial Sequence Synthetic polypeptide
GVPSRFSGSGSGTDFSLTISSLQPEDFATYYC

SEQ ID NO: 101 - protein, 9 aa, Artificial Sequence
  Description of Artificial Sequence Synthetic peptide
QHHYGTPFT

SEQ ID NO: 102 - protein, 10 aa, Artificial Sequence
  Description of Artificial Sequence Synthetic peptide
FGSGTKLEIK

SEQ ID NO: 103 - protein, 120 aa, Artificial Sequence [sequence omitted]

SEQ ID NO: 104 - protein, 30 aa, Artificial Sequence
  Description of Artificial Sequence Synthetic polypeptide
EVQLQESGPGLVKPGGSLSLSCAASGFVFS

SEQ ID NO: 105 - protein, 5 aa, Artificial Sequence
  Description of Artificial Sequence Synthetic peptide
SYDMS

SEQ ID NO: 106 - protein, 14 aa, Artificial Sequence
  Description of Artificial Sequence Synthetic peptide
WVRQTPERGLEWVA

SEQ ID NO: 107 - protein, 17 aa, Artificial Sequence
  Description of Artificial Sequence Synthetic peptide
YISSGGGITYAPSTVKG

SEQ ID NO: 108 - protein, 32 aa, Artificial Sequence
  Description of Artificial Sequence Synthetic polypeptide
RFTVSRDNAKNTLYLQMNSLTSEDTAVYYCAA

SEQ ID NO: 109 - protein, 11 aa, Artificial Sequence
  Description of Artificial Sequence Synthetic peptide
HYFGSSGPFAY

SEQ ID NO: 110 - protein, 11 aa, Artificial Sequence
  Description of Artificial Sequence Synthetic peptide
WGQGTLVTVSS

SEQ ID NO: 111 - protein, 116 aa, Artificial Sequence
  Description of Artificial Sequence Synthetic polypeptide
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 112 - protein, 22 aa, Artificial Sequence
  Description of Artificial Sequence Synthetic peptide
QAVLTQPASLSASPGASASLTC

SEQ ID NO: 113 - protein, 14 aa, Artificial Sequence
  Description of Artificial Sequence Synthetic peptide
TLRRGINVGAYSIY

SEQ ID NO: 114 - protein, 15 aa, Artificial Sequence
  Description of Artificial Sequence Synthetic peptide
WYQQKPGSPPQYLLR

SEQ ID NO: 115 - protein, 11 aa, Artificial Sequence
  Description of Artificial Sequence Synthetic peptide
YKSDSDKQQGS

SEQ ID NO: 116 - protein, 34 aa, Artificial Sequence
  Description of Artificial Sequence Synthetic polypeptide
GVSSRFSASKDASANAGILLISGLQSEDEADYYC

SEQ ID NO: 117 - protein, 10 aa, Artificial Sequence
  Description of Artificial Sequence Synthetic peptide
MIWHSGASAV

SEQ ID NO: 118 - protein, 10 aa, Artificial Sequence
  Description of Artificial Sequence Synthetic peptide
FGGGTKLTVL

SEQ ID NO: 119 - protein, 121 aa, Artificial Sequence
  Description of Artificial Sequence Synthetic polypeptide
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 120 - protein, 30 aa, Artificial Sequence
  Description of Artificial Sequence Synthetic polypeptide
EVQLVESGGGLVQPGRSLRLSCAASGFTVS

SEQ ID NO: 121 - protein, 5 aa, Artificial Sequence
  Description of Artificial Sequence Synthetic peptide
SYWMH

SEQ ID NO: 122 - protein, 14 aa, Artificial Sequence
  Description of Artificial Sequence Synthetic peptide
WVRQAPGKGLEWVG

SEQ ID NO: 123 - protein, 19 aa, Artificial Sequence
  Description of Artificial Sequence Synthetic peptide
FIRNKANGGTTEYAASVKG

SEQ ID NO: 124 - protein, 32 aa, Artificial Sequence
  Description of Artificial Sequence Synthetic polypeptide
RFTISRDDSKNTLYLQMNSLRAEDTAVYYCAR

SEQ ID NO: 125 - protein, 10 aa, Artificial Sequence
  Description of Artificial Sequence Synthetic peptide
DRGLRFYFDY

SEQ ID NO: 126 - protein, 11 aa, Artificial Sequence
  Description of Artificial Sequence Synthetic peptide
WGQGTTVTVSS

SEQ ID NO: 127 - protein, 121 aa, Artificial Sequence
  Description of Artificial Sequence Synthetic polypeptide
EVQLVESGGGLVQPGRSLRLSCAASGFTVSSYWMHWVRQAPGKGLEWVGFILNKANGGTTEYAASVKGRFTISRDDSKNTLYLQMNSLRAEDTAVYYCARDRGLRFYFDYWGQGTTVTVSS

SEQ ID NO: 128 - protein, 30 aa, Artificial Sequence; identical to SEQ ID NO: 120
  Description of Artificial Sequence Synthetic polypeptide

SEQ ID NO: 129 - protein, 5 aa, Artificial Sequence
  Description of Artificial Sequence Synthetic peptide
SYWMH

SEQ ID NO: 130 - protein, 14 aa, Artificial Sequence
  Description of Artificial Sequence Synthetic peptide
WVRQAPGKGLEWVG

SEQ ID NO: 131 - protein, 19 aa, Artificial Sequence
  Description of Artificial Sequence Synthetic peptide
FILNKANGGTTEYAASVKG

SEQ ID NO: 132 - protein, 32 aa, Artificial Sequence; identical to SEQ ID NO: 124
  Description of Artificial Sequence Synthetic polypeptide

SEQ ID NO: 133 - protein, 10 aa, Artificial Sequence
  Description of Artificial Sequence Synthetic peptide
DRGLRFYFDY

SEQ ID NO: 134 - protein, 11 aa, Artificial Sequence
  Description of Artificial Sequence Synthetic peptide
WGQGTTVTVSS

(19) United States
(12) Patent Application Publication
(10) Pub. No.: US 2022/0347310 A1
(43) Pub. Date: Nov. 3, 2022

(54) AMIDE-LINKED, AMINOBENZAZEPINE IMMUNOCONJUGATES, AND USES THEREOF

(71) Applicant: BOLT BIOTHERAPEUTICS, INC., Redwood City, CA (US)

(72) Inventors: Romas KUDIRKA, Redwood City, CA (US); Brian SAFINA, Redwood City, CA (US); Matthew ZHOU, Redwood City, CA (US)

(73) Assignee: BOLT BIOTHERAPEUTICS, INC., Redwood City, CA (US)

(21) Appl. No.: 17/764,503

(22) PCT Filed: Sep. 29, 2020
(86) PCT No.: PCT/US2020/053224

§ 371 Date: Mar. 28, 2022

Related U.S. Application Data
(60) Provisional application No. 62/908,253, filed on Sep. 30, 2019.

(51) Int. Cl. A61K 47/68

(52) U.S. Cl. CPC A61K 47/6803; A61K 47/6889; A61K 47/6853; A61K 47/6851; A61K 47/6849

(57) ABSTRACT

The invention provides immunoconjugates of Formula I comprising an antibody linked by conjugation to one or more 8-amido-2-aminobenzazepine derivatives. The invention also provides 8-amido-2-aminobenzazepine derivative intermediate compositions comprising a reactive functional group. Such intermediate compositions are suitable substrates for formation of the immunoconjugates through a linker or linking moiety. The invention further provides methods of treating cancer with the immunoconjugates.

### CROSS REFERENCE TO RELATED APPLICATIONS

This non-provisional application claims the benefit of priority to U.S. Provisional Application No. 62/908,253, filed 30 Sep. 2019, which is incorporated by reference in its entirety.

### SEQUENCE LISTING

The instant application contains a Sequence Listing which has been submitted electronically in ASCII format and is hereby incorporated by reference in its entirety. Said ASCII copy, created on Sep. 21, 2020, is named 17019_004WO1_SL.txt and is 54,747 bytes in size.

### FIELD OF THE INVENTION

The invention relates generally to an immunoconjugate comprising an antibody conjugated to one or more 8-amido-2-aminobenzazepine molecules.

### BACKGROUND OF THE INVENTION

New compositions and methods for the delivery of antibodies and dendritic cell/myeloid cell adjuvants are needed in order to reach inaccessible tumors and/or to expand treatment options for cancer patients and other subjects. The invention provides such compositions and methods.

### SUMMARY OF THE INVENTION

The invention is generally directed to immunoconjugates comprising an antibody linked by conjugation to one or more 8-amido-2-aminobenzazepine derivatives. The invention is further directed to 8-amido-2-aminobenzazepine derivative intermediate compositions comprising a reactive functional group. Such intermediate compositions are suitable substrates for formation of immunoconjugates wherein an antibody may be covalently bound by a linker L to the 8-position of an 8-amido-2-aminobenzazepine moiety having the formula:

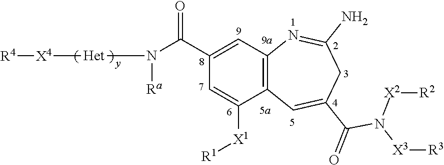

where one of R¹, R², R³ and R⁴ is attached to L, y is 0 or 1, and Het is a 5- or 6-membered monocyclic heterocyclyldiyl or a 5- or 6-membered monocyclic heteroaryldiyl. The positions of the 3H-benzo[b]azepine structure are numbered according to IUPAC conventions. The R^{a}, X¹⁻⁴ and R¹⁻⁴ substituents are defined herein.

The invention is further directed to use of such an immunoconjugates in the treatment of an illness, in particular cancer.

An aspect of the invention is an immunoconjugate comprising an antibody covalently attached to a linker which is covalently attached to one or more 8-amido-2-aminobenzazepine moieties.

Another aspect of the invention is an 8-amido-2-aminobenzazepine-linker compound.

Another aspect of the invention is a method for treating cancer comprising administering a therapeutically effective amount of an immunoconjugate comprising an antibody linked by conjugation to one or more 8-amido-2-aminobenzazepine moieties.

Another aspect of the invention is a use of an immunoconjugate comprising an antibody linked by conjugation to one or more 8-amido-2-aminobenzazepine moieties for treating cancer.

Another aspect of the invention is a method of preparing an immunoconjugate by conjugation of one or more 8-amido-2-aminobenzazepine moieties with an antibody.

### BRIEF DESCRIPTION OF THE DRAWINGS

FIG. 1A shows in vitro TLR8 potency of BZA-1 and BZA-2, agonists in Human HEK293 reporter cells. BZA-1: 2-amino-8-(3-((3-(hydroxymethyl)azetidin-1-yl)sulfonyl)phenyl)-N,N-dipropyl-3H-benzo[b]azepine-4-carboxamide. BZA-2: tert-butyl (3-(2-amino-8-(3-((3-(hydroxymethyl)azetidin-1-yl)sulfonyl)phenyl)-N-propyl-3H-benzo[b]azepine-4-carboxamido)propyl)carbamate.

FIG. 1B shows in vitro TLR7 potency of BZA-1 and BZA-2, agonists in Human HEK293 reporter cells.

FIG. 1C shows in vitro TLR8 potency of BZA-3 and BZA-4, agonists in Human HEK293 reporter cells. BZA-3: 2-amino-8-benzamido-N,N-dipropyl-3H-benzo[b]azepine-4-carboxamide. BZA-4: tert-butyl (3-(2-amino-8-benzamido-N-propyl-3H-benzo[b]azepine-4-carboxamido)propyl)carbamate.

FIG. 1D shows in vitro TLR7 potency of BZA-3 and BZA-4, agonists in Human HEK293 reporter cells.

FIG. 2 shows a computational docking image of BZA-2 docked, highlighting interactions with TLR8 Asp and TLR7 Leu residues.

FIG. 3A shows a computational docking solution image of BZA-2 to TLR8.

FIG. 3B shows a computational docking solution image of BZA-2 to TLR7, with the hydrophobic tert-butyl group of BZA-2 interacting with Leu 557 in TLR7.

FIG. 3C shows a computational docking solution image of BZA-4 to TLR8.

FIG. 3D shows a computational docking solution image of BZA-4 to TLR7, with the hydrophobic tert-butyl group of BZA-4 interacting with Leu 557 in TLR7.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the invention as defined by the claims.

One skilled in the art will recognize many methods and materials similar or equivalent to those described herein, which could be used in the practice of the present invention. The invention is in no way limited to the methods and materials described.

### Definitions

The term “immunoconjugate” refers to an antibody construct that is covalently bonded to an adjuvant moiety via a linker. the term “adjuvant” refers to a substance capable of eliciting an immune response in a subject exposed to the adjuvant. The phrase “adjuvant moiety” refers to an adjuvant that is covalently bonded to an antibody construct, e.g., through a linker, as described herein. The adjuvant moiety can elicit the immune response while bonded to the antibody construct or after cleavage (e.g., enzymatic cleavage) from the antibody construct following administration of an immunoconjugate to the subject.

“Adjuvant” refers to a substance capable of eliciting an immune response in a subject exposed to the adjuvant. The phrase “adjuvant moiety” refers to an adjuvant that is covalently bonded to an antibody construct, e.g., through a linker, as described herein. The adjuvant moiety can elicit the immune response while bonded to the antibody construct or after cleavage (e.g., enzymatic cleavage) from the antibody construct following administration of an immunoconjugate to the subject.

The terms “Toll-like receptor” and “TLR” refer to any member of a family of highly-conserved mammalian proteins which recognizes pathogen-associated molecular patterns and acts as key signaling elements in innate immunity. TLR polypeptides share a characteristic structure that includes an extracellular domain that has leucine-rich repeats, a transmembrane domain, and an intracellular domain that is involved in TLR signaling.

The terms “Toll-like receptor 7” and “TLR7” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ99026 for human TLR7 polypeptide, or GenBank accession number AAK62676 for murine TLR7 polypeptide.

The terms “Toll-like receptor 8” and “TLR8” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ95441 for human TLR8 polypeptide, or GenBank accession number AAK62677 for murine TLR8 polypeptide.

A “TLR agonist” is a substance that binds, directly or indirectly, to a TLR (e.g., TLR7 and/or TLR8) to induce TLR signaling. Any detectable difference in TLR signaling can indicate that an agonist stimulates or activates a TLR. Signaling differences can be manifested, for example, as changes in the expression of target genes, in the phosphorylation of signal transduction components, in the intracellular localization of downstream elements such as nuclear factor-κB (NF-κB), in the association of certain components (such as IL-1 receptor associated kinase (IRAK)) with other proteins or intracellular structures, or in the biochemical activity of components such as kinases (such as mitogen-activated protein kinase (MAPK)).

“Antibody” refers to a polypeptide comprising an antigen binding region (including the complementarity determining region (CDRs)) from an immunoglobulin gene or fragments thereof. The term “antibody” specifically encompasses monoclonal antibodies (including full length monoclonal antibodies), polyclonal antibodies, multispecific antibodies (e.g., bispecific antibodies), and antibody fragments that exhibit the desired biological activity. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (V_{L} and V_{H}, respectively) and constant domains or regions on the light and heavy chains (C_{L} and C_{H}, respectively). The N-terminus of each chain defines a variable region of about 100 to 110 or more amino acids, referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class γ heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding domain. There are four IgG subclasses (IgG1, IgG2, IgG3, and IgG4) in humans, named in order of their abundance in serum (i.e., IgG1 is the most abundant). Typically, the antigen binding domain of an antibody will be most critical in specificity and affinity of binding to cancer cells.

“Antibody construct” refers to an antibody or a fusion protein comprising (i) an antigen binding domain and (ii) an Fc domain.

In some embodiments, the binding agent is an antigen-binding antibody “fragment,” which is a construct that comprises at least an antigen-binding region of an antibody, alone or with other components that together constitute the antigen-binding construct. Many different types of antibody “fragments” are known in the art, including, for instance, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a single chain Fv (scFv), which is a monovalent molecule consisting of the two domains of the Fv fragment (i.e., V_{L} and V_{H}) joined by a synthetic linker which enables the two domains to be synthesized as a single polypeptide chain.

The antibody or antibody fragments can be part of a larger construct, for example, a conjugate or fusion construct of the antibody fragment to additional regions. For instance, in some embodiments, the antibody fragment can be fused to an Fc region as described herein. In other embodiments, the antibody fragment (e.g., a Fab or scFv) can be part of a chimeric antigen receptor or chimeric T-cell receptor, for instance, by fusing to a transmembrane domain (optionally with an intervening linker or “stalk” (e.g., hinge region)) and optional intercellular signaling domain. For instance, the antibody fragment can be fused to the gamma and/or delta chains of a t-cell receptor, so as to provide a T-cell receptor like construct that binds PD-L1. In yet another embodiment, the antibody fragment is part of a bispecific T-cell engager (BiTEs) comprising a CD1 or CD3 binding domain and linker.

“Epitope” means any antigenic determinant or epitopic determinant of an antigen to which an antigen binding domain binds (i.e., at the paratope of the antigen binding domain). Antigenic determinants usually consist of chemically active surface groupings of molecules, such as amino acids or sugar side chains, and usually have specific three dimensional structural characteristics, as well as specific charge characteristics.

The terms “Fc receptor” or “FcR” refer to a receptor that binds to the Fc region of an antibody. There are three main classes of Fc receptors: (1) FcγR which bind to IgG, (2) FcαR which binds to IgA, and (3) FcR which binds to IgE. The FcγR family includes several members, such as FcγI (CD64), FcγRIIA (CD32A), FcγRIIB (CD32B), FcγRIIIA (CD16A), and FcγRIIIB (CD16B). The Fcγ receptors differ in their affinity for IgG and also have different affinities for the IgG subclasses (e.g., IgG1, IgG2, IgG3, and IgG4).

“Biosimilar” refers to an approved antibody construct that has active properties similar to, for example, a PD-L1-targeting antibody construct previously approved such as atezolizumab (TECENTRIQ™, Genentech, Inc.), durvalumab (IMFINZI™, AstraZeneca), and avelumab (BAVENCIO™, EMD Serono, Pfizer); a HER2-targeting antibody construct previously approved such as trastuzumab (HERCEPTIN™, Genentech, Inc.), and pertuzumab (PERJETA™, Genentech, Inc.); or a CEA-targeting antibody such as labetuzumab (CEA-CIDE™, MN-14, hMN14, Immunomedics) CAS Reg. No. 219649-07-7).

“Biobetter” refers to an approved antibody construct that is an improvement of a previously approved antibody construct, such as atezolizumab, durvalumab, avelumab, trastuzumab, pertuzumab, and labetuzumab. The biobetter can have one or more modifications (e.g., an altered glycan profile, or a unique epitope) over the previously approved antibody construct.

“Amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid). The amino acids can be glycosylated (e.g., N-linked glycans, O-linked glycans, phosphoglycans, C-linked glycans, or glypication) or deglycosylated. Amino acids may be referred to herein by either the commonly known three letter symbols or by the one-letter symbols recommended by the IUPAC-IUB Biochemical Nomenclature Commission.

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Naturally-occurring amino acids include those formed in proteins by post-translational modification, such as citrulline (Cit).

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally-occurring amino acid.

“Linker” refers to a functional group that covalently bonds two or more moieties in a compound or material. For example, the linking moiety can serve to covalently bond an adjuvant moiety to an antibody construct in an immunoconjugate.

“Linking moiety” refers to a functional group that covalently bonds two or more moieties in a compound or material. For example, the linking moiety can serve to covalently bond an adjuvant moiety to an antibody in an immunoconjugate. Useful bonds for connecting linking moieties to proteins and other materials include, but are not limited to, amides, amines, esters, carbamates, ureas, thioethers, thiocarbamates, thiocarbonates, and thioureas.

“Divalent” refers to a chemical moiety that contains two points of attachment for linking two functional groups; polyvalent linking moieties can have additional points of attachment for linking further functional groups. Divalent radicals may be denoted with the suffix “diyl”. For example, divalent linking moieties include divalent polymer moieties such as divalent poly(ethylene glycol), divalent cycloalkyl, divalent heterocycloalkyl, divalent aryl, and divalent heteroaryl group. A “divalent cycloalkyl, heterocycloalkyl, aryl, or heteroaryl group” refers to a cycloalkyl, heterocycloalkyl, aryl, or heteroaryl group having two points of attachment for covalently linking two moieties in a molecule or material. Cycloalkyl, heterocycloalkyl, aryl, or heteroaryl groups can be substituted or unsubstituted. Cycloalkyl, heterocycloalkyl, aryl, or heteroaryl groups can be substituted with one or more groups selected from halo, hydroxy, amino, alkylamino, amido, acyl nitro cyano, and alkoxy.

A wavy line (“ [symbol] ”) represents a point of attachment of the specified chemical moiety. If the specified chemical moiety has two wavy lines (“ [symbol] ”) present, it will be understood that the chemical moiety can be used bilaterally, i.e., as read from left to right or from right to left. In some embodiments, a specified moiety having two wavy lines (“ [symbol] ”) present is considered to be used as read from left to right.

“Alkyl” refers to a straight (linear) or branched, saturated, aliphatic radical having the number of carbon atoms indicated. Alkyl can include any number of carbons, for example from one to twelve. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, and the like. Alkyl groups can be substituted or unsubstituted. “Substituted alkyl” groups can be substituted with one or more groups selected from halo, hydroxy, amino, oxo (═O), alkylamino, amido, acyl, nitro, cyano, and alkoxy.

The term “alkyldiyl” refers to a divalent alkyl radical. Examples of alkyldiyl groups include, but are not limited to, methylene (—CH₂—), ethylene (—CH₂CH₂—), propylene (—CH₂CH₂CH₂—), and the like. An alkyldiyl group may also be referred to as an “alkylene” group.

“Alkenyl” refers to a straight (linear) or branched, unsaturated, aliphatic radical having the number of carbon atoms indicated and at least one carbon-carbon double bond, sp2. Alkenyl can include from two to about 12 or more carbons atoms. Alkenyl groups are radicals having “cis” and “trans” orientations, or alternatively, “E” and “Z” orientations. Examples include, but are not limited to, ethylenyl or vinyl (—CH═CH₂), allyl (—CH₂CH═CH₂). butenyl, pentenyl, and isomers thereof. Alkenyl groups can be substituted or unsubstituted. “Substituted alkenyl” groups can be substituted with one or more groups selected from halo, hydroxy, amino, oxo (═O), alkylamino, amido, acyl, nitro, cyano, and alkoxy.

The terms “alkenylene” or “alkenyldiyl” refer to a linear or branched-chain divalent hydrocarbon radical. Examples include, but are not limited to, ethylenylene or vinylene (—CH═CH—), allyl (—CH₂CH═CH—), and the like.

“Alkynyl” refers to a straight (linear) or branched, unsaturated, aliphatic radical having the number of carbon atoms indicated and at least one carbon-carbon triple bond, sp. Alkynyl can include from two to about 12 or more carbons atoms. For example, C₂-C₆ alkynyl includes, but is not limited to ethynyl (—C≡CH), propynyl (propargyl, —CH₂C≡CH), butynyl, pentynyl, hexynyl, and isomers thereof. Alkynyl groups can be substituted or unsubstituted. “Substituted alkynyl” groups can be substituted with one or more groups selected from halo, hydroxy, amino, oxo (═O), alkylamino, amido, acyl, nitro, cyano, and alkoxy.

The term “alkynylene” or “alkynyldiyl” refer to a divalent alkynyl radical.

The terms “carbocycle”, “carbocyclyl”, “carbocyclic ring” and “cycloalkyl” refer to a saturated or partially unsaturated, monocyclic, fused bicyclic, or bridged polycyclic ring assembly containing from 3 to 12 ring atoms, or the number of atoms indicated. Saturated monocyclic carbocyclic rings include, for example, cyclopropyl, cyclobutyl, cyclopentyl, cyclohexyl, and cyclooctyl. Saturated bicyclic and polycyclic carbocyclic rings include, for example, norbornane, [2.2.2] bicyclooctane, decahydronaphthalene and adamantane. Carbocyclic groups can also be partially unsaturated, having one or more double or triple bonds in the ring. Representative carbocyclic groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene.

The term “cycloalkyldiyl” refers to a divalent cycloalkyl radical.

“Aryl” refers to a monovalent aromatic hydrocarbon radical of 6-20 carbon atoms (C₆-C₂₀) derived by the removal of one hydrogen atom from a single carbon atom of a parent aromatic ring system. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl.

The terms “arylene” or “aryldiyl” mean a divalent aromatic hydrocarbon radical of 6-20 carbon atoms (C₆-C₂₀) derived by the removal of two hydrogen atom from a two carbon atoms of a parent aromatic ring system. Some aryldiyl groups are represented in the exemplary structures as “Ar”. Aryldiyl includes bicyclic radicals comprising an aromatic ring fused to a saturated, partially unsaturated ring, or aromatic carbocyclic ring. Typical aryldiyl groups include, but are not limited to, radicals derived from benzene (phenyldiyl), substituted benzenes, naphthalene, anthracene, biphenylene, indenylene, indanylene, 1,2-dihydronaphthalene, 1,2,3,4-tetrahydronaphthyl, and the like. Aryldiyl groups are also referred to as “arylene”, and are optionally substituted with one or more substituents described herein.

The terms “heterocycle,” “heterocyclyl” and “heterocyclic ring” are used interchangeably herein and refer to a saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents described below. A heterocycle may be a monocycle having 3 to 7 ring members (2 to 6 carbon atoms and 1 to 4 heteroatoms selected from N, O, P, and S) or a bicycle having 7 to 10 ring members (4 to 9 carbon atoms and 1 to 6 heteroatoms selected from N, O, P, and S), for example: a bicyclo [4,5], [5,5], [5,6], or [6,6] system. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. “Heterocyclyl” also includes radicals where heterocycle radicals are fused with a saturated, partially unsaturated ring, or aromatic carbocyclic or heterocyclic ring. Examples of heterocyclic rings include, but are not limited to, morpholin-4-yl, piperidin-1-yl, piperazinyl, piperazin-4-yl-2-one, piperazin-4-yl-3-one, pyrrolidin-1-yl, thiomorpholin-4-yl, S-dioxothiomorpholin-4-yl, azocan-1-yl, azetidin-1-yl, octahydropyrido[1,2-a]pyrazin-2-yl, [1,4]diazepan-1-yl, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl quinolizinyl and N-pyridyl ureas. Spiro heterocyclyl moieties are also included within the scope of this definition. Examples of spiro heterocyclyl moieties include azaspiro[2.5]octanyl and azaspiro[2.4]heptanyl. Examples of a heterocyclic group wherein 2 ring atoms are substituted with oxo (═O) moieties are pyrimidinonyl and 1,1-dioxo-thiomorpholinyl. The heterocycle groups herein are optionally substituted independently with one or more substituents described herein.

The term “heterocyclyldiyl” refers to a divalent, saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents as described. Examples of 5-membered and 6-membered heterocyclyldiyls include morpholinyldiyl, piperidinyldiyl, piperazinyldiyl, pyrrolidinyldiyl, dioxanyldiyl, thiomorpholinyldiyl, and S-dioxothiomorpholinyldiyl.

The term “heteroaryl” refers to a monovalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituents described herein.

The term “heteroaryldiyl” refers to a divalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of 5-membered and 6-membered heteroaryldiyls include pyridyldiyl, imidazolyldiyl, pyrimidinyldiyl, pyrazolyldiyl, triazolyldiyl, pyrazinyldiyl, tetrazolyldiyl, furyldiyl, thienyldiyl, isoxazolyldiyldiyl, thiazolyldiyl, oxadiazolyldiyl, oxazolyldiyl, isothiazolyldiyl, and pyrrolyldiyl.

The heterocycle or heteroaryl groups may be carbon (carbon-linked), or nitrogen (nitrogen-linked) bonded where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

The terms “halo” and “halogen,” by themselves or as part of another substituent, refer to a fluorine, chlorine, bromine, or iodine atom.

The term “carbonyl,” by itself or as part of another substituent, refers to C(═O) or —C(═O)—, i.e., a carbon atom double-bonded to oxygen and bound to two other groups in the moiety having the carbonyl.

As used herein, the phrase “quaternary ammonium salt” refers to a tertiary amine that has been quaternized with an alkyl substituent (e.g., a C₁-C₄ alkyl such as methyl, ethyl, propyl, or butyl).

The terms “treat,” “treatment,” and “treating” refer to any indicia of success in the treatment or amelioration of an injury, pathology, condition (e.g., cancer), or symptom (e.g., cognitive impairment), including any objective or subjective parameter such as abatement; remission; diminishing of symptoms or making the symptom, injury, pathology, or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination.

The terms “cancer,” “neoplasm,” and “tumor” are used herein to refer to cells which exhibit autonomous, unregulated growth, such that the cells exhibit an aberrant growth phenotype characterized by a significant loss of control over cell proliferation. Cells of interest for detection, analysis, and/or treatment in the context of the invention include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer cell volume in a subject. The term “cancer cell” as used herein refers to any cell that is a cancer cell (e.g., from any of the cancers for which an individual can be treated, e.g., isolated from an individual having cancer) or is derived from a cancer cell, e.g., clone of a cancer cell. For example, a cancer cell can be from an established cancer cell line, can be a primary cell isolated from an individual with cancer, can be a progeny cell from a primary cell isolated from an individual with cancer, and the like. In some embodiments, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, and myelomas, and circulating cancers such as leukemias.

As used herein, the term “cancer” includes any form of cancer, including but not limited to, solid tumor cancers (e.g., skin, lung, prostate, breast, gastric, bladder, colon, ovarian, pancreas, kidney, liver, glioblastoma, medulloblastoma, leiomyosarcoma, head & neck squamous cell carcinomas, melanomas, and neuroendocrine) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors.

“PD-L1 expression” refers to a cell that has a PD-L1 receptor on the cell's surface. As used herein “PD-L1 overexpression” refers to a cell that has more PD-L1 receptors as compared to corresponding non-cancer cell.

“HER2” refers to the protein human epidermal growth factor receptor 2.

“HER2 expression” refers to a cell that has a HER2 receptor on the cell's surface. For example, a cell may have from about 20,000 to about 50,000 HER2 receptors on the cell's surface. As used herein “HER2 overexpression” refers to a cell that has more than about 50,000 HER2 receptors. For example, a cell 2, 5, 10, 100, 1,000, 10,000, 100,000, or 1,000,000 times the number of HER2 receptors as compared to corresponding non-cancer cell (e.g., about 1 or 2 million HER2 receptors). It is estimated that HER2 is overexpressed in about 25% to about 30% of breast cancers.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, and invasion of surrounding or distant tissues or organs, such as lymph nodes.

As used herein, the phrases “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs, therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part that is not directly connected to the organ of the original cancerous tumor. Metastasis can also be defined as several steps of a process, such as the departure of cancer cells from an original tumor site, and migration and/or invasion of cancer cells to other parts of the body.

The phrases “effective amount” and “therapeutically effective amount” refer to a dose or amount of a substance such as an immunoconjugate that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)). In the case of cancer, the therapeutically effective amount of the immunoconjugate may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the immunoconjugate may prevent growth and/or kill existing cancer cells, it may be cytostatic and/or cytotoxic. For cancer therapy, efficacy can, for example, be measured by assessing the time to disease progression (TTP) and/or determining the response rate (RR)

“Recipient,” “individual,” “subject,” “host,” and “patient” are used interchangeably and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In certain embodiments, the mammal is human.

The phrase “synergistic adjuvant” or “synergistic combination” in the context of this invention includes the combination of two immune modulators such as a receptor agonist, cytokine, and adjuvant polypeptide, that in combination elicit a synergistic effect on immunity relative to either administered alone. Particularly, the immunoconjugates disclosed herein comprise synergistic combinations of the claimed adjuvant and antibody construct. These synergistic combinations upon administration elicit a greater effect on immunity, e.g., relative to when the antibody construct or adjuvant is administered in the absence of the other moiety. Further, a decreased amount of the immunoconjugate may be administered (as measured by the total number of antibody constructs or the total number of adjuvants administered as part of the immunoconjugate) compared to when either the antibody construct or adjuvant is administered alone.

As used herein, the term “administering” refers to parenteral, intravenous, intraperitoneal, intramuscular, intratumoral, intralesional, intranasal, or subcutaneous administration, oral administration, administration as a suppository, topical contact, intrathecal administration, or the implantation of a slow-release device, e.g., a mini-osmotic pump, to the subject.

The terms “about” and “around,” as used herein to modify a numerical value, indicate a close range surrounding the numerical value. Thus, if “X” is the value, “about X” or “around X” indicates a value of from 0.9X to 1.1X, e.g., from 0.95X to 1.05X or from 0.99X to 1.01X. A reference to “about X” or “around X” specifically indicates at least the values X, 0.95X, 0.96X, 0.97X, 0.98X, 0.99X, 1.01X, 1.02X, 1.03X, 1.04X, and 1.05X. Accordingly, “about X” and “around X” are intended to teach and provide written description support for a claim limitation of, e.g., “0.98X.”

### Antibodies

The immunoconjugate of the invention comprises an antibody. Included in the scope of the embodiments of the invention are functional variants of the antibody constructs or antigen binding domain described herein. The term “functional variant” as used herein refers to an antibody construct having an antigen binding domain with substantial or significant sequence identity or similarity to a parent antibody construct or antigen binding domain, which functional variant retains the biological activity of the antibody construct or antigen binding domain of which it is a variant. Functional variants encompass, for example, those variants of the antibody constructs or antigen binding domain described herein (the parent antibody construct or antigen binding domain) that retain the ability to recognize target cells expressing PD-L1, HER2 or CEA to a similar extent, the same extent, or to a higher extent, as the parent antibody construct or antigen binding domain.

In reference to the antibody construct or antigen binding domain, the functional variant can, for instance, be at least about 30%, about 50%, about 75%, about 80%, about 85%, about 90%, about 91%, about 92%, about 93%, about 94%, about 95%, about 96%, about 97%, about 98%, about 99% or more identical in amino acid sequence to the antibody construct or antigen binding domain.

A functional variant can, for example, comprise the amino acid sequence of the parent antibody construct or antigen binding domain with at least one conservative amino acid substitution. Alternatively, or additionally, the functional variants can comprise the amino acid sequence of the parent antibody construct or antigen binding domain with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent antibody construct or antigen binding domain.

Amino acid substitutions of the inventive antibody constructs or antigen binding domains are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g., Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The antibody construct or antigen binding domain can consist essentially of the specified amino acid sequence or sequences described herein, such that other components, e.g., other amino acids, do not materially change the biological activity of the antibody construct or antigen binding domain functional variant.

In some embodiments, the antibodies in the immunoconjugates contain a modified Fc region, wherein the modification modulates the binding of the Fc region to one or more Fc receptors.

In some embodiments, the antibodies in the immunoconjugates (e.g., antibodies conjugated to at least two adjuvant moieties) contain one or more modifications (e.g., amino acid insertion, deletion, and/or substitution) in the Fc region that results in modulated binding (e.g., increased binding or decreased binding) to one or more Fc receptors (e.g., FcγRI (CD64), FcγRIIA (CD32A), FcγRIIB (CD32B), FcγRIIIA (CD16a), and/or FcγRIIIB (CD16b)) as compared to the native antibody lacking the mutation in the Fc region. In some embodiments, the antibodies in the immunoconjugates contain one or more modifications (e.g., amino acid insertion, deletion, and/or substitution) in the Fc region that reduce the binding of the Fc region of the antibody to FcγRIIB. In some embodiments, the antibodies in the immunoconjugates contain one or more modifications (e.g., amino acid insertion, deletion, and/or substitution) in the Fc region of the antibody that reduce the binding of the antibody to FcγRIIB while maintaining the same binding or having increased binding to FcγRI (CD64), FcγRIIA (CD32A), and/or FcRγIIIA (CD16a) as compared to the native antibody lacking the mutation in the Fc region. In some embodiments, the antibodies in the immunoconjugates contain one of more modifications in the Fc region that increase the binding of the Fc region of the antibody to FcγRIIB.

In some embodiments, the modulated binding is provided by mutations in the Fc region of the antibody relative to the native Fc region of the antibody. The mutations can be in a CH2 domain, a CH3 domain, or a combination thereof. A “native Fc region” is synonymous with a “wild-type Fc region” and comprises an amino acid sequence that is identical to the amino acid sequence of an Fc region found in nature or identical to the amino acid sequence of the Fc region found in the native antibody (e.g., cetuximab). Native sequence human Fc regions include a native sequence human IgG1 Fc region, native sequence human IgG2 Fc region, native sequence human IgG3 Fc region, and native sequence human IgG4 Fc region, as well as naturally occurring variants thereof. Native sequence Fe includes the various allotypes of Fes (Jefferis et al., (2009) mAbs, 1(4):332-338).

In some embodiments, the mutations in the Fc region that result in modulated binding to one or more Fc receptors can include one or more of the following mutations: SD (S239D), SDIE (S239D/I332E), SE (S267E), SELF (S267E/L328F), SDIE (S239D/I332E), SDIEAL (S239D/I332E/A330L), GA (G236A), ALIE (A330L/I332E), GASDALIE (G236A/S239D/A330L/I332E), V9 (G237D/P238D/P271G/A330R), and V11 (G237D/P238D/H268D/P271G/A330R), and/or one or more mutations at the following amino acids: E233, G237, P238, H268, P271, L328 and A330. Additional Fc region modifications for modulating Fc receptor binding are described in, for example, US 2016/0145350 and U.S. Pat. Nos. 7,416,726 and 5,624,821, which are hereby incorporated by reference in their entireties.

In some embodiments, the Fc region of the antibodies of the immunoconjugates are modified to have an altered glycosylation pattern of the Fc region compared to the native non-modified Fc region.

Human immunoglobulin is glycosylated at the Asn297 residue in the Cγ2 domain of each heavy chain. This N-linked oligosaccharide is composed of a core heptasaccharide, N-acetylglucosamine4Mannose3 (GlcNAc4Man3). Removal of the heptasaccharide with endoglycosidase or PNGase F is known to lead to conformational changes in the antibody Fc region, which can significantly reduce antibody-binding affinity to activating FcγR and lead to decreased effector function. The core heptasaccharide is often decorated with galactose, bisecting GlcNAc, fucose, or sialic acid, which differentially impacts Fc binding to activating and inhibitory FcγR. Additionally, it has been demonstrated that α2,6-sialyation enhances anti-inflammatory activity in vivo, while defucosylation leads to improved FcγRIIIa binding and a 10-fold increase in antibody-dependent cellular cytotoxicity and antibody-dependent phagocytosis. Specific glycosylation patterns, therefore, can be used to control inflammatory effector functions.

In some embodiments, the modification to alter the glycosylation pattern is a mutation. For example, a substitution at Asn297. In some embodiments, Asn297 is mutated to glutamine (N297Q). Methods for controlling immune response with antibodies that modulate FcγR-regulated signaling are described, for example, in U.S. Pat. No. 7,416,726 and U.S. Patent Application Publications 2007/0014795 and 2008/0286819, which are hereby incorporated by reference in their entireties.

In some embodiments, the antibodies of the immunoconjugates are modified to contain an engineered Fab region with a non-naturally occurring glycosylation pattern. For example, hybridomas can be genetically engineered to secrete afucosylated mAb, desialylated mAb or deglycosylated Fc with specific mutations that enable increased FcRγIIIa binding and effector function. In some embodiments, the antibodies of the immunoconjugates are engineered to be afucosylated.

In some embodiments, the entire Fc region of an antibody in the immunoconjugates is exchanged with a different Fc region, so that the Fab region of the antibody is conjugated to a non-native Fc region. For example, the Fab region of cetuximab, which normally comprises an IgG1 Fc region, can be conjugated to IgG2, IgG3, IgG4, or IgA, or the Fab region of nivolumab, which normally comprises an IgG4 Fc region, can be conjugated to IgG1, IgG2, IgG3, IgA1, or IgG2. In some embodiments, the Fc modified antibody with a non-native Fc domain also comprises one or more amino acid modification, such as the S228P mutation within the IgG4 Fc, that modulate the stability of the Fc domain described. In some embodiments, the Fc modified antibody with a non-native Fc domain also comprises one or more amino acid modifications described herein that modulate Fc binding to FcR.

In some embodiments, the modifications that modulate the binding of the Fc region to FcR do not alter the binding of the Fab region of the antibody to its antigen when compared to the native non-modified antibody. In other embodiments, the modifications that modulate the binding of the Fc region to FcR also increase the binding of the Fab region of the antibody to its antigen when compared to the native non-modified antibody.

In an exemplary embodiment, the immunoconjugates of the invention comprise an antibody construct that comprises an antigen binding domain that specifically recognizes and binds Programmed Death-Ligand 1 (PD-L1, cluster of differentiation 274, CD274, B7-homolog 1, or B7-H1) belongs to the B7 protein superfamily, and is a ligand of programmed cell death protein 1 (PD-1, PDCD1, cluster of differentiation 279, or CD279). PD-L1 can also interact with B7.1 (CD80) and such interaction is believed to inhibit T cell priming. The PD-L1/PD-1 axis plays a large role in suppressing the adaptive immune response. More specifically, it is believed that engagement of PD-L1 with its receptor, PD-1, delivers a signal that inhibits activation and proliferation of T-cells. Agents that bind to PD-L1 and prevent the ligand from binding to the PD-1 receptor prevent this immunosuppression, and can, therefore, enhance an immune response when desired, such as for the treatment of cancers, or infections. PD-L1/PD-1 pathway also contributes to preventing autoimmunity and therefore agonistic agents against PD-L1 or agents that deliver immune inhibitory payloads may help treatment of autoimmune disorders.

Several antibodies targeting PD-L1 have been developed for the treatment of cancer, including atezolizumab (TECENTRIQ™), durvalumab (IMFINZI™), and avelumab (BAVENCIO™). Nevertheless, there continues to be a need for new PD-L1-binding agents, including agents that bind PD-L1 with high affinity and effectively prevent PD-L1/PD-1 signaling and agents that can deliver therapeutic payloads to PD-L1 expressing cells. In addition, there is a need for new PD-L1-binding agents to treat autoimmune disorders and infections.

A method is provided of delivering an 8-amido-2-aminobenzazepine payload to a cell expressing PD-L1 comprising administering to the cell, or mammal comprising the cell, an immunoconjugate comprising an anti-PD-L1 antibody covalently attached to a linker which is covalently attached to one or more 8-amido-2-aminobenzazepine moieties.

Also provided is a method for enhancing or reducing or inhibiting an immune response in a mammal, and a method for treating a disease, disorder, or condition in a mammal that is responsive to PD-L1 inhibition, which methods comprise administering a PD-L1 immunoconjugate thereof, to the mammal.

The invention provides a PD-L1 binding agent comprising an immunoglobulin heavy chain variable region polypeptide and an immunoglobulin light chain variable region polypeptide.

The PD-L1 binding agent specifically binds PD-L1. The binding specificity of the agent allows for targeting PD-L1 expressing cells, for instance, to deliver therapeutic payloads to such cells.

In an exemplary embodiment, the immunoconjugates of the invention comprise an antibody construct that comprises an antigen binding domain that specifically recognizes and binds HER2. In one embodiment of the invention, an anti-HER2 antibody of an immunoconjugate of the invention comprises a humanized anti-HER2 antibody, e.g., huMAb4D5-1, huMAb4D5-2, huMAb4D5-3, huMAb4D5-4, huMAb4D5-5, huMAb4D5-6, huMAb4D5-7 and huMAb4D5-8, as described in Table 3 of U.S. Pat. No. 5,821,337, which is specifically incorporated by reference herein. Those antibodies contain human framework regions with the complementarity-determining regions of a murine antibody (4D5) that binds to HER2. The humanized antibody huMAb4D5-8 is also referred to as trastuzumab, commercially available under the tradename HERCEPTIN™ (Genentech, Inc.).

Trastuzumab (CAS 180288-69-1, HERCEPTIN®, huMAb4D5-8, rhuMAb HER2, Genentech) is a recombinant DNA-derived, IgG1 kappa, monoclonal antibody that is a humanized version of a murine anti-HER2 antibody (4D5) that selectively binds with high affinity in a cell-based assay (Kd=5 nM) to the extracellular domain of HER2 (U.S. Pat. Nos. 5,677,171; 5,821,337; 6,054,297; 6,165,464; 6,339,142; 6,407,213; 6,639,055; 6,719,971; 6,800,738; 7,074,404; Coussens et al (1985) Science 230:1132-9; Slamon et al (1989) Science 244:707-12; Slamon et al (2001) New Engl. J. Med. 344:783-792).

In an embodiment of the invention, the antibody construct or antigen binding domain comprises the CDR regions of trastuzumab. In an embodiment of the invention, the anti-HER2 antibody further comprises the framework regions of the trastuzumab. In an embodiment of the invention, the anti-HER2 antibody further comprises one or both variable regions of trastuzumab.

In another embodiment of the invention, an anti-HER2 antibody of an immunoconjugate of the invention comprises a humanized anti-HER2 antibody, e.g., humanized 2C4, as described in U.S. Pat. No. 7,862,817. An exemplary humanized 2C4 antibody is pertuzumab (CAS Reg. No. 380610-27-5), PERJETA™ (Genentech, Inc.). Pertuzumab is a HER dimerization inhibitor (HDI) and functions to inhibit the ability of HER2 to form active heterodimers or homodimers with other HER receptors (such as EGFR/HER1, HER2, HER3 and HER4). See, for example, Harari and Yarden, Oncogene 19:6102-14 (2000); Yarden and Sliwkowski. Nat Rev Mol Cell Biol 2:127-37 (2001); Sliwkowski Nat Struct Biol 10:158-9 (2003); Cho et al. Nature 421:756-60 (2003); and Malik et al. Pro Am Soc Cancer Res 44:176-7 (2003). PERJETA™ is approved for the treatment of breast cancer.

In an embodiment of the invention, the antibody construct or antigen binding domain comprises the CDR regions of pertuzumab. In an embodiment of the invention, the anti-HER2 antibody further comprises the framework regions of the pertuzumab. In an embodiment of the invention, the anti-HER2 antibody further comprises one or both variable regions of pertuzumab.

In an exemplary embodiment, the immunoconjugates of the invention comprise an antibody construct that comprises an antigen binding domain that specifically recognizes and binds Caprin-1 (Ellis J A, Luzio J P (1995) J Biol Chem. 270(35):20717-23; Wang B, et al (2005) J Immunol. 175 (7):4274-82; Solomon S, et al (2007) Mol Cell Biol. 27(6):2324-42). Caprin-1 is also known as GPIAP1, GPIP137, GRIP137, M11S1, RNG105, p137GPI, and cell cycle associated protein 1.

Cytoplasmic activation/proliferation-associated protein-1 (caprin-1) is an RNA-binding protein that participates in the regulation of cell cycle control-associated genes. Caprin-1 selectively binds to c-Myc and cyclin D2 mRNAs, which accelerates cell progression through the G₁ phase into the S phase, enhances cell viability and promotes cell growth, indicating that it may serve an important role in tumorigenesis (Wang B, et al (2005) J Immunol. 175:4274-4282). Caprin-1 acts alone or in combination with other RNA-binding proteins, such as RasGAP SH3-domain-binding protein 1 and fragile X mental retardation protein. In the tumorigenesis process, caprin-1 primarily functions by activating cell proliferation and upregulating the expression of immune checkpoint proteins. Through the formation of stress granules, caprin-1 is also involved in the process by which tumor cells adapt to adverse conditions, which contributes to radiation and chemotherapy resistance. Given its role in various clinical malignancies, caprin-1 holds the potential to be used as a biomarker and a target for the development of novel therapeutics (Yang, Z-S, et al (2019) Oncology Letters 18:15-21).

Antibodies that target caprin-1 for treatment and detection have been described (WO 2011/096519; WO 2013/125654; WO 2013/125636; WO 2013/125640; WO 2013/125630; WO 2013/018889; WO 2013/018891; WO 2013/018883; WO 2013/018892; WO 2014/014082; WO 2014/014086; WO 2015/020212; WO 2018/079740).

In an exemplary embodiment, the immunoconjugates of the invention comprise an antibody construct that comprises an antigen binding domain that specifically recognizes and binds CEA.

Elevated expression of carcinoembryonic antigen (CEA, CD66e, CEACAM5) has been implicated in various biological aspects of neoplasia, especially tumor cell adhesion, metastasis, the blocking of cellular immune mechanisms, and having antiapoptosis functions. CEA is also used as a blood marker for many carcinomas. Labetuzumab (CEA-CIDE™, Immunomedics, CAS Reg. No. 219649-07-7), also known as MN-14 and hMN14, is a humanized IgG1 monoclonal antibody and has been studied for the treatment of colorectal cancer (Blumenthal, R. et al (2005) Cancer Immunology Immunotherapy 54(4):315-327). Labetuzumab conjugated to a camptothecin analog (labetuzumab govitecan, IMMU-130) targets carcinoembryonic antigen-related cell adhesion mol. 5 (CEACAM5) and is being studied in patients with relapsed or refractory metastatic colorectal cancer (Sharkey, R. et al, (2018), Molecular Cancer Therapeutics 17(1):196-203; Cardillo, T. et al (2018) Molecular Cancer Therapeutics 17(1):150-160).

In an embodiment of the invention, the CEA-targeting antibody construct or antigen binding domain comprises the Variable light chain (VL kappa) of hMN-14/labetuzumab SEQ ID NO. 1 (U.S. Pat. No. 6,676,924).

SEQ ID NO. 1
DIQLTQSPSSLSASVGDRVTITCKASQDVGTSVAWYQQKPGKAPKLLIYW
TSTRHTGVPSRFSGSGSGTDFTFTISSLQPEDIATYYCQQYSLYRSFGQG
TKVEIK

In an embodiment of the invention, the CEA-targeting antibody construct or antigen binding domain comprises the light chain CDR (complementarity determining region) or light chain framework (LFR) sequences of hMN-14/labetuzumab SEQ ID NO. 2-8 (U.S. Pat. No. 6,676,924).

| Region | Sequence Fragment | Residues | Length | SEQ ID NO. |
|---|---|---|---|---|
| LFR1 | DIQLTQSPSSLSASVGDRVTITC | 1-23 | 23 | 2 |
| CDR-L1 | KASQDVGTSVA | 24-34 | 11 | 3 |
| LFR2 | WYQQKPGKAPKLLIY | 35-49 | 15 | 4 |
| CDR-L2 | WTSTRHT | 50-56 | 7 | 5 |
| LFR3 | GVPSRFSGSGSGTDFTFTISSLQPEDIATYYC | 57-88 | 32 | 6 |
| CDR-L3 | QQYSLYRS | 89-96 | 8 | 7 |
| LFR4 | FGQGTKVEIK | 97-106 | 10 | 8 |

In an embodiment of the invention, the CEA-targeting antibody construct or antigen binding domain comprises the Variable heavy chain (V) of hMN-14/labetuzumab SEQ ID NO. 9 (U.S. Pat. No. 6,676,924).

SEQ ID NO. 9
EVQLVESGGGVVQPGRSLRLSCSSSGFDFTTYWMSWVRQAPGKGLEWVAE
IHPDSSTINYAPSLKDRFTISRDNSKNTLFLQMDSLRPEDTGVYFCASLY
FGFPWFAYWGQGTPVTVSS

In an embodiment of the invention, the CEA-targeting antibody construct or antigen binding domain comprises the heavy chain CDR (complementarity determining region) or heavy chain framework (HFR) sequences of hMN-14/labetuzumab SEQ ID NO. 10-16 (U.S. Pat. No. 6,676,924).

| Region | Sequence Fragment | Residues | Length | SEQ ID NO. |
|---|---|---|---|---|
| HFR1 | EVQLVESGGGVVQPGRSLRLSCSSSGFDFT | 1-30 | 30 | 10 |
| CDR-H1 | TYWMS | 31-35 | 5 | 11 |
| HFR2 | WVRQAPGKGLEWVA | 36-49 | 14 | 12 |
| CDR-H2 | EIHPDSSTINYAPSLKD | 50-66 | 17 | 13 |
| HFR3 | RFTISRDNSKNTLFLQMDSLRPEDTGVYFCAS | 67-98 | 32 | 14 |
| CDR-H3 | LYFGFPWFAY | 99-108 | 10 | 15 |
| HFR4 | WGQGTPVTVSS | 109-119 | 11 | 16 |

In an embodiment of the invention, the CEA-targeting antibody construct or antigen binding domain comprises the Variable light chain (VL kappa) of hPR1A3 SEQ ID NO. 17 (U.S. Pat. No. 8,642,742).

SEQ ID NO. 17
DIQMTQSPSSLSASVGDRVTITCKASAAVGTYVAWYQQKPGKAPKLLIYS
ASYRKRGVPSRFSGSGSGTDFTLTISSLQPEDFATYYCHQYYTYPLFTFG
QGTKLEIK

In an embodiment of the invention, the CEA-targeting antibody construct or antigen binding domain comprises the light chain CDR (complementarity determining region) or light chain framework (LFR) sequences of hPR1A3 SEQ ID NO. 18-24 (U.S. Pat. No. 8,642,742).

| Region | Sequence Fragment | Residues | Length | SEQ ID NO. |
|---|---|---|---|---|
| LFR1 | DIQMTQSPSSLSASVGDRVTITC | 1-23 | 23 | 18 |
| CDR-L1 | KASAAVGTYVA | 24-34 | 11 | 19 |
| LFR2 | WYQQKPGKAPKLLIY | 35-49 | 15 | 20 |
| CDR-L2 | SASYRKR | 50-56 | 7 | 21 |
| LFR3 | GVPSRFSGSGSGTDFTLTISSLQPEDFATYYC | 57-88 | 32 | 22 |
| CDR-L3 | HQYYTYPLFT | 89-98 | 10 | 23 |
| LFR4 | FGQGTKLEIK | 99-108 | 10 | 24 |

In an embodiment of the invention, the CEA-targeting antibody construct or antigen binding domain comprises the heavy chain CDR (complementarity determining region) or heavy chain framework (HFR) sequences of hPR1A3 SEQ ID NO. 25-31 (U.S. Pat. No. 8,642,742).

| Region | Sequence Fragment | Residues | Length | SEQ ID NO. |
|---|---|---|---|---|
| HFR1 | QVQLVQSGAEVKKPGASVKVSCKASGYTFT | 1-30 | 30 | 25 |
| CDR-H1 | EFGMN | 31-35 | 5 | 26 |
| HFR2 | WVRQAPGQGLEWMG | 36-49 | 14 | 27 |
| CDR-H2 | WINTKTGEATYVEEFKG | 50-66 | 17 | 28 |
| HFR3 | RVTFTTDTSTSTAYMELRSLRSDDTAVYYCAR | 67-98 | 32 | 29 |
| CDR-H3 | WDFAYYVEAMDY | 99-110 | 12 | 30 |
| HFR4 | WGQGTTVTVSS | 111-121 | 11 | 31 |

In an embodiment of the invention, the CEA-targeting antibody construct or antigen binding domain comprises the Variable light chain (VL kappa) of hMFE-23 SEQ ID NO. 32 (U.S. Pat. No. 723,288).

SEQ ID NO. 32
ENVLTQSPSSMSASVGDRVNIACSASSSVSYMHWFQQKPGKSPKLWIYST
SNLASGVPSRFSGSGSGTDYSLTISSMQPEDAATYYCQQRSSYPLTFGGG
TKLEIK

In an embodiment of the invention, the CEA-targeting antibody construct or antigen binding domain comprises the light chain CDR (complementarity determining region) or light chain framework (LFR) sequences of hMFE-23 SEQ ID NO. 33-39 (U.S. Pat. No. 723,288).

| Region | Sequence Fragment | Residues | Length | SEQ ID NO. |
|---|---|---|---|---|
| LFR1 | ENVLTQSPSSMSASVGDRVNIAC | 1-23 | 23 | 33 |
| CDR-L1 | SASSSVSYMH | 24-33 | 10 | 34 |
| LFR2 | WFQQKPGKSPKLWIY | 34-48 | 15 | 35 |
| CDR-L2 | STSNLAS | 49-55 | 7 | 36 |
| LFR3 | GVPSRFSGSGSGTDYSLTISSMQPEDAATYYC | 56-87 | 32 | 37 |
| CDR-L3 | QQRSSYPLT | 88-96 | 9 | 38 |
| LFR4 | FGGGTKLEIK | 97-106 | 10 | 39 |

In an embodiment of the invention, the CEA-targeting antibody construct or antigen binding domain comprises the Variable heavy chain (VH) of hMFE-23 SEQ ID NO. 40 (U.S. Pat. No 723,288).

SEQ ID NO. 40
QVKLEQSGAEVVKPGASVKLSCKASGFNIKDSYMHWLRQGPGQRLEWIG
WIDPENGDTEYAPKFQGKATFTTDTSANTAYLGLSSLRPEDTAVYYCNE
GTPTGPYYFDYWGQGTLVTVSS

In an embodiment of the invention, the CEA-targeting antibody construct or antigen binding domain comprises the heavy chain CDR (complementarity determining region) or heavy chain framework (HFR) sequences of hMFE-23 SEQ ID NO. 41-47 (U.S. Pat. No. 723,288).

| Region | Sequence Fragment | Residues | Length | SEQ ID NO. |
|---|---|---|---|---|
| HFR1 | QVKLEQSGAEVVKPGASVKLSCKASGFNIK | 1-30 | 30 | 41 |
| CDR-H1 | DSYMH | 31-35 | 5 | 42 |
| HFR2 | WLRQGPGQRLEWIG | 36-49 | 14 | 43 |
| CDR-H2 | WIDPENGDTEYAPKFQG | 50-66 | 17 | 44 |
| HFR3 | KATFTTDTSANTAYLGLSSLRPEDTAVYYCNE | 67-98 | 32 | 45 |
| CDR-H3 | GTPTGPYYFDY | 99-109 | 11 | 46 |
| HFR4 | WGQGTLVTVSS | 110-120 | 11 | 47 |

In an embodiment of the invention, the CEA-targeting antibody construct or antigen binding domain comprises the Variable light chain (VL kappa) of SM3E SEQ ID NO. 48 (U.S. Pat. No. 723,288).

SEQ ID NO. 48
ENVLTQSPSSMSVSVGDRVTIACSASSSVPYMHWLQQKPGKSPKLLIYL
TSNLASGVPSRFSGSGSGTDYSLTISSVQPEDAATYYCQQRSSYPLTFG
GGTKLEIK

In an embodiment of the invention, the CEA-targeting antibody construct or antigen binding domain comprises the light chain CDR (complementarity determining region) or light chain framework (LFR) sequences of SM3E SEQ ID NO. 49-55 (U.S. Pat. No. 723,288).

| Region | Sequence Fragment | Residues | Length | SEQ ID NO. |
|---|---|---|---|---|
| LFR1 | ENVLTQSPSSMSVSVGDRVTIAC | 1-23 | 23 | 49 |
| CDR-L1 | SASSSVPYMH | 24-33 | 10 | 50 |
| LFR2 | WLQQKPGKSPKLLIY | 34-48 | 15 | 51 |
| CDR-L2 | LTSNLAS | 49-55 | 7 | 52 |
| LFR3 | GVPSRFSGSGSGTDYSLTISSVQPEDAATYYC | 56-87 | 32 | 53 |
| CDR-L3 | QQRSSYPLT | 88-96 | 9 | 54 |
| LFR4 | FGGGTKLEIK | 97-106 | 10 | 55 |

In an embodiment of the invention, the CEA-targeting antibody construct or antigen binding domain comprises the Variable heavy chain (VH) of SM3E SEQ ID NO. 56 (U.S. Pat. No. 723,288).

SEQ ID NO. 56
QVKLEQSGAEVVKPGASVKLSCKASGFNIKDSYMHWLRQGPGQRLEWIG
WIDPENGDTEYAPKFQGKATFTTDTSANTAYLGLSSLRPEDTAVYYCNE
GTPTGPYYFDYWGQGTLVTVSS

In an embodiment of the invention, the CEA-targeting antibody construct or antigen binding domain comprises the heavy chain CDR (complementarity determining region) or heavy chain framework (HFR) sequences of SM3E SEQ ID NO. 57-63 (U.S. Pat. No. 723,288).

| Region | Sequence Fragment | Residues | Length | SEQ ID NO. |
|---|---|---|---|---|
| HFR1 | QVKLEQSGAEVVKPGASVKLSCKASGFNIK | 1-30 | 30 | 57 |
| CDR-H1 | DSYMH | 31-35 | 5 | 58 |
| HFR2 | WLRQGPGQRLEWIG | 36-49 | 14 | 59 |
| CDR-H2 | WIDPENGDTEYAPKFQG | 50-66 | 17 | 60 |
| HFR3 | KATFTTDTSANTAYLGLSSLRPEDTAVYYCNE | 67-98 | 32 | 61 |
| CDR-H3 | GTPTGPYYFDY | 99-109 | 11 | 62 |
| HFR4 | WGQGTLVTVSS | 110-120 | 11 | 63 |

In an embodiment of the invention, the CEA-targeting antibody construct or antigen binding domain comprises the light chain CDR (complementarity determining region) or light chain framework (LFR) sequences of NP-4/arcitumomab SEQ ID NO. 64-70.

| Region | Sequence Fragment | Residues | Length | SEQ ID NO. |
|---|---|---|---|---|
| LFR1 | QTVLSQSPAILSASPGEKVTMTC | 1-23 | 23 | 64 |
| CDR-L1 | RASSSVTYIH | 24-33 | 10 | 65 |
| LFR2 | WYQQKPGSSPKSWIY | 34-48 | 15 | 66 |
| CDR-L2 | ATSNLAS | 49-55 | 7 | 67 |
| LFR3 | GVPARFSGSGSGTSYSLTISRVEAEDAATYYC | 56-87 | 32 | 68 |
| CDR-L3 | QHWSSKPPT | 88-96 | 9 | 69 |
| LFR4 | FGGGTKLEIK | 97-106 | 10 | 70 |

In an embodiment of the invention, the CEA-targeting antibody construct or antigen binding domain comprises the Variable heavy chain (VH) of NP-4/arcitumomab SEQ ID NO. 71.

SEQ ID NO. 71
EVKLVESGGGLVQPGGSLRLSCATSGFTFTDYYMNWVRQPPGKALEWLGF
IGNKANGYTTEYSASVKGRFTISRDKSQSILYLQMNTLRAEDSATYYCTR
DRGLRFYFDYWGQGTTLTVSS.

In an embodiment of the invention, the CEA-targeting antibody construct or antigen binding domain comprises the heavy chain CDR (complementarity determining region) or heavy chain framework (HFR) sequences of NP-4 SEQ ID NO. 72-78.

| Region | Sequence Fragment | Residues | Length | SEQ ID NO. |
|---|---|---|---|---|
| HFR1 | EVKLVESGGGLVQPGGSLRLSCATSGFTFT | 1-30 | 30 | 72 |
| CDR-H1 | DYYMN | 31-35 | 5 | 73 |
| HFR2 | WVRQPPGKALEWLG | 36-49 | 14 | 74 |
| CDR-H2 | FIGNKANGYTTEYSASVKG | 50-68 | 19 | 75 |
| HFR3 | RFTISRDKSQSILYLQMNTLRAEDSATYYCTR | 69-100 | 32 | 76 |
| CDR-H3 | DRGLRFYFDY | 101-110 | 10 | 77 |
| HFR4 | WGQGTTLTVSS | 111-121 | 11 | 78 |

In an embodiment of the invention, the CEA-targeting antibody construct or antigen binding domain comprises the Variable light chain (VL kappa) of M5A/hT84.66 SEQ ID NO. 79 (U.S. Pat. No. 7,776,330).

SEQ ID NO. 79
DIQLTQSPSSLSASVGDRVTITCRAGESVDIFGVGFLHWYQQKPGKAPKLL
IYRASNLESGVPSRFSGSGSRTDFTLTISSLQPEDFATYYCQQTNEDPYTF
GQGTKVEIK

In an embodiment of the invention, the CEA-targeting antibody construct or antigen binding domain comprises the light chain CDR (complementarity determining region) or light chain framework (LFR) sequences of M5A/hT84.66 SEQ ID NO. 80-86 (U.S. Pat. No. 7,776,330).

| Region | Sequence Fragment | Residues | Length | SEQ ID NO. |
|---|---|---|---|---|
| LFR1 | DIQLTQSPSSLSASVGDRVTITC | 1-23 | 23 | 80 |
| CDR-L1 | RAGESVDIFGVGFLH | 24-38 | 15 | 81 |
| LFR2 | WYQQKPGKAPKLLIY | 39-53 | 15 | 82 |
| CDR-L2 | RASNLES | 54-60 | 7 | 83 |
| LFR3 | GVPSRFSGSGSRTDFTLTISSLQPEDFATYYC | 61-92 | 32 | 84 |
| CDR-L3 | QQTNEDPYT | 93-101 | 9 | 85 |
| LFR4 | FGQGTKVEIK | 102-111 | 10 | 86 |

In an embodiment of the invention, the CEA-targeting antibody construct or antigen binding domain comprises the Variable heavy chain (VH) of M5A/hT84.66 SEQ ID NO. 87 (U.S. Pat. No. 7,776,330).

SEQ ID NO. 87
EVQLVESGGGLVQPGGSLRLSCAASGFNIKDTYMHWVRQAPGKGLEWVARI
DPANGNSKYADSVKGRFTISADTSKNTAYLQMNSLRAEDTAVYYCAPFGYY
VSDYAMAYWGQGTLVTVSS

In an embodiment of the invention, the CEA-targeting antibody construct or antigen binding domain comprises the heavy chain CDR (complementarity determining region) or heavy chain framework (HFR) sequences of M5A/hT84.66 SEQ ID NO. 88-94 (U.S. Pat. No. 7,776,330).

| Region | Sequence Fragment | Residues | Length | SEQ ID NO. |
|---|---|---|---|---|
| HFR1 | EVQLVESGGGLVQPGGSLRLSCAASGFNIK | 1-30 | 30 | 88 |
| CDR-H1 | DTYMH | 31-35 | 5 | 89 |
| HFR2 | WVRQAPGKGLEWVA | 36-49 | 14 | 90 |
| CDR-H2 | RIDPANGNSKYADSVKG | 50-66 | 17 | 91 |
| HFR3 | RFTISADTSKNTAYLQMNSLRAEDTAVYYCAP | 67-98 | 32 | 92 |
| CDR-H3 | FGYYVSDYAMAY | 99-110 | 12 | 93 |
| HFR4 | WGQGTLVTVSS | 111-121 | 11 | 94 |

In an embodiment of the invention, the CEA-targeting antibody construct or antigen binding domain comprises the Variable light chain (VL kappa) of hAb2-3 SEQ ID NO. 95 (U.S. Pat. No. 9,617,345).

SEQ ID NO. 95
DIQMTQSPASLSASVGDRVTITCRASENIFSYLAWYQQKPGKSPKLLVYNT
RTLAEGVPSRFSGSGSGTDFSLTISSLQPEDFATYYCQHHYGTPFTFGSGT
KLEIK

In an embodiment of the invention, the CEA-targeting antibody construct or antigen binding domain comprises the light chain CDR (complementarity determining region) or light chain framework (LFR) sequences of hAb2-3 SEQ ID NO. 96-102 (U.S. Pat. No. 9,617,345).

| Region | Sequence Fragment | Residues | Length | SEQ ID NO. |
|---|---|---|---|---|
| LFR1 | DIQMTQSPASLSASVGDRVTITC | 1-23 | 23 | 96 |
| CDR-L1 | RASENIFSYLA | 24-34 | 11 | 97 |
| LFR2 | WYQQKPGKSPKLLVY | 35-49 | 15 | 98 |
| CDR-L2 | NTRTLAE | 50-56 | 7 | 99 |
| LFR3 | GVPSRFSGSGSGTDFSLTISSLQPEDFATYYC | 57-88 | 32 | 100 |
| CDR-L3 | QHHYGTPFT | 89-97 | 9 | 101 |
| LFR4 | FGSGTKLEIK | 98-107 | 10 | 102 |

In an embodiment of the invention, the CEA-targeting antibody construct or antigen binding domain comprises the Variable heavy chain (VH) of SEQ ID NO. 103 (U.S. Pat. No. 9,617,345).

SEQ ID NO. 103
EVQLQESGPGLVKPGGSLSLSCAASGFVFSSYDMSWVRQTPERGLEWVAYI
SSGGGITYAPSTVKGRFTVSRDNAKNTLYLQMNSLTSEDTAVYYCAAHYFG
SSGPFAYWGQGTLVTVSS

In an embodiment of the invention, the CEA-targeting antibody construct or antigen binding domain comprises the heavy chain CDR (complementarity determining region) or heavy chain framework HER sequences of hAb2-3 SE TD NO. 104-110.

| Region | Sequence Fragment | Residues | Length | SEQ ID NO. |
|---|---|---|---|---|
| HFR1 | EVQLQESGPGLVKPGGSLSLSCAASGFVFS | 1-30 | 30 | 104 |
| CDR-H1 | SYDMS | 31-35 | 5 | 105 |
| HFR2 | WVRQTPERGLEWVA | 36-49 | 14 | 106 |
| CDR-H2 | YISSGGGITYAPSTVKG | 50-66 | 17 | 107 |
| HFR3 | RFTVSRDNAKNTLYLQMNSLTSEDTAVYYCAA | 67-98 | 32 | 108 |
| CDR-H3 | HYFGSSGPFAY | 99-109 | 11 | 109 |
| HFR4 | WGQGTLVTVSS | 110-120 | 11 | 110 |

In an embodiment of the invention, the CEA-targeting antibody construct or antigen binding domain comprises the Variable light chain (VL kappa) of A240VL-B9VH/AMVG-211 SEQ ID NO. 111 (U.S. Pat. No. 9,982,063).

SEQ ID NO. 111
QAVLTQPASLSASPGASASLTCTLRRGINVGAYSIYWYQQKPGSPPQYLLR
YKSDSDKQQGSGVSSRFSASKDASANAGILLISGLQSEDEADYYCMIWHSG
ASAVFGGGTKLTVL

In an embodiment of the invention, the CEA-targeting antibody construct or antigen binding domain comprises the light chain CDR (complementarity determining region) or light chain framework (LFR) sequences of A240VL-B9V7/AMG-211 SEQ ID NO. 112-118 (U.S. Pat. No. 9,982,063)

| Region | Sequence Fragment | Residues | Length | SEQ ID NO. |
|---|---|---|---|---|
| LFR1 | QAVLTQPASLSASPGASASLTC | 1-22 | 22 | 112 |
| CDR-L1 | TLRRGINVGAYSIY | 23-36 | 14 | 113 |
| LFR2 | WYQQKPGSPPQYLLR | 37-51 | 15 | 114 |
| CDR-L2 | YKSDSDKQQGS | 52-62 | 11 | 115 |
| LFR3 | GVSSRFSASKDASANAGILLISGLQSEDEADYYC | 63-96 | 34 | 116 |
| CDR-L3 | MIWHSGASAV | 97-106 | 10 | 117 |
| LFR4 | FGGGTKLTVL | 107-116 | 10 | 118 |

In an embodiment of the invention, the CEA-targeting antibody construct or antigen binding domain comprises the Variable heavy chain (VH) of B9VH SEQ ID NO. 119 (U.S. Pat. No. 9,982,063).

SEQ ID NO. 119
EVQLVESGGGLVQPGRSLRLSCAASGFTVSSYWMHWVRQAPGKGLEWVGFI
RNKANGGTTEYAASVKGRFTISRDDSKNTLYLQMNSLRAEDTAVYYCARDR
GLRFYFDYWGQGTTVTVSS

In an embodiment of the invention, the CEA-targeting antibody construct or antigen binding domain comprises the heavy chain CDR (complementarity determining region) or heavy chain framework (HFR) sequences of SEQ ID NO. 120-126 (U.S. Pat. No. 9,982,063).

| Region | Sequence Fragment | Residues | Length | SEQ ID NO. |
|---|---|---|---|---|
| HFR1 | EVQLVESGGGLVQPGRSLRLSCAASGFTVS | 1-30 | 30 | 120 |
| CDR-H1 | SYWMH | 31-35 | 5 | 121 |
| HFR2 | WVRQAPGKGLEWVG | 36-49 | 14 | 122 |
| CDR-H2 | FIRNKANGGTTEYAASVKG | 50-68 | 19 | 123 |
| HFR3 | RFTISRDDSKNTLYLQMNSLRAEDTAVYYCAR | 69-100 | 32 | 124 |
| CDR-H3 | DRGLRFYFDY | 101-110 | 10 | 125 |
| HFR4 | WGQGTTVTVSS | 111-121 | 11 | 126 |

In an embodiment of the invention, the CEA-targeting antibody construct or antigen binding domain comprises the Variable heavy chain (VH) of E12VH SEQ ID NO. 127 (U.S. Pat. No. 9,982,063).

SEQ ID NO. 127
EVQLVESGGGLVQPGRSLRLSCAASGFTVSSYWMHWVRQAPGKGLEWVGFI
LNKANGGTTEYAASVKGRFTISRDDSKNTLYLQMNSLRAEDTAVYYCARDR
GLRFYFDYWGQGTTVTVSS

In an embodiment of the invention, the CEA-targeting antibody construct or antigen binding domain comprises the heavy chain CDR (complementarity determining region) or heavy chain framework (HFR) sequences of SEQ ID NO. 128-134 (U.S. Pat. No. 9,982,063).

| Region | Sequence Fragment | Residues | Length | SEQ ID NO. |
|---|---|---|---|---|
| HFR1 | EVQLVESGGGLVQPGRSLRLSCAASGFTVS | 1-30 | 30 | 128 |
| CDR-H1 | SYWMH | 31-35 | 5 | 129 |
| HFR2 | WVRQAPGKGLEWVG | 36-49 | 14 | 130 |
| CDR-H2 | FILNKANGGTTEYAASVKG | 50-68 | 19 | 131 |
| HFR3 | RFTISRDDSKNTLYLQMNSLRAEDTAVYYCAR | 69-100 | 32 | 132 |
| CDR-H3 | DRGLRFYFDY | 101-110 | 10 | 133 |
| HFR4 | WGQGTTVTVSS | 111-121 | 11 | 134 |

In some embodiments, the antibody construct further comprises an Fc domain. In certain embodiments, the antibody construct is an antibody. In certain embodiments, the antibody construct is a fusion protein. The antigen binding domain can be a single-chain variable region fragment (scFv). A single-chain variable region fragment (scFv), which is a truncated Fab fragment including the variable (V) domain of an antibody heavy chain linked to a V domain of a light antibody chain via a synthetic peptide, can be generated using routine recombinant DNA technology techniques. Similarly, disulfide-stabilized variable region fragments (dsFv) can be prepared by recombinant DNA technology. The antibody construct or antigen binding domain may comprise one or more variable regions (e.g., two variable regions) of an antigen binding domain of an anti-PD-L1 antibody, an anti-HER2 antibody, or an anti-CEA antibody, each variable region comprising a CDR1, a CDR2, and a CDR3.

In some embodiments, the antibodies in the immunoconjugates contain a modified Fc region, wherein the modification modulates the binding of the Fc region to one or more Fc receptors.

In some embodiments, the Fc region is modified by inclusion of a transforming growth factor beta 1 (TGFβ1) receptor, or a fragment thereof, that is capable of binding TGFβ1. For example, the receptor can be TGFβ receptor II (TGFβRII). In some embodiments, the TGFβ receptor is a human TGFβ receptor. In some embodiments, the IgG has a C-terminal fusion to a TGFβRII extracellular domain (ECD) as described in U.S. Pat. No. 9,676,863, incorporated herein. An “Fc linker” may be used to attach the IgG to the TGFβRII extracellular domain, for example, a G₄S₄G Fc linker. The Fc linker may be a short, flexible peptide that allows for the proper three-dimensional folding of the molecule while maintaining the binding-specificity to the targets. In some embodiments, the N-terminus of the TGFβ receptor is fused to the Fc of the antibody construct (with or without an Fc linker). In some embodiments, the C-terminus of the antibody construct heavy chain is fused to the TGFβ receptor (with or without an Fc linker). In some embodiments, the C-terminal lysine residue of the antibody construct heavy chain is mutated to alanine.

In some embodiments, the antibodies in the immunoconjugates are glycosylated.

In some embodiments, the antibodies in the immunoconjugates is a cysteine-engineered antibody which provides for site-specific conjugation of an adjuvant, label, or drug moiety to the antibody through cysteine substitutions at sites where the engineered cysteines are available for conjugation but do not perturb immunoglobulin folding and assembly or alter antigen binding and effector functions (Junutula, et al., 2008b Nature Biotech., 26(8):925-932; Dornan et al. (2009) Blood 114(13):2721-2729; U.S. Pat. Nos. 7,521,541; 7,723,485; US 2012/0121615; WO 2009/052249). A “cysteine engineered antibody” or “cysteine engineered antibody variant” is an antibody in which one or more residues of an antibody are substituted with cysteine residues. Cysteine-engineered antibodies can be conjugated to the 8-amido-2-aminobenzazepine adjuvant moiety as an 8-amido-2-aminobenzazepine-linker compound with uniform stoichiometry (e.g., up to 2 8-amido-2-aminobenzazepine moieties per antibody in an antibody that has a single engineered cysteine site).

In some embodiments, cysteine-engineered antibodies used to prepare the immunoconjugates of Table 3 have a cysteine residue introduced at the 149-lysine site of the light chain (LC K149C). In other embodiments, the cysteine-engineered antibodies have a cysteine residue introduced at the 118-alanine site (EU numbering) of the heavy chain (HC A118C). This site is alternatively numbered 121 by Sequential numbering or 114 by Kabat numbering. In other embodiments, the cysteine-engineered antibodies have a cysteine residue introduced in the light chain at G64C or R142C according to Kabat numbering, or in the heavy chain at D101C, V184C or T205C according to Kabat numbering.

### 8-Amido-2-Aminobenzazepine Adjuvant Compounds

The immunoconjugate of the invention comprises an 8-amido-2-aminobenzazepine adjuvant moiety. The adjuvant moiety described herein is a compound that elicits an immune response (i.e., an immunostimulatory agent). Generally, the adjuvant moiety described herein is a TLR agonist. TLRs are type-I transmembrane proteins that are responsible for the initiation of innate immune responses in vertebrates. TLRs recognize a variety of pathogen-associated molecular patterns from bacteria, viruses, and fungi and act as a first line of defense against invading pathogens. TLRs elicit overlapping yet distinct biological responses due to differences in cellular expression and in the signaling pathways that they initiate. Once engaged (e.g., by a natural stimulus or a synthetic TLR agonist), TLRs initiate a signal transduction cascade leading to activation of nuclear factor-κB (NF-κB) via the adapter protein myeloid differentiation primary response gene 88 (MyD88) and recruitment of the IL-1 receptor associated kinase (IRAK). Phosphorylation of IRAK then leads to recruitment of TNF-receptor associated factor 6 (TRAF6), which results in the phosphorylation of the NF-κB inhibitor I-κB. As a result, NF-κB enters the cell nucleus and initiates transcription of genes whose promoters contain NF-κB binding sites, such as cytokines. Additional modes of regulation for TLR signaling include TIR-domain containing adapter-inducing interferon-β (TRIF)-dependent induction of TNF-receptor associated factor 6 (TRAF6) and activation of MyD88 independent pathways via TRIF and TRAF3, leading to the phosphorylation of interferon response factor three (IRF3). Similarly, the MyD88 dependent pathway also activates several TRF family members, including IRF5 and IRF7 whereas the TRIF dependent pathway also activates the NF-κB pathway.

Typically, the adjuvant moiety described herein is a TLR7 and/or TLR8 agonist. TLR7 and TLR8 are both expressed in monocytes and dendritic cells. In humans, TLR7 is also expressed in plasmacytoid dendritic cells (pDCs) and B cells. TLR8 is expressed mostly in cells of myeloid origin, i.e., monocytes, granulocytes, and myeloid dendritic cells. TLR7 and TLR8 are capable of detecting the presence of “foreign” single-stranded RNA within a cell, as a means to respond to viral invasion. Treatment of TLR8-expressing cells, with TLR8 agonists can result in production of high levels of IL-12, IFN-γ, IL-1, TNF-α, IL-6, and other inflammatory cytokines. Similarly, stimulation of TLR7-expressing cells, such as pDCs, with TLR7 agonists can result in production of high levels of IFN-α and other inflammatory cytokines. TLR7/TLR8 engagement and resulting cytokine production can activate dendritic cells and other antigen-presenting cells, driving diverse innate and acquired immune response mechanisms leading to tumor destruction.

### Computational Modelling of Related Compounds Binding to TLR 7/8

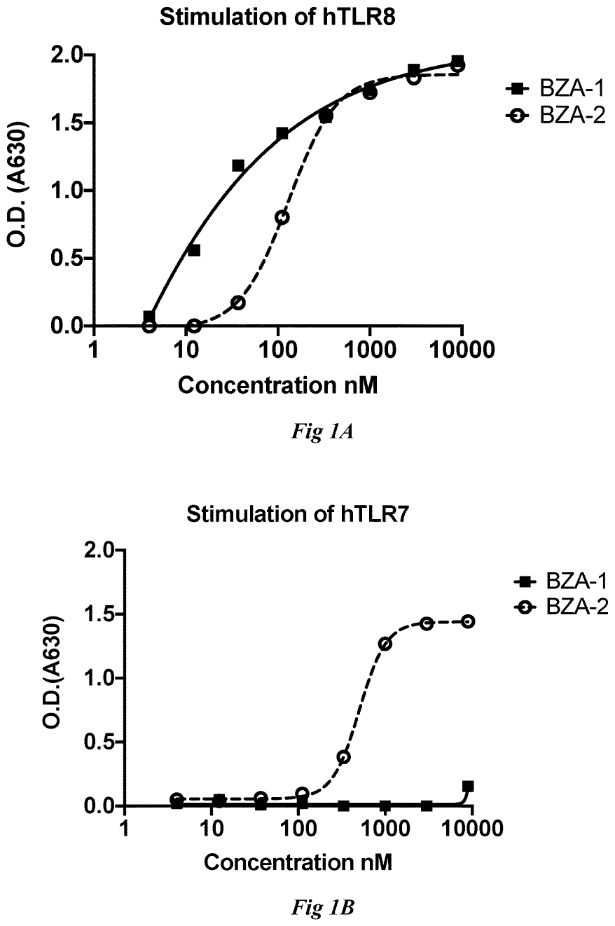

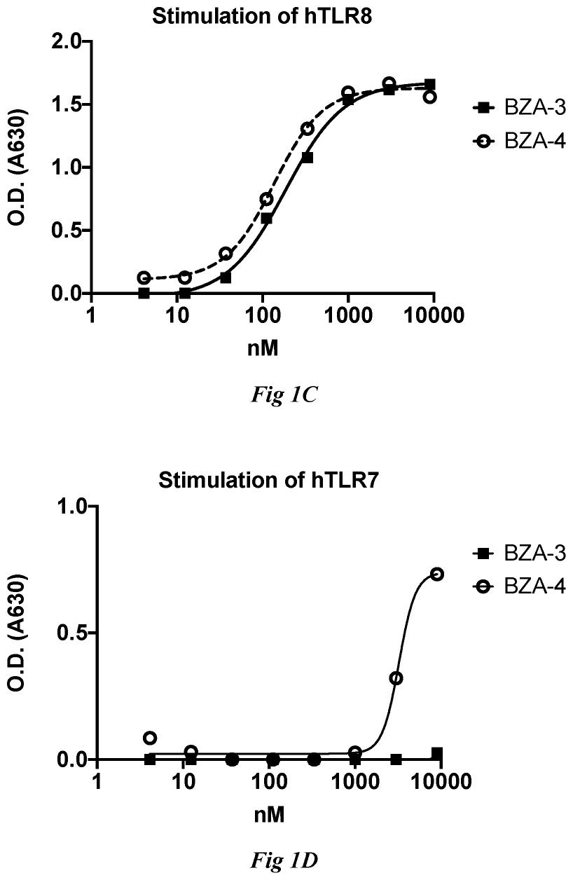

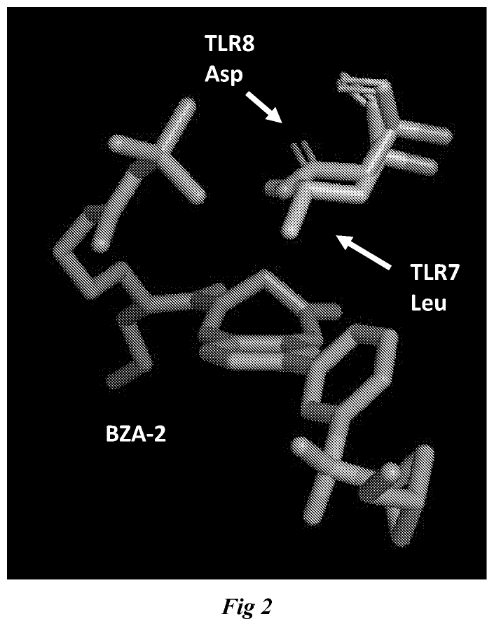

Structural modifications of the 4-amide side chain in the benzazepine scaffold may affect the potency and selectivity of 8-amido-2-aminobenzazepine adjuvant binding to TLR7 and TLR8. Certain structural alterations can change a TLR8-selective agonist to a dual TLR7/8 agonist. Modification of the dipropylamide on BZA-1 with a NHBoc group (BZA-2) minimally perturbs TLR8 activity (FIG. 1A) while significantly increasing TLR7 activity (FIG. 1). Additionally, this same structural modification applied to BZA-3 to generate BZA-4, a positional isomer of 8AmBza-9, increases TLR 7 activity (FIG. 1D) and does not affect TLR 8 activity (FIG. 1C).

The BZA-2 and BZA-4 molecules were conformationally enumerated using the Merck Molecular Force Field (MMFF94) by RDKit, Open-Source Cheminformatics Software (Halgren, T. A. (1999) J. Comput. Chem., 20:720-729). Those conformations were then docked into TLR8 (3w3n) by rDock followed by a molecular mechanics minimization (simplex minimization) of the poses in TLR8. rDock (previously RiboDock) is an open-source molecular docking software useful for docking small molecules against proteins and nucleic acids. rDock is primarily designed for high throughput virtual screening and prediction of binding mode (Morley, S. D. et al (2004) Journal of Computer-Aided Molecular Design 18 (3):189-208; Ruiz-Carmona, S. (2014) PLoS Computational Biology 10 (4): e1003571). Strain energies were determined by taking the final orientations from docking, and then performing a QM Optimization and Minimization in Psi4.

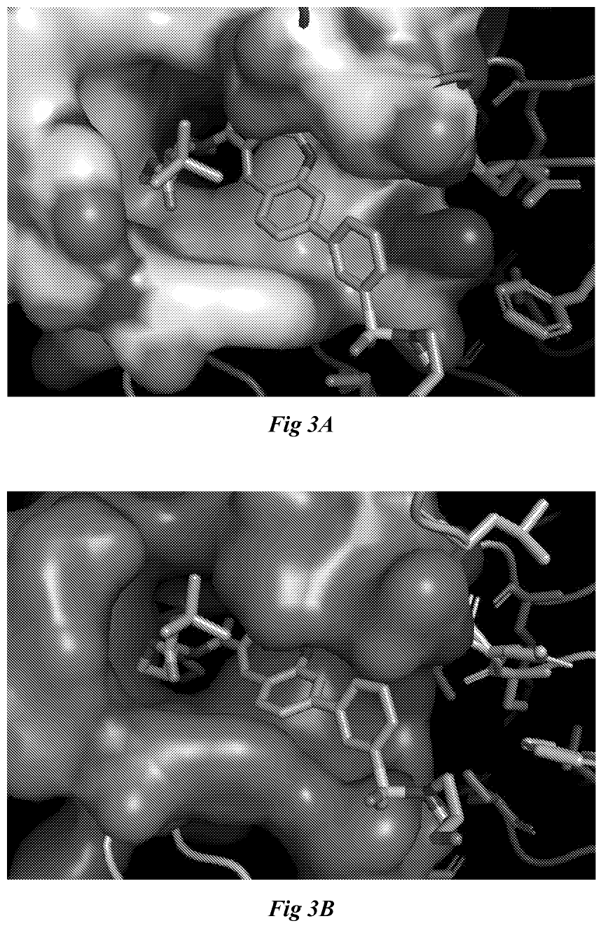

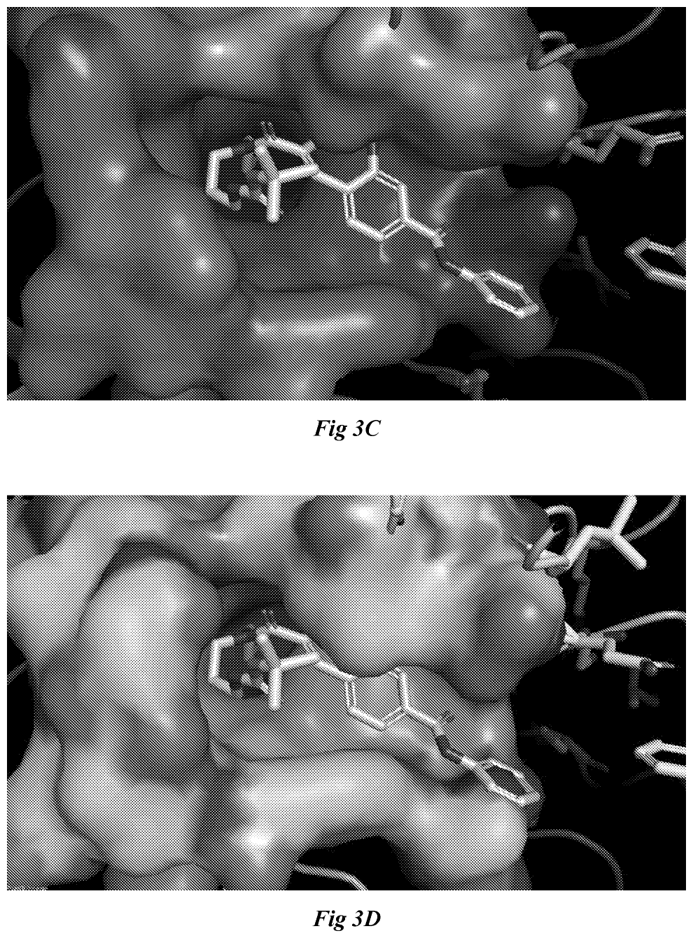

FIG. 2 shows a computational docking image of BZA-2 docked, highlighting interactions with TLR8 Asp and TLR7 Leu residues. The origin of this effect may be attributed to differing amino acid residues between TLR8 and TLR7: Asp(545) for TLR8; Leu(557) for TLR7. FIG. 3A shows a computational docking solution image of BZA-2 to TLR8. FIG. 3B shows a computational docking solution image of BZA-2 to TLR7, with the hydrophobic tert-butyl group interacting with Leu 557 in TLR7 thereby increasing TLR7 potency. In contrast, TLR8 protein conformation is capable of accommodating the NHBoc structural motif and preserving modest TLR8 potency (FIG. 3A). The same observations hold when examining the docked structures of BZA-4, as seen in FIGS. 3C and 3D. This surprising and unexpected property of the NHBoc structural motif may enable the design of potent 8-amido-2-aminobenzazepine TLR 7/8 agonists. Potency and selectivity of 8-amido-2-aminobenzazepine adjuvant binding to TLR7 and TLR8 can also be expected for adjuvants with a hydroxamate group such as 8AmBza-15 and 8AmBza-18 in Table 1b. Computational docking solution images suggest interactions with Tyr 348.

| | | |
|---|---|---|
| BZA-1 | 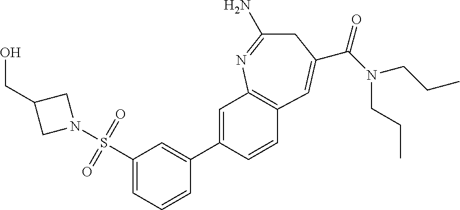 [SMILES: CCCN(CCC)C(=O)C1=Cc2ccc(-c3cccc(S(=O)(=O)N4CC(CO)C4)c3)cc2N=C(N)C1] | 2-amino-8-(3-((3- (hydroxymethyl)azetidin-1- yl)sulfonyl)phenyl)-N,N- dipropyl-3H-benzo[b]azepine- 4-carboxamide |
| BZA-2 | 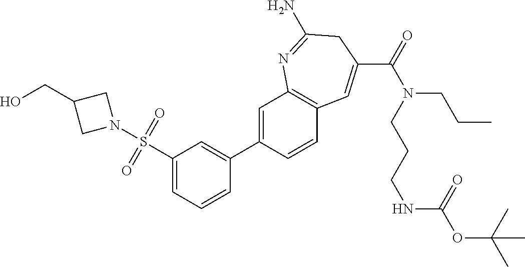 [SMILES: CCCN(CCCNC(=O)OC(C)(C)C)C(=O)C1=Cc2ccc(-c3cccc(S(=O)(=O)N4CC(CO)C4)c3)cc2N=C(N)C1] | tert-butyl (3-(2-amino-8-(3- ((3-(hydroxymethyl)azetidin-1- yl)sulfonyl)phenyl)-N-propyl- 3H-benzo[b]azepine-4- carboxamido)propyl)carbamate |
| BZA-3 | 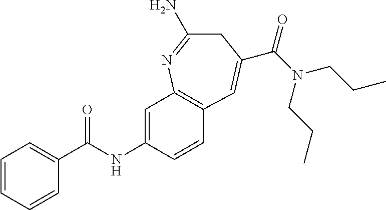 [SMILES: CCCN(CCC)C(=O)C1=Cc2ccc(NC(=O)c3ccccc3)cc2N=C(N)C1] | 2-amino-8-benzamido-N,N- dipropyl-3H-benzo[b]azepine- 4-carboxamide |
| BZA-4 | 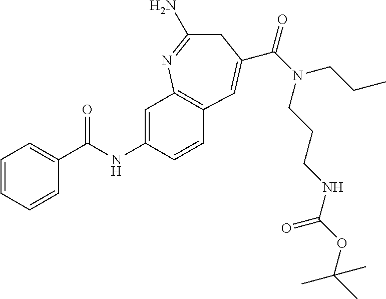 [SMILES: CCCN(CCCNC(=O)OC(C)(C)C)C(=O)C1=Cc2ccc(NC(=O)c3ccccc3)cc2N=C(N)C1] | tert-butyl (3-(2-amino-8- benzamido-N-propyl-3H- benzo[b]azepine-4- carboxamido)propyl)carbamate |

Exemplary 8-amido-2-aminobenzazepine compounds (8AmBza) of the invention are shown in Tables 1a and 1b. Each compound was characterized by mass spectrometry and shown to have the mass indicated. Activity against HEK293 NFKB reporter cells expressing human TLR7 or human TLR8 was measured according to Example 30.

TABLE 1a
8-Amido-2-aminobenzazepine compounds (8AmBza)

| | | | HEK293 | HEK293 |
|---|---|---|---|---|
| | | | hTLR7 | hTLR8 |
| 8AmBza | | | EC50 | EC50 |
| No. | Structure | MW | (nM) | (nM) |
| 8AmBza-1 | 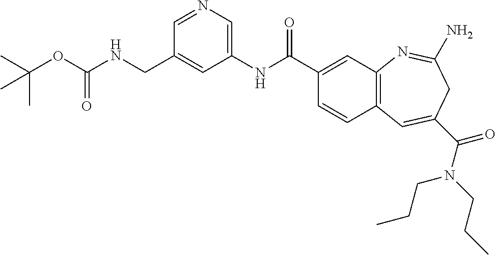 [SMILES: CCCN(CCC)C(=O)C1=Cc2ccc(C(=O)Nc3cncc(CNC(=O)OC(C)(C)C)c3)cc2N=C(N)C1] | 534.7 | >9000 | 4.283 |
| 8AmBza-2 | 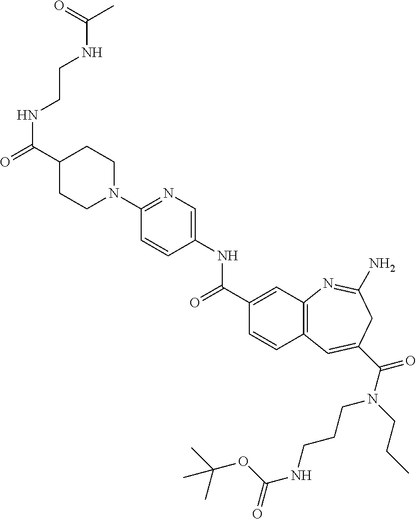 [SMILES: CCCN(CCCNC(=O)OC(C)(C)C)C(=O)C1=Cc2ccc(C(=O)Nc3ccc(N4CCC(C(=O)NCCNC(C)=O)CC4)nc3)cc2N=C(N)C1] | 731.9 | >9000 | 1047 |
| 8AmBza-3 | 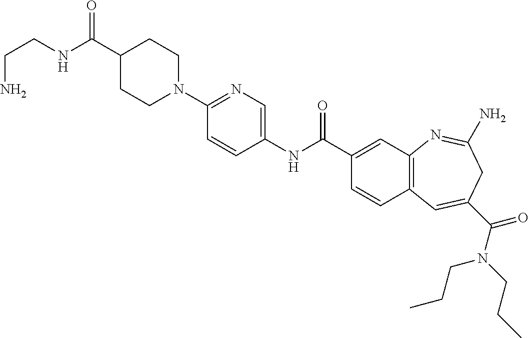 [SMILES: CCCN(CCC)C(=O)C1=Cc2ccc(C(=O)Nc3ccc(N4CCC(C(=O)NCCN)CC4)nc3)cc2N=C(N)C1] | 574.7 | NA | NA |
| 8AmBza-4 | 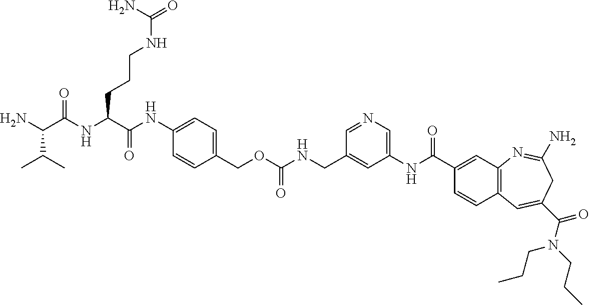 [SMILES: CCCN(CCC)C(=O)C1=Cc2ccc(C(=O)Nc3cncc(CNC(=O)OCc4ccc(NC(=O)[C@H](CCCNC(N)=O)NC(=O)[C@@H](N)C(C)C)cc4)c3)cc2N=C(N)C1] | 840.0 | NA | NA |
| 8AmBza-5 | 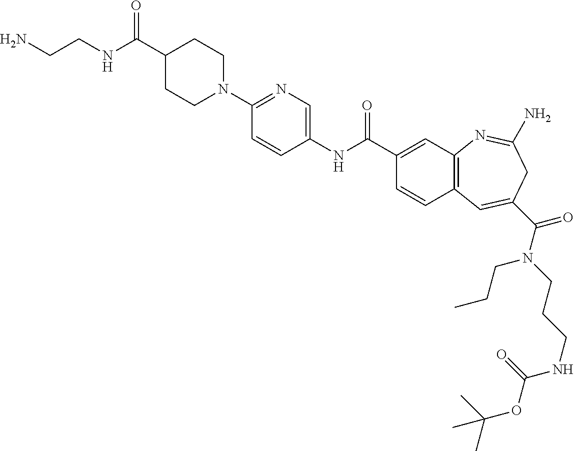 [SMILES: CCCN(CCCNC(=O)OC(C)(C)C)C(=O)C1=Cc2ccc(C(=O)Nc3ccc(N4CCC(C(=O)NCCN)CC4)nc3)cc2N=C(N)C1] | 689.9 | NA | NA |
| 8AmBza-6 | 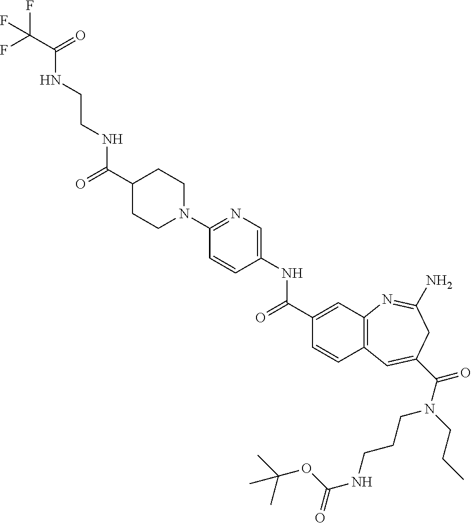 [SMILES: CCCN(CCCNC(=O)OC(C)(C)C)C(=O)C1=Cc2ccc(C(=O)Nc3ccc(N4CCC(C(=O)NCCNC(=O)C(F)(F)F)CC4)nc3)cc2N=C(N)C1] | 785.9 | NA | NA |
| 8AmBza-7 | 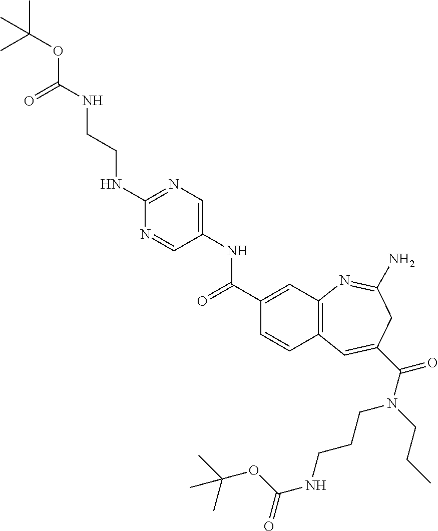 [SMILES: CCCN(CCCNC(=O)OC(C)(C)C)C(=O)C1=Cc2ccc(C(=O)Nc3cnc(NCCNC(=O)OC(C)(C)C)nc3)cc2N=C(N)C1] | 679.8 | >9000 | 1764 |
| 8AmBza-8 | 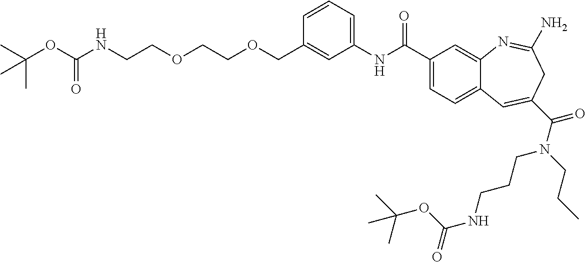 [SMILES: CCCN(CCCNC(=O)OC(C)(C)C)C(=O)C1=Cc2ccc(C(=O)Nc3cccc(COCCOCCNC(=O)OC(C)(C)C)c3)cc2N=C(N)C1] | 736.9 | >9000 | 862 |
| 8AmBza-9 | 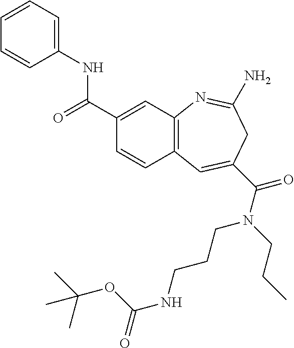 [SMILES: CCCN(CCCNC(=O)OC(C)(C)C)C(=O)C1=Cc2ccc(C(=O)Nc3ccccc3)cc2N=C(N)C1] | 519.6 | >9000 | 127 |
| 8AmBza-10 | 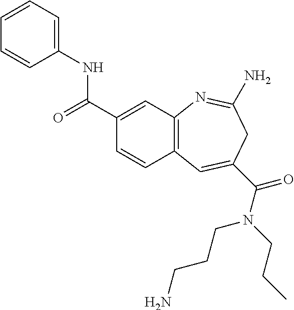 [SMILES: CCCN(CCCN)C(=O)C1=Cc2ccc(C(=O)Nc3ccccc3)cc2N=C(N)C1] | 419.5 | NA | NA |
| 8AmBza-11 | 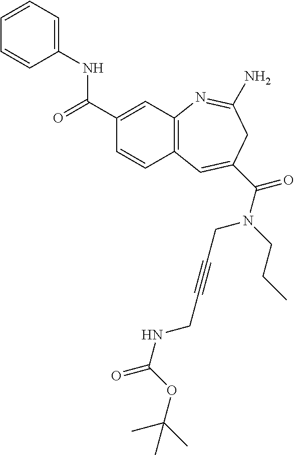 [SMILES: CCCN(CC#CCNC(=O)OC(C)(C)C)C(=O)C1=Cc2ccc(C(=O)Nc3ccccc3)cc2N=C(N)C1] | 529.6 | NA | NA |
| 8AmBza-12 | 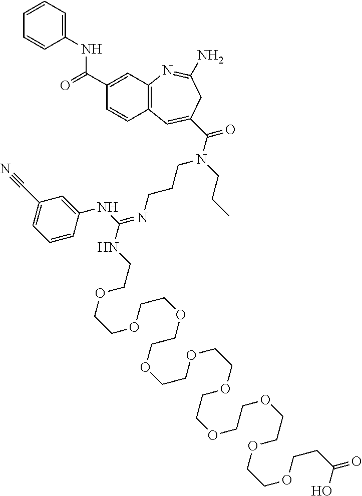 [SMILES: CCCN(CCC/N=C(/NCCOCCOCCOCCOCCOCCOCCOCCOCCOCCOCCC(=O)O)Nc1cccc(C#N)c1)C(=O)C1=Cc2ccc(C(=O)Nc3ccccc3)cc2N=C(N)C1] | 1075.3 | NA | NA |

TABLE 1b
8-Amido-2-aminobenzazepine compounds (8AmBza)

| | | | HEK293 | HEK293 |
|---|---|---|---|---|
| | | | hTLR7 | hTLR8 |
| 8AmBza | | | EC50 | EC50 |
| No. | Structure | MW | (nM) | (nM) |
| 8AmBza-13 | 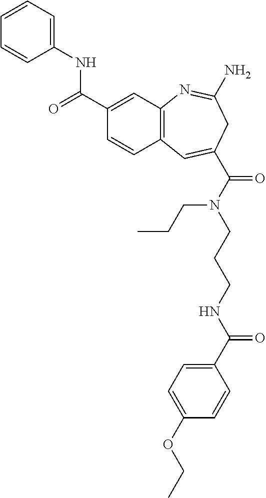 [SMILES: CCCN(CCCNC(=O)c1ccc(OCC)cc1)C(=O)C1=Cc2ccc(C(=O)Nc3ccccc3)cc2N=C(N)C1] | 567.7 | >9000 | 1040 |
| 8AmBza-14 | 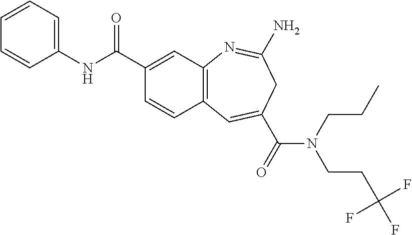 [SMILES: CCCN(CCC(F)(F)F)C(=O)C1=Cc2ccc(C(=O)Nc3ccccc3)cc2N=C(N)C1] | 458.5 | >9000 | 245 |
| 8AmBza-15 | 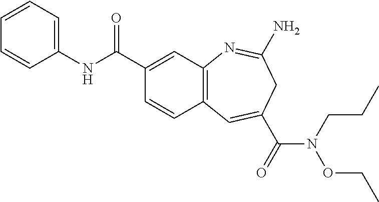 [SMILES: CCCN(OCC)C(=O)C1=Cc2ccc(C(=O)Nc3ccccc3)cc2N=C(N)C1] | 406.5 | 4860 | 34 |
| 8AmBza-16 | 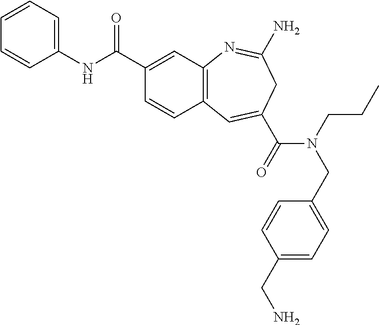 [SMILES: CCCN(Cc1ccc(CN)cc1)C(=O)C1=Cc2ccc(C(=O)Nc3ccccc3)cc2N=C(N)C1] | 481.6 | >9000 | 77 |
| 8AmBza-17 | 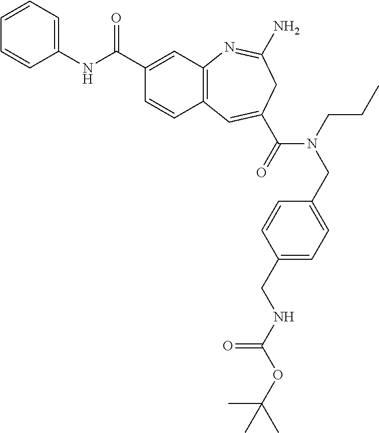 [SMILES: CCCN(Cc1ccc(CNC(=O)OC(C)(C)C)cc1)C(=O)C1=Cc2ccc(C(=O)Nc3ccccc3)cc2N=C(N)C1] | 581.7 | >9000 | 782 |
| 8AmBza-18 | 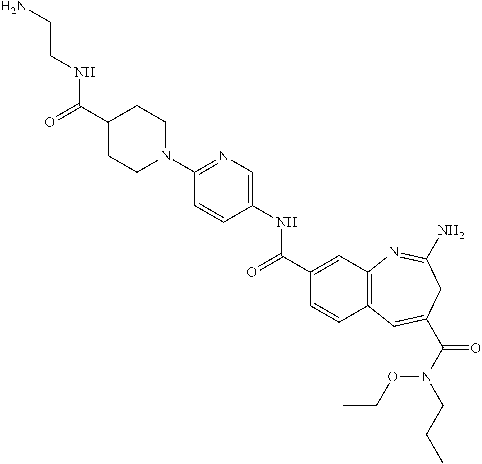 [SMILES: CCCN(OCC)C(=O)C1=Cc2ccc(C(=O)Nc3ccc(N4CCC(C(=O)NCCN)CC4)nc3)cc2N=C(N)C1] | 576.7 | >9000 | 5618 |
| 8AmBza-19 | 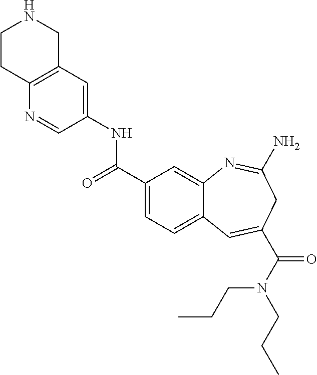 [SMILES: CCCN(CCC)C(=O)C1=Cc2ccc(C(=O)Nc3cnc4c(c3)CNCC4)cc2N=C(N)C1] | 460.6 | NA | 2182 |
| 8AmBza-20 | 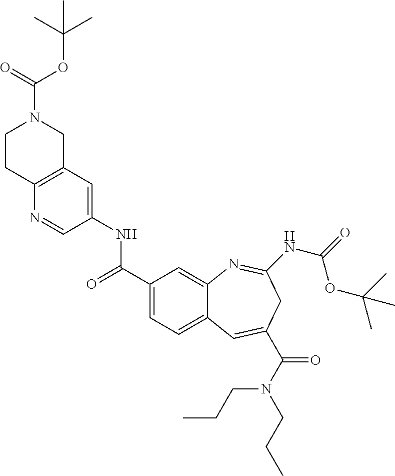 [SMILES: CCCN(CCC)C(=O)C1=Cc2ccc(C(=O)Nc3cnc4c(c3)CN(C(=O)OC(C)(C)C)CC4)cc2N=C(NC(=O)OC(C)(C)C)C1] | 660.8 | 8130 | 6080 |
| 8AmBza-21 | 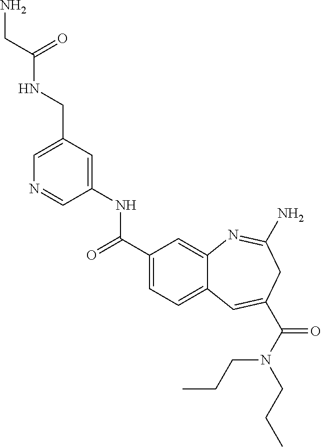 [SMILES: CCCN(CCC)C(=O)C1=Cc2ccc(C(=O)Nc3cncc(CNC(=O)CN)c3)cc2N=C(N)C1] | 491.6 | >9000 | 4324 |
| 8AmBza-22 | 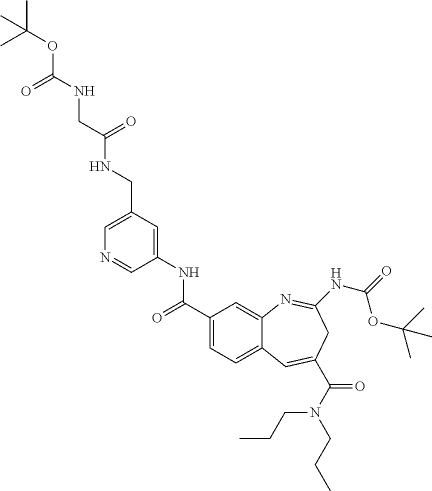 [SMILES: CCCN(CCC)C(=O)C1=Cc2ccc(C(=O)Nc3cncc(CNC(=O)CNC(=O)OC(C)(C)C)c3)cc2N=C(NC(=O)OC(C)(C)C)C1] | 691.8 | >9000 | >9000 |
| 8AmBza-23 | 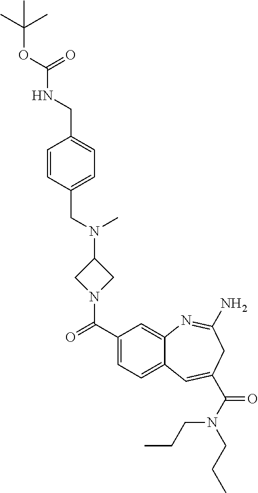 [SMILES: CCCN(CCC)C(=O)C1=Cc2ccc(C(=O)N3CC(N(C)Cc4ccc(CNC(=O)OC(C)(C)C)cc4)C3)cc2N=C(N)C1] | | | |
| 8AmBza-24 | 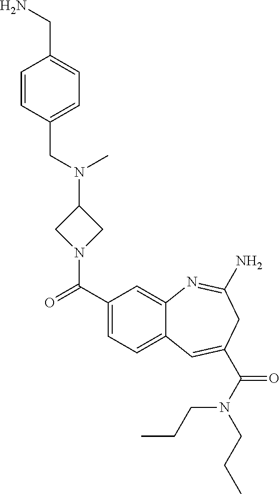 [SMILES: CCCN(CCC)C(=O)C1=Cc2ccc(C(=O)N3CC(N(C)Cc4ccc(CN)cc4)C3)cc2N=C(N)C1] | | | |
| 8AmBza-25 | 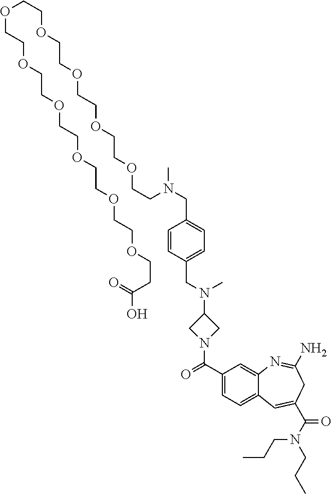 [SMILES: CCCN(CCC)C(=O)C1=Cc2ccc(C(=O)N3CC(N(C)Cc4ccc(CN(C)CCOCCOCCOCCOCCOCCOCCOCCOCCOCCOCCC(=O)O)cc4)C3)cc2N=C(N)C1] | | | |

### 8-Amido-2-Aminobenzazepine-Linker Compounds

The immunoconjugates of the invention are prepared by conjugation of an antibody with an 8-amido-2-aminobenzazepine-linker compound. The 8-amido-2-aminobenzazepine-linker compounds comprise an 8-amido-2-aminobenzazepine (8AmBza) moiety covalently attached to a linker unit, L. The linker units comprise functional groups and subunits which affect stability, permeability, solubility, and other pharmacokinetic, safety, and efficacy properties of the immunoconjugates. The linker unit includes a reactive functional group which reacts, i.e. conjugates, with a reactive functional group of the antibody. For example, a nucleophilic group such as a lysine side chain amino of the antibody reacts with an electrophilic reactive functional group of the 8AmBza-linker compound to form the immunoconjugate. Also, for example, a cysteine thiol of the antibody reacts with a maleimide or bromoacetamide group of the 8AmBza-linker compound to form the immunoconjugate.

Electrophilic reactive functional group suitable for the 8AmBza-linker compounds include, but are not limited to, N-hydroxysuccinimidyl (NHS) esters and N-hydroxysulfosuccinimidyl (sulfo-NHS) esters (amine reactive); carbodiimides (amine and carboxyl reactive); hydroxymethyl phosphines (amine reactive); maleimides (thiol reactive); halogenated acetamides such as N-iodoacetamides (thiol reactive); aryl azides (primary amine reactive); fluorinated aryl azides (reactive via carbon-hydrogen (C—H) insertion); pentafluorophenyl (PFP) esters (amine reactive); tetrafluorophenyl (TFP) esters (amine reactive); imidoesters (amine reactive); isocyanates (hydroxyl reactive); vinyl sulfones (thiol, amine, and hydroxyl reactive); pyridyl disulfides (thiol reactive); and benzophenone derivatives (reactive via C—H bond insertion). Further reagents include, but are not limited, to those described in Hermanson, Bioconjugate Techniques 2nd Edition, Academic Press, 2008.

The invention provides solutions to the limitations and challenges to the design, preparation and use of immunoconjugates. Some linkers may be labile in the blood stream, thereby releasing unacceptable amounts of the adjuvant/drug prior to internalization in a target cell (Khot, A. et al (2015) Bioanalysis 7(13):1633-1648). Other linkers may provide stability in the bloodstream, but intracellular release effectiveness may be negatively impacted. Linkers that provide for desired intracellular release typically have poor stability in the bloodstream. Alternatively stated, bloodstream stability and intracellular release are typically inversely related. In addition, in standard conjugation processes, the amount of adjuvant/drug moiety loaded on the antibody, i.e. drug loading, the amount of aggregate that is formed in the conjugation reaction, and the yield of final purified conjugate that can be obtained are interrelated. For example, aggregate formation is generally positively correlated to the number of equivalents of adjuvant/drug moiety and derivatives thereof conjugated to the antibody. Under high drug loading, formed aggregates must be removed for therapeutic applications. As a result, drug loading-mediated aggregate formation decreases immunoconjugate yield and can render process scale-up difficult.

Exemplary embodiments include an 8-amido-2-aminobenzazepine-linker compound of Formula II:

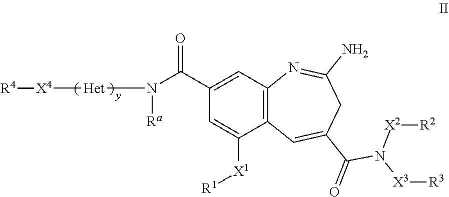

wherein

y is 0 or 1;

Het is selected from the group consisting of heterocyclyl, heterocyclyldiyl, heteroaryl, and heteroaryldiyl;

R^{a} is H or forms Het with the nitrogen atom it is bound to;

R¹, R², R³, and R⁴ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
—(C₁-C₁₂ alkyldiyl)-N(R⁵)—*;
—(C₁-C₁₂ alkyldiyl)-N(R⁵)₂;
—(C₁-C₁₂ alkyldiyl)-OR⁵;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁵—*;
—(C₃-C₁₂ carbocyclyl)-(C₁-C₁₂ alkyldiyl)-N(R⁵)₂;
—(C₃-C₁₂ carbocyclyl)-NR—C(═NR⁵)NR⁵—*;
—(C₆-C₂₀ aryl);
—(C₆-C₂₀ aryl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁵)₂;
—(C₆-C₂₀ aryldiyl)-(C₁-C₁₂ alkyldiyl)-NR⁵—C(═NR^{5a})N(R⁵)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁵—*;
—(C₂-C₉ heterocyclyl)-(C₁-C₁₂ alkyldiyl)-N(R⁵)₂;
—(C₂-C₉ heterocyclyl)-NR⁵—C(═NR^{5a})NR⁵—*;
—(C₁-C₂₀ heteroaryl);
—(C₁-C₂₀ heteroaryl)-*;
—(C₁-C₂₀ heteroaryl)-(C₁-C₁₂ alkyldiyl)-N(R⁵)—*;
—(C₁-C₂₀ heteroaryl)-(C₁-C₁₂ alkyldiyl)-N(R⁵)₂;
—(C₁-C₂₀ heteroaryl)-NR⁵—C(═NR^{5a})N(R⁵)—*;
—C(═O)—*;
—C(═O)—(C₁-C₁₂ alkyldiyl)-N(R⁵)—*;
—C(═O)—(C₂-C₂₀ heterocyclyldiyl)-*;
—C(═O)N(R⁵)₂;
—C(═O)N(R⁵)—*;
—C(═O)N(R⁵)—(C₁-C₁₂ alkyldiyl)-N(R⁵)C(═O)R⁵;
—C(═O)N(R⁵)—(C₁-C₁₂ alkyldiyl)-N(R⁵)C(═O)N(R⁵)₂;
—C(═O)NR⁵—(C₁-C₁₂ alkyldiyl)-N(R⁵)CO₂R⁵;
—C(═O)NR⁵—(C₁-C₁₂ alkyldiyl)-N(R⁵)C(═NR^{5a})N(R⁵)₂;
—C(═O)NR⁵—(C₁-C₁₂ alkyldiyl)-NR⁵C(═NR^{5a})R⁵;
—C(═O)NR⁵—(C₁-C₈ alkyldiyl)-NR⁵(C₂-C₅ heteroaryl);
—C(═O)NR⁵—(C₁-C₂₀ heteroaryldiyl)-N(R⁵)—*;
—C(═O)NR⁵—(C₁-C₂₀ heteroaryldiyl)-*;
—C(═O)NR⁵—(C₁-C₂₀ heteroaryldiyl)-(C₁-C₁₂ alkyldiyl)-N(R⁵)₂;
—C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵—*;
—N(R⁵)₂;
—N(R⁵)—*;
—N(R⁵)C(═O)R⁵;
—N(R⁵)C(═O)—*;
—N(R⁵)C(═O)N(R⁵)₂;
—N(R⁵)C(═O)N(R⁵)—*;
—N(R⁵)CO₂R⁵;
—NR⁵C(═NR^{5a})N(R⁵)₂;
—NR⁵C(═NR^{5a})N(R⁵)—*;
—NR⁵C(═NR^{5a})R⁵;
—N(R⁵)—(C₂-C₅ heteroaryl);
—O—(C₁-C₁₂ alkyl);
—O—(C₁-C₁₂ alkyldiyl)-N(R⁵)₂;
—O—(C₁-C₁₂ alkyldiyl)-N(R⁵)—*;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁵)₂;
—S(═O)₂—(C₂-C₂₀ heterocyclyldiyl)-(C₁-C₁₂ alkyldiyl)-NR⁵—*; and
—S(═O)₂—(C₂-C₂₀ heterocyclyldiyl)-(C₁-C₁₂ alkyldiyl)-OH;

or R² and R³ together form a 5- or 6-membered heterocyclyl ring;

X¹, X², X³, and X⁴ are independently selected from the group consisting of a bond, C(═O), C(═O)N(R⁵), O, N(R⁵), S, S(O)₂, and S(O)₂N(R⁵);

R⁵ is selected from the group consisting of H, C₆-C₂₀ aryl, C₆-C₂₀ aryldiyl, C₁-C₁₂ alkyl, and C₁-C₁₂ alkyldiyl, or two R⁵ groups together form a 5- or 6-membered heterocyclyl ring;

R^{5a} is selected from the group consisting of C₆-C₂₀ aryl and C₁-C₂₀ heteroaryl;

where the asterisk * indicates the attachment site of L, and where one of R¹, R², R³ and R⁴ is attached to L;

L is the linker selected from the group consisting of:
Q-C(═O)-(PEG)-C(═O)-(PEP)-;
Q-C(═O)-(PEG)-NR⁵—;
Q-C(═O)-(PEG)-NR⁵-(PEG)-C(═O)-(PEP)-;
Q-C(═O)-(PEG)-N⁺(R⁵)₂-(PEG)-C(═O)-(PEP)-;
Q-C(═O)-(PEG)-C(═O)—;
Q-C(═O)-(PEG)-NR⁵CH(AA₁)C(═O)-(PEG)-C(═O)-(PEP)-;
Q-C(═O)-(PEG)-SS—(C₁-C₁₂ alkyldiyl)-OC(═O)—;
Q-C(═O)-(PEG)-SS—(C₁-C₁₂ alkyldiyl)-C(═O)—;
Q-C(═O)-(PEG)-;
Q-C(═O)-(PEG)-C(═O)NR⁵(C₁-C₁₂ alkyldiyl)NR⁵C(═O)—(C₂-C₅ monoheterocyclyldiyl)-;
Q-C(═O)-(PEG)-C(═O)NR⁵(C₁-C₁₂ alkyldiyl)-;
Q-C(═O)—(C₁-C₁₂ alkyldiyl)-C(═O)-(PEP)-;
Q-C(═O)—(C₁-C₁₂ alkyldiyl)-C(═O)-(PEP)-NR⁵(C₁-C₁₂ alkyldiyl)-;
Q-C(═O)—(C₁-C₁₂ alkyldiyl)-C(═O)-(PEP)-NR⁵(C₁-C₁₂ alkyldiyl)NR⁵—C(═O);
Q-C(═O)—(C₁-C₁₂ alkyldiyl)-C(═O)-(PEP)-NR⁵(C₁-C₁₂ alkyldiyl)NR⁵C(═O)—(C₂-C₅ monoheterocyclyldiyl)-;
Q-C(═O)—CH₂CH₂OCH₂CH₂—(C₁-C₂₀ heteroaryldiyl)-CH₂O—(PEG)-C(═O)-(MCgluc)-;
Q-C(═O)—CH₂CH₂OCH₂CH₂—(C₁-C₂₀ heteroaryldiyl)-CH₂O—(PEG)-C(═O)-(MCgluc)-NR⁵(C₁-C₁₂ alkyldiyl)NR⁵C(═O)—(C₂-C₅ monoheterocyclyldiyl)-;
Q-C(═O)-(PEG)-C(═O)—NR⁵(C₁-C₁₂ alkyldiyl)-;
Q-C(═O)-(PEG)-C(═O)—NR⁵(C₁-C₁₂ alkyldiyl)NR⁵C(═O)—(C₂-C₅ monoheterocyclyldiyl)-;
Q-C(═O)-(PEG)-C(═O)-(PEP)-NR⁵(C₁-C₁₂ alkyldiyl)-;
Q-C(═O)-(PEG)-C(═O)-(PEP)-NR⁵(C₁-C₁₂ alkyldiyl)NR⁵C(═O)—(C₂-C₅ monoheterocyclyldiyl)-; and
Q-(CH₂)ₘC(═O)-(PEP)-NR⁵(C₁-C₁₂ alkyldiyl)NR⁵C(═O)—(C₂-C₅ monoheterocyclyldiyl)-;

where PEG has the formula: —(CH₂CH₂O)ₙ—(CH₂)ₘ—; m is an integer from 1 to 5, and n is an integer from 2 to 50;

PEP has the formula:

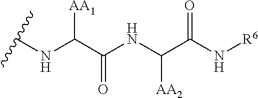

where AA₁ and AA₂ are independently selected from an amino acid side chain, or AA₁ or AA₂ and an adjacent nitrogen atom form a 5-membered ring proline amino acid, and the wavy line indicates a point of attachment and;

R⁶ is selected from the group consisting of C₆-C₂₀ aryldiyl and C₁-C₂₀ heteroaryldiyl, substituted with —CH₂O—C(═O)— and optionally with:

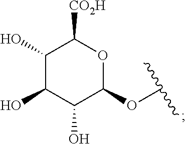 [SMILES: *O[C@@H]1O[C@H](C(=O)O)[C@@H](O)[C@H](O)[C@H]1O]

and

MCgluc is selected from the groups:

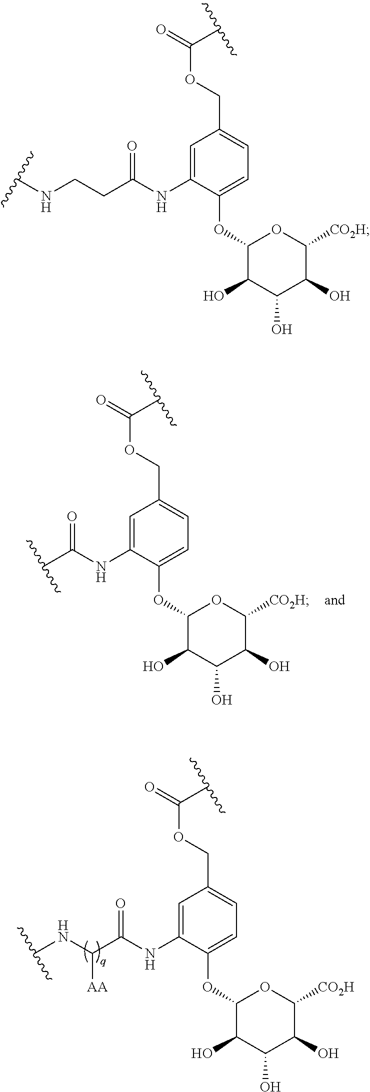

where q is 1 to 8, and AA is an amino acid side chain; and

Q is selected from the group consisting of N-hydroxysuccinimidyl, N-hydroxysulfosuccinimidyl, maleimide, and phenoxy substituted with one or more groups independently selected from F, Cl, NO₂, and SO₃;

where alkyl, alkyldiyl, alkenyl, alkenyldiyl, alkynyl, alkynyldiyl, aryl, aryldiyl carbocyclyl, carbocyclyldiyl, heterocyclyl, heterocyclyldiyl, heteroaryl, and heteroaryldiyl are optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OP(O)(OH)₂, —S(O)₂N(CH₃)₂, —SCH₃, —S(O)₂CH₃, and —S(O)₃H.

An exemplary embodiment of the 8-amido-2-aminobenzazepine-linker compound of Formula II includes wherein y is 0.

An exemplary embodiment of the 8-amido-2-aminobenzazepine-linker compound of Formula II includes wherein y is 1.

An exemplary embodiment of the 8-amido-2-aminobenzazepine-linker compound of Formula II includes wherein PEP has the formula:

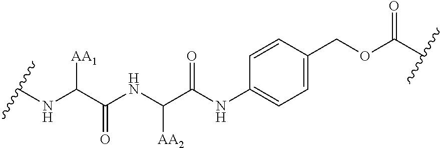

wherein AA₁ and AA₂ are independently selected from a side chain of a naturally-occurring amino acid.

An exemplary embodiment of the 8-amido-2-aminobenzazepine-linker compound of Formula II includes wherein AA₁ or AA₂ with an adjacent nitrogen atom form a 5-membered ring to form a proline amino acid.

An exemplary embodiment of the 8-amido-2-aminobenzazepine-linker compound of Formula II includes wherein PEP has the formula:

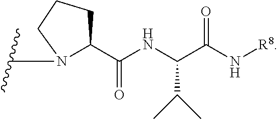

An exemplary embodiment of the 8-amido-2-aminobenzazepine-linker compound of Formula II includes wherein MCgluc has the formula:

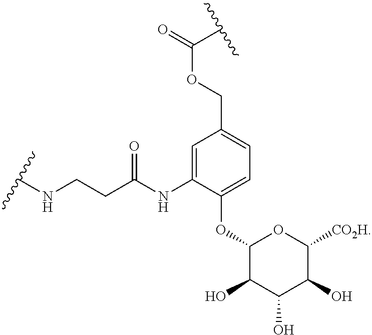 [SMILES: *NCCC(=O)Nc1cc(COC(*)=O)ccc1O[C@@H]1O[C@H](C(=O)O)[C@@H](O)[C@H](O)[C@H]1O]

An exemplary embodiment of the 8-amido-2-aminobenzazepine-linker compound of Formula II includes wherein AA₁ and AA₂ are independently selected from H, —CH₃, —CH(CH₃)₂, —CH₂(C₆H5), —CH₂CH₂CH₂CH₂NH₂, —CH₂CH₂CH₂NHC(NH)NH₂, —CHCH(CH₃)CH₃, —CH₂SO₃H, and —CH₂CH₂CH₂NHC(O)NH₂.

An exemplary embodiment of the 8-amido-2-aminobenzazepine-linker compound of Formula II includes wherein AA₁ is —CH(CH₃)₂, and AA₂ is —CH₂CH₂CH₂NHC(O)NH₂.

An exemplary embodiment of the 8-amido-2-aminobenzazepine-linker compound of Formula II includes wherein AA₁ and AA₂ are independently selected from GlcNAc aspartic acid, —CH₂SO₃H, and —CH₂OPO₃H.

An exemplary embodiment of the 8-amido-2-aminobenzazepine-linker compound of Formula II includes wherein X¹ is a bond, and R¹ is H.

An exemplary embodiment of the 8-amido-2-aminobenzazepine-linker compound of Formula II includes wherein X² is a bond, and R² is C₁-C₈ alkyl.

An exemplary embodiment of the 8-amido-2-aminobenzazepine-linker compound of Formula II includes wherein X² and X³ are each a bond, and R² and R³ are independently selected from C₁-C₈ alkyl, —O—(C₁-C₁₂ alkyl), —(C₁-C₁₂ alkyldiyl)-OR⁵, —(C₁-C₈ alkyldiyl)-N(R⁵)CO₂R⁵, and —O—(C₁-C₁₂ alkyl)-N(R⁵)CO₂R⁵.

An exemplary embodiment of the 8-amido-2-aminobenzazepine-linker compound of Formula II includes wherein R² and R³ are each independently selected from —CH₂CH₂CH₃, —OCH₂CH₃, —CH₂CH₂CF₃, and —CH₂CH₂CH₂OH.

An exemplary embodiment of the 8-amido-2-aminobenzazepine-linker compound of Formula II includes wherein R² is C₁-C₈ alkyl and R³ is —(C₁-C₈ alkyldiyl)-N(R⁵)CO₂R⁴.

An exemplary embodiment of the 8-amido-2-aminobenzazepine-linker compound of Formula II includes wherein R² is —CH₂CH₂CH₃ and R³ is —CH₂CH₂CH₂NHCO₂(t-Bu).

An exemplary embodiment of the 8-amido-2-aminobenzazepine-linker compound of Formula II includes wherein R² and R³ are each —CH₂CH₂CH₃.

An exemplary embodiment of the 8-amido-2-aminobenzazepine-linker compound of Formula II includes wherein NR⁵(C₂-C₅ heteroaryl) of R¹ or R³ is selected from:

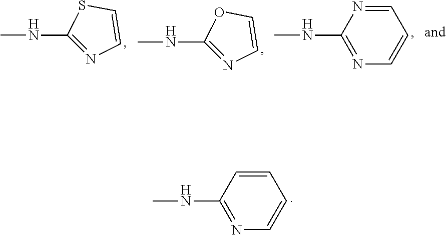

An exemplary embodiment of the 8-amido-2-aminobenzazepine-linker compound of Formula II includes wherein X³—R³ is selected from the group consisting of:

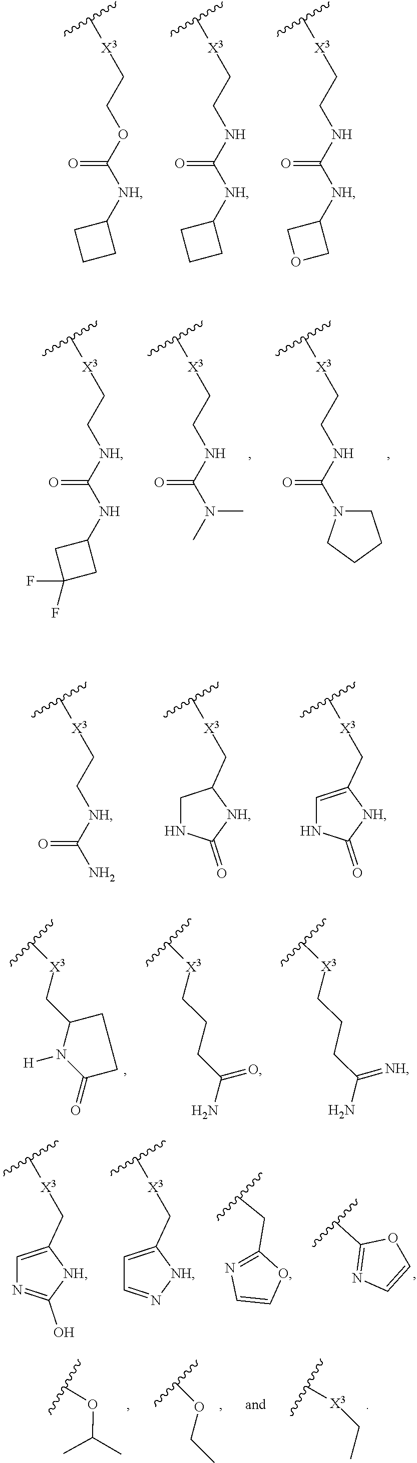

An exemplary embodiment of the 8-amido-2-aminobenzazepine-linker compound of Formula II includes wherein Het is a 5- or 6-membered monocyclic heteroaryldiyl selected from the group consisting of pyridyldiyl, imidazolyldiyl, pyrimidinyldiyl, pyrazolyldiyl, triazolyldiyl, pyrazinyldiyl, tetrazolyldiyl, furyldiyl, thienyldiyl, isoxazolyldiyldiyl, thiazolyldiyl, oxadiazolyldiyl, oxazolyldiyl, isothiazolyldiyl, and pyrrolyldiyl.

An exemplary embodiment of the 8-amido-2-aminobenzazepine-linker compound of Formula II includes wherein Het is a 5- or 6-membered monocyclic heterocyclyldiyl selected from the group consisting of morpholinyldiyl, piperidinyldiyl, piperazinyldiyl, pyrrolidinyldiyl, dioxanyldiyl, thiomorpholinyldiyl, and S-dioxothiomorpholinyldiyl.

An exemplary embodiment of the 8-amido-2-aminobenzazepine-linker compound of Formula II includes wherein Het is 1,6-naphthyridyl or 1,6-naphthyridiyl.

An exemplary embodiment of the 8-amido-2-aminobenzazepine-linker compound of Formula II includes wherein L is selected from the group consisting of:
Q-C(═O)—CH₂CH₂OCH₂CH₂—(C₁-C₂₀ heteroaryldiyl)-CH₂O—(PEG)-C(═O)-(MCgluc)-NR⁵(C₁-C₁₂ alkyldiyl)NR⁵C(═O)—(C₂-C₅ monoheterocyclyldiyl)-;
Q-C(═O)-(PEG)-C(═O)—NR⁵(C₁-C₁₂ alkyldiyl)-;
Q-C(═O)-(PEG)-C(═O)—NR⁵(C₁-C₁₂ alkyldiyl)NR⁵C(═O)—(C₂-C₅ monoheterocyclyldiyl)-;
Q-C(═O)-(PEG)-C(═O)-(PEP)-NR⁵(C₁-C₁₂ alkyldiyl)-;
Q-C(═O)-(PEG)-C(═O)-(PEP)-NR⁵(C₁-C₁₂ alkyldiyl)NR⁵C(═O)—(C₂-C₅ monoheterocyclyldiyl)-; and
Q-(CH₂)ₘ—C(═O)-(PEP)-NR⁵(C₁-C₁₂ alkyldiyl)NR⁵C(═O)—(C₂-C₅ monoheterocyclyldiyl)-.

An exemplary embodiment of the 8-amido-2-aminobenzazepine-linker compound of Formula IIa includes wherein Q is selected from:

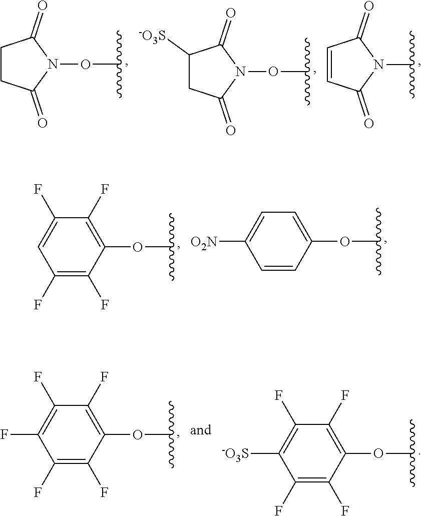

An exemplary embodiment of the 8-amido-2-aminobenzazepine-linker compound of Formula IIa includes wherein Q is phenoxy substituted with one or more F.

An exemplary embodiment of the 8-amido-2-aminobenzazepine-linker compound of Formula IIa includes wherein Q is 2,3,5,6-tetrafluorophenoxy.

An exemplary embodiment of the 8-amido-2-aminobenzazepine-linker compound of Formula II selected from Formulae IIa-d:

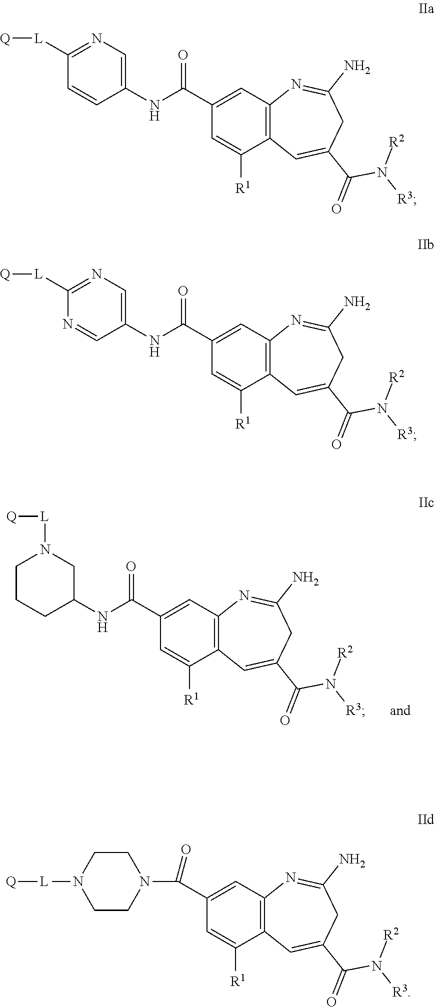

An exemplary embodiment of the 8-amido-2-aminobenzazepine-linker compound of Formulae IIa-d includes wherein R² is C₁-C₈ alkyl and R³ is —(C₁-C₈ alkyldiyl)-N(R⁵)CO₂R⁴.

An exemplary embodiment of the 8-amido-2-aminobenzazepine-linker compound of Formulae IIa-d includes wherein R² is —CH₂CH₂CH₃ and R³ is —CH₂CH₂CH₂NHCO₂(t-Bu).

An exemplary embodiment of the 8-amido-2-aminobenzazepine-linker compound of Formulae IIa-d includes wherein R² and R³ are —CH₂CH₂CH₃.

An exemplary embodiment of the 8-amido-2-aminobenzazepine-linker compound of Formulae IIa-d includes wherein Q is tetrafluorophenyl.

An exemplary embodiment of the 8-amido-2-aminobenzazepine-linker compound is selected from Table 2. Each compound was characterized by mass spectrometry and shown to have the mass indicated.

TABLE 2a
8-Amido-2-aminobenzazepine-linker (8AmBza-L) Formula II compounds

| 8AmBza-L | Structure | MW |
|---|---|---|
| 8AmBza-L-1 | 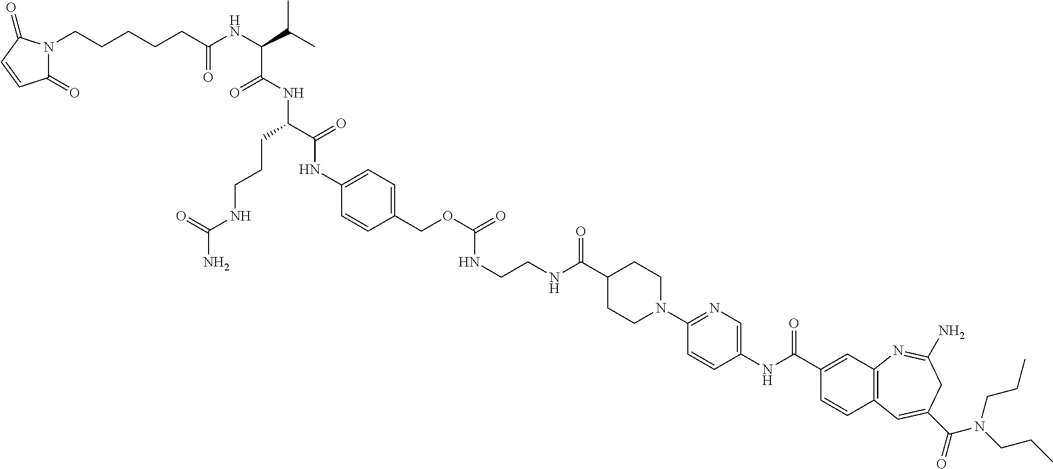 [SMILES: CCCN(CCC)C(=O)C1=Cc2ccc(C(=O)Nc3ccc(N4CCC(C(=O)NCCNC(=O)OCc5ccc(NC(=O)[C@H](CCCNC(N)=O)NC(=O)[C@@H](NC(=O)CCCCCN6C(=O)C=CC6=O)C(C)C)cc5)CC4)nc3)cc2N=C(N)C1] | 1173.4 |
| 8AmBza-L-2 | 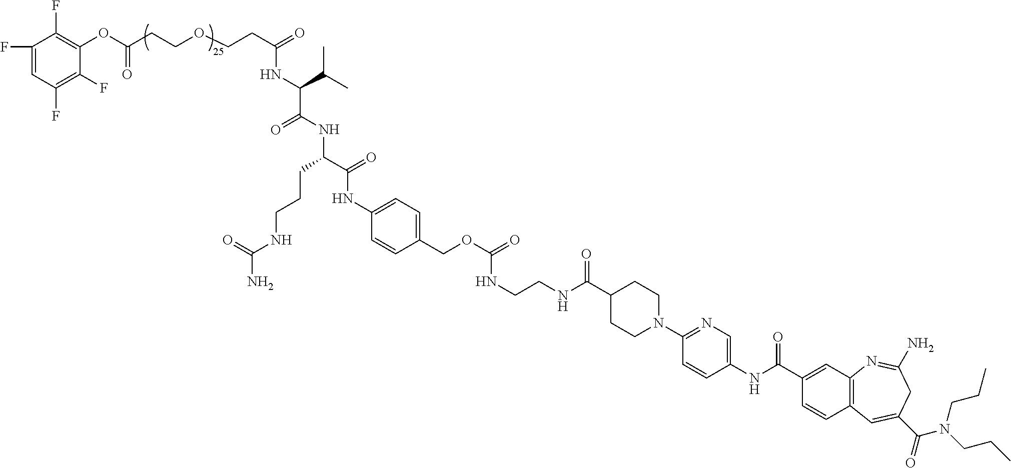 | 2329.6 |
| 8AmBza-L-3 | 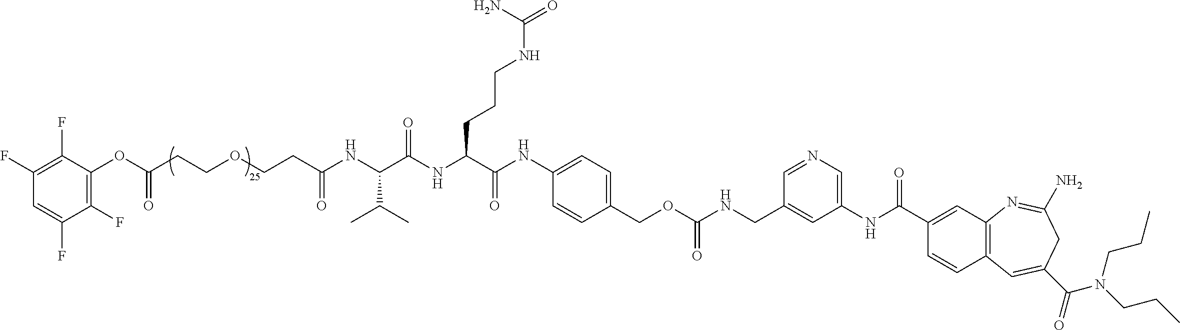 | 2189.4 |
| 8AmBza-L-4 | 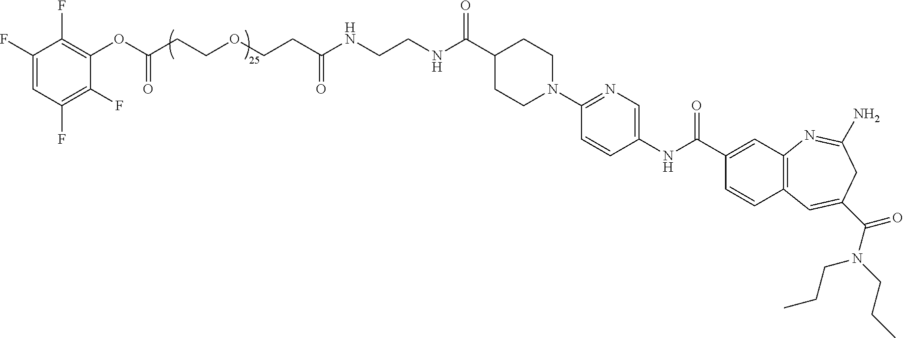 | 1924.2 |
| 8AmBza-L-5 | 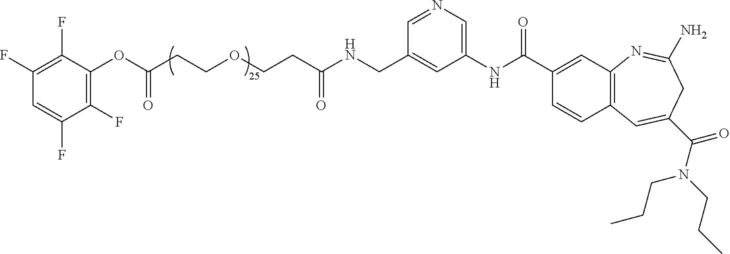 | 1784 |
| 8AmBza-L-6 | 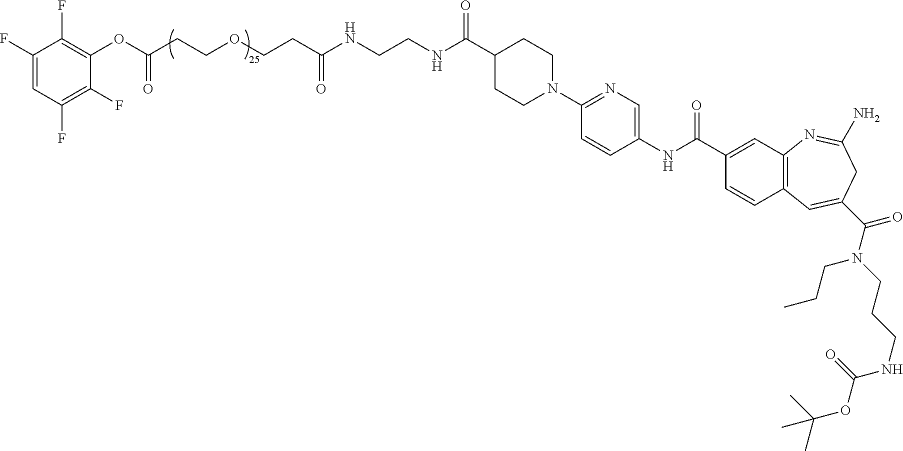 | 2039.3 |
| 8AmBza-L-7 | 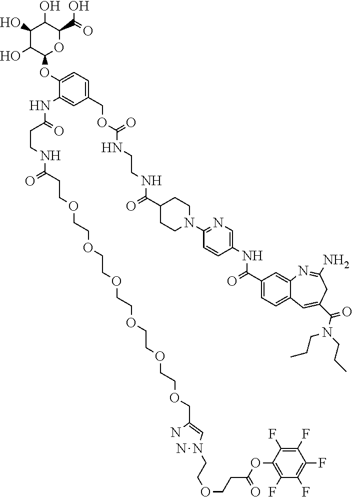 [SMILES: CCCN(CCC)C(=O)C1=Cc2ccc(C(=O)Nc3ccc(N4CCC(C(=O)NCCNC(=O)OCc5ccc(O[C@@H]6O[C@H](C(=O)O)C(O)[C@H](O)C6O)c(NC(=O)CCNC(=O)CCOCCOCCOCCOCCOCCOCc6cn(CCOCCC(=O)Oc7c(F)c(F)c(F)c(F)c7F)nn6)c5)CC4)nc3)cc2N=C(N)C1] | 1642.6 |
| 8AmBza-L-8 | 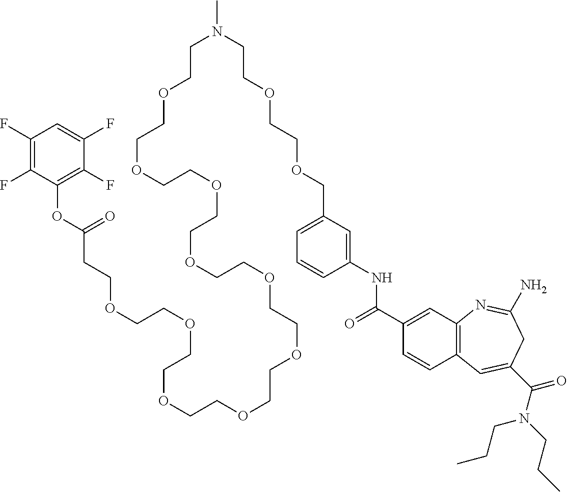 [SMILES: CCCN(CCC)C(=O)C1=Cc2ccc(C(=O)Nc3cccc(COCCOCCN(C)CCOCCOCCOCCOCCOCCOCCOCCOCCOCCOCCC(=O)Oc4c(F)c(F)cc(F)c4F)c3)cc2N=C(N)C1] | 1196.3 |
| 8AmBza-L-9 | 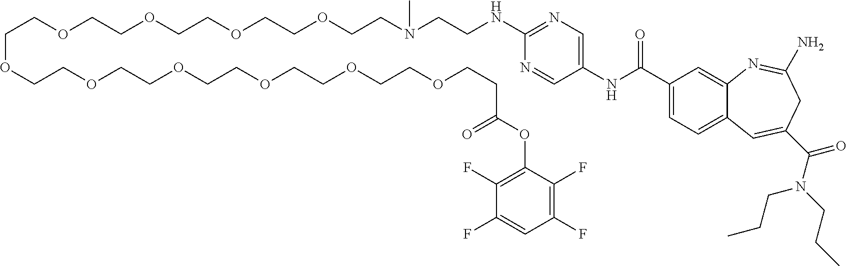 [SMILES: CCCN(CCC)C(=O)C1=Cc2ccc(C(=O)Nc3cnc(NCCN(C)CCOCCOCCOCCOCCOCCOCCOCCOCCOCCOCCC(=O)Oc4c(F)c(F)cc(F)c4F)nc3)cc2N=C(N)C1] | 1139.2 |
| 8AmBza-L-10 | 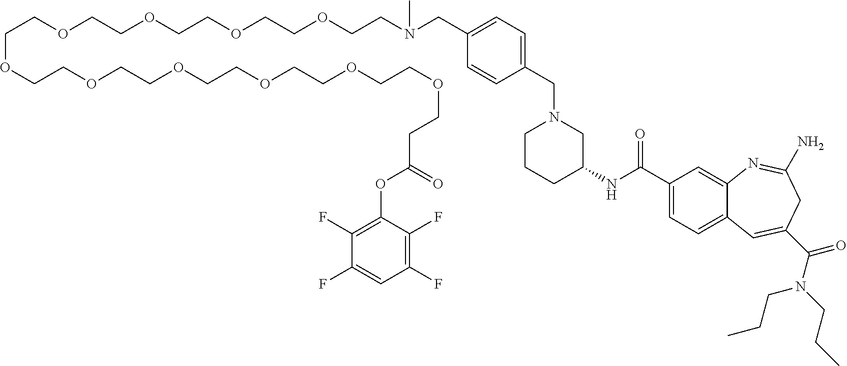 [SMILES: CCCN(CCC)C(=O)C1=Cc2ccc(C(=O)N[C@@H]3CCCN(Cc4ccc(CN(C)CCOCCOCCOCCOCCOCCOCCOCCOCCOCCOCCC(=O)Oc5c(F)c(F)cc(F)c5F)cc4)C3)cc2N=C(N)C1] | 1205.4 |
| 8AmBza-L-11 | 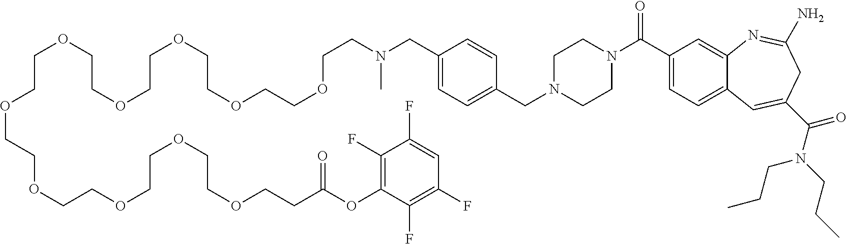 [SMILES: CCCN(CCC)C(=O)C1=Cc2ccc(C(=O)N3CCN(Cc4ccc(CN(C)CCOCCOCCOCCOCCOCCOCCOCCOCCOCCOCCC(=O)Oc5c(F)c(F)cc(F)c5F)cc4)CC3)cc2N=C(N)C1] | 1191.4 |

TABLE 2b
8-Amido-2-aminobenzazepine-linker (8AmBza-L) Formula II compounds

| 8AmBza-L | Structure | MW |
|---|---|---|
| 8AmBza-L-12 | 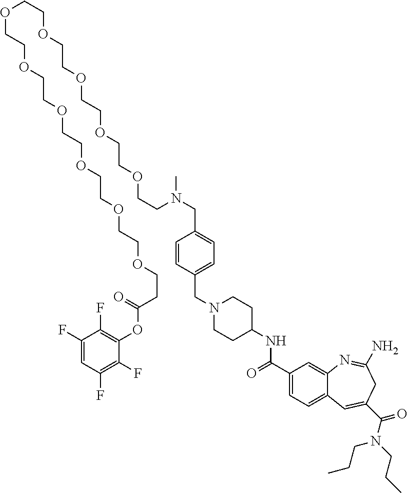 [SMILES: CCCN(CCC)C(=O)C1=Cc2ccc(C(=O)NC3CCN(Cc4ccc(CN(C)CCOCCOCCOCCOCCOCCOCCOCCOCCOCCOCCC(=O)Oc5c(F)c(F)cc(F)c5F)cc4)CC3)cc2N=C(N)C1] | 1205.4 |
| 8AmBza-L-13 | 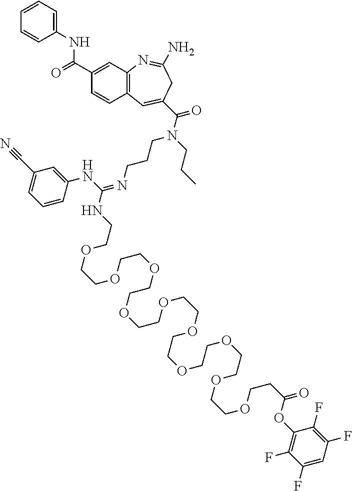 [SMILES: CCCN(CCC/N=C(/NCCOCCOCCOCCOCCOCCOCCOCCOCCOCCOCCC(=O)Oc1c(F)c(F)cc(F)c1F)Nc1cccc(C#N)c1)C(=O)C1=Cc2ccc(C(=O)Nc3ccccc3)cc2N=C(N)C1] | 1223.3 |
| 8AmBza-L-14 | 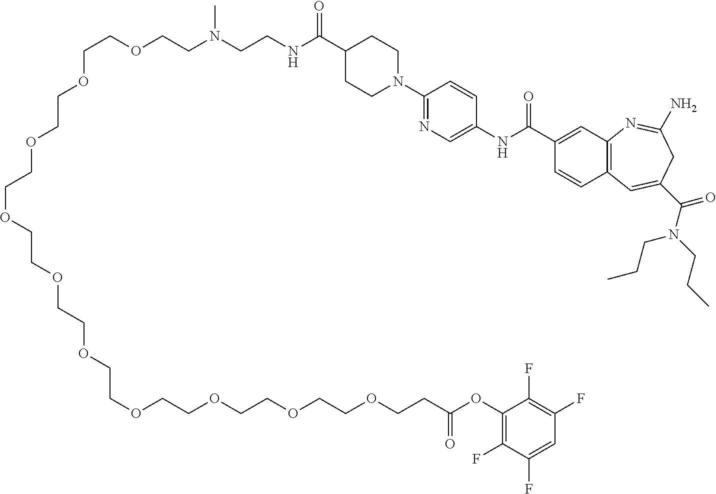 [SMILES: CCCN(CCC)C(=O)C1=Cc2ccc(C(=O)Nc3ccc(N4CCC(C(=O)NCCN(C)CCOCCOCCOCCOCCOCCOCCOCCOCCOCCOCCC(=O)Oc5c(F)c(F)cc(F)c5F)CC4)nc3)cc2N=C(N)C1] | 1249.4 |
| 8AmBza-L-15 | 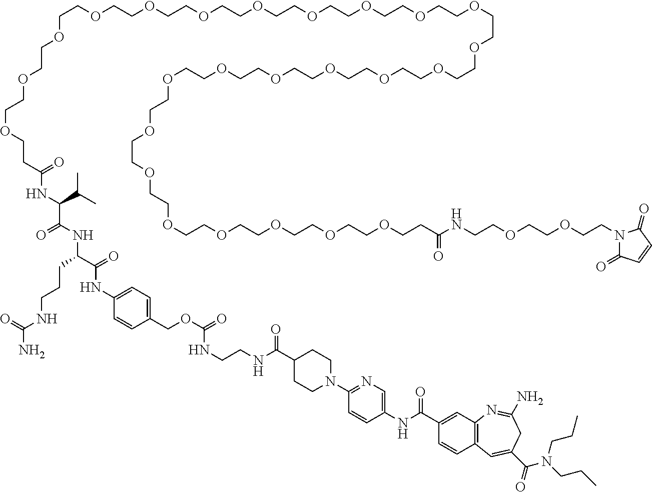 [SMILES: CCCN(CCC)C(=O)C1=Cc2ccc(C(=O)Nc3ccc(N4CCC(C(=O)NCCNC(=O)OCc5ccc(NC(=O)[C@H](CCCNC(N)=O)NC(=O)[C@@H](NC(=O)CCOCCOCCOCCOCCOCCOCCOCCOCCOCCOCCOCCOCCOCCOCCOCCOCCOCCOCCOCCOCCOCCOCCOCCOCCOCCC(=O)NCCOCCOCCN6C(=O)C=CC6=O)C(C)C)cc5)CC4)nc3)cc2N=C(N)C1] | 2391.8 |
| 8AmBza-L-16 | 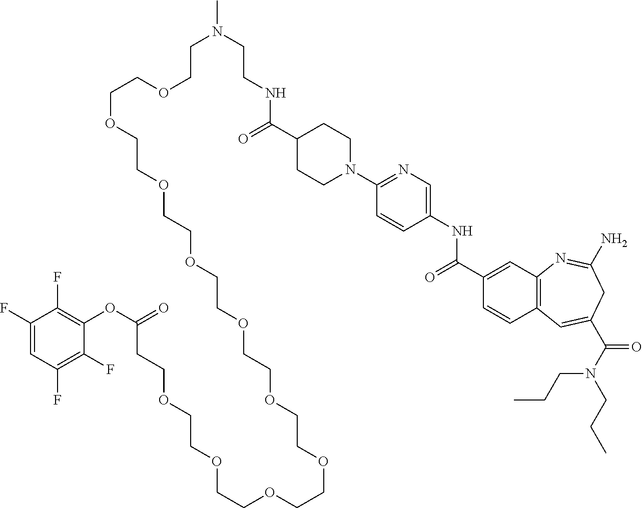 [SMILES: CCCN(CCC)C(=O)C1=Cc2ccc(C(=O)Nc3ccc(N4CCC(C(=O)NCCN(C)CCOCCOCCOCCOCCOCCOCCOCCOCCOCCOCCC(=O)Oc5c(F)c(F)cc(F)c5F)CC4)nc3)cc2N=C(N)C1] | 1251.4 |
| 8AmBza-L-17 | 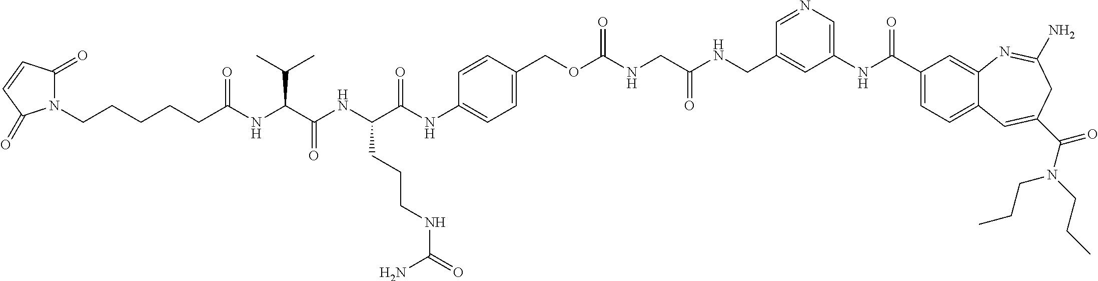 [SMILES: CCCN(CCC)C(=O)C1=Cc2ccc(C(=O)Nc3cncc(CNC(=O)CNC(=O)OCc4ccc(NC(=O)[C@H](CCCNC(N)=O)NC(=O)[C@@H](NC(=O)CCCCCN5C(=O)C=CC5=O)C(C)C)cc4)c3)cc2N=C(N)C1] | 1090.2 |
| 8AmBza-L-18 | 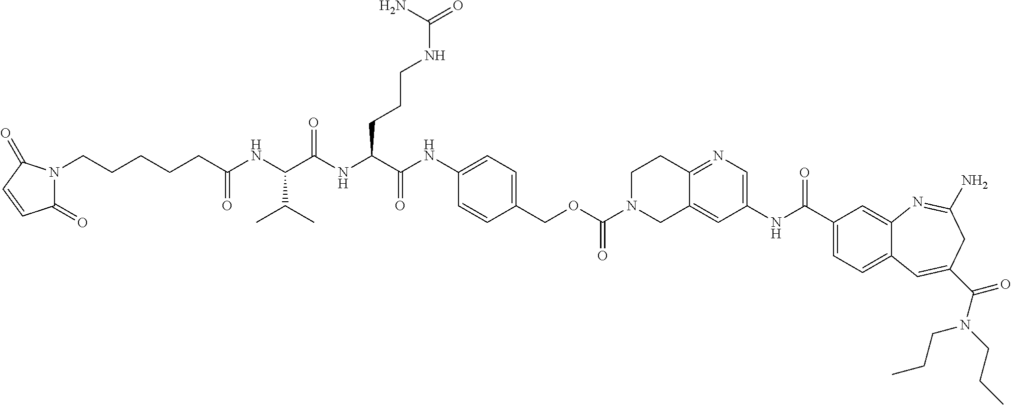 [SMILES: CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1] | 1059.2 |

### Immunoconjugates

Exemplary embodiments of immunoconjugates comprise an antibody covalently attached to one or more 8-amido-2-aminobenzazepine (8AmBza) moieties by a linker, and having Formula:

Ab-[L-8AmBza]ₚ I

or a pharmaceutically acceptable salt thereof,

wherein:

Ab is the antibody;

p is an integer from 1 to 8;

8AmBza is the 8-amido-2-aminobenzazepine moiety having the formula:

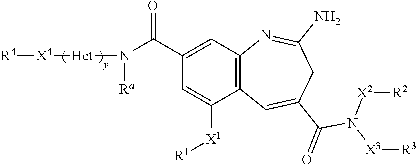

y is 0 or 1;

Het is selected from the group consisting of heterocyclyl, heterocyclyldiyl, heteroaryl, and heteroaryldiyl;

R^{a} is H or forms Het with the nitrogen atom it is bound to;

R¹, R², R³, and R⁴ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
—(C₁-C₁₂ alkyldiyl)-N(R⁵)—*;
—(C₁-C₁₂ alkyldiyl)-N(R⁵)₂;
—(C₁-C₁₂ alkyldiyl)-OR⁵;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁵—*;
—(C₃-C₁₂ carbocyclyl)-(C₁-C₁₂ alkyldiyl)-N(R⁵)₂;
—(C₃-C₁₂ carbocyclyl)-NR—C(═NR⁵)NR⁵—*;
—(C₆-C₂₀ aryl);
—(C₆-C₂₀ aryl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁵)₂;
—(C₆-C₂₀ aryldiyl)-(C₁-C₁₂ alkyldiyl)-NR⁵—C(═NR^{5a})N(R⁵)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁵—*;
—(C₂-C₉ heterocyclyl)-(C₁-C₁₂ alkyldiyl)-N(R⁵)₂;
—(C₂-C₉ heterocyclyl)-NR⁵—C(═NR^{5a})NR⁵—*;
—(C₁-C₂₀ heteroaryl);
—(C₁-C₂₀ heteroaryl)-*;
—(C₁-C₂₀ heteroaryl)-(C₁-C₁₂ alkyldiyl)-N(R⁵)—*;
—(C₁-C₂₀ heteroaryl)-(C₁-C₁₂ alkyldiyl)-N(R⁵)₂;
—(C₁-C₂₀ heteroaryl)-NR⁵—C(═NR^{5a})N(R⁵)—*;
—C(═O)—*;
—C(═O)—(C₁-C₁₂ alkyldiyl)-N(R⁵)—*;
—C(═O)—(C₂-C₂₀ heterocyclyldiyl)-*;
—C(═O)N(R⁵)₂;
—C(═O)N(R⁵)—*;
—C(═O)N(R⁵)—(C₁-C₁₂ alkyldiyl)-N(R⁵)C(═O)R⁵;
—C(═O)N(R⁵)—(C₁-C₁₂ alkyldiyl)-N(R⁵)C(═O)N(R⁵)₂;
—C(═O)NR⁵—(C₁-C₁₂ alkyldiyl)-N(R⁵)CO₂R⁵;
—C(═O)NR⁵—(C₁-C₁₂ alkyldiyl)-N(R⁵)C(═NR^{5a})N(R⁵)₂;
—C(═O)NR⁵—(C₁-C₁₂ alkyldiyl)-NR⁵C(═NR^{5a})R⁵;
—C(═O)NR⁵—(C₁-C₈ alkyldiyl)-NR⁵(C₂-C₅ heteroaryl);
—C(═O)NR⁵—(C₁-C₂₀ heteroaryldiyl)-N(R⁵)—*;
—C(═O)NR⁵—(C₁-C₂₀ heteroaryldiyl)-*;
—C(═O)NR⁵—(C₁-C₂₀ heteroaryldiyl)-(C₁-C₁₂ alkyldiyl)-N(R⁵)₂;
—C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵—*;
—N(R⁵)₂;
—N(R⁵)—*;
—N(R⁵)C(═O)R⁵;
—N(R⁵)C(═O)—*;
—N(R⁵)C(═O)N(R⁵)₂;
—N(R⁵)C(═O)N(R⁵)—*;
—N(R⁵)CO₂R⁵;
—NR⁵C(═NR^{5a})N(R⁵)₂;
—NR⁵C(═NR^{5a})N(R⁵)—*;
—NR⁵C(═NR^{5a})R⁵;
—N(R⁵)—(C₂-C₅ heteroaryl);
—O—(C₁-C₁₂ alkyl);
—O—(C₁-C₁₂ alkyldiyl)-N(R⁵)₂;
—O—(C₁-C₁₂ alkyldiyl)-N(R⁵)—*;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁵)₂;
—S(═O)₂—(C₂-C₂₀ heterocyclyldiyl)-(C₁-C₁₂ alkyldiyl)-NR⁵—*; and
—S(═O)₂—(C₂-C₂₀ heterocyclyldiyl)-(C₁-C₁₂ alkyldiyl)-OH;

or R² and R³ together form a 5- or 6-membered heterocyclyl ring;

X¹, X², X³, and X⁴ are independently selected from the group consisting of a bond, C(═O), C(═O)N(R⁵), O, N(R⁵), S, S(O)₂, and S(O)₂N(R⁵);

R⁵ is selected from the group consisting of H, C₆-C₂₀ aryl, C₆-C₂₀ aryldiyl, C₁-C₁₂ alkyl, and C₁-C₁₂ alkyldiyl, or two R⁵ groups together form a 5- or 6-membered heterocyclyl ring;

R^{5a} is selected from the group consisting of C₆-C₂₀ aryl and C₁-C₂₀ heteroaryl;

where the asterisk * indicates the attachment site of L, and where one of R¹, R², R³ and R⁴ is attached to L;

L is the linker selected from the group consisting of:
—C(═O)-(PEG)-C(═O)-(PEP)-;
—C(═O)-(PEG)-NR⁵—;
—C(═O)-(PEG)-NR⁵-(PEG)-C(═O)-(PEP)-;
—C(═O)-(PEG)-N⁺(R⁵)₂-(PEG)-C(═O)-(PEP)-;
—C(═O)-(PEG)-C(═O)—;
—C(═O)-(PEG)-NR⁵CH(AA₁)C(═O)-(PEG)-C(═O)-(PEP)-;
—C(═O)-(PEG)-SS—(C₁-C₁₂ alkyldiyl)-OC(═O)—;
—C(═O)-(PEG)-SS—(C₁-C₁₂ alkyldiyl)-C(═O)—;
—C(═O)-(PEG)-;
—C(═O)-(PEG)-C(═O)NR⁵(C₁-C₁₂ alkyldiyl)NR⁵C(═O)—(C₂-C₅ monoheterocyclyldiyl)-;
—C(═O)-(PEG)-C(═O)NR⁵(C₁-C₁₂ alkyldiyl)-;
—C(═O)—(C₁-C₁₂ alkyldiyl)-C(═O)-(PEP)-;
—C(═O)—(C₁-C₁₂ alkyldiyl)-C(═O)-(PEP)-NR⁵(C₁-C₁₂ alkyldiyl)-;
—C(═O)—(C₁-C₁₂ alkyldiyl)-C(═O)-(PEP)-NR⁵(C₁-C₁₂ alkyldiyl)NR⁵—C(═O);
—C(═O)—(C₁-C₁₂ alkyldiyl)-C(═O)-(PEP)-NR⁵(C₁-C₁₂ alkyldiyl)NR⁵C(═O)—(C₂-C₅ monoheterocyclyldiyl)-;
—C(═O)—CH₂CH₂OCH₂CH₂—(C₁-C₂₀ heteroaryldiyl)-CH₂O—(PEG)-C(═O)-(MCgluc)-;
—C(═O)—CH₂CH₂OCH₂CH₂—(C₁-C₂₀ heteroaryldiyl)-CH₂O—(PEG)-C(═O)-(MCgluc)-NR⁵(C₁-C₁₂ alkyldiyl)NR⁵C(═O)—(C₂-C₅ monoheterocyclyldiyl)-;
—C(═O)-(PEG)-C(═O)—NR⁵(C₁-C₁₂ alkyldiyl)-;
—C(═O)-(PEG)-C(═O)—NR⁵(C₁-C₁₂ alkyldiyl)NR⁵C(═O)—(C₂-C₅ monoheterocyclyldiyl)-;
—C(═O)-(PEG)-C(═O)-(PEP)-NR⁵(C₁-C₁₂ alkyldiyl)-;
—C(═O)-(PEG)-C(═O)-(PEP)-NR⁵(C₁-C₁₂ alkyldiyl)NR⁵C(═O)—(C₂-C₅ monoheterocyclyldiyl)-; and
-(succinimidyl)-(CH₂)ₘ—C(═O)-(PEP)-NR⁵(C₁-C₁₂ alkyldiyl)NR⁵C(═O)—(C₂-C₅ monoheterocyclyldiyl)-;

PEG has the formula: —(CH₂CH₂O)ₙ—(CH₂)ₘ—; m is an integer from 1 to 5, and n is an integer from 2 to 50;

PEP has the formula:

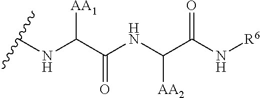

where AA₁ and AA₂ are independently selected from an amino acid side chain, or AA₁ or AA₂ and an adjacent nitrogen atom form a 5-membered ring proline amino acid, and the wavy line indicates a point of attachment and;

R⁶ is selected from the group consisting of C₆-C₂₀ aryldiyl and C₁-C₂₀ heteroaryldiyl, substituted with —CH₂O—C(═O)— and optionally with:

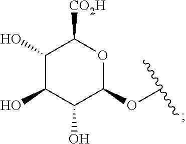 [SMILES: *O[C@@H]1O[C@H](C(=O)O)[C@@H](O)[C@H](O)[C@H]1O]

and

MCgluc is selected from the groups:

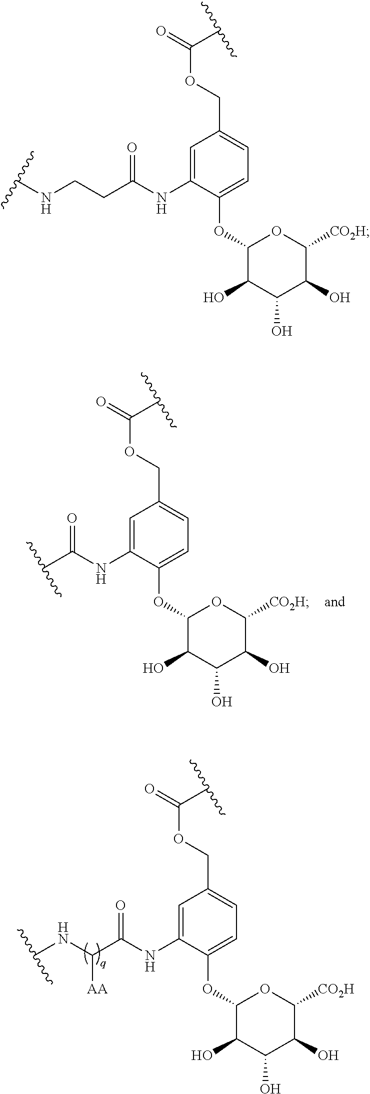

where q is 1 to 8, and AA is an amino acid side chain;

where alkyl, alkyldiyl, alkenyl, alkenyldiyl, alkynyl, alkynyldiyl, aryl, aryldiyl carbocyclyl, carbocyclyldiyl, heterocyclyl, heterocyclyldiyl, heteroaryl, and heteroaryldiyl are optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OP(O)(OH)₂, —S(O)₂N(CH₃)₂, —SCH₃, —S(O)₂CH₃, and —S(O)₃H.

An exemplary embodiment of the immunoconjugate of Formula I includes wherein y is 0.

An exemplary embodiment of the immunoconjugate of Formula I includes wherein y is 1.

An exemplary embodiment of the immunoconjugate of Formula I includes wherein the antibody is an antibody construct that has an antigen binding domain that binds PD-L1.

An exemplary embodiment of the immunoconjugate of Formula I includes wherein the antibody is selected from the group consisting of atezolizumab, durvalumab, and avelumab, or a biosimilar or a biobetter thereof.

An exemplary embodiment of the immunoconjugate of Formula I includes wherein the antibody is an antibody construct that has an antigen binding domain that binds HER2.

An exemplary embodiment of the immunoconjugate of Formula I includes wherein the antibody is selected from the group consisting of trastuzumab and pertuzumab, or a biosimilar or a biobetter thereof.

An exemplary embodiment of the immunoconjugate of Formula I includes wherein the antibody is an antibody construct that has an antigen binding domain that binds CEA.

An exemplary embodiment of the immunoconjugate of Formula I includes wherein the antibody is labetuzumab, or a biosimilar or a biobetter thereof.

An exemplary embodiment of the immunoconjugate of Formula I includes wherein PEP has the formula:

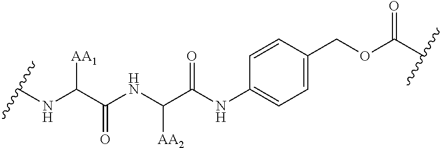

wherein AA₁ and AA₂ are independently selected from a side chain of a naturally-occurring amino acid.

An exemplary embodiment of the immunoconjugate of Formula I includes wherein AA₁ or AA₂ with an adjacent nitrogen atom form a 5-membered ring proline amino acid.

An exemplary embodiment of the immunoconjugate of Formula I includes wherein PEP has the formula:

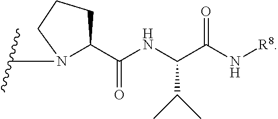

An exemplary embodiment of the immunoconjugate of Formula I includes wherein MCgluc has the formula:

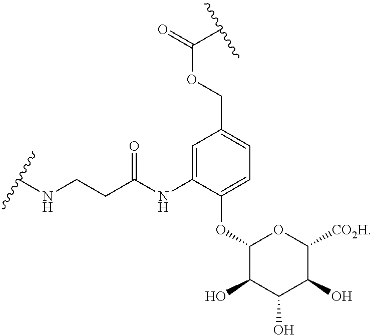 [SMILES: *NCCC(=O)Nc1cc(COC(*)=O)ccc1O[C@@H]1O[C@H](C(=O)O)[C@@H](O)[C@H](O)[C@H]1O]

An exemplary embodiment of the immunoconjugate of Formula I includes wherein AA₁ and AA₂ are independently selected from H, —CH₃, —CH(CH₃)₂, —CH₂(C₆H₅), —CH₂CH₂CH₂CH₂NH₂, —CH₂CH₂CH₂NHC(NH)NH₂, —CHCH(CH₃)CH₃, —CH₂SO₃H, and —CH₂CH₂CH₂NHC(O)NH₂.

An exemplary embodiment of the immunoconjugate of Formula I includes wherein AA₁ is —CH(CH₃)₂, and AA₂ is —CH₂CH₂CH₂NHC(O)NH₂.

An exemplary embodiment of the immunoconjugate of Formula I includes wherein AA₁ and AA₂ are independently selected from GlcNAc aspartic acid, —CH₂SO₃H, and —CH₂OPO₃H.

An exemplary embodiment of the immunoconjugate of Formula I includes wherein X¹ is a bond, and R¹ is H.

An exemplary embodiment of the immunoconjugate of Formula I includes wherein X² is a bond, and R² is C₁-C₈ alkyl.

An exemplary embodiment of the immunoconjugate of Formula I includes wherein X² and X³ are each a bond, and R² and R³ are independently selected from C₁-C₈ alkyl, —O—(C₁-C₁₂ alkyl), —(C₁-C₁₂ alkyldiyl)-OR⁵, —(C₁-C₈ alkyldiyl)-N(R⁵)CO₂R⁵, and —O—(C₁-C₁₂ alkyl)-N(R⁵)CO₂R⁵.

An exemplary embodiment of the immunoconjugate of Formula I includes wherein R² and R³ are each independently selected from —CH₂CH₂CH₃, —OCH₂CH₃, —CH₂CH₂CF₃, and —CH₂CH₂CH₂OH.

An exemplary embodiment of the immunoconjugate of Formula I includes wherein R² is C₁-C₈ alkyl and R³ is —(C₁-C₈ alkyldiyl)-N(R⁵)CO₂R⁴.

An exemplary embodiment of the immunoconjugate of Formula I includes wherein R² is —CH₂CH₂CH₃ and R³ is —CH₂CH₂CH₂NHCO₂(t-Bu).

An exemplary embodiment of the immunoconjugate of Formula I includes wherein R² and R³ are each —CH₂CH₂CH₃.

An exemplary embodiment of the immunoconjugate of Formula I includes wherein X³—R³ is selected from the group consisting of:

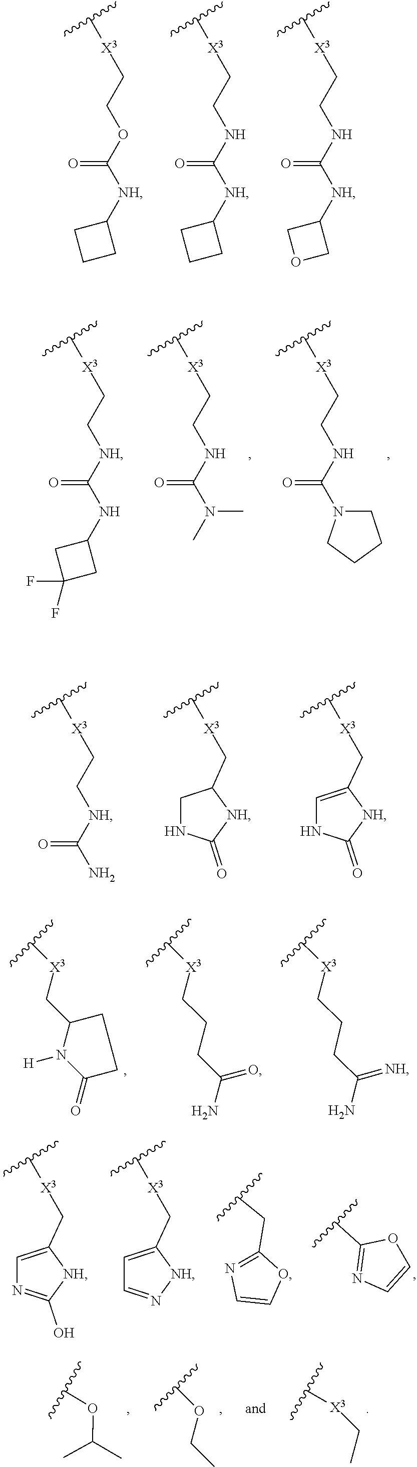

An exemplary embodiment of the immunoconjugate of Formula I includes wherein NR⁵(C₂-C₅ heteroaryl) of R¹ or R³ is selected from:

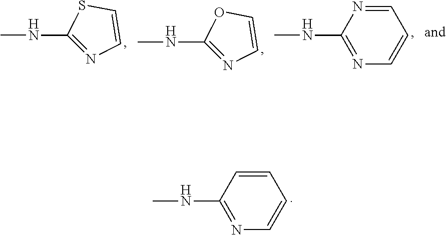

An exemplary embodiment of the immunoconjugate of Formula I includes wherein Het is a 5- or 6-membered monocyclic heteroaryldiyl selected from the group consisting of pyridyldiyl, imidazolyldiyl, pyrimidinyldiyl, pyrazolyldiyl, triazolyldiyl, pyrazinyldiyl, tetrazolyldiyl, furyldiyl, thienyldiyl, isoxazolyldiyldiyl, thiazolyldiyl, oxadiazolyldiyl, oxazolyldiyl, isothiazolyldiyl, and pyrrolyldiyl.

An exemplary embodiment of the immunoconjugate of Formula I includes wherein Het is a 5- or 6-membered monocyclic heterocyclyldiyl selected from the group consisting of morpholinyldiyl, piperidinyldiyl, piperazinyldiyl, pyrrolidinyldiyl, dioxanyldiyl, thiomorpholinyldiyl, and S-dioxothiomorpholinyldiyl.

An exemplary embodiment of the immunoconjugate of Formula I includes wherein Het is 1,6-naphthyridyl or 1,6-naphthyridiyl.

An exemplary embodiment of the immunoconjugate of Formula I includes wherein L is selected from the group consisting of:
—C(═O)—CH₂CH₂OCH₂CH₂—(C₁-C₂₀ heteroaryldiyl)-CH₂O—(PEG)-C(═O)-(MCgluc)-NR⁵(C₁-C₁₂ alkyldiyl)NR⁵C(═O)—(C₂-C₅ monoheterocyclyldiyl)-;
—C(═O)-(PEG)-C(═O)—NR⁵(C₁-C₁₂ alkyldiyl)-;
—C(═O)-(PEG)-C(═O)—NR⁵(C₁-C₁₂ alkyldiyl)NR⁵C(═O)—(C₂-C₅ monoheterocyclyldiyl)-;
—C(═O)-(PEG)-C(═O)-(PEP)-NR⁵(C₁-C₁₂ alkyldiyl)-;
—C(═O)-(PEG)-C(═O)-(PEP)-NR⁵(C₁-C₁₂ alkyldiyl)NR⁵C(═O)—(C₂-C₅ monoheterocyclyldiyl)-; and
-(succinimidyl)-(CH₂)ₘ—C(═O)-(PEP)-NR⁵(C₁-C₁₂ alkyldiyl)NR⁵C(═O)—(C₂-C₅ monoheterocyclyldiyl)-.

An exemplary embodiment of the immunoconjugate of Formula I selected from Formulae Ia-d:

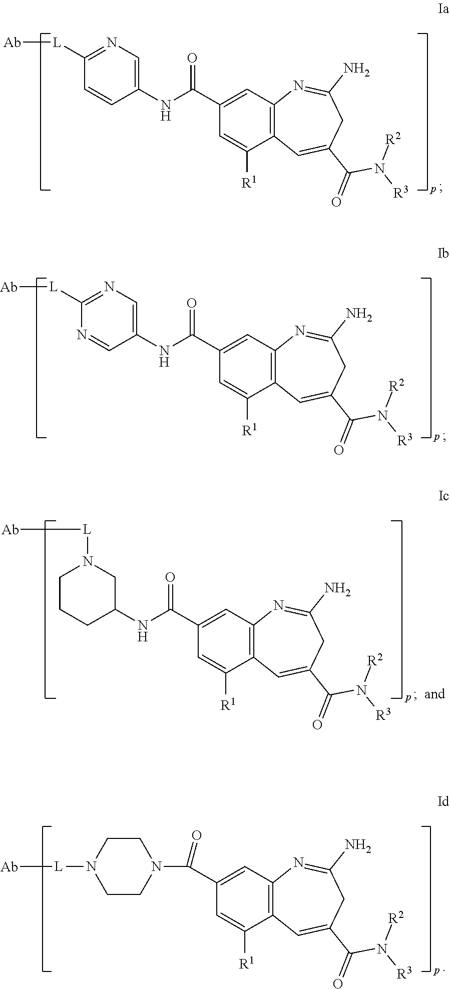

The invention includes all reasonable combinations, and permutations of the features, of the Formula I embodiments.

In certain embodiments, the immunoconjugate compounds of the invention include those with immunostimulatory activity. The antibody-drug conjugates of the invention selectively deliver an effective dose of an 8-amido-2-aminobenzazepine drug to tumor tissue, whereby greater selectivity (i.e., a lower efficacious dose) may be achieved while increasing the therapeutic index (“therapeutic window”) relative to unconjugated 8-amido-2-aminobenzazepine.

Drug loading is represented by p, the number of 8AmBza moieties per antibody in an immunoconjugate of Formula I. Drug (8AmBza) loading may range from 1 to about 8 drug moieties (D) per antibody. Immunoconjugates of Formula I include mixtures or collections of antibodies conjugated with a range of drug moieties, from 1 to about 8. In some embodiments, the number of drug moieties that can be conjugated to an antibody is limited by the number of reactive or available amino acid side chain residues such as lysine and cysteine. In some embodiments, free cysteine residues are introduced into the antibody amino acid sequence by the methods described herein. In such aspects, p may be 1, 2, 3, 4, 5, 6, 7, or 8, and ranges thereof, such as from 1 to 8 or from 2 to 5. In any such aspect, p and n are equal (i.e., p=n=1, 2, 3, 4, 5, 6, 7, or 8, or some range there between). Exemplary antibody-drug conjugates of Formula I include, but are not limited to, antibodies that have 1, 2, 3, or 4 engineered cysteine amino acids (Lyon, R. et al. (2012) Methods in Enzym. 502:123-138). In some embodiments, one or more free cysteine residues are already present in an antibody forming intrachain disulfide bonds, without the use of engineering, in which case the existing free cysteine residues may be used to conjugate the antibody to a drug. In some embodiments, an antibody is exposed to reducing conditions prior to conjugation of the antibody in order to generate one or more free cysteine residues.

For some immunoconjugates, p may be limited by the number of attachment sites on the antibody. For example, where the attachment is a cysteine thiol, as in certain exemplary embodiments described herein, an antibody may have only one or a limited number of cysteine thiol groups, or may have only one or a limited number of sufficiently reactive thiol groups, to which the drug may be attached. In other embodiments, one or more lysine amino groups in the antibody may be available and reactive for conjugation with an 8AmBza-linker compound of Formula II. In certain embodiments, higher drug loading, e.g. p>5, may cause aggregation, insolubility, toxicity, or loss of cellular permeability of certain antibody-drug conjugates. In certain embodiments, the average drug loading for an immunoconjugate ranges from 1 to about 8; from about 2 to about 6; or from about 3 to about 5. In certain embodiments, an antibody is subjected to denaturing conditions to reveal reactive nucleophilic groups such as lysine or cysteine.

The loading (drug/antibody ratio) of an immunoconjugate may be controlled in different ways, and for example, by: (i) limiting the molar excess of the 8AmBza-linker intermediate compound relative to antibody, (ii) limiting the conjugation reaction time or temperature, and (iii) partial or limiting reductive denaturing conditions for optimized antibody reactivity.

It is to be understood that where more than one nucleophilic group of the antibody reacts with a drug, then the resulting product is a mixture of antibody-drug conjugate compounds with a distribution of one or more drug moieties attached to an antibody. The average number of drugs per antibody may be calculated from the mixture by a dual ELISA antibody assay, which is specific for antibody and specific for the drug. Individual immunoconjugate molecules may be identified in the mixture by mass spectroscopy and separated by HPLC, e.g. hydrophobic interaction chromatography (see, e.g., McDonagh et al. (2006) Prot. Engr. Design & Selection 19(7):299-307; Hamblett et al. (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous immunoconjugate with a single loading value may be isolated from the conjugation mixture by electrophoresis or chromatography.

An exemplary embodiment of the immunoconjugate of Formula I is selected from the Table 3a and 3b Immunoconjugates.

TABLE 3a
Immunoconjugates (IC)

| | | | | Myeloid |
|---|---|---|---|---|
| Immuno- | 8AmBza- | | | TNFα |
| conjugate | linker | Ab | | Secretion |
| No. | Table 2a | Antigen | DAR | EC50 [nM] |
| IC-1 | 8AmBza-L-2 | trastuzumab | 2.53 | 273.3 |
| | | HER2 | | |
| IC-2 | 8AmBza-L-3 | trastuzumab | 0.8 | 58.6 |
| | | HER2 | | |
| IC-3 | 8AmBza-L-4 | trastuzumab | 2.49 | >1000 |
| | | HER2 | | |
| IC-4 | 8AmBza-L-5 | trastuzumab | 2.49 | >1000 |
| | | HER2 | | |
| IC-5 | 8AmBza-L-6 | trastuzumab | 1.61 | >1000 |
| | | HER2 | | |
| IC-6 | 8AmBza-L-8 | trastuzumab | 2.24 | 364 |
| | | HER2 | | |
| IC-7 | 8AmBza-L-9 | trastuzumab | 2.50 | NA |
| | | HER2 | | |
| IC-8 | 8AmBza-L-10 | trastuzumab | 2.49 | NA |
| | | HER2 | | |
| IC-9 | 8AmBza-L-11 | trastuzumab | 3.36 | NA |
| | | HER2 | | |

TABLE 3b
Immunoconjugates (IC)

| Immuno- | 8AmBza- | | | Myeloid TNFα |
|---|---|---|---|---|
| conjugate | linker | Ab | | Secretion |
| No. | Table 2b | Antigen | DAR | EC50 [nM] |
| IC-10 | | trastuzumab | 2.26 | NA |
| | | HER2 | | |
| IC-11 | | trastuzumab | 2.24 | NA |
| | | HER2 | | |
| IC-12 | | trastuzumab | 1.91 | NA |
| | | HER2 | | |
| IC-13 | | trastuzumab | 2.30 | NA |
| | | HER2 | | |

### Compositions of Immunoconjugates

The invention provides a composition, e.g., a pharmaceutically or pharmacologically acceptable composition or formulation, comprising a plurality of immunoconjugates as described herein and optionally a carrier therefor, e.g., a pharmaceutically or pharmacologically acceptable carrier. The immunoconjugates can be the same or different in the composition, i.e., the composition can comprise immunoconjugates that have the same number of adjuvants linked to the same positions on the antibody construct and/or immunoconjugates that have the same number of 8AmBza adjuvants linked to different positions on the antibody construct, that have different numbers of adjuvants linked to the same positions on the antibody construct, or that have different numbers of adjuvants linked to different positions on the antibody construct.

In an exemplary embodiment, a composition comprising the immunoconjugate compounds comprises a mixture of the immunoconjugate compounds, wherein the average drug (8AmBza) loading per antibody in the mixture of immunoconjugate compounds is about 2 to about 5.

A composition of immunoconjugates of the invention can have an average adjuvant to antibody construct ratio of about 0.4 to about 10. A skilled artisan will recognize that the number of 8AmBza adjuvants conjugated to the antibody construct may vary from immunoconjugate to immunoconjugate in a composition comprising multiple immunoconjugates of the invention, and, thus, the adjuvant to antibody construct (e.g., antibody) ratio can be measured as an average, which may be referred to as the drug to antibody ratio (DAR). The adjuvant to antibody construct (e.g., antibody) ratio can be assessed by any suitable means, many of which are known in the art.

The average number of adjuvant moieties per antibody (DAR) in preparations of immunoconjugates from conjugation reactions may be characterized by conventional means such as mass spectrometry, ELISA assay, and HPLC. The quantitative distribution of immunoconjugates in a composition in terms of p may also be determined. In some instances, separation, purification, and characterization of homogeneous immunoconjugates where p is a certain value from immunoconjugates with other drug loadings may be achieved by means such as reverse phase HPLC or electrophoresis.

In some embodiments, the composition further comprises one or more pharmaceutically or pharmacologically acceptable excipients. For example, the immunoconjugates of the invention can be formulated for parenteral administration, such as IV administration or administration into a body cavity or lumen of an organ. Alternatively, the immunoconjugates can be injected intra-tumorally. Compositions for injection will commonly comprise a solution of the immunoconjugate dissolved in a pharmaceutically acceptable carrier. Among the acceptable vehicles and solvents that can be employed are water and an isotonic solution of one or more salts such as sodium chloride, e.g., Ringer's solution. In addition, sterile fixed oils can conventionally be employed as a solvent or suspending medium. For this purpose, any bland fixed oil can be employed, including synthetic monoglycerides or diglycerides. In addition, fatty acids such as oleic acid can likewise be used in the preparation of injectables. These compositions desirably are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The composition can contain any suitable concentration of the immunoconjugate. The concentration of the immunoconjugate in the composition can vary widely, and will be selected primarily based on fluid volumes, viscosities, body weight, and the like, in accordance with the particular mode of administration selected and the patient's needs. In certain embodiments, the concentration of an immunoconjugate in a solution formulation for injection will range from about 0.1% (w/w) to about 10% (w/w).

Method of Treating Cancer with Immunoconjugates

The invention provides a method for treating cancer. The method includes administering a therapeutically effective amount of an immunoconjugate as described herein (e.g., as a composition as described herein) to a subject in need thereof, e.g., a subject that has cancer and is in need of treatment for the cancer. The method includes administering a therapeutically effective amount of an immunoconjugate (IC) selected from Table 3.

It is contemplated that the immunoconjugate of the present invention may be used to treat various hyperproliferative diseases or disorders, e.g. characterized by the overexpression of a tumor antigen. Exemplary hyperproliferative disorders include benign or malignant solid tumors and hematological disorders such as leukemia and lymphoid malignancies.

In another aspect, an immunoconjugate for use as a medicament is provided. In certain embodiments, the invention provides an immunoconjugate for use in a method of treating an individual comprising administering to the individual an effective amount of the immunoconjugate. In one such embodiment, the method further comprises administering to the individual an effective amount of at least one additional therapeutic agent, e.g., as described herein.

In a further aspect, the invention provides for the use of an immunoconjugate in the manufacture or preparation of a medicament. In one embodiment, the medicament is for treatment of cancer, the method comprising administering to an individual having cancer an effective amount of the medicament. In one such embodiment, the method further comprises administering to the individual an effective amount of at least one additional therapeutic agent, e.g., as described herein.

Carcinomas are malignancies that originate in the epithelial tissues. Epithelial cells cover the external surface of the body, line the internal cavities, and form the lining of glandular tissues. Examples of carcinomas include, but are not limited to, adenocarcinoma (cancer that begins in glandular (secretory) cells such as cancers of the breast, pancreas, lung, prostate, stomach, gastroesophageal junction, and colon) adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, and skin. In some embodiments, methods for treating non-small cell lung carcinoma include administering an immunoconjugate containing an antibody construct that is capable of binding PD-L1 (e.g., atezolizumab, durvalumab, avelumab, biosimilars thereof, or biobetters thereof). In some embodiments, methods for treating breast cancer include administering an immunoconjugate containing an antibody construct that is capable of binding PD-L1 (e.g., atezolizumab, durvalumab, avelumab, biosimilars thereof, or biobetters thereof). In some embodiments, methods for treating triple-negative breast cancer include administering an immunoconjugate containing an antibody construct that is capable of binding PD-L1 (e.g., atezolizumab, durvalumab, avelumab, biosimilars thereof, or biobetters thereof).

Soft tissue tumors are a highly diverse group of rare tumors that are derived from connective tissue. Examples of soft tissue tumors include, but are not limited to, alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; pleomorphic adenoma of soft tissue; and neoplasias derived from fibroblasts, myofibroblasts, histiocytes, vascular cells/endothelial cells, and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to, askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; and undifferentiated pleomorphic sarcoma).

A teratoma is a type of germ cell tumor that may contain several different types of tissue (e.g., can include tissues derived from any and/or all of the three germ layers: endoderm, mesoderm, and ectoderm), including, for example, hair, muscle, and bone. Teratomas occur most often in the ovaries in women, the testicles in men, and the tailbone in children.

Melanoma is a form of cancer that begins in melanocytes (cells that make the pigment melanin). Melanoma may begin in a mole (skin melanoma), but can also begin in other pigmented tissues, such as in the eye or in the intestines.

Merkel cell carcinoma is a rare type of skin cancer that usually appears as a flesh-colored or bluish-red nodule on the face, head or neck. Merkel cell carcinoma is also called neuroendocrine carcinoma of the skin. In some embodiments, methods for treating Merkel cell carcinoma include administering an immunoconjugate containing an antibody construct that is capable of binding PD-L1 (e.g., atezolizumab, durvalumab, avelumab, biosimilars thereof, or biobetters thereof). In some embodiments, the Merkel cell carcinoma has metastasized when administration occurs.

Leukemias are cancers that start in blood-forming tissue, such as the bone marrow, and cause large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to, Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One category of lymphoma is Hodgkin lymphoma (HL), which is marked by the presence of a type of cell called the Reed-Sternberg cell. There are currently 6 recognized types of HL. Examples of Hodgkin lymphomas include nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to, AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to, gliomas (e.g., glioblastomas, astrocytomas, oligodendrogliomas, ependymomas, and the like), meningiomas, pituitary adenomas, and vestibular schwannomas, primitive neuroectodermal tumors (medulloblastomas).

Immunoconjugates of the invention can be used either alone or in combination with other agents in a therapy. For instance, an immunoconjugate may be co-administered with at least one additional therapeutic agent, such as a chemotherapeutic agent. Such combination therapies encompass combined administration (where two or more therapeutic agents are included in the same or separate formulations), and separate administration, in which case, administration of the immunoconjugate can occur prior to, simultaneously, and/or following, administration of the additional therapeutic agent and/or adjuvant. Immunoconjugates can also be used in combination with radiation therapy.

The immunoconjugates of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Atezolizumab, durvalumab, avelumab, biosimilars thereof, and biobetters thereof are known to be useful in the treatment of cancer, particularly breast cancer, especially triple negative (test negative for estrogen receptors, progesterone receptors, and excess HER2 protein) breast cancer, bladder cancer, and Merkel cell carcinoma. The immunoconjugate described herein can be used to treat the same types of cancers as atezolizumab, durvalumab, avelumab, biosimilars thereof, and biobetters thereof, particularly breast cancer, especially triple negative (test negative for estrogen receptors, progesterone receptors, and excess HER2 protein) breast cancer, bladder cancer, and Merkel cell carcinoma.

The immunoconjugate is administered to a subject in need thereof in any therapeutically effective amount using any suitable dosing regimen, such as the dosing regimens utilized for atezolizumab, durvalumab, avelumab, biosimilars thereof, and biobetters thereof. For example, the methods can include administering the immunoconjugate to provide a dose of from about 100 ng/kg to about 50 mg/kg to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

In another aspect, the invention provides a method for preventing cancer. The method comprises administering a therapeutically effective amount of an immunoconjugate (e.g., as a composition as described above) to a subject. In certain embodiments, the subject is susceptible to a certain cancer to be prevented. For example, the methods can include administering the immunoconjugate to provide a dose of from about 100 ng/kg to about 50 mg/kg to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

Some embodiments of the invention provide methods for treating cancer as described above, wherein the cancer is breast cancer. Breast cancer can originate from different areas in the breast, and a number of different types of breast cancer have been characterized. For example, the immunoconjugates of the invention can be used for treating ductal carcinoma in situ; invasive ductal carcinoma (e.g., tubular carcinoma; medullary carcinoma; mucinous carcinoma; papillary carcinoma; or cribriform carcinoma of the breast); lobular carcinoma in situ; invasive lobular carcinoma; inflammatory breast cancer; and other forms of breast cancer such as triple negative (test negative for estrogen receptors, progesterone receptors, and excess HER2 protein) breast cancer. In some embodiments, methods for treating breast cancer include administering an immunoconjugate containing an antibody construct that is capable of binding HER2 (e.g. trastuzumab, pertuzumab, biosimilars, or biobetters thereof) and PD-L1 (e.g., atezolizumab, durvalumab, avelumab, biosimilars, or biobetters thereof). In some embodiments, methods for treating colon cancer lung cancer, renal cancer, pancreatic cancer, gastric cancer, and esophageal cancer include administering an immunoconjugate containing an antibody construct that is capable of binding CEA, or tumors over-expressing CEA (e.g. labetuzumab, biosimilars, or biobetters thereof).

In some embodiments, the cancer is susceptible to a pro-inflammatory response induced by TLR7 and/or TLR8.

### EXAMPLES

### Preparation of 8-amido-2-aminobenzazepine Compounds (8AmBza) and Intermediates

### Example 1 Synthesis of tert-butyl ((5-(2-amino-4-(dipropylcarbamoyl)-3H-benzo[b]azepine-8-carboxamido)pyridin-3-yl)methyl)carbamate, 8AmBza-1

8AmBza-1 was prepared and characterized according to the procedures described herein.

### Example 2 Synthesis of tert-butyl (3-(8-((6-(4-((2-acetamidoethyl)carbamoyl)piperidin-1-yl)pyridin-3-yl)carbamoyl)-2-amino-N-propyl-3H-benzo[b]azepine-4-carboxamido)propyl)carbamate, 8AmBza-2

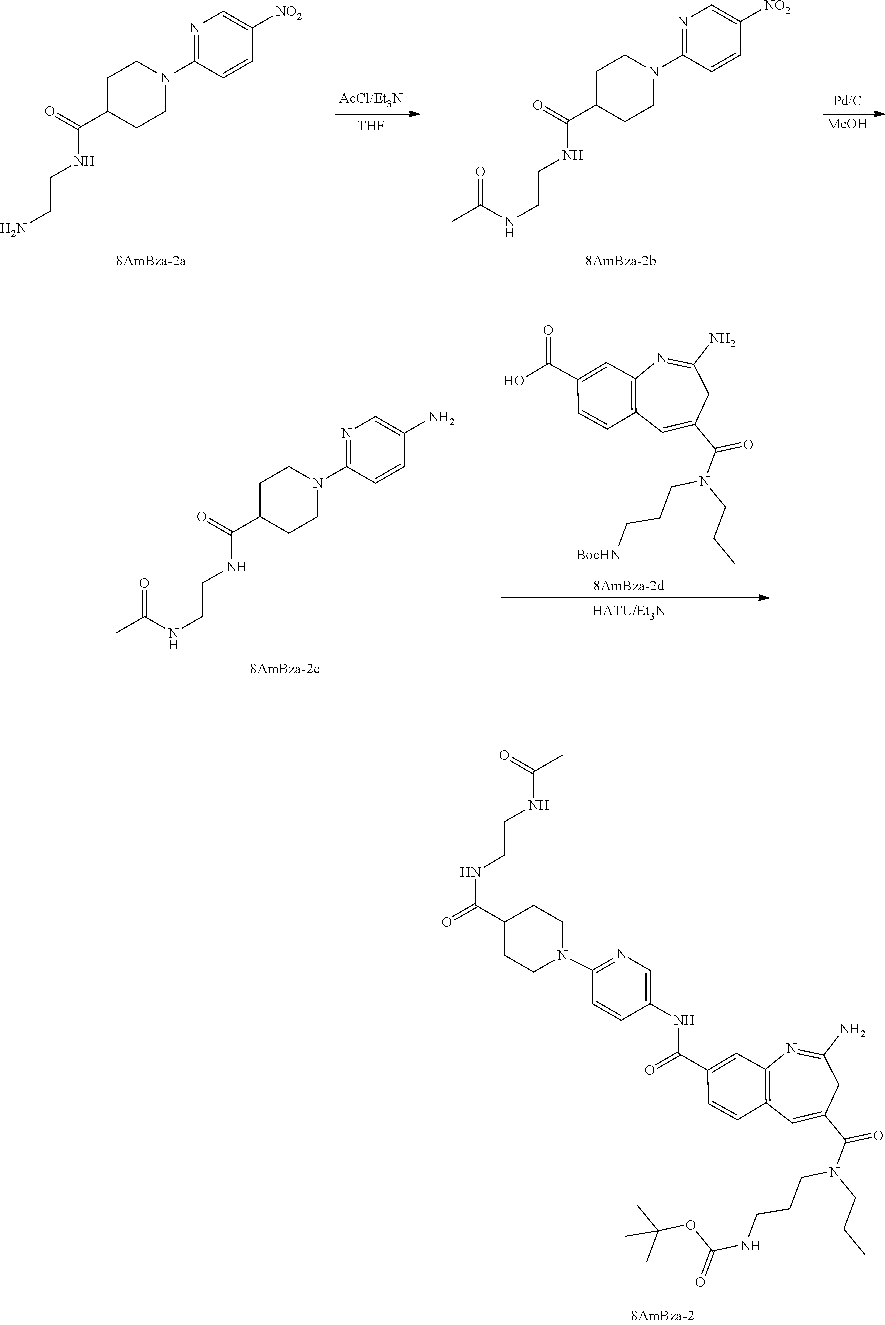

### Preparation of N-(2-acetamidoethyl)-1-(5-nitropyridin-2-yl) piperidine-4-carboxamide, 8AmBza-2b

To a mixture of acetyl chloride (142.82 mg, 1.82 mmol, 129.83 μL, 3 eq) and N-(2-aminoethyl)-1-(5-nitro-2-pyridyl)piperidine-4-carboxamide, 8AmBza-2a (0.2 g, 606.46 μmol, 1 eq, HCl) in THF (10 mL) was added Et₃N (245.47 mg, 2.43 mmol, 337.65 μL, 4 eq) at 25° C. under N₂. The mixture was stirred at 25° C. for 1 hour. LCMS showed the reaction was completed. The mixture was pour into water (20 mL). The mixture was filtered to give 8AmBza-2b (0.2 g, 596.38 μmol, 98.34% yield) as a yellow solid. ¹H NMR (DMSO-d₆, 400 MHz) δ 8.95 (d, J=2.4 Hz, 1H), 8.19 (dd, J=9.6, 2.4 Hz, 1H), 7.78-7.98 (m, 2H), 6.95 (d, J=9.6 Hz, 1H), 4.50 (d, J=9.6 Hz, 2H), 2.93-3.15 (m, 7H), 1.73-1.80 (m, 5H), 1.43-1.62 (m, 2H), 1.07-1.28 (m, 3H).

### Preparation of N-(2-acetamidoethyl)-1-(5-aminopyridin-2-yl) piperidine-4-carboxamide, 8AmBza-2c

To a solution of N-(2-acetamidoethyl)-1-(5-nitro-2-pyridyl)piperidine-4-carboxamide, 8AmBza-2b (0.2, 596.38 μmol, 1 eq) in MeOH (20 mL) was added Pd/C (0.2 g, 5% purity) under N₂. The suspension was degassed under vacuum and purged with H₂ several times. The mixture was stirred under H₂ (15 psi) at 25° C. for 4 hours. LCMS showed the reaction was completed. The mixture was filtered and concentrated to give 8AmBza-2c (0.18 g, 589.44 μmol, 98.84% yield) as yellow solid.

### Preparation of tert-butyl (3-(8-((6-(4-((2-acetamidoethyl)carbamoyl)piperidin-1-yl)pyridin-3-yl)carbamoyl)-2-amino-N-propyl-3H-benzo[b]azepine-4-carboxamido)propyl)carbamate, 8AmBza-2

To a mixture of 2-amino-4-[3-(tert-butoxycarbonylamino) propyl-propyl-carbamoyl]-3H-1-benzazepine-8-carboxylic acid, 8AmBza-2d (0.22 g, 494.91 μmol, 1 eq) 1-[Bis(dimethylamino)methylene]-1H-1,2,3-triazolo[4,5-b]pyridinium 3-oxide hexafluorophosphate, Hexafluorophosphate Azabenzotriazole Tetramethyl Uronium, HATU, CAS Reg. No. 148893-10-1 (225.82 mg, 593.90 μmol, 1.2 eq) in DMF (5 mL) was added Et₃N (150.24 mg, 1.48 mmol, 206.66 μL, 3 eq) at 25° C. The mixture was stirred at 25° C. for 5 min, then N-(2-acetamidoethyl)-1-(5-amino-2-pyridyl)piperidine-4-carboxamide, 8AmBza-2c (151.13 mg, 494.91 μmol, 1 eq) was added to the mixture, stirred for 30 min. The mixture was poured into water (50 mL). The aqueous phase was extracted with ethyl acetate (50 mL*1). The combined organic phase was washed with brine (50 mL*1), dried with anhydrous Na₂SO₄, filtered and concentrated in vacuum. The residue was purified by prep-HPLC column: Welch Xtimate C18 150*25 mm*5 um; mobile phase: [water (10 mM NH4HCO3)-ACN]; B %: 30%-50%, 10.5 min to afford 8AmBza-2 (96 mg, 131.17 μmol, 26.50% yield) as an off-white solid. ¹H NMR (MeOD, 400 MHz) δ 8.39 (d, J=2.6 Hz, 1H), 7.90 (dd, J=9.2, 2.6 Hz, 1H), 7.69 (d, J=1.2 Hz, 1H), 7.54-7.60 (m, 1H), 7.46 (br d, J=8.0 Hz, 1H), 6.85-6.95 (m, 2H), 4.30 (d, J=13.6 Hz, 2H), 3.39-3.53 (m, 4H), 3.28 (s, 2H), 3.08-3.12 (m, 2H), 2.83-2.93 (m, 2H), 2.37-2.47 (m, 1H), 1.94 (s, 3H), 1.60-1.90 (m, 8H), 1.24-1.50 (m, 9H). LC/MS [M+H] 732.42 (calculated); LC/MS [M+H] 732.40 (observed).

### Example 3 Synthesis of 2-amino-N8-(6-(4-((2-aminoethyl)carbamoyl)piperidin-1-yl)pyridin-3-yl)-N4,N4-dipropyl-3H-benzo[b]azepine-4,8-dicarboxamide, 8AmBza-3

8AmBza-3 was prepared and characterized according to the procedures described herein.

### Example 4 Synthesis of 4-((S)-2-((S)-2-amino-3-methylbutanamido)-5-ureidopentanamido)benzyl ((5-(2-amino-4-(dipropylcarbamoyl)-3H-benzo[b]azepine-8-carboxamido)pyridin-3-yl)methyl)carbamate, 8AmBza-4

8AmBza-4 was prepared and characterized according to the procedures described herein.

### Example 5 Synthesis of tert-butyl (3-(2-amino-8-((6-(4-((2-aminoethyl)carbamoyl)piperidin-1-yl)pyridin-3-yl)carbamoyl)-N-propyl-3H-benzo[b]azepine-4-carboxamido)propyl)carbamate, 8AmBza-5

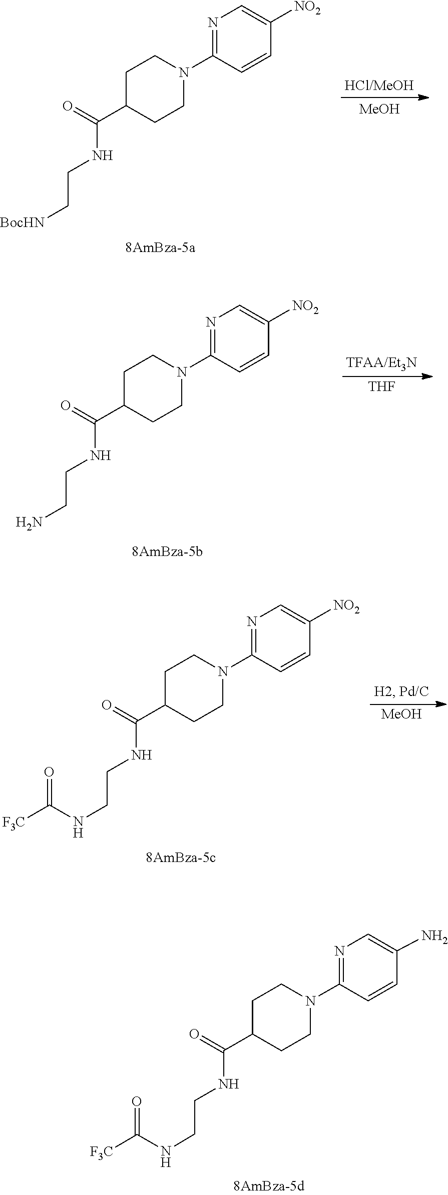

### Preparation of N-(2-aminoethyl)-1-(5-nitropyridin-2-yl)piperidine-4-carboxamide, 8AmBza-5b

To a mixture of tert-butyl N-[2-[[1-(5-nitro-2-pyridyl)piperidine-4-carbonyl]amino]ethyl]carbamate, 8AmBza-5a (0.5 g, 1.27 mmol, 1 eq) in EtOAc (10 mL) was added HCl/EtOAc (4 M, 3.18 mL, 10 eq) at 25° C. The mixture was stirred at 25° C. for 2 hours. LCMS showed the reaction was completed. The reaction was concentrated in vacuum to give 8AmBza-5b (0.4 g, 1.21 mmol, 95.44% yield, HCl) as a yellow solid.

### Preparation of 1-(5-nitropyridin-2-yl)-N-(2-(2,2,2-trifluoroacetamido) ethyl)piperidine-4-carboxamide, 8AmBza-5c

To a mixture of N-(2-aminoethyl)-1-(5-nitro-2-pyridyl)piperidine-4-carboxamide, 8AmBza-5b (0.4 g, 1.21 mmol, 1 eq, HCl) in THF (10 mL) was added Et₃N (368.21 mg, 3.64 mmol, 506.47 μL, 3 eq) and (2,2,2-trifluoroacetyl) 2,2,2-trifluoroacetate (382.13 mg, 1.82 mmol, 253.06 μL, 1.5 eq) at 25° C. The mixture was stirred at 25° C. for 1 hours. LCMS showed major as desired. The mixture was poured into water (50 mL). The aqueous phase was extracted with ethyl acetate (30 mL*3). The combined organic phase was washed with brine (30 mL*1), dried with anhydrous Na₂SO₄, filtered and concentrated in vacuum. The residue was used to next step directly, containing 8AmBza-5c (0.4 g, 1.03 mmol, 84.71% yield) as a yellow solid. ¹H NMR (DMSO-d₆, 400 MHz) δ 9.37-9.45 (m, 1H), 8.95 (d, J=2.8 Hz, 1H), 8.19 (dd, J=9.6, 2.8 Hz, 1H), 8.03 (br t, J=5.2 Hz, 1H), 6.96 (d, J=9.6 Hz, 1H), 4.47-4.53 (m, 2H), 2.99-3.25 (m, 6H), 2.38-2.47 (m, 3H), 1.73-1.80 (m, 2H), 1.41-1.58 (m, 2H)

### Preparation of 1-(5-aminopyridin-2-yl)-N-(2-(2,2,2-trifluoroacetamido) ethyl)piperidine-4-carboxamide, 8AmBza-5d

To a solution of 1-(5-nitro-2-pyridyl)-N-[2-[(2,2,2-trifluoroacetyl)amino]ethyl]piperidine-4-carboxamide, 8AmBza-5c (0.4 g, 1.03 mmol, 1 eq) in MeOH (30 mL) was added Pd/C (0.5 g, 5% purity) under N₂. The suspension was degassed under vacuum and purged with H₂ several times. The mixture was stirred under H₂ (50 psi) at 25° C. for 2 hours. TLC showed the reaction was completed. The mixture was filtered and concentrated in vacuum to give 8AmBza-5d (0.3 g, 834.85 μmol, 81.26% yield) as a gray solid. ¹H NMR (DMSO-d₆, 400 MHz) δ 9.39-9.46 (m, 1H), 7.97 (t, J=5.2 Hz, 1H), 7.59 (d, J=2.8 Hz, 1H), 6.90 (dd, J=8.8, 2.8 Hz, 1H), 6.64 (d, J=8.8 Hz, 1H), 3.99 (d, J=12.8 Hz, 2H), 3.15-3.26 (m, 6H), 2.54-2.63 (m, 2H), 2.16-2.26 (m, 1H), 1.65-1.71 (m, 2H), 1.48-1.60 (m, 2H)

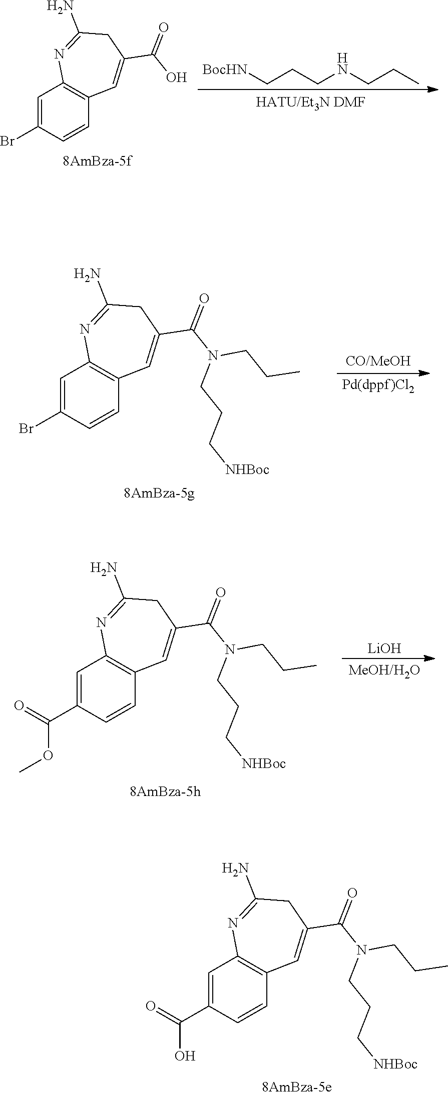

### Preparation of tert-butyl(3-(2-amino-8-bromo-N-propyl-3H-benzo[b]azepine-4-carboxamido)propyl)carbamate, 8AmBza-5g

To a mixture of 2-amino-8-bromo-3H-1-benzazepine-4-carboxylic acid, 8AmBza-5f (4.09 g, 14.56 mmol, 1 eq) and tert-butyl N-[3-(propylamino)propyl]carbamate (3.78 g, 17.47 mmol, 1.2 eq) in DMF (10 mL) was added HATU (6.64 g, 17.47 mmol, 1.2 eq) and Et₃N (2.95 g, 29.12 mmol, 4.05 mL, 2 eq) in one portion at 25 C. The mixture was stirred at 25° C. for 1 h. LCMS showed the reaction was finished. The mixture was diluted with water and extracted with EtOAc (50 mL×3). The organic layer was washed with brine, dried over Na₂SO₄, filtered and concentrated. The residue was purified by silica gel chromatography (column height: 250 mm, diameter: 100 mm, 100-200 mesh silica gel, Petroleum ether/Ethyl acetate=1/0, 0/1) to afford 8AmBza-5g (6 g, 12.52 mmol, 85.95% yield) as a yellow oil.

### Preparation of methyl 2-amino-4-[3-(tert-butoxycarbonylamino)propyl-propyl-carbamoyl]-3H-1-benzazepine-8-carboxylate, 8AmBza-5h

To a solution of tert-butyl N-[3-[(2-amino-8-bromo-3H-1-benzazepine-4-carbonyl)-propyl-amino]propyl] carbamate, Bz-39g (5 g, 10.43 mmol, 1 eq) in MeOH (50 mL) was added Et₃N (3.17 g, 31.29 mmol, 4.35 mL, 3 eq) and [1,1′-bis(diphenylphosphino)ferrocene]dichloropalladium(II), Pd(dppf)Cl₂, CAS Reg. No. 72287-26-4 (763.13 mg, 1.04 mmol, 0.1 eq) under N₂. The suspension was degassed under vacuum and purged with CO (10.43 mmol, 1 eq) several times. The mixture was stirred under CO (50 psi) at 80° C. for 12 hours. LCMS showed the reaction was finished. The mixture was filtered and concentrated to give 8AmBza-5h (7 g, crude) as yellow oil.

### Preparation of 2-amino-4-((3-((tert-butoxycarbonyl)amino)propyl)(propyl)carbamoyl)-3H-benzo[b]azepine-8-carboxylic acid, 8AmBza-5e

To a mixture of methyl 2-amino-4-[3-(tert-butoxycarbonylamino)propyl-propyl-carbamoyl]-3H-1-benzazepine-8-carboxylate, Bz-39h (6 g, 13.08 mmol, 1 eq) in MeOH (80 mL) was added LiOH (1.25 g, 52.34 mmol, 4 eq) in one portion at 30° C. The mixture was stirred at 30° C. for 12 h. LCMS showed the reaction was finished. The mixture was adjusted pH 6 with aq (aqueous) HCl (1 M) at 25° C. The mixture was concentrated. The mixture was further purification by pre-HPLC (column: Phenomenex Luna® C18 250*50 mm*10 um; mobile phase: [water (0.1% TFA)-ACN]; B %: 10%-40%, 20 min) to give 8AmBza-5e (1.4 g, 3.09 mmol, 23.64% yield, 98.23% purity) as yellow oil. ¹H NMR (MeOD, 400 MHz) δ 8.06 (d, J=1.2 Hz, 1H), 8.02 (dd, J=1.6, 8.0 Hz, 1H), 7.68 (s, 1H), 7.14 (s, 1H), 3.58-3.44 (m, 4H), 3.37 (s, 2H), 3.10 (m, 2H), 1.85 (m, 2H), 1.71 (m, 2H), 1.51-1.33 (m, 9H), 0.92-0.98 (m, 3H). LC/MS [M+H] 445.25 (calculated); LC/MS [M+H] 445.10 (observed).

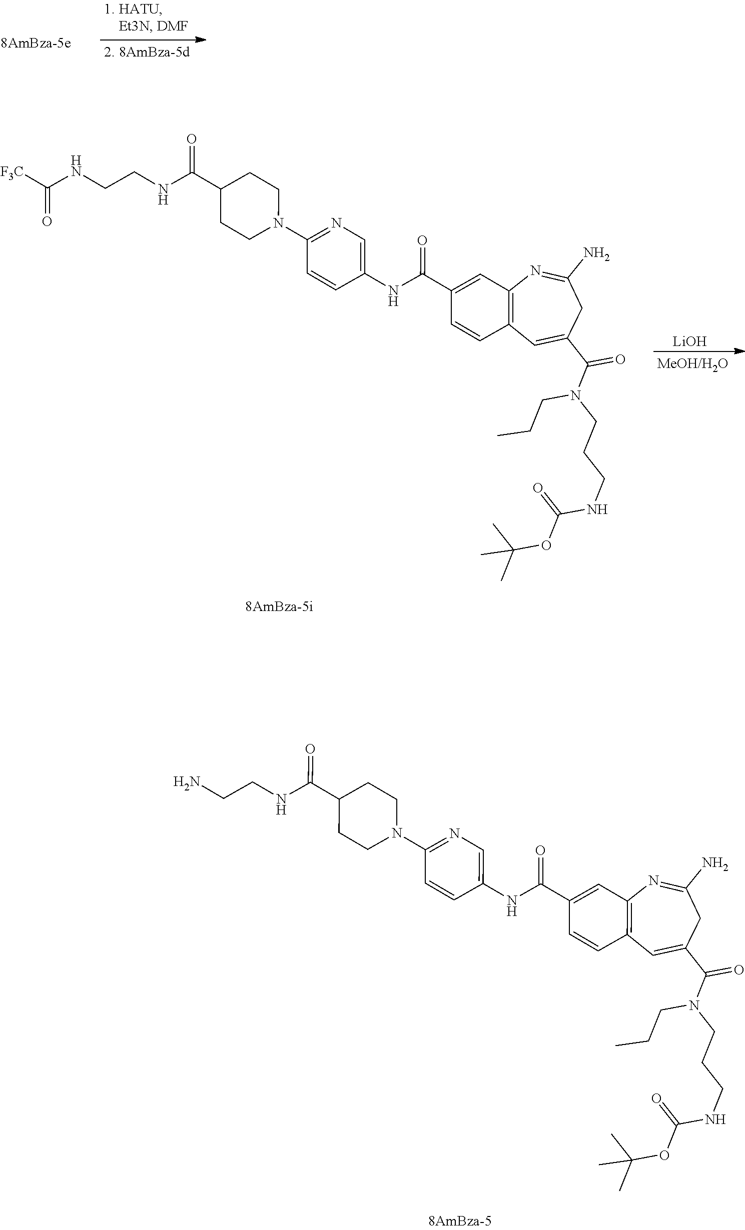

### Preparation of tert-butyl (3-(2-amino-N-propyl-8-((6-(4-((2-(2,2,2-trifluoroacetamido)ethyl)carbamoyl)piperidin-1-yl)pyridin-3-yl)carbamoyl)-3H-benzo[b]azepine-4-carboxamido)propyl)carbamate, 8AmBza-5i

To a mixture of 2-amino-4-[3-(tert-butoxycarbonylamino)propyl-propyl-carbamoyl]-3H-1-benzazepine-8-carboxylic acid, 8AmBza-5e (200 mg, 449.92 μmol, 1 eq) HATU (205.29 mg, 539.90 μmol, 1.2 eq) in DMF (3 mL) was added Et₃N (136.58 mg, 1.35 mmol, 187.87 μL, 3 eq) at 25° C. The mixture was stirred at 25° C. for 5 min, then 1-(5-amino-2-pyridyl)-N-[2-[(2,2,2-trifluoroacetyl)amino]ethyl]piperidine-4-carboxamide, 8AmBza-5d (161.68 mg, 449.92 μmol, 1 eq) was added to the mixture, stirred for 30 min. LCMS showed major as desired. The mixture was poured into water (50 mL). The aqueous phase was extracted with ethyl acetate (50 mL*1). The combined organic phase was washed with brine (50 mL), dried with anhydrous Na₂SO₄, filtered and concentrated in vacuum to give 8AmBza-5i (0.3 g, 381.75 μmol, 84.85% yield) as yellow oil.

### Preparation of tert-butyl (3-(2-amino-8-((6-(4-((2-aminoethyl)carbamoyl)piperidin-1-yl)pyridin-3-yl)carbamoyl)-N-propyl-3H-benzo[b]azepine-4-carboxamido)propyl)carbamate, 8AmBza-5

To a mixture of tert-butyl N-[3-[[2-amino-8-[[6-[4-[2-[(2,2,2-trifluoroacetyl) amino]ethylcarbamoyl]-1-piperidyl]-3-pyridyl]carbamoyl]-3H-1-benzazepine-4-carbonyl]-propyl-amino]propyl]carbamate, 8AmBza-5i (0.25 g, 318.13 μmol, 1 eq) in MeOH (10 mL) was added LiOH.H₂O (40.05 mg, 954.38 μmol, 3 eq) in H₂O (1 mL) at 25° C. The mixture was stirred at 40° C. for 12 hours. LCMS showed major as desired. The mixture was concentrated in vacuum. The residue was purified by prep-HPLC column: Nano-micro Kromasil C18 100*30 mm 5 um; mobile phase: [water (0.1% TFA)-ACN]; B %: 15%-45%, 10 min to give 8AmBza-5 (45 mg, 65.23 μmol, 20.51% yield) as a white solid. ¹H NMR (MeOD, 400 MHz) δ 8.73 (d, J=2.4 Hz, 1H), 8.24 (dd, J=9.8, 2.4 Hz, 1H), 7.75 (br s, 1H), 7.45 (d, J=9.8 Hz, 1H), 7.15 (br s, 1H), 4.24 (br d, J=13.6 Hz, 2H), 3.35-3.62 (m, 9H), 3.05-3.12 (m, 4H), 2.59-2.72 (m, 1H), 1.99-2.09 (m, 2H), 1.65-1.94 (m, 6H), 1.45 (s, 9H), 0.90-0.98 (m, 3H). LC/MS [M+H] 690.41 (calculated); LC/MS [M+H] 690.40 (observed).

### Example 6 Synthesis of tert-butyl N-[3-[[2-amino-8-[[6-[4-[2-[(2,2,2-trifluoroacetyl)amino]ethylcarbamoyl]-1-piperidyl]-3-pyridyl] carbamoyl]-3H-1-benzazepine-4-carbonyl]-propyl-amino]propyl]carbamate, 8AmBza-6

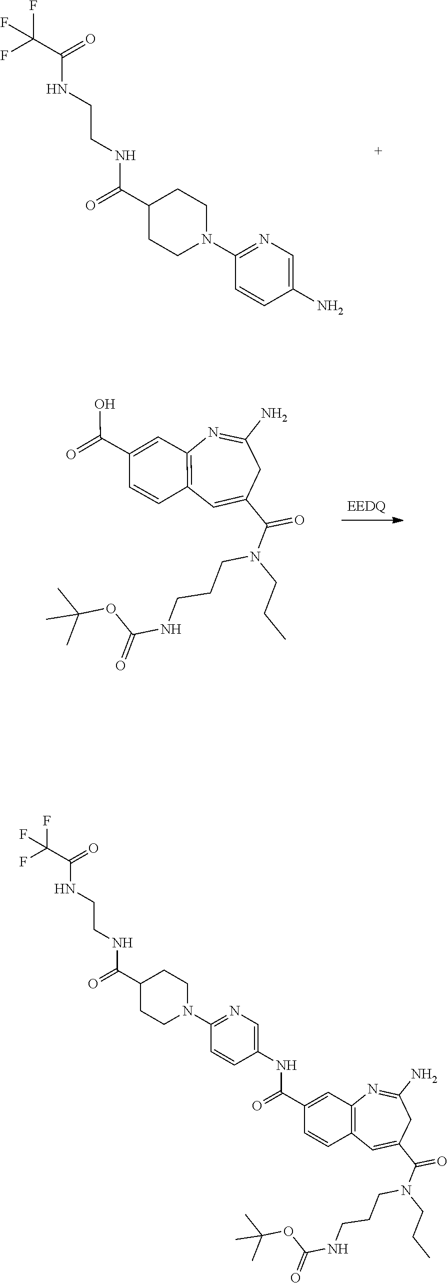 [SMILES: Nc1ccc(N2CCC(C(=O)NCCNC(=O)C(F)(F)F)CC2)nc1 ; CCCN(CCCNC(=O)OC(C)(C)C)C(=O)C1=Cc2ccc(C(=O)O)cc2N=C(N)C1 ; CCCN(CCCNC(=O)OC(C)(C)C)C(=O)C1=Cc2ccc(C(=O)Nc3ccc(N4CCC(C(=O)NCCNC(=O)C(F)(F)F)CC4)nc3)cc2N=C(N)C1]

To a mixture of 2-amino-4-[3-(tert-butoxycarbonylamino) propyl-propyl-carbamoyl]-3H-1-benzazepine-8-carboxylic acid (0.43 g, 976 μmol, 1.0 eq) and 1-(5-amino-2-pyridyl)-N-[2-[(2,2,2-trifluoroacetyl)amino]ethyl]piperidine-4-carboxamide (526.26 mg, 1.46 mmol, 1.5 eq) in MeOH (2 mL) and DCM (4 mL) was added N-Ethoxycarbonyl-2-ethoxy-1,2-dihydroquinoline, EEDQ (362 mg, 1.46 mmol, 1.5 eq) at 25° C. and stirred for 12 hours at this temperature. The mixture was then concentrated under reduced pressure, and the residue was purified by column chromatography (SiO₂, Petroleum ether/Ethyl acetate=30/1 to 0:1). 8AmBza-6 (0.58 g, 687 μmol, 70.4% yield, 93.14% purity) was obtained as a yellow solid. ¹H NMR (MeOD, 400 MHz) δ8.70 (d, J=2.4 Hz, 1H), 8.19 (dd, J=2.4, 9.8 Hz, 1H), 8.05-7.89 (m, 2H), 7.74 (s, 1H), 7.42 (d, J=9.8 Hz, 1H), 7.14 (s, 1H), 4.21 (d, J=13.6 Hz, 1H), 3.59-3.32 (m, 10H), 3.28-3.24 (m, 2H), 3.16-3.11 (m, 2H), 2.63-2.53 (m, 1H), 2.06-1.90 (m, 2H), 1.89-1.78 (m, 3H), 1.74-1.61 (m, 2H), 1.53-1.25 (m, 9H), 1.06-0.84 (m, 3H). LC/MS [M+H] 785.38 (calculated); LC/MS [M+H] 786.0 (observed).

### Example 7 Synthesis of tert-butyl N-[3-[[2-amino-8-[[2-[2-(tert-butoxycarbonylamino)ethylamino]pyrimidin-5-yl]carbamoyl]-3H-1-benzazepine-4-carbonyl]-propyl-amino]propyl]carbamate, 8AmBza-7

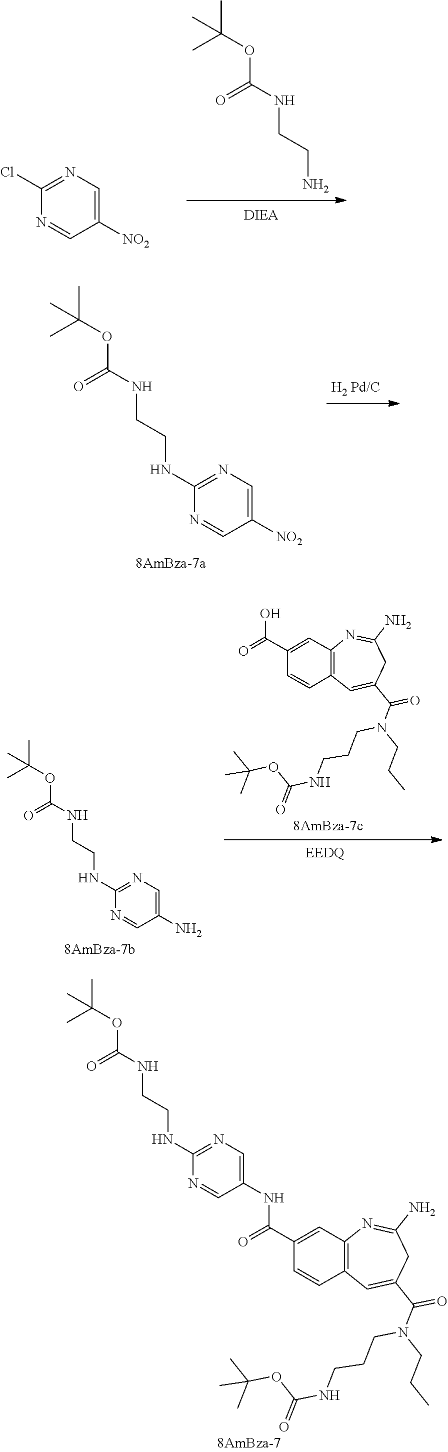 [SMILES: O=[N+]([O-])c1cnc(Cl)nc1 ; CC(C)(C)OC(=O)NCCN ; CC(C)(C)OC(=O)NCCNc1ncc([N+](=O)[O-])cn1 ; CC(C)(C)OC(=O)NCCNc1ncc(N)cn1 ; CCCN(CCCNC(=O)OC(C)(C)C)C(=O)C1=Cc2ccc(C(=O)O)cc2N=C(N)C1 ; CCCN(CCCNC(=O)OC(C)(C)C)C(=O)C1=Cc2ccc(C(=O)Nc3cnc(NCCNC(=O)OC(C)(C)C)nc3)cc2N=C(N)C1]

To a mixture of 2-chloro-5-nitro-pyrimidine (2.9 g, 18.2 mmol, 1.0 eq) and tert-butyl N-(2-aminoethyl)carbamate (3.2 g, 20.0 mmol, 3.14 mL, 1.1 eq) in THF (50 mL) was added DIEA (4.7 g, 36.4 mmol, 6.33 mL, 2.0 eq) at 25° C. and it was stirred for 2 hours at this temperature. The mixture was added water (100 mL) and extracted with ethyl acetate (50 mL×3). The combined organic phase was washed with brine (50 mL), dried with anhydrous Na₂SO₄, filtered and concentrated in vacuum. Compound tert-butyl N-[2-[(5-nitropyrimidin-2-yl)amino]ethyl]carbamate, 8AmBza-7a (5.7 g, crude) was obtained as a yellow solid. ¹H NMR (CDCl₃, 400 MHz) δ9.11 (d, J=2.8 Hz, 1H), 9.05 (d, J=2.8 Hz, 1H), 6.59 (s, 1H), 4.85 (s, 1H), 3.66 (q, J=5.6 Hz, 2H), 3.44-3.41 (m, 2H), 1.45 (s, 9H).

To a solution of 8AmBza-7a (1.0 g, 3.53 mmol, 1.0 eq) in MeOH (30 mL) was added Pd/C (0.5 g, 10% purity) under N₂. The suspension was degassed under vacuum and purged with H₂ several times. The mixture was stirred under H₂ (15 psi) at 25° C. for 12 hours, then filtered and the filtrate was concentrated in vacuum. 8AmBza-7b (0.8 g, crude) was obtained as a yellow solid.

To a mixture of 2-amino-4-[3-(tert-butoxycarbonylamino) propyl-propyl-carbamoyl]-3H-1-benzazepine-8-carboxylic acid, 8AmBza-7c (60 mg g, 135 μmol, 1.0 eq) and 8AmBza-7b (103 mg, 405 μmol, 3 eq) in MeOH (5 mL) and DCM (10 mL) was added EEDQ (50 mg, 202 μmol, 1.5 eq) at 25° C. and it was stirred for 12 hours at this temperature. The mixture was concentrated under reduced pressure, and then the residue was purified by prep-HPLC (column: Welch Xtimate C18 100×25 mm×3 um; mobile phase: [water (0.1% TFA)-ACN]; B %: 25%-45%, 12 min). 8AmBza-7 (13 mg, 16.8 μmol, 12.4% yield, 87.7% purity) was obtained as a yellow solid. ¹H NMR (MeOD, 400 MHz) δ8.64 (s, 2H), 8.05-7.90 (m, 2H), 7.73 (s, 1H), 7.14 (s, 1H), 3.53-3.48 (m, 6H), 3.37-3.34 (m, 2H), 3.31 (s, 2H), 3.29-3.13 (m, 2H), 1.90-1.78 (m, 2H), 1.75-1.64 (m, 2H), 1.56-1.40 (m, 18H), 1.02-0.87 (m, 3H). LC/MS [M+H] 680.4 (calculated); LC/MS [M+H] 680.3 (observed).

### Example 8 Synthesis of tert-butyl N-[3-[[2-amino-8-[[3-[2-[2-(tert-butoxycarbonylamino)ethoxy]ethoxymethyl]phenyl]carbamoyl]-3H-1-benzazepine-4-carbonyl]-propyl-amino]propyl]carbamate, 8AmBza-8

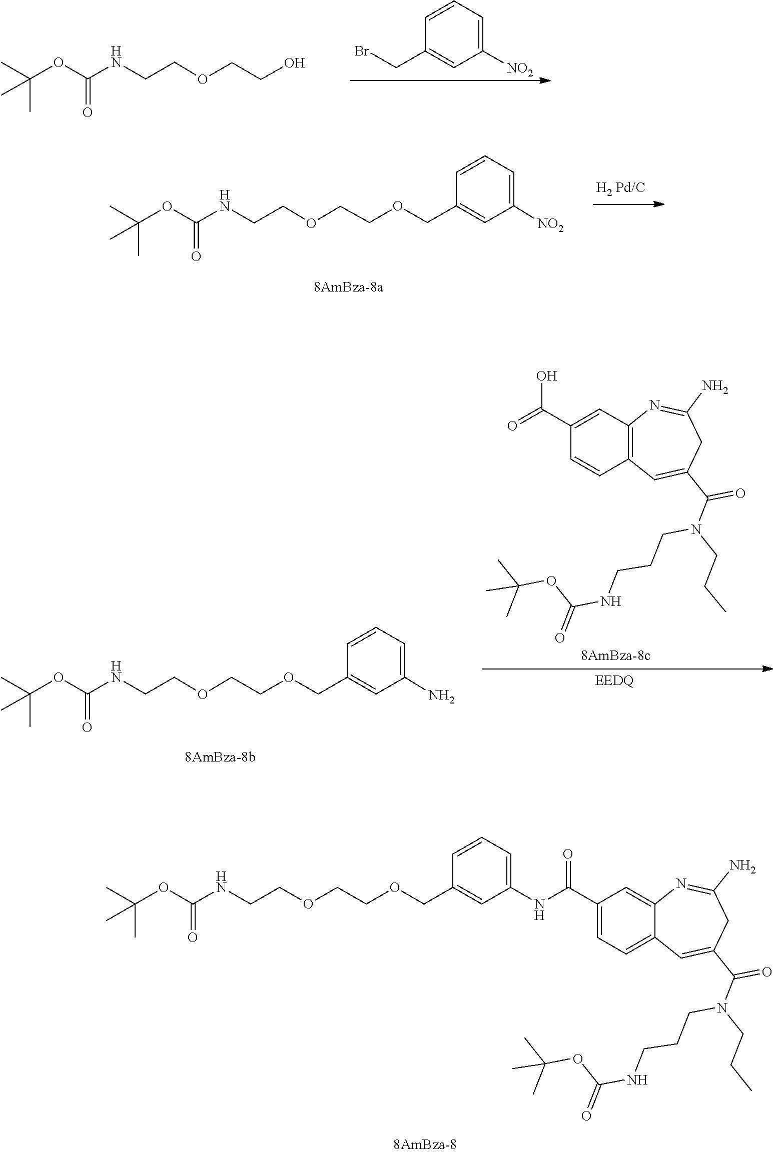 [SMILES: CC(C)(C)OC(=O)NCCOCCO ; O=[N+]([O-])c1cccc(CBr)c1 ; CC(C)(C)OC(=O)NCCOCCOCc1cccc([N+](=O)[O-])c1 ; CC(C)(C)OC(=O)NCCOCCOCc1cccc(N)c1 ; CCCN(CCCNC(=O)OC(C)(C)C)C(=O)C1=Cc2ccc(C(=O)O)cc2N=C(N)C1 ; CCCN(CCCNC(=O)OC(C)(C)C)C(=O)C1=Cc2ccc(C(=O)Nc3cccc(COCCOCCNC(=O)OC(C)(C)C)c3)cc2N=C(N)C1]

To a mixture of tert-butyl N-[2-(2-hydroxyethoxy)ethyl]carbamate (2.9 g, 14.1 mmol, 1.0 eq) in DMF (10 mL) was added sodium hydride, NaH (565 mg, 14.1 mmol, 60% purity, 1.0 eq) slowly at 0° C. and it was stirred for 1 h at this temperature, then 1-(bromomethyl)-3-nitro-benzene (3.05 g, 14.13 mmol, 1.0 eq) was added to the mixture and stirred for 0.5 h. The mixture was diluted with water (30 ml) and extracted with ethylacetate, EtOAc (30 mL×3). The organic layer was washed with brine, dried over Na₂SO₄, filtered and concentrated in vacuum. The residue was purified by silica gel chromatography (Petroleum ether/Ethyl acetate=10/1 to 1/1) to afford tert-butyl N-[2-[2-[(3-nitrophenyl)methoxy]ethoxy]ethyl]carbamate, 8AmBza-8a (2.2 g, 6.46 mmol, 45.75% yield) as yellow oil. ¹H NMR (CDCl₃, 400 MHz) δ8.24 (s, 1H), 8.15 (d, J=8.4 Hz, 1H), 7.68 (d, J=8.0 Hz, 1H), 7.53 (t, J=8.0 Hz, 1H), 4.96 (s, 1H), 4.67 (s, 2H), 3.71-3.64 (m, 4H), 3.59-3.52 (m, 2H), 3.37-3.28 (m, 2H), 1.43 (s, 9H).

To a solution of 8AmBza-8a (400 mg, 1.18 mmol, 1.0 eq) in EtOAc (10 mL) was added Pd/C (0.3 g, 10% purity) under N₂. The suspension was degassed under vacuum and purged with H₂ several times. The mixture was stirred under H₂ (15 psi) at 25° C. for 3 hours, then filtered and concentrated in vacuum to afford tert-butyl N-[2-[2-[(3-aminophenyl)methoxy]ethoxy]ethyl]carbamate, 8AmBza-8b (0.35 g, crude) as yellow oil.

To a mixture of 8AmBza-8b (42 mg, 135 μmol, 1.2 eq) and 2-amino-4-[3-(tert-butoxycarbonylamino)propyl-propyl-carbamoyl]-3H-1-benzazepine-8-carboxylic acid, 8AmBza-8c (50 mg, 112 μmol, 1.0 eq) in MeOH (0.5 mL) and DCM (1 mL) was added EEDQ (42 mg, 168 μmol, 1.5 eq) at 25° C. The mixture was stirred at 25° C. for 12 h, and then concentrated in vacuum. The residue was purified by prep-HPLC (column: Welch Xtimate C18 100*25 mm*3 um; mobile phase: [water (0.1% TFA)-ACN]; B %: 30%-50%, 12 min) to give 8AmBza-8 (8 mg, 10.9 μmol, 9.6% yield) as white solid. ¹H NMR (MeOD, 400 MHz) δ8.02-7.95 (m, 2H), 7.80-7.71 (m, 2H), 7.68 (d, J=8.8 Hz, 1H), 7.40 (t, J=7.6 Hz, 1H), 7.21 (d, J=8.0 Hz, 1H), 7.16 (s, 1H), 4.62 (s, 2H), 3.73-3.65 (m, 4H), 3.55 (t, J=5.6 Hz, 4H), 3.50 (s, 2H), 3.39 (s, 2H), 3.25 (t, J=5.6 Hz, 2H), 3.12 (d, J=18.4 Hz, 2H), 1.92-1.81 (m, 2H), 1.77-1.64 (m, 2H), 1.43 (s, 18H), 0.94 (s, 3H). LC/MS [M+H] 737.4 (calculated); LC/MS [M+H] 737.4 (observed).

### Example 9 Synthesis of tert-butyl (3-(2-amino-8-(phenylcarbamoyl)-N-propyl-3H-benzo [b]azepine-4-carboxamido)propyl)carbamate, 8AmBza-9

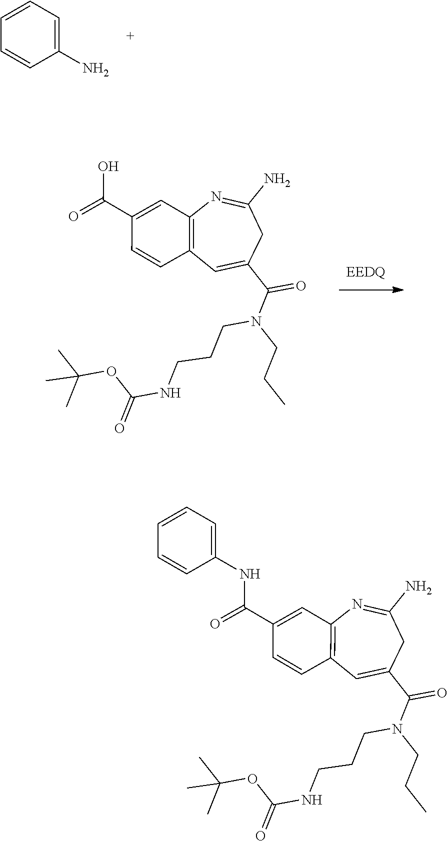 [SMILES: Nc1ccccc1 ; CCCN(CCCNC(=O)OC(C)(C)C)C(=O)C1=Cc2ccc(C(=O)O)cc2N=C(N)C1 ; CCCN(CCCNC(=O)OC(C)(C)C)C(=O)C1=Cc2ccc(C(=O)Nc3ccccc3)cc2N=C(N)C1]

To a mixture of aniline (25 mg, 270 μmol, 2.0 eq) and 2-amino-4-[3-(tert-butoxycarbonylamino)propyl-propyl-carbamoyl]-3H-1-benzazepine-8-carboxylic acid (60 mg, 135 μmol, 1.0 eq) in DCM (2 mL) and MeOH (0.5 mL) was added EEDQ (50 mg, 202 μmol, 1.5 eq) at 25 under N₂. The mixture was stirred at 25° C. for 2 hours and then concentrated in vacuum. The residue was purified by prep-HPLC (column: Welch Xtimate C18 150*25 mm*5 um; mobile phase: [water (10 mM NH₄HCO₃)-ACN]; B %: 40%-70%, 10.5 min) to afford 8AmBza-9 (10 mg, 19.2 μmol, 14.26% yield) as a white solid. ¹H NMR (MeOD, 400 MHz) δ 7.73-7.66 (m, 3H), 7.57 (dd, J=1.6, 8.0 Hz, 1H), 7.47 (br d, J=8.0 Hz, 1H), 7.37 (t, J=8.0 Hz, 2H), 7.20-7.12 (m, 1H), 6.93 (s, 1H), 3.50 (br t, J=7.2 Hz, 2H), 3.45-3.38 (m, 2H), 3.21-2.96 (m, 2H), 2.85 (s, 2H), 1.89-1.77 (m, 2H), 1.70-1.62 (m, 2H), 1.44 (s, 9H), 1.05-0.8 (m, 3H). LC/MS [M+H] 520.3 (calculated); LC/MS [M+H] 520.3 (observed).

### Example 10 Synthesis of 2-amino-N4-(3-aminopropyl)-N8-phenyl-N4-propyl-3H-1-benzazepine-4,8-dicarboxamide, 8AmBza-10

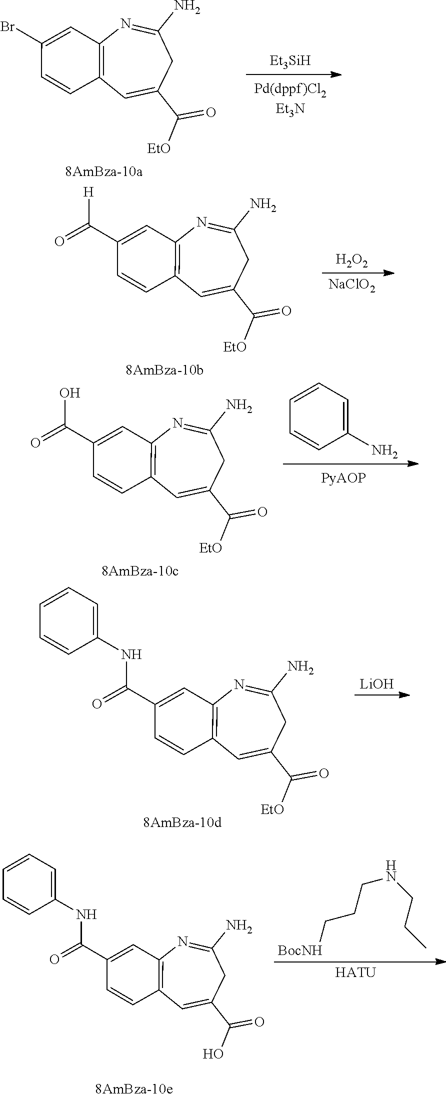

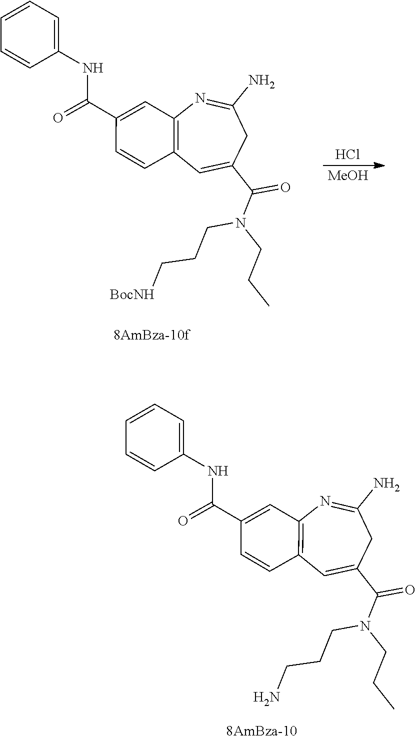 [SMILES: "MeOH" ; CCCN(CCCNC(=O)OC(C)(C)C)C(=O)C1=Cc2ccc(C(=O)Nc3ccccc3)cc2N=C(N)C1 ; Cl ; CCCN(CCCN)C(=O)C1=Cc2ccc(C(=O)Nc3ccccc3)cc2N=C(N)C1]

### Preparation of ethyl 2-amino-8-formyl-3H-1-benzazepine-4-carboxylate, 8AmBza-10b

To a solution of ethyl 2-amino-8-bromo-3H-1-benzazepine-4-carboxylate, 8AmBza-10a (10 g, 32.4 mmol, 1 eq) in DMF (100 mL) was added Et₃SiH (72.8 g, 626.09 mmol, 100 mL, 19.36 eq), Et₃N (6.5 g, 64.69 mmol, 9.00 mL, 2 eq) and Pd(dppf)Cl₂ (1.18 g, 1.62 mmol, 0.05 eq) under N₂. The suspension was degassed under vacuum and purged with CO several times and it was stirred under CO (50 psi) at 80° C. for 12 h (hours). The mixture was diluted with water (300 mL) and extracted with EtOAc (80 mL×3). The organic layer was washed with brine (50 mL), dried over Na₂SO₄, filtered and concentrated, and the residue was purified by flash silica gel chromatography (ISCO®; 15 g SepaFlash® Silica Flash Column, Eluent of 0-100% Ethyl acetate/Petroleum ether gradient at 65 mL/min) to give 8AmBza-10b (3 g, 11.6 mmol, 35.9% yield) as yellow solid. ¹H NMR (DMSO-d₆, 400 MHz) δ10.00 (s, 1H) 7.79 (s, 1H) 7.61 (d, J=8.4 Hz, 1H) 7.55 (d, J=1.2 Hz, 1H) 7.40 (dd, J=8.0, 1.2 Hz, 1H) 7.07 (s, 2H) 4.25 (q, J=6.8 Hz, 2H) 2.91 (s, 2H) 1.31 (t, J=6.8 Hz, 3H).

### Preparation of 2-amino-4-ethoxycarbonyl-3H-1-benzazepine-8-carboxylic acid, 8AmBza-10c

To a solution of 8AmBza-10b (2.6 g, 10.1 mmol, 1.0 eq) in CH₃CN (15 mL) was added NaH₂PO₄ (362 mg, 3.02 mmol, 0.3 eq), H₂O₂ (5.71 g, 50.33 mmol, 4.84 mL, 30% purity, 5 eq) and NaClO₂ (1.46 g, 16.1 mmol, 1.6 eq) at 0° C. and it was stirred at 25° C. for 5 h. The reaction mixture was quenched with Na₂SO₃ (aq) and diluted with H₂O (30 mL) and EtOAc (30 ml), the pH of the mixture was adjusted to 4 with aq HCl (1 M), then filtered to give desired solid The solid was dried under vacuum to give 8AmBza-10c (2.1 g, 7.66 mmol, 76.1% yield) as white solid. ¹H NMR (DMSO-d₆, 400 MHz) δ7.87 (s, 1H), 7.81 (s, 1H), 7.72-7.67 (m, 2H), 4.27 (q, J=7.2 Hz, 2H), 3.28 (s, 2H), 1.31 (t, J=7.2 Hz, 3H).

### Preparation of ethyl 2-amino-8-(phenylcarbamoyl)-3H-1-benzazepine-4-carboxylate, 8AmBza-10d

To a mixture of 8AmBza-10c (1.0 g, 3.65 mmol, 1.0 eq) in DMF (20 mL) was added (7-Aza-benzotriazol-1-yloxy-tripyrrolidino-phosphonium hexafluorophosphate), PyAOP (2.28 g, 4.38 mmol, 1.2 eq) and DIEA (2.36 g, 18.2 mmol, 3.18 mL, 5.0 eq) at 25° C. and it was stirred at 25° C. for 10 min, then aniline (373 mg, 4.01 mmol, 366 μL, 1.1 eq) was then added and stirred for 1 hour at 25° C. The mixture was poured into ice water (50 mL) and stirred for 2 min. The aqueous phase was extracted with ethyl acetate (20 mL×3). The combined organic phase was washed with brine (20 mL), dried with anhydrous Na₂SO₄, filtered and concentrated in vacuum and the residue was purified by silica gel chromatography (Petroleum ether/Ethyl acetate=0/1 to EtOAc/MeOH=2/1) to afford 8AmBza-10d (0.5 g, 1.43 mmol, 39.25% yield) as yellow solid. ¹H NMR (MeOD, 400 MHz) δ 7.89 (s, 1H), 7.76-7.65 (m, 3H), 7.62-7.56 (m, 1H), 7.37 (t, J=8.0 Hz, 2H), 7.16 (t, J=8.0 Hz, 1H), 4.35 (q, J=7.2 Hz, 2H), 3.32 (s, 2H), 1.38 (t, J=7.2 Hz, 3H).

### Preparation of 2-amino-8-(phenylcarbamoyl)-3H-1-benzazepine-4-carboxylic acid, 8AmBza-10e

To a mixture of 8AmBza-10d (0.36 g, 1.03 mmol, 1.0 eq) in EtOH (10 mL) was added a solution of LiOH.H₂O (216 mg, 5.15 mmol, 5.0 eq) in H₂O (1 mL) at 25° C. and it was stirred for 16 hours at this temperature. The mixture was quenched with HCl (4M) until pH to 5 and concentrated under reduced pressure at 40° C. to remove EtOH. Water (10 mL) was added to the mixture and then filtered to give desired solid 8AmBza-10e (0.2 g, 622 μmol, 60.41% yield) was obtained as yellow solid which was used into the next step without further purification. ¹H NMR (DMSO-d₆, 400 MHz) δ7.84-7.74 (m, 3H), 7.66 (s, 1H), 7.56-7.47 (m, 2H), 7.34 (t, J=8.0 Hz, 2H), 7.09 (t, J=7.2 Hz, 2H), 2.92 (s, 2H).

### Preparation of tert-butyl N-[3-[[2-amino-8-(phenylcarbamoyl)-3H-1-benzazepine-4-carbonyl]-propyl-amino]propyl]carbamate, 8AmBza-10f

To a solution of 8AmBza-10e (0.2 g, 622 μmol, 1.0 eq) in DMF (5 mL) was added HATU (284 mg, 746 μmol, 1.2 eq) and DIEA (241 mg, 1.87 mmol, 325 μL, 3.0 eq) at 25° C. and it was stirred for 10 min at this temperature, then tert-butyl N-[3-(propylamino)propyl]carbamate (161 mg, 746 μmol, 1.2 eq) was added to the mixture and stirred at 25° C. for 3 hours. The mixture was poured into ice water (30 mL) and stirred for 10 min. The aqueous phase was extracted with EtOAc (10 mL×3), and the combined organic phase was washed with H₂O (10 mL×2) and brine (10 mL), dried by Na₂SO₄ and concentrated to give 8AmBza-10f (0.3 g, 577 μmol, 92.76% yield) as yellow oil.

### Preparation of 2-amino-N4-(3-aminopropyl)-N8-phenyl-N4-propyl-3H-1-benzazepine-4,8-dicarboxamide, 8AmBza-10

To a solution of 8AmBza-10f (0.4 g, 769 μmol, 1.0 eq) in MeOH (10 mL) was added HCl/MeOH (4 M, 9.62 mL, 50 eq) at 25° C. The mixture was stirred at 25° C. for 1 hour, and then concentrated under reduced pressure at 40° C. The residue was purified by prep-HPLC (column: Nano-micro Kromasil C18 100*30 mm 8 um; mobile phase: [water (0.1% TFA)-ACN]; B %: 5%-30%, 10 min) to afford 8AmBza-10 (0.23 g, 431 μmol, 56.0% yield, TFA salt) as yellow solid. ¹H NMR (MeOD, 400 MHz) δ8.01-7.94 (m, 2H), 7.76-7.70 (m, 3H), 7.41 (t, J=8.0 Hz, 2H), 7.21 (t, J=7.6 Hz, 2H), 3.63 (t, J=7.2 Hz, 2H), 3.58-3.49 (m, 2H), 3.41 (s, 2H), 3.10-2.95 (m, 2H), 2.12-1.99 (m, 2H), 1.82-1.68 (m, 2H), 0.95 (t, J=7.2 Hz, 3H). LC/MS [M+H] 420.2 (calculated); LC/MS [M+H] 420.2 (observed).

### Example 11 Synthesis of tert-butyl N-[4-[[2-amino-8-(phenylcarbamoyl)-3H-1-benzazepine-4-carbonyl]-propyl-amino]but-2-ynyl]carbamate, 8AmBza-11

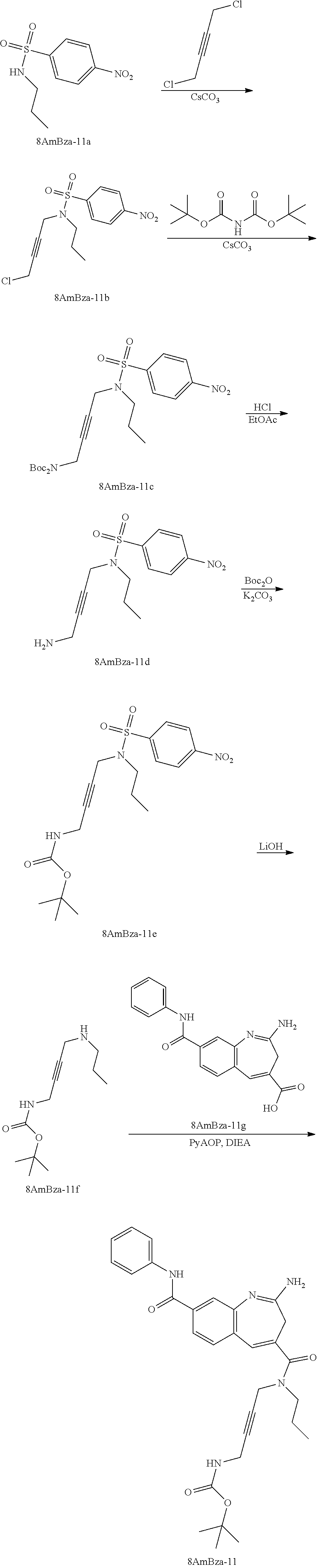

### Preparation of N-(4-chlorobut-2-ynyl)-4-nitro-N-propyl-benzenesulfonamide, 8AmBza-11b

To a solution of propan-1-amine (7 g, 118 mmol, 9.74 mL, 1.0 eq) and Et₃N (24 g, 237 mmol, 33 mL, 2.0 eq) in DCM (50 mL) was added 4-nitrobenzenesulfonyl chloride (26.2 g, 118 mmol, 1.0 eq) and it was stirred at 25° C. for 0.5 h. The reaction mixture was poured into water (60 mL) and extracted with DCM (100 mL*3). The combined organic phases was washed with brine (50 mL), dried over Na₂SO₄, filtered and concentrated under reduced pressure to give the crude product 4-nitro-N-propyl-benzenesulfonamide, 8AmBza-11a (28 g, 114.6 mmol, 96.8% yield) as yellow solid which was used into the next step without further purification. ¹H NMR (CDCl₃, 400 MHz) δ8.38 (d, J=8.8 Hz, 2H), 8.07 (d, J=8.8 Hz, 2H), 4.77 (s, 1H), 3.02-2.99 (m, 2H), 1.57-1.48 (m, 2H), 0.89 (t, J=7.6 Hz, 3H)

To a solution of 8AmBza-11a (28 g, 115 mmol, 1.0 eq) in DMF (300 mL) was added Cs₂CO₃ (56 g, 172 mmol, 1.5 eq) and 1, 4-dichlorobut-2-yne (28.2 g, 229 mmol, 2.0 eq) and it was stirred at 25° C. for 16 h. The reaction mixture was poured into water (300 mL) and extracted with MTBE (150 mL*3). The combined organic phases was washed with brine (150 mL), dried over Na₂SO₄, filtered and concentrated under reduced pressure, and the residue was purified by column chromatography (SiO₂, Petroleum ether/Ethyl acetate=50/1 to 5/1) to give 8AmBza-11b (28 g, 84.6 mmol, 73.84% yield) as yellow oil. ¹H NMR (CDCl₃, 400 MHz) δ8.37 (d, J=8.8 Hz, 2H), 8.05 (d, J=8.8 Hz, 2H), 4.22 (t, J=2.0 Hz, 2H), 3.85 (t, J=2.0 Hz, 2H), 3.17 (t, J=7.6 Hz, 2H), 1.65-1.56 (m, 2H), 0.94 (t, J=7.6 Hz, 3H).

### Preparation of tert-butyl (tert-butoxycarbonyl)(4-((4-nitro-N-propylphenyl)sulfonamido)but-2-yn-1-yl)carbamate, 8AmBza-11c

To a solution of 8AmBza-11b (23.5 g, 71.0 mmol, 1.0 eq) in DMF (250 mL) was added Cs₂CO₃ (46.3 g, 142 mmol, 2.0 eq) and tert-butyl N-tert-butoxycarbonylcarbamate (23.1 g, 106 mmol, 1.5 eq). The mixture was stirred at 25° C. for 16 h, and then poured into water (300 mL) and extracted with MTBE (150 mL*3). The combined organic phases was washed with brine (200 mL), dried over Na₂SO₄, filtered and concentrated under reduced pressure to give a residue. The residue was purified by column chromatography (SiO₂, Petroleum ether/Ethyl acetate=50/1 to 5/1) to give 8AmBza-11c (32 g, crude) as yellow oil. ¹H NMR (CDCl₃, 400 MHz) δ 8.39 (d, J=8.8 Hz, 2H), 8.05 (d, J=8.8 Hz, 2H), 4.21 (s, 2H), 4.11 (s, 2H), 3.14 (t, J=7.2 Hz, 2H), 1.66-1.54 (m, 2H), 1.49 (s, 18H), 0.93 (t, J=7.2 Hz, 3H).

### Preparation of N-(4-aminobut-2-ynyl)-4-nitro-N-propyl-benzenesulfonamide, 8AmBza-11d

To a solution of 8AmBza-11c (32 g, 62.5 mmol, 1.0 eq) in EtOAc (50 mL) was added HCl/EtOAc (4 M, 60 mL, 3.8 eq). The mixture was stirred at 25° C. for 1 h and then concentrated under reduced pressure to give 8AmBza-11d (27 g, crude, HCl salt) as yellow solid.

### Preparation of tert-butyl N-[4-[(4-nitrophenyl)sulfonyl-propyl-amino]but-2-ynyl]carbamate, 8AmBza-11e

To a solution of 8AmBza-11d (27 g, 77.6 mmol, 1.0 eq, HCl) in THF (100 mL) and water (10 mL) was added Boc₂O (13.5 g, 62.1 mmol, 14.3 mL, 0.8 eq) and K₂CO₃ (21.5 g, 155 mmol, 2 eq). The mixture was stirred at 25° C. for 1 hr and then poured into water (100 mL) and extracted with EtOAc (100 mL*3). The combined organic phases was washed with brine (100 mL), dried over Na₂SO₄, filtered and concentrated under reduced pressure to give a residue. The residue was purified by column chromatography (SiO₂, Petroleum ether/Ethyl acetate=80/1 to 3/1) to give 8AmBza-11e (20 g, 48.6 mmol, 62.6% yield) as yellow solid. ¹H NMR (CDCl₃, 400 MHz) δ8.37 (d, J=8.8 Hz, 2H), 8.05 (d, J=8.8 Hz, 2H), 4.42 (s, 1H), 4.19 (s, 2H), 3.67 (d, J=5.2 Hz, 2H), 3.17 (t, J=7.2 Hz, 2H), 1.64-1.59 (m, 2H), 1.44 (s, 9H), 0.95 (t, J=7.6 Hz, 3H).

### Preparation of tert-butyl N-[4-(propylamino)but-2-ynyl]carbamate, 8AmBza-11f

To a solution of 8AmBza-11e (20 g, 48.6 mmol, 1.0 eq) and LiOH.H₂O (12.2 g, 291 mmol, 6.0 eq) in MeCN (100 mL) was added methyl 2-mercaptoacetate (15.5 g, 146 mmol, 13.2 mL, 3.0 eq) at 0° C. The mixture was stirred at 25° C. for 2 hr. Water (100 mL) was added to the mixture and adjusted the pH of aqueous phase to 2 with 1N HCl at 0° C. The mixture was extracted with MTBE (100 mL*2), the pH of aqueous phase was adjusted to 9 with sat. NaHCO₃ and then extracted with EtOAc (50 mL×3). The organic layers were washed with brine (40 mL), dried over Na₂SO₄, filtered and concentrated under reduced pressure to give crude product 8AmBza-1 if (10 g, 44.2 mmol, 90.91% yield) as brown oil which was used into the next step without further purification. ¹H NMR (CDCl₃, 400 MHz) δ3.95 (s, 2H), 3.46 (s, 2H), 2.67 (t, J=7.2 Hz, 2H), 1.59-1.50 (m, 2H), 1.47 (s, 9H), 0.96 (t, J=7.2 Hz, 3H).

### Preparation of tert-butyl N-[4-[[2-amino-8-(phenylcarbamoyl)-3H-1-benzazepine-4-carbonyl]-propyl-amino]but-2-ynyl]carbamate, 8AmBza-11

To a mixture of 2-amino-8-(phenylcarbamoyl)-3H-benzo[b]azepine-4-carboxylic acid, 8AmBza-11 g (0.1 g, 311 μmol, 1.0 eq) in DMF (3 mL) was added PYAOP (194 mg, 373 μmol, 1.2 eq) and DIEA (120 mg, 933 μmol, 162 μL, 3.0 eq) at 25° C. Then 8AmBza-1 if (84 mg, 373 μmol, 1.2 eq) was added to the mixture and stirred at 25° C. for 1 h. The mixture filtered and concentrated, the residue was purified by prep-HPLC (column: Xtimate C18 100*30 mm*3 um; mobile phase: [water (0.1% TFA)-ACN]; B %: 25%-55%, 10 min) to give 8AmBza-11 (13 mg, 24.6 μmol, 7.89% yield) as white solid. H NMR (MeOD, 400 MHz) δ7.98-7.93 (m, 2H), 7.71 (d, J=8.0 Hz, 3H), 7.39 (t, J=8.0 Hz, 2H), 7.19 (t, J=8.0 Hz, 1H), 4.33 (s, 2H), 3.86 (s, 2H), 3.61-3.47 (m, 2H), 3.39 (s, 2H), 1.80-1.70 (m, 2H), 1.43 (s,

### Example 12 Synthesis of 3-[2-[2-[2-[2-[2-[2-[2-[2-[2-[2-[[(Z)—N′-[3-[[2-amino-8-(phenylcarbamoyl)-3H-1-benzazepine-4-carbonyl]-propyl-amino]propyl]-N-(3-cyanophenyl)carbamimidoyl]amino]ethoxy]ethoxy]ethoxy]ethoxy]ethoxy]ethoxy]ethoxy]ethoxy]ethoxy]ethoxy]propanoic acid, 8AmBza-12

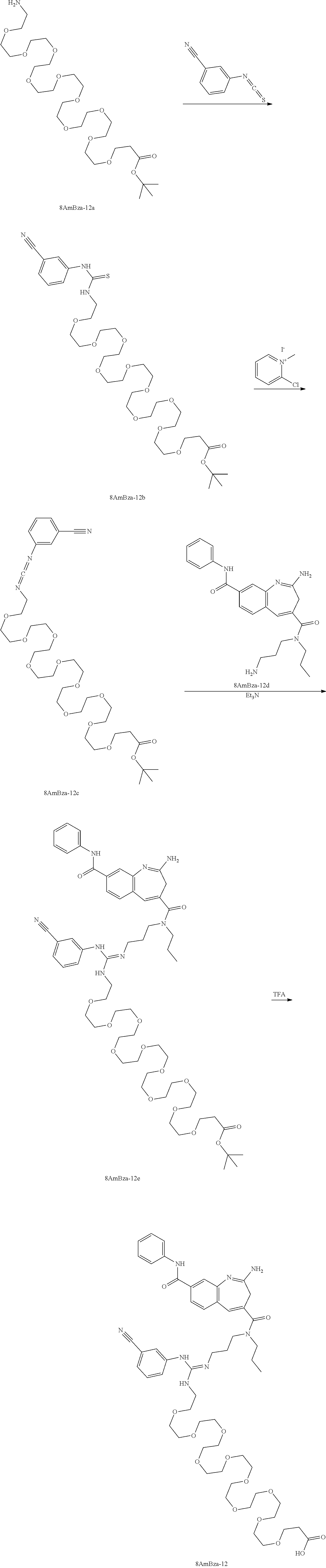

### Preparation of tert-butyl 3-[2-[2-[2-[2-[2-[2-[2-[2-[2-[2-[(3-cyanophenyl)carbamothioylamino]ethoxy]ethoxy]ethoxy]ethoxy]ethoxy]ethoxy]ethoxy]ethoxy]ethoxy]ethoxy]propanoate, 8AmBza-12b

To a mixture of tert-butyl 3-[2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]propanoate, 8AmBza-12a (2.7 g, 4.61 mmol, 1.0 eq) in THF (20 mL) was added Et₃N (700 mg, 6.91 mmol, 960 μL, 1.5 eq) and 3-isothiocyanatobenzonitrile (1.48 g, 9.22 mmol, 2.0 eq) at 25° C. and it was stirred for 1 hour at this temperature. Then the mixture was diluted with water (30 mL) and extracted with EtOAc (50 mL×3). The organic layer was washed with brine, dried over Na₂SO₄, filtered and concentrated. The residue was purified by silica gel chromatography (MeOH/Ethyl acetate=0/1, 1/10) to afford 8AmBza-12b (0.5 g, 670 μmol, 14.54% yield) as yellow oil. ¹H NMR (CDCl₃, 400 MHz) δ7.99 (s, 1H), 7.89 (d, J=8.0 Hz, 1H), 7.44-7.39 (m, 2H), 3.76-3.58 (m, 42H), 2.55-2.46 (m, 2H), 1.45 (s, 9H)

### Preparation of tert-butyl 3-[2-[2-[2-[2-[2-[2-[2-[2-[2-[2-[(3-cyanophenyl)iminomethyleneamino]ethoxy]ethoxy]ethoxy]ethoxy]ethoxy]ethoxy]ethoxy]ethoxy]ethoxy]ethoxy]propanoate, 8AmBza-12c

To a mixture of 8AmBza-12b (0.4 g, 536 μmol, 1.0 eq) and Et₃N (163 mg, 1.61 mmol, 223 μL, 3.0 eq) in DCM (10 mL) and DMF (0.4 mL) was added 2-chloro-1-methylpyridin-1-ium iodide (164 mg, 643 μmol, 1.2 eq) at 25° C. under N₂. The mixture was stirred at 25° C. for 1 hour and then concentrated under reduce pressure. The residue was purified by silica gel chromatography (CH₃CN/Ethyl acetate=0/1 to 1/1) to afford 8AmBza-12c (0.29 g, 407 μmol, 75.9% yield) as yellow oil. ¹H NMR (CDCl₃, 400 MHz) δ7.43-7.33 (m, 4H), 3.70-3.62 (m, 42H), 2.51 (t, J=6.4 Hz, 2H), 1.45 (s, 9H).

### Preparation of tert-butyl 3-[2-[2-[2-[2-[2-[2-[2-[2-[2-[2-[[(Z)—N′-[3-[[2-amino-8-(phenylcarbamoyl)-3H-1-benzazepine-4-carbonyl]-propyl-amino]propyl]-N-(3-cyanophenyl)carbamimidoyl]amino]ethoxy]ethoxy]ethoxy]ethoxy]ethoxy]ethoxy]ethoxy]ethoxy]ethoxy]ethoxy]propanoate 8AmBza-12e

To a mixture of 2-amino-N4-(3-aminopropyl)-N8-phenyl-N4-propyl-3H-1-benzazepine-4,8-dicarboxamide, 8AmBza-12d (0.06 g, 112 μmol, 1.0 eq, TFA salt) in DMF (1 mL) was added Et₃N (28 mg, 281 μmol, 2.5 eq) and 8AmBza-12c (88 mg, 123 μmol, 1.1 eq) at 25° C. The mixture was stirred at 25° C. for 1 hour and then filtered and purified by prep-HPLC (column: Nano-micro Kromasil C18 100*30 mm 8 um; mobile phase: [water (0.1% TFA)-ACN]; B %: 20%-50%, 10 min) to afford 8AmBza-12e (0.08 g, 70.7 μmol, 62.9% yield) as colorless oil.

### Preparation of 3-[2-[2-[2-[2-[2-[2-[2-[2-[2-[2-[[(Z)—N′-[3-[[2-amino-8-(phenylcarbamoyl)-3H-1-benzazepine-4-carbonyl]-propyl-amino]propyl]-N-(3-cyanophenyl)carbamimidoyl]amino]ethoxy]ethoxy]ethoxy]ethoxy]ethoxy]ethoxy]ethoxy]ethoxy]ethoxy]ethoxy]propanoic acid, 8AmBza-12

To a solution of 8AmBza-12e (0.07 g, 61 μmol, 1.0 eq) in H₂O (5 mL) and CH₃CN (1 mL) was added TFA (211 mg, 1.86 mmol, 30 eq) at 25° C. The mixture was stirred at 80° C. for 2 hours and then concentrated under reduced pressure at 50° C. The residue was freeze-dried to give 8AmBza-12 (51 mg, 42.9 μmol, 69.3% yield, TFA salt) as light yellow oil. ¹H NMR (MeOD, 400 MHz) δ8.01-7.94 (m, 2H), 7.79-7.75 (m, 1H), 7.72 (d, J=8.0 Hz, 2H), 7.66-7.64 (m, 4H), 7.39 (t, J=7.6 Hz, 2H), 7.19 (t, J=7.6 Hz, 1H), 7.13 (s, 1H), 3.76-3.52 (m, 46H), 3.42-3.40 (m, 4H), 2.53 (t, J=6.4 Hz, 2H), 2.04 (m, 2H), 1.79-1.65 (m, 2H), 0.93 (t, J=7.2 Hz, 3H). LC/MS [M+H] 1075.6 (calculated); LC/MS [M+H] 1075.6 (observed).

### Example 18 Synthesis of 2-amino-N8-[6-[4-(2-aminoethylcarbamoyl)-1-piperidyl]-3-pyridyl]-N4-ethoxy-N4-propyl-3H-1-benzazepine-4,8-dicarboxamide, 8AmBza-18

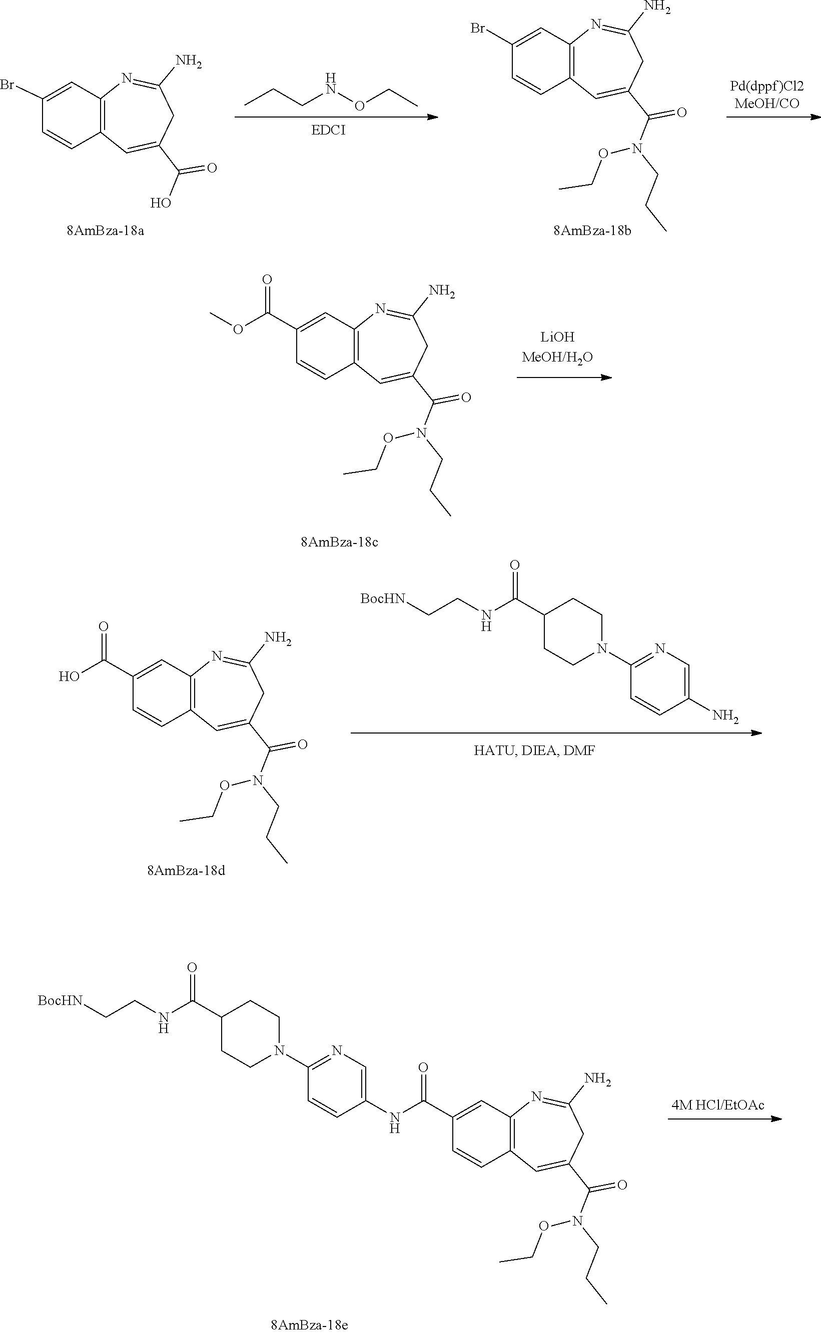

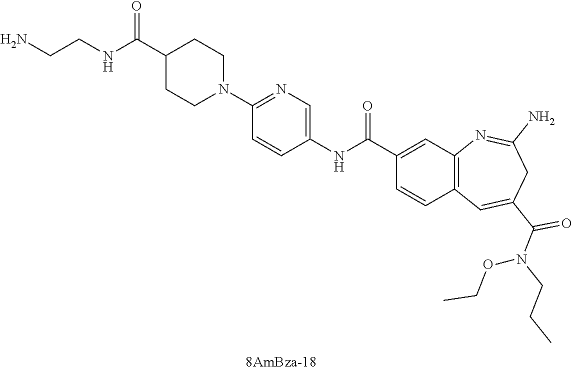 [SMILES: CCCN(OCC)C(=O)C1=Cc2ccc(C(=O)Nc3ccc(N4CCC(C(=O)NCCN)CC4)nc3)cc2N=C(N)C1]

### Preparation of 2-amino-8-bromo-N-ethoxy-N-propyl-3H-1-benzazepine-4-carboxamide, 8AmBza-18b

To a mixture of 2-amino-8-bromo-3H-1-benzazepine-4-carboxylic acid, 8AmBza-18a (9.00 g, 32.0 mmol, 1.0 eq) and N-ethoxypropan-1-amine (5.81 g, 41.6 mmol, 1.3 eq, HCl) in DCM (150 mL) and DMA (150 mL) was added 1-ethyl-3-(3-dimethylaminopropyl)carbodiimide hydrochloride, EDCI, CAS Reg. No. 1892-57-5 (24.5 g, 128 mmol, 4.0 eq) in one portion at 20° C. under N₂, and then stirred at 20° C. for 10 hours. The mixture was concentrated in vacuum to remove DCM, then water (200 mL) was added and the aqueous phase was extracted with ethyl acetate (100 mL*4), the combined organic phase was washed with brine (200 mL*1), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=10/1, 0/1) to afford 8AmBza-18b (6.00 g, 16.3 mmol, 51.1% yield) as white solid. ¹H NMR (400 MHz, MeOD) δ7.32 (d, J=2.0 Hz, 1H), 7.27-7.23 (m, 1H), 7.20 (s, 1H), 7.19-7.16 (m, 1H), 3.94 (q, J=7.2 Hz, 2H), 3.73 (t, J=7.2 Hz, 2H), 3.33 (s, 2H), 1.82-1.72 (m, 2H), 1.17 (t, J=7.2 Hz, 3H), 0.99 (t, J=7.2 Hz, 3H).

### Preparation of methyl 2-amino-4-[ethoxy(propyl)carbamoyl]-3H-1-benzazepine-8-carboxylate, 8AmBza-18c

To a solution of 2-amino-8-bromo-N-ethoxy-N-propyl-3H-1-benzazepine-4-carboxamide (340 mg, 928 umol, 1.0 eq) in MeOH (10 mL) was added Pd(dppf)Cl₂ (34.0 mg, 46.4 umol, 0.05 eq) and Et₃N (282 mg, 2.78 mmol, 388 uL, 3.0 eq) under N₂, the suspension was degassed under vacuum and purged with CO several times, the mixture was stirred under CO (50 psi) at 80° C. for 10 hours. The reaction mixture was concentrated in vacuum, then water (10 mL) was added and the aqueous phase was extracted with ethyl acetate (10 mL*3), the combined organic phase was washed with brine (10 mL*1), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=10/1, 0/1) to afford 8AmBza-18c (180 mg, 521 umol, 56.1% yield) as yellow solid. ¹H NMR (400 MHz, CDCl₃) δ7.84 (d, J=1.2 Hz, 1H), 7.69-7.65 (m, 1H), 7.46 (d, J=8.0 Hz, 1H), 7.28 (s, 1H), 3.96 (t, J=14.4 Hz, 2H), 3.93 (s, 3H), 3.74 (t, J=7.2 Hz, 2H), 3.33 (s, 2H), 1.83-1.72 (m, 2H), 1.18 (t, J=7.2 Hz, 3H), 1.00 (t, J=7.2 Hz, 3H).

### Preparation of 2-amino-4-[ethoxy(propyl)carbamoyl]-3H-1-benzazepine-8-carboxylic acid, 8AmBza-18d

To a solution of 8AmBza-18c (180 mg, 521 umol, 1.0 eq) in MeOH (1 mL) and H₂O (3 mL) was added LiOH.H₂O (65.6 mg, 1.56 mmol, 3.0 eq) in one portion at 20° C. under N₂, the mixture was stirred at 20° C. for 7 hours. The mixture was quenched with HCl (4M) until pH=7, desired solid precipitated from the mixture and then filtered to afford 8AmBza-18d (150 mg, 452 umol, 86.8% yield) as gray solid.

### Preparation of tert-butyl N-[2-[[1-[5-[[2-amino-4-[ethoxy(propyl)carbamoyl]-3H-1-benzazepine-8-carbonyl]amino]-2-pyridyl]piperidine-4-carbonyl]amino]ethyl]carbamate, 8AmBza-18e

To a solution of 8AmBza-18d (137 mg, 413 umol, 1.0 eq) in DMF (2 mL) was added HATU (141 mg, 372 umol, 0.9 eq) and DIEA (160 mg, 1.24 mmol, 216 uL, 3.0 eq) in one portion at 20° C. under N₂. The mixture was stirred at 20° C. for 30 min, then tert-butyl N-[2-[[1-(5-amino-2-pyridyl)piperidine-4-carbonyl]amino]ethyl]carbamate (195 mg, 537 umol, 1.3 eq) was added and stirred at 20° C. for another 10 hours. The reaction mixture was filtered and the filtrate was purified by prep-HPLC (column: Phenomenex Synergi C18 150*25*10 um; mobile phase: [water (0.1% TFA)-ACN]; B %: 10%-40%, 8 min) to afford 8AmBza-18e (20.0 mg, crude) as brown solid.

### Preparation of 8AmBza-18

To a solution of 8AmBza-18e (20 mg, 29.5 umol, 1.0 eq) in EtOAc (2 mL) was added HCl/EtOAc (4 M, 369 uL, 50 eq) in one portion at 20° C. under N₂, and then stirred at 20° C. for 3 hours. The reaction mixture was concentrated in vacuum and the residue was purified by prep-HPLC (column: Phenomenex Synergi C18 150*25*10 um; mobile phase: [water (0.1% TFA)-ACN]; B %: 1%-25%, 8 min) to afford 8AmBza-18 (12.6 mg, 17.5 umol, 59.2% yield, 95.98% purity, TFA) as white solid. ¹H NMR (400 MHz, MeOD) δ8.57 (d, J=2.4 Hz, 1H), 8.07 (dd, J=2.4, 9.6 Hz, 1H), 8.00-7.96 (m, 2H), 7.74 (d, J=8.4 Hz, 1H), 7.47 (s, 1H), 7.18 (d, J=9.6 Hz, 1H), 4.30 (d, J=13.6 Hz, 2H), 4.00 (q, J=7.2 Hz, 2H), 3.78 (t, J=7.2 Hz, 2H), 3.51-3.44 (m, 5H), 3.17-3.05 (m, 4H), 2.62-2.53 (m, 1H), 1.96 (d, J=3.6 Hz, 2H), 1.87-1.75 (m, 4H), 1.22 (t, J=7.2 Hz, 3H), 1.03 (t, J=7.2 Hz, 3H). LC/MS [M+H] 577.3 (calculated); LC/MS [M+H] 577.2 (observed).

### Example L-1 Synthesis of 4-((S)-2-((S)-2-(6-(2,5-dioxo-2,5-dihydro-1H-pyrrol-1-yl)hexanamido)-3-methylbutanamido)-5-ureidopentanamido)benzyl (2-(1-(5-(2-amino-4-(dipropylcarbamoyl)-3H-benzo[b]azepine-8-carboxamido)pyridin-2-yl)piperidine-4-carboxamido)ethyl)carbamate, 8AmBza-L-1

8AmBza-L-1 was prepared and characterized according to the procedures described herein.

### Example L-2 Synthesis of rac-2,3,5,6-tetrafluorophenyl (6R,9R)-1-amino-6-((4-((((2-(1-(5-(2-amino-4-(dipropylcarbamoyl)-3H-benzo[b]azepine-8-carboxamido)pyridin-2-yl)piperidine-4-carboxamido)ethyl)carbamoyl)oxy)methyl)phenyl)carbamoyl)-9-isopropyl-1,8,11-trioxo-14,17,20,23,26,29,32,35,38,41,44,47,50,53,56,59,62,65,68,71,74,77,80,83,86-pentacosaoxa-2,7,10-triazanonaoctacontan-89-oate, 8AmBza-L-2

### Preparation of bis(2,3,5,6-tetrafluorophenyl) 4,7,10,13,16,19,22,25,28,31,34,37,40,43,46,49,52,55,58,61,64,67,70,73,76-pentacosaoxanonaheptacontanedioate, TFP-PEG25-TFP

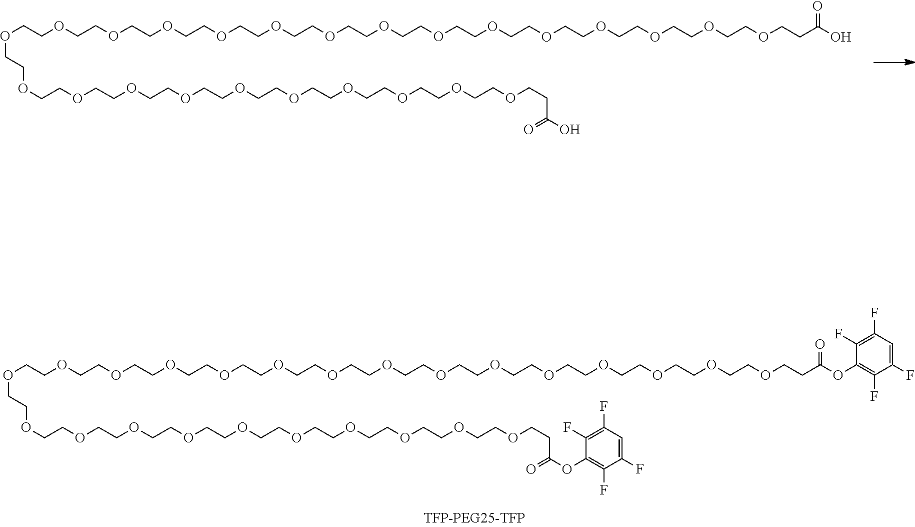 [SMILES: O=C(O)CCOCCOCCOCCOCCOCCOCCOCCOCCOCCOCCOCCOCCOCCOCCOCCOCCOCCOCCOCCOCCOCCOCCOCCOCCOCCC(=O)O ; O=C(CCOCCOCCOCCOCCOCCOCCOCCOCCOCCOCCOCCOCCOCCOCCOCCOCCOCCOCCOCCOCCOCCOCCOCCOCCOCCC(=O)Oc1c(F)c(F)cc(F)c1F)Oc1c(F)c(F)cc(F)c1F]

A vial was charged with 4,7,10,13,16,19,22,25,28,31,34,37,40,43,46,49,52,55,58,61,64,67,70,73,76-pentacosaoxanonaheptacontanedioic acid (269 mg, 0.221 mmol), 2,3,5,6-tetrafluorophenol (110 mg, 0.662 mmol), collidine (176 μL, 1.33 mmol), 1-ethyl-3-(3-dimethylaminopropyl)carbodiimide (127 mg, 0.221 mmol) and 3 mL DMF. The reaction was stirred for 16 h, then purified by reverse phase preparative HPLC utilizing a 25-75% gradient of acetonitrile:water containing 0.1% trifluoroacetic acid. The purified fractions were combined and lyophilized to afford 266 mg of TFP-PEG25-TFP in 79% yield. LC/MS [M+H] 1515.68 (calculated); LC/MS [M+H] 1516.00 (observed).

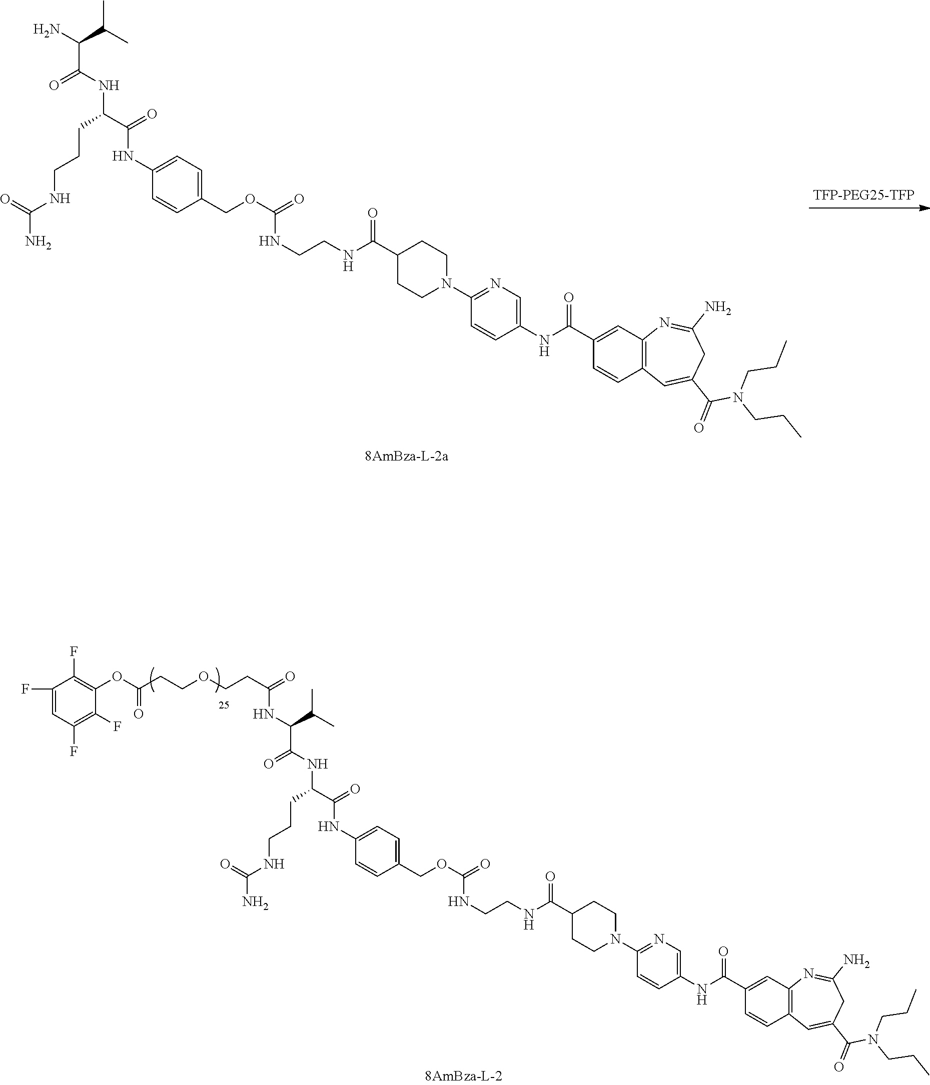

4-((S)-2-((S)-2-amino-3-methylbutanamido)-5-ureidopentanamido)benzyl (2-(1-(5-(2-amino-4-(dipropylcarbamoyl)-3H-benzo[b]azepine-8-carboxamido)pyridin-2-yl)piperidine-4-carboxamido)ethyl)carbamate, 8AmBza-L-2a and TFP-PEG25-TFP were reacted in collidine and DMF, and purified by reverse phase preparative HPLC utilizing a 25-75% gradient of acetonitrile:water containing 0.1% trifluoroacetic acid. The purified fractions were combined and lyophilized to afford 8 AmBza-L-2. LC/MS [M+2H/2] 1165.10 (calculated); LC/MS [M+H] 1165.91 (observed).

### Example L-3 Synthesis of 2,3,5,6-Tetrafluorophenyl (6S,9S)-1-amino-6-((4-(((((6-(2-amino-4-(dipropylcarbamoyl)-3H-benzo[b]azepine-8-carboxamido)pyridin-3-yl)methyl)carbamoyl)oxy)methyl)phenyl)carbamoyl)-9-isopropyl-1,8,11-trioxo-14,17,20,23,26,29,32,35,38,41,44,47,50,53,56,59,62,65,68,71,74,77,80,83,86-pentacosaoxa-2,7,10-triazanonaoctacontan-89-oate, 8AmBza-L-3

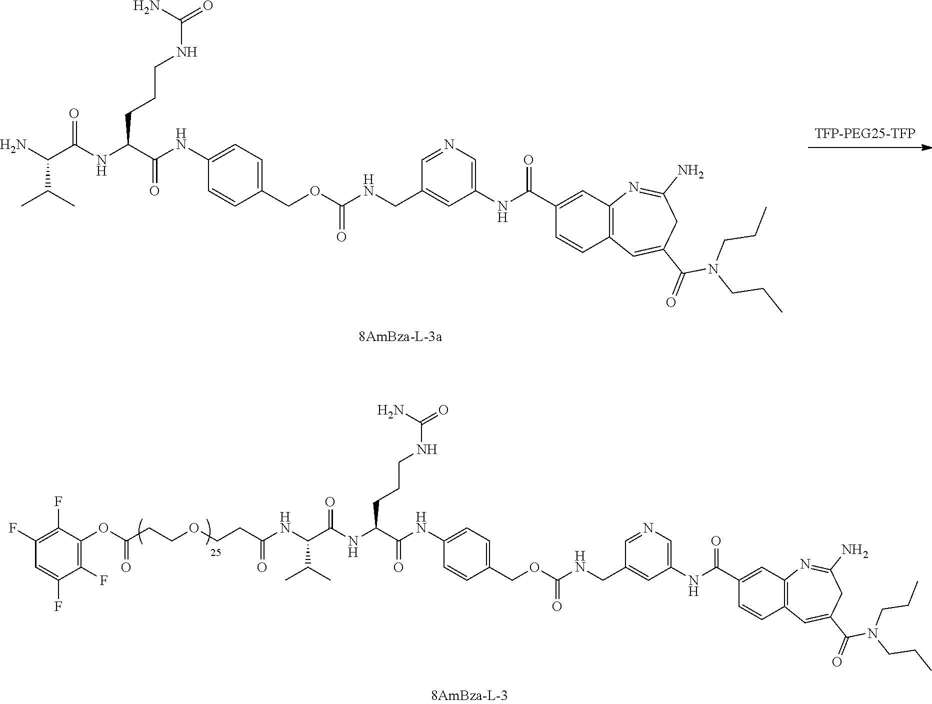

4-((S)-2-((S)-2-Amino-3-methylbutanamido)-5-ureidopentanamido)benzyl ((5-(2-amino-4-(dipropylcarbamoyl)-3H-benzo[b]azepine-8-carboxamido)pyridin-3-yl)methyl)carbamate, 8AmBza-L-3 and TFP-PEG25-TFP were reacted in collidine and DMF, and purified by reverse phase preparative HPLC utilizing a 25-75% gradient of acetonitrile:water containing 0.1% trifluoroacetic acid. The purified fractions were combined and lyophilized to afford 8 AmBza-L-3. LC/MS [M+2H/2] 1095.06 (calculated); LC/MS [M+H] 1095.87 (observed).

### Example L-4 Synthesis of 2,3,5,6-tetrafluorophenyl 1-(1-(5-(2-amino-4-(dipropylcarbamoyl)-3H-benzo[b]azepine-8-carboxamido)pyridin-2-yl)piperidin-4-yl)-1,6-dioxo-9,12,15,18,21,24,27,30,33,36,39,42,45,48,51,54,57,60,63,66,69,72,75,78,81-pentacosaoxa-2,5-diazatetraoctacontan-84-oate, 8AmBza-L-4

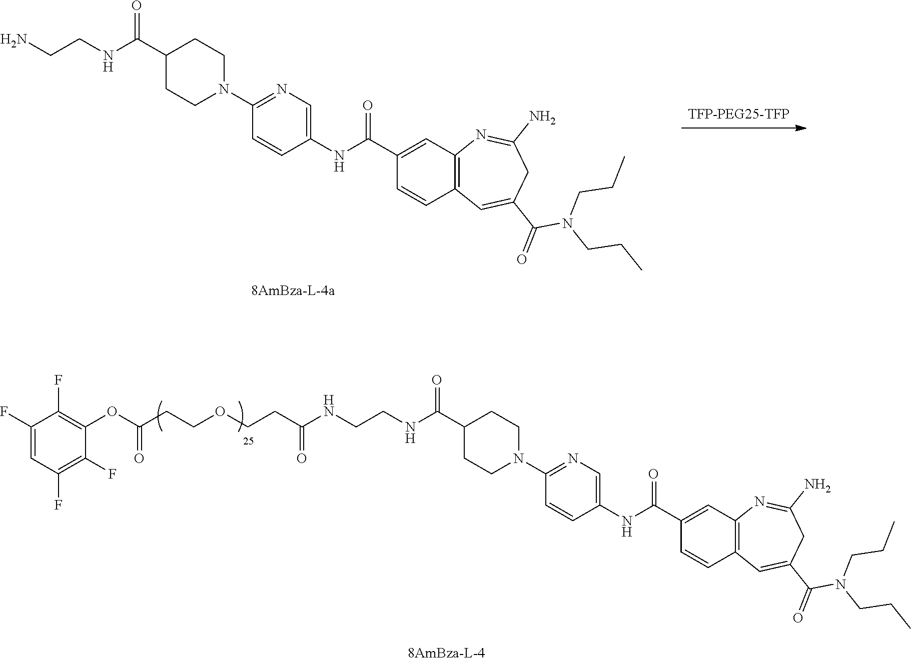

2-Amino-N8-(6-(4-((2-aminoethyl)carbamoyl)piperidin-1-yl)pyridin-3-yl)-N4,N4-dipropyl-3H-benzo[b]azepine-4,8-dicarboxamide, 8 AmBza-L-4a and TFP-PEG25-TFP reacted in collidine and DMF, and purified by reverse phase preparative HPLC utilizing a 25-75% gradient of acetonitrile:water containing 0.1% trifluoroacetic acid. The purified fractions were combined and lyophilized to afford 8 AmBza-L-4. LC/MS [M+H] 1924.01 (calculated); LC/MS [M+H] 1925.23 (observed).

### Example L-5 Synthesis of 2,3,5,6-Tetrafluorophenyl 1-(6-(2-amino-4-(dipropylcarbamoyl)-3H-benzo[b]azepine-8-carboxamido)pyridin-3-yl)-3-oxo-6,9,12,15,18,21,24,27,30,33,36,39,42,45,48,51,54,57,60,63,66,69,72,75,78-pentacosaoxa-2-azahenoctacontan-81-oate, 8AmBza-L-5

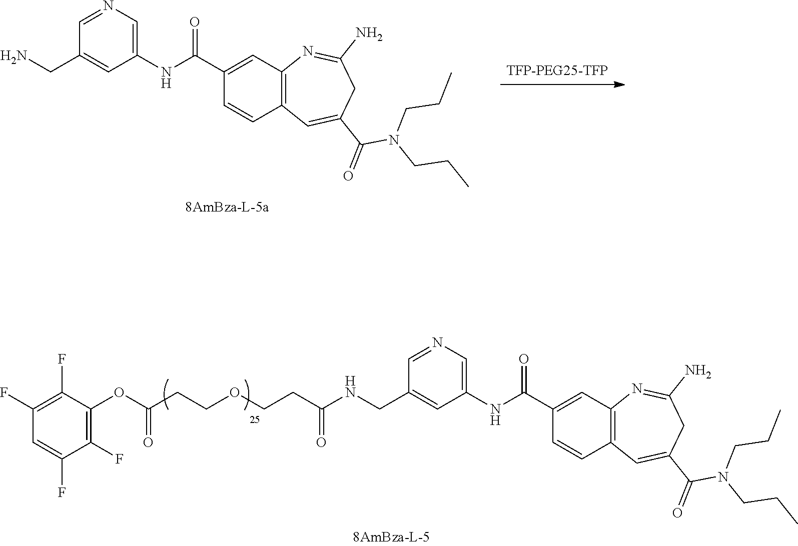

2-Amino-N8-(5-(aminomethyl)pyridin-3-yl)-N4,N4-dipropyl-3H-benzo[b]azepine-4,8-dicarboxamide, 8 AmBza-L-5a and TFP-PEG25-TFP reacted in collidine and DMF, and purified by reverse phase preparative HPLC utilizing a 25-75% gradient of acetonitrile:water containing 0.1% trifluoroacetic acid. The purified fractions were combined and lyophilized to afford 8 AmBza-L-5. LC/MS [M+H] 1783.92 (calculated); LC/MS [M+H] 1784.19 (observed).

### Example L-6 Synthesis of 2,3,5,6-Tetrafluorophenyl 1-(1-(5-(2-amino-4-((3-((tert-butoxycarbonyl)amino)propyl)(propyl)carbamoyl)-3H-benzo[b]azepine-8-carboxamido)pyridin-2-yl)piperidin-4-yl)-1,6-dioxo-9,12,15,18,21,24,27,30,33,36,39,42,45,48,51,54,57,60,63,66,69,72,75,78,81-pentacosaoxa-2,5-diazatetraoctacontan-84-oate, 8AmBza-L-6

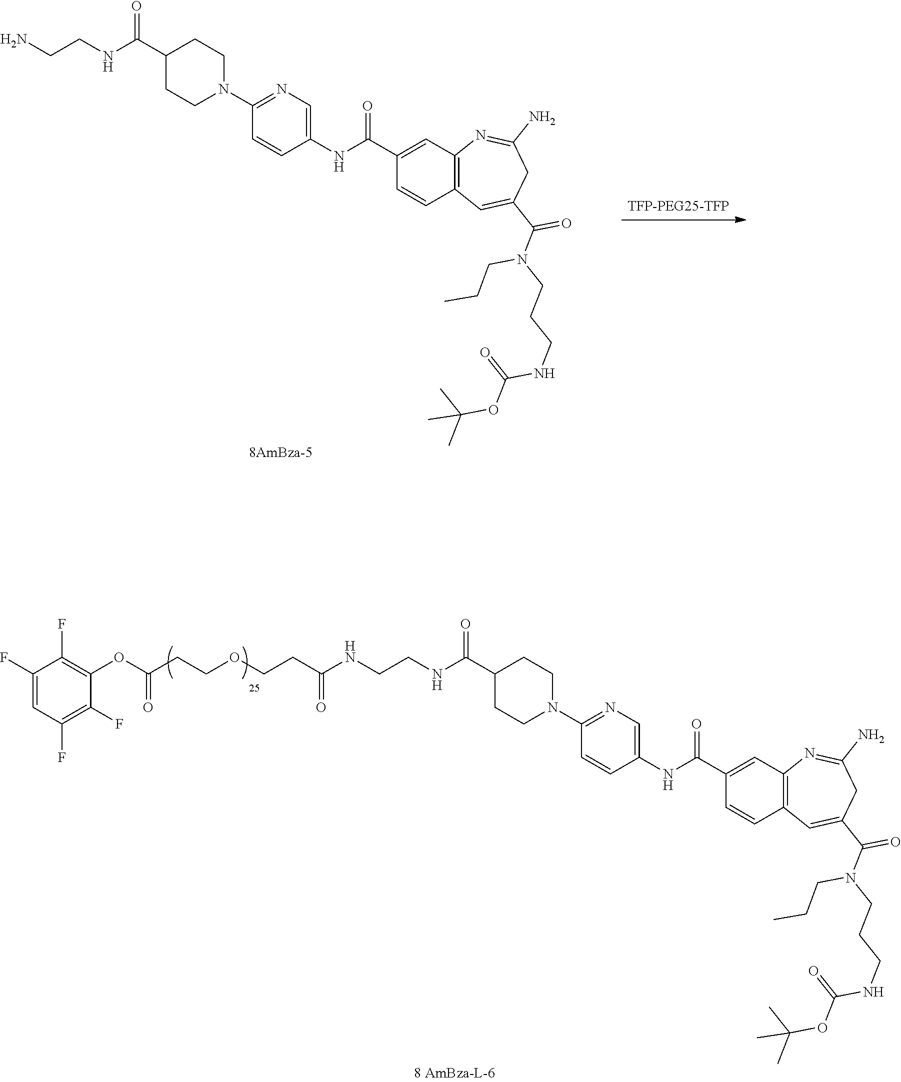

tert-Butyl (3-(2-amino-8-((6-(4-((2-aminoethyl)carbamoyl)piperidin-1-yl)pyridin-3-yl)carbamoyl)-N-propyl-3H-benzo[b]azepine-4-carboxamido)propyl)carbamate, 8AmBza-5 from Example 5 and TFP-PEG25-TFP were reacted in collidine and DMF, and purified by reverse phase preparative HPLC utilizing a 25-75% gradient of acetonitrile:water containing 0.1% trifluoroacetic acid. The purified fractions were combined and lyophilized to afford 8 AmBza-L-6. LC/MS [M+H] 2039.07 (calculated); LC/MS [M+H] 2039.40 (observed).

### Example L-7 Synthesis of (2S,4S,6S)-6-(4-((((2-(1-(5-(2-amino-4-(dipropylcarbamoyl)-3H-benzo[b]azepine-8-carboxamido)pyridin-2-yl)piperidine-4-carboxamido)ethyl)carbamoyl)oxy)methyl)-2-(20-oxo-1-(1-(2-(3-oxo-3-(perfluorophenoxy)propoxy)ethyl)-1H-1,2,3-triazol-4-yl)-2,5,8,11,14,17-hexaoxa-21-azatetracosan-24-amido)phenoxy)-3,4,5-trihydroxytetrahydro-2H-pyran-2-carboxylic acid, 8AmBza-L-7

8AmBza-L-7 was prepared and characterized according to the procedures described herein.

### Example L-8 Synthesis of 2,3,5,6-tetrafluorophenyl 1-(3-(2-amino-4-(dipropylcarbamoyl)-3H-benzo[b]azepine-8-carboxamido)phenyl)-8-methyl-2,5,11,14,17,20,23,26,29,32,35,38-dodecaoxa-8-azahentetracontan-41-oate, 8AmBza-L-8

8AmBza-L-8 was prepared and characterized according to the procedures described herein.

### Example L-9 Synthesis of 2,3,5,6-tetrafluorophenyl 1-((5-(2-amino-4-(dipropylcarbamoyl)-3H-benzo[b]azepine-8-carboxamido)pyrimidin-2-yl)amino)-3-methyl-6,9,12,15,18,21,24,27,30,33-decaoxa-3-azahexatriacontan-36-oate, 8AmBza-L-9

8AmBza-L-9 was prepared and characterized according to the procedures described herein.

### Example L-10 Synthesis of 2,3,5,6-tetrafluorophenyl (R)-1-(4-((3-(2-amino-4-(dipropylcarbamoyl)-3H-benzo[b]azepine-8-carboxamido)piperidin-1-yl)methyl)phenyl)-2-methyl-5,8,11,14,17,20,23,26,29,32-decaoxa-2-azapentatriacontan-35-oate, 8AmBza-L-10

8AmBza-L-10 was prepared and characterized according to the procedures described herein.

### Example L-11 Synthesis of 2,3,5,6-tetrafluorophenyl 1-(4-((4-(2-amino-4-(dipropylcarbamoyl)-3H-benzo[b]azepine-8-carbonyl)piperazin-1-yl)methyl)phenyl)-2-methyl-5,8,11,14,17,20,23,26,29,32-decaoxa-2-azapentatriacontan-35-oate, 8AmBza-L-11

8AmBza-L-11 was prepared and characterized according to the procedures described herein.

### Example L-16 Synthesis of (2,3,5,6-tetrafluorophenyl) 3-[2-[2-[2-[2-[2-[2-[2-[2-[2-[2-[2-[[1-[5-[[2-amino-4-[ethoxy(propyl)carbamoyl]-3H-1-benzazepine-8-carbonyl]amino]-2-pyridyl]piperidine-4-carbonyl]amino]ethyl-methyl-amino]ethoxy]ethoxy]ethoxy]ethoxy]ethoxy]ethoxy]ethoxy]ethoxy]ethoxy]ethoxy]propanoate, 8AmBza-L-16

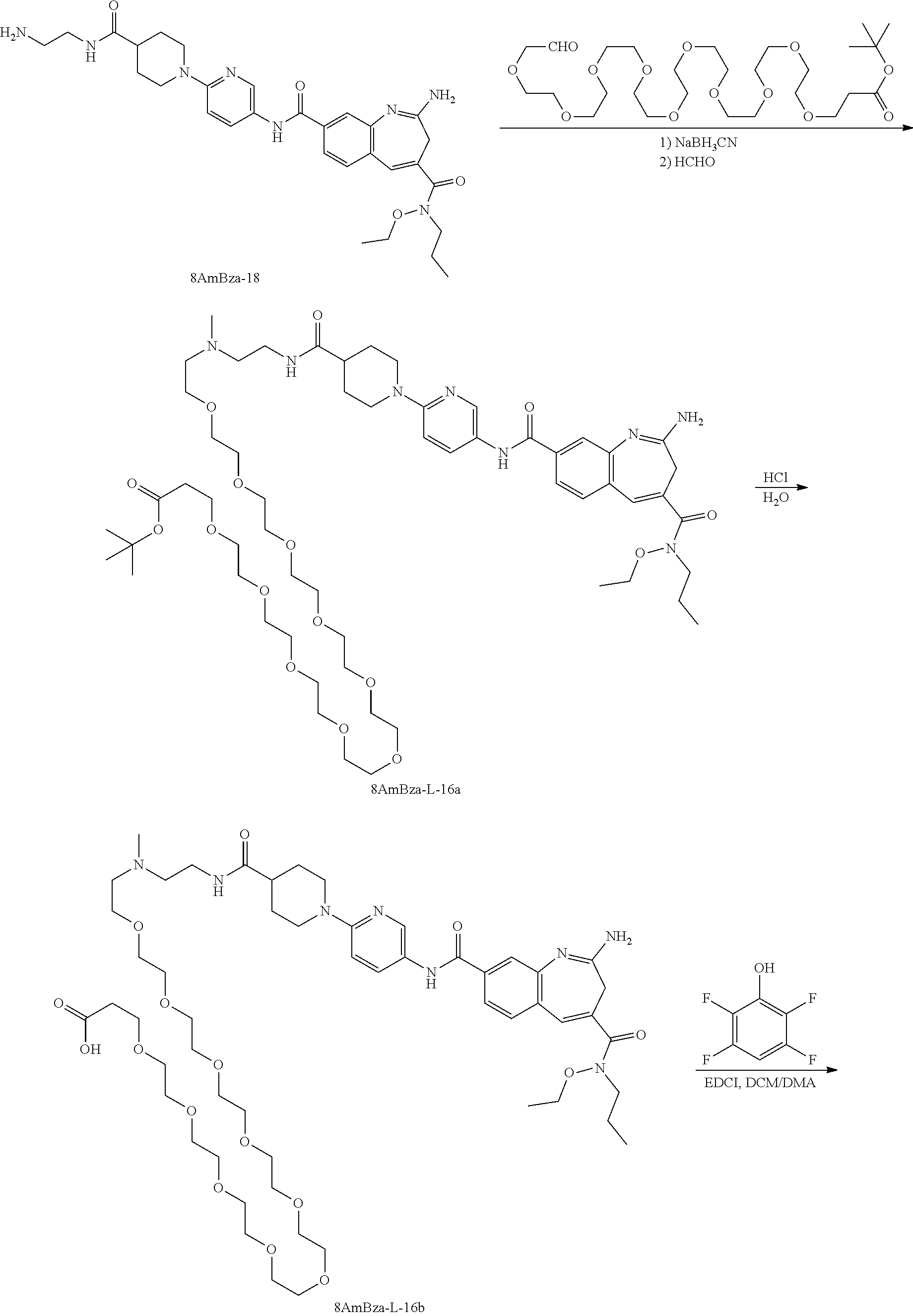

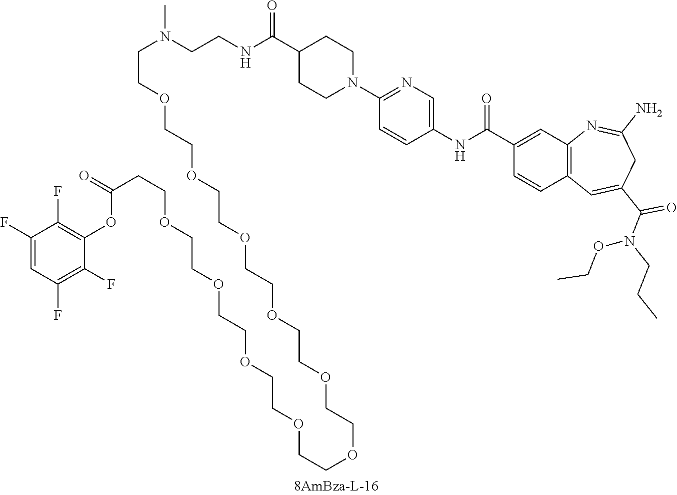 [SMILES: CCCN(OCC)C(=O)C1=Cc2ccc(C(=O)Nc3ccc(N4CCC(C(=O)NCCN(C)CCOCCOCCOCCOCCOCCOCCOCCOCCOCCOCCC(=O)Oc5c(F)c(F)cc(F)c5F)CC4)nc3)cc2N=C(N)C1]

### Preparation of tert-butyl 3-[2-[2-[2-[2-[2-[2-[2-[2-[2-[2-[2-[[1-[5-[[2-amino-4-[ethoxy(propyl)carbamoyl]-3H-1-benzazepine-8-carbonyl]amino]-2-pyridyl]piperidine-4-carbonyl]amino]ethyl-methyl-amino]ethoxy]ethoxy]ethoxy]ethoxy]ethoxy]ethoxy]ethoxy]ethoxy]ethoxy]ethoxy]propanoate, 8AmBza-L-16a

To a mixture of 2-amino-N8-[6-[4-(2-aminoethylcarbamoyl)-1-piperidyl]-3-pyridyl]-N4-ethoxy-N4-propyl-3H-1-benzazepine-4,8-dicarboxamide, 8AmBza-18 (130 mg, 225 umol, 1.0 eq) and tert-butyl 3-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]propanoate (395 mg, 676 umol, 3.0 eq) in MeOH (5 mL) was added NaBH₃CN (42.5 mg, 676 umol, 3.0 eq) and Et₃N (22.8 mg, 225 umol, 31.3 uL, 1.0 eq) in one portion at 20° C. under N₂, the mixture was stirred at 20° C. for 40 hours, then HCHO (91.4 mg, 1.13 mmol, 83.9 uL, 37% purity, 5.0 eq) was added and stirred for another 3 hours at 20° C. The reaction mixture was concentrated in vacuum and the residue was purified by prep-HPLC (column: Phenomenex Synergi C18 150*30 mm*4 um; mobile phase: [water (0.1% TFA)-ACN]; B %: 20%-45%, 8 min) to afford 8AmBza-L-16a (50.0 mg, 43.1 umol, 19.1% yield) as brown oil.

### Preparation of 3-[2-[2-[2-[2-[2-[2-[2-[2-[2-[2-[2-[[1-[5-[[2-amino-4-[ethoxy(propyl)carbamoyl]-3H-1-benzazepine-8-carbonyl]amino]-2-pyridyl]piperidine-4-carbonyl]amino]ethyl-methyl-amino]ethoxy]ethoxy]ethoxy]ethoxy]ethoxy]ethoxy]ethoxy]ethoxy]ethoxy]ethoxy]propanoic acid, 8AmBza-L-16b

To a solution of 8AmBza-L-16a (50.0 mg, 43.1 umol, 1.0 eq) in MeCN (0.5 mL) and H₂O (2 mL) was added HCl (12 M, 107 uL, 30 eq) in one portion at 20° C. under N₂, the mixture was stirred at 80° C. for 1 hour. The reaction mixture was concentrated in vacuum to afford 8AmBza-L-16b (45 mg, 40.79 umol, 94.6% yield) as colorless oil.

### Preparation of 8AmBza-L-16

To a mixture of 8AmBza-L-16b (45.0 mg, 40.7 umol, 1.0 eq) and 2,3,5,6-tetrafluorophenol (67.7 mg, 407 umol, 10 eq) in DCM (2 mL) and DMA (0.5 mL) was added EDCI (39.0 mg, 203 umol, 5.0 eq) in one portion at 20° C. under N₂, the mixture was stirred at 20° C. for 1 hour. DCM (2 mL) was removed in vacuum and the mixture was filtered, the filtrate was purified by prep-HPLC (column: Phenomenex Synergi C18 150*30 mm*4 um; mobile phase: [water (0.1% TFA)-ACN]; B %: 20%-45%, 8 min) to afford 8AmBza-L-16 (15.0 mg, 11.9 umol, 29.3% yield, 99.7% purity) as brown oil. ¹H NMR (400 MHz, MeOD) δ8.55 (d, J=1.8 Hz, 1H), 8.03 (dd, J=2.4, 9.2 Hz, 1H), 7.98 (s, 2H), 7.74 (d, J=9.2 Hz, 1H), 7.47 (s, 1H), 7.16-7.09 (m, 1H), 4.34-4.28 (m, 2H), 4.00 (d, J=7.0 Hz, 2H), 3.91-3.85 (m, 4H), 3.74-3.59 (m, 42H), 3.50 (s, 2H), 3.45 (s, 3H), 3.17-3.07 (m, 2H), 3.01 (s, 3H), 1.96 (d, J=10.6 Hz, 2H), 1.86-1.75 (m, 4H), 1.22 (t, J=7.2 Hz, 3H), 1.06-0.99 (m, 3H). LC/MS [M+H] 1251.6 (calculated); LC/MS [M+H] 1251.4 (observed).

### Example 201 Preparation of Immunoconjugates (IC)

In an exemplary procedure, an antibody is buffer exchanged into a conjugation buffer containing 100 mM boric acid, 50 mM sodium chloride, 1 mM ethylenediaminetetraacetic acid at pH 8.3, using G-25 SEPHADEX™ desalting columns (Sigma-Aldrich, St. Louis, Mo.). The eluates are then each adjusted to a concentration of about 1-10 mg/ml using the buffer and then sterile filtered. The antibody is pre-warmed to 20-30° C. and rapidly mixed with 2-20 (e.g 7-10) molar equivalents of 8AmBza-linker compound of Formula II. The reaction is allowed to proceed for about 16 hours at 30° C. and the immunoconjugate (IC) is separated from reactants by running over two successive G-25 desalting columns equilibrated in phosphate buffered saline (PBS) at pH 7.2 to provide the Immunoconjugate (IC) of Table 3. Adjuvant-antibody ratio (DAR) is determined by liquid chromatography mass spectrometry analysis using a C4 reverse phase column on an ACQUITY™ UPLC H-class (Waters Corporation, Milford, Mass.) connected to a XEVO™ G2-XS TOF mass spectrometer (Waters Corporation).

For conjugation, the antibody may be dissolved in a aqueous buffer system known in the art that will not adversely impact the stability or antigen-binding specificity of the antibody. Phosphate buffered saline may be used. The 8AmBza-linker intermediate compound is dissolved in a solvent system comprising at least one polar aprotic solvent as described elsewhere herein. In some such aspects, 8AmBza-linker intermediate is dissolved to a concentration of about 5 mM, about 10 mM, about 20 mM, about 30 mM, about 40 mM or about 50 mM, and ranges thereof such as from about 5 mM to about 50 mM or from about 10 mM to about 30 mM in pH 8 Tris buffer (e.g., 50 mM Tris). In some aspects, the 8AmBza-linker intermediate is dissolved in DMSO (dimethylsulfoxide), DMA (dimethylacetamide) or acetonitrile, or another suitable dipolar aprotic solvent.

Alternatively in the conjugation reaction, an equivalent excess of 8AmBza-linker intermediate solution may be diluted and combined with antibody solution. The 8AmBza-linker intermediate solution may suitably be diluted with at least one polar aprotic solvent and at least one polar protic solvent, examples of which include water, methanol, ethanol, n-propanol, and acetic acid. The molar equivalents of 8AmBza-linker intermediate to antibody may be about 1.5:1, about 3:1, about 5:1, about 10:1, about 15:1, or about 20:1, and ranges thereof, such as from about 1.5:1 to about 20:1 from about 1.5:1 to about 15:1, from about 1.5:1 to about 10:1, from about 3:1 to about 15:1, from about 3:1 to about 10:1, from about 5:1 to about 15:1 or from about 5:1 to about 10:1. The reaction may suitably be monitored for completion by methods known in the art, such as LC-MS. The conjugation reaction is typically complete in a range from about 1 hour to about 16 hours. After the reaction is complete, a reagent may be added to the reaction mixture to quench the reaction. If antibody thiol groups are reacting with a thiol-reactive group such as maleimide of the 8AmBza-linker intermediate, unreacted antibody thiol groups may be reacted with a capping reagent. An example of a suitable capping reagent is ethylmaleimide.

Following conjugation, the immunoconjugates may be purified and separated from unconjugated reactants and/or conjugate aggregates by purification methods known in the art such as, for example and not limited to, size exclusion chromatography, hydrophobic interaction chromatography, ion exchange chromatography, chromatofocusing, ultrafiltration, centrifugal ultrafiltration, tangential flow filtration, and combinations thereof. For instance, purification may be preceded by diluting the immunoconjugate, such in 20 mM sodium succinate, pH 5. The diluted solution is applied to a cation exchange column followed by washing with, e.g., at least 10 column volumes of 20 mM sodium succinate, pH 5. The conjugate may be suitably eluted with a buffer such as PBS.

### Example 202 HEK Reporter Assay

HEK293 reporter cells expressing human TLR7 or human TLR8 were purchased from Invivogen and vendor protocols were followed for cellular propagation and experimentation. Briefly, cells were grown to 80-85% confluence at 5% CO₂ in DMEM supplemented with 10% FBS, Zeocin, and Blasticidin. Cells were then seeded in 96-well flat plates at 4×10⁴ cells/well with substrate containing HEK detection medium and immunostimulatory molecules. Activity was measured using a plate reader at 620-655 nm wavelength.

### Example 203 Assessment of Immunoconjugate Activity In Vitro

This example shows that Immunoconjugates of the invention are effective at eliciting myeloid activation, and therefore are useful for the treatment of cancer.

Isolation of Human Antigen Presenting Cells: Human myeloid antigen presenting cells (APCs) were negatively selected from human peripheral blood obtained from healthy blood donors (Stanford Blood Center, Palo Alto, Calif.) by density gradient centrifugation using a ROSETTESEP™ Human Monocyte Enrichment Cocktail (Stem Cell Technologies, Vancouver, Canada) containing monoclonal antibodies against CD14, CD16, CD40, CD86, CD123, and HLA-DR. Immature APCs were subsequently purified to >90% purity via negative selection using an EASYSEP™ Human Monocyte Enrichment Kit (Stem Cell Technologies) without CD16 depletion containing monoclonal antibodies against CD14, CD16, CD40, CD86, CD123, and HLA-DR.

Myeloid APC Activation Assay: 2×10⁵ APCs were incubated in 96-well plates (Corning, Corning, N.Y.) containing iscove's modified dulbecco's medium, IMDM (Lonza) supplemented with 10% FBS, 100 U/mL penicillin, 100 μg/mL (micrograms per milliliter) streptomycin, 2 mM L-glutamine, sodium pyruvate, non-essential amino acids, and where indicated, various concentrations of unconjugated (naked) PD-L1 or HER2 antibodies and immunoconjugates of the invention (as prepared according to the Example above). Trastuzumab and avelumab were used as the antibody constructs. Cell-free supernatants were analyzed after 18 hours via ELISA to measure TNFα secretion as a readout of a proinflammatory response.

Activation of myeloid cell types can be measured using various screen assays in which different myeloid populations are utilized. These may include the following: monocytes isolated from healthy donor blood, M-CSF differentiated Macrophages, GM-CSF differentiated Macrophages, GM-CSF+IL-4 monocyte-derived Dendritic Cells, classical Dendritic Cells isolated from healthy donor blood, and myeloid cells polarized to an immunosuppressive state (also referred to as myeloid derived suppressor cells or MDSCs). Examples of MDSC polarized cells include monocytes differentiated toward immunosuppressive state such as M2a MΦ (IL4/IL13), M2c MΦ (IL10/TGFb), GM-CSF/IL6 MDSCs and tumor-educated monocytes (TEM). TEM differentiation can be performed using tumor-conditioned media (e.g. 786.O, MDA-MB-231, HCC1954). Primary tumor-associated myeloid cells may also include primary cells present in dissociated tumor cell suspensions (Discovery Life Sciences).

Assessment of activation of the described populations of myeloid cells may be performed as a mono-culture or as a co-culture with cells expressing the antigen of interest which the ISAC may bind to via the CDR region of the antibody. Following incubation for 18-48 hours, activation may be assessed by upregulation of cell surface co-stimulatory molecules using flow cytometry or by measurement of secreted proinflammatory cytokines. For cytokine measurement, cell-free supernatant is harvested and analyzed by cytokine bead array (e.g. LegendPlex from Biolegend) using flow cytometry.

All references, including publications, patent applications, and patents, cited herein are hereby incorporated by reference to the same extent as if each reference were individually and specifically indicated to be incorporated by reference and were set forth in its entirety herein.

1. An immunoconjugate comprising an antibody covalently attached to one or more 8-amido-2-aminobenzazepine moieties by a linker, and having Formula I:
Ab-[L-8AmBza]ₚ I
or a pharmaceutically acceptable salt thereof,
wherein:
Ab is the antibody;
p is an integer from 1 to 8;
8AmBza is the 8-amido-2-aminobenzazepine moiety having the formula: 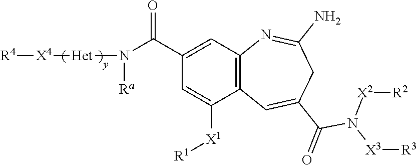
y is 0 or 1;
Het is selected from the group consisting of heterocyclyl, heterocyclyldiyl, heteroaryl, and heteroaryldiyl;
R^{a} is H or forms Het with the nitrogen atom it is bound to;
R¹, R², R³, and R⁴ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
—(C₁-C₁₂ alkyldiyl)-N(R⁵)—*;
—(C₁-C₁₂ alkyldiyl)-N(R⁵)₂;
—(C₁-C₁₂ alkyldiyl)-OR⁵;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁵—*;
—(C₃-C₁₂ carbocyclyl)-(C₁-C₁₂ alkyldiyl)-N(R⁵)₂;
—(C₃-C₁₂ carbocyclyl)-NR—C(═NR⁵)NR⁵—*;
—(C₆-C₂₀ aryl);
—(C₆-C₂₀ aryl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁵)₂;
—(C₆-C₂₀ aryldiyl)-(C₁-C₁₂ alkyldiyl)-NR⁵—C(═NR^{5a})N(R⁵)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁵—*;
—(C₂-C₉ heterocyclyl)-(C₁-C₁₂ alkyldiyl)-N(R⁵)₂;
—(C₂-C₉ heterocyclyl)-NR⁵—C(═NR^{5a})NR⁵—*;
—(C₁-C₂₀ heteroaryl);
—(C₁-C₂₀ heteroaryl)-*;
—(C₁-C₂₀ heteroaryl)-(C₁-C₁₂ alkyldiyl)-N(R⁵)—*;
—(C₁-C₂₀ heteroaryl)-(C₁-C₁₂ alkyldiyl)-N(R⁵)₂;
—(C₁-C₂₀ heteroaryl)-NR⁵—C(═NR^{5a})N(R⁵)—*;
—C(═O)—*;
—C(═O)—(C₁-C₁₂ alkyldiyl)-N(R⁵)—*;
—C(═O)—(C₂-C₂₀ heterocyclyldiyl)-*;
—C(═O)N(R⁵)₂;
—C(═O)N(R⁵)—*;
—C(═O)N(R⁵)—(C₁-C₁₂ alkyldiyl)-N(R⁵)C(═O)R⁵;
—C(═O)N(R⁵)—(C₁-C₁₂ alkyldiyl)-N(R⁵)C(═O)N(R⁵)₂;
—C(═O)NR⁵—(C₁-C₁₂ alkyldiyl)-N(R⁵)CO₂R⁵;
—C(═O)NR⁵—(C₁-C₁₂ alkyldiyl)-N(R⁵)C(═NR^{5a})N(R⁵)₂;
—C(═O)NR⁵—(C₁-C₁₂ alkyldiyl)-NR⁵C(═NR^{5a})R⁵;
—C(═O)NR⁵—(C₁-C₈ alkyldiyl)-NR⁵(C₂-C₅ heteroaryl);
—C(═O)NR⁵—(C₁-C₂₀ heteroaryldiyl)-N(R⁵)—*;
—C(═O)NR⁵—(C₁-C₂₀ heteroaryldiyl)-*;
—C(═O)NR⁵—(C₁-C₂₀ heteroaryldiyl)-(C₁-C₁₂ alkyldiyl)-N(R⁵)₂;
—C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵—*;
—N(R⁵)₂;
—N(R⁵)—*;
—N(R⁵)C(═O)R⁵;
—N(R⁵)C(═O)—*;
—N(R⁵)C(═O)N(R⁵)₂;
—N(R⁵)C(═O)N(R⁵)—*;
—N(R⁵)CO₂R⁵;
—NR⁵C(═NR^{5a})N(R⁵)₂;
—NR⁵C(═NR^{5a})N(R⁵)—*;
—NR⁵C(═NR^{5a})R⁵;
—N(R⁵)—(C₂-C₅ heteroaryl);
—O—(C₁-C₁₂ alkyl);
—O—(C₁-C₁₂ alkyldiyl)-N(R⁵)₂;
—O—(C₁-C₁₂ alkyldiyl)-N(R⁵)—*;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁵)₂;
—S(═O)₂—(C₂-C₂₀ heterocyclyldiyl)-(C₁-C₁₂ alkyldiyl)-NR⁵—*; and
—S(═O)₂—(C₂-C₂₀ heterocyclyldiyl)-(C₁-C₁₂ alkyldiyl)-OH;
or R² and R³ together form a 5- or 6-membered heterocyclyl ring;
X¹, X², X³, and X⁴ are independently selected from the group consisting of a bond, C(═O), C(═O)N(R⁵), O, N(R⁵), S, S(O)₂, and S(O)₂N(R⁵);
R⁵ is selected from the group consisting of H, C₆-C₂₀ aryl, C₆-C₂₀ aryldiyl, C₁-C₁₂ alkyl, and C₁-C₁₂ alkyldiyl, or two R⁵ groups together form a 5- or 6-membered heterocyclyl ring;
R^{5a} is selected from the group consisting of C₆-C₂₀ aryl and C₁-C₂₀ heteroaryl;
where the asterisk * indicates the attachment site of L, and where one of R¹, R², R³ and R⁴ is attached to L;
L is the linker selected from the group consisting of:
—C(═O)-(PEG)-C(═O)-(PEP)-;
—C(═O)-(PEG)-NR⁵—;
—C(═O)-(PEG)-NR⁵-(PEG)-C(═O)-(PEP)-;
—C(═O)-(PEG)-N⁺(R⁵)₂-(PEG)-C(═O)-(PEP)-;
—C(═O)-(PEG)-C(═O)—;
—C(═O)-(PEG)-NR⁵CH(AA₁)C(═O)-(PEG)-C(═O)-(PEP)-;
—C(═O)-(PEG)-SS—(C₁-C₁₂ alkyldiyl)-OC(═O)—;
—C(═O)-(PEG)-SS—(C₁-C₁₂ alkyldiyl)-C(═O)—;
—C(═O)-(PEG)-;
—C(═O)-(PEG)-C(═O)NR⁵(C₁-C₁₂ alkyldiyl)NR⁵C(═O)—(C₂-C₈ monoheterocyclyldiyl)-;
—C(═O)-(PEG)-C(═O)NR⁵(C₁-C₁₂ alkyldiyl)-;
—C(═O)—(C₁-C₁₂ alkyldiyl)-C(═O)-(PEP)-;
—C(═O)—(C₁-C₁₂ alkyldiyl)-C(═O)-(PEP)-NR(C₁-C₁₂ alkyldiyl)-;
—C(═O)—(C₁-C₁₂ alkyldiyl)-C(═O)-(PEP)-NR(C₁-C₁₂ alkyldiyl)NR⁵—C(═O);
—C(═O)—(C₁-C₁₂ alkyldiyl)-C(═O)-(PEP)-NR(C₁-C₁₂ alkyldiyl)NR⁵C(═O)—(C₂-C₅ monoheterocyclyldiyl)-;
—C(═O)—CH₂CH₂OCH₂CH₂—(C₁-C₂₀ heteroaryldiyl)-CH₂O—(PEG)-C(═O)-(MCgluc)-;
—C(═O)—CH₂CH₂OCH₂CH₂—(C₁-C₂₀ heteroaryldiyl)-CH₂O—(PEG)-C(═O)-(MCgluc)-NR⁵(C₁-C₁₂ alkyldiyl)NR⁵C(═O)—(C₂-C₅ monoheterocyclyldiyl)-;
—C(═O)-(PEG)-C(═O)—NR(C₁-C₁₂ alkyldiyl)-;
—C(═O)-(PEG)-C(═O)—NR⁵(C₁-C₁₂ alkyldiyl)NR⁵C(═O)—(C₂-C₅ monoheterocyclyldiyl)-;
—C(═O)-(PEG)-C(═O)-(PEP)-NR⁵(C₁-C₁₂ alkyldiyl)-;
—C(═O)-(PEG)-C(═O)-(PEP)-NR⁵(C₁-C₁₂ alkyldiyl)NR⁵C(═O)—(C₂-C₅ monoheterocyclyldiyl)-; and
-(succinimidyl)-(CH₂)ₘ—C(═O)-(PEP)-NR⁵(C₁-C₁₂ alkyldiyl)NR⁵C(═O)—(C₂-C₅ monoheterocyclyldiyl)-;
PEG has the formula: —(CH₂CH₂O)ₙ—(CH₂)ₘ—; m is an integer from 1 to 5, and n is an integer from 2 to 50;
PEP has the formula: 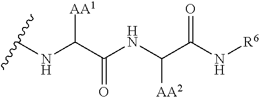
where AA₁ and AA₂ are independently selected from an amino acid side chain, or AA₁ or AA₂ and an adjacent nitrogen atom form a 5-membered ring proline amino acid, and the wavy line indicates a point of attachment and;
R⁶ is selected from the group consisting of C₆-C₂₀ aryldiyl and C₁-C₂₀ heteroaryldiyl, substituted with —CH₂O—C(═O)— and optionally with: 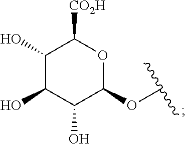 [SMILES: *O[C@@H]1O[C@H](C(=O)O)[C@@H](O)[C@H](O)[C@H]1O]
and
MCgluc is selected from the groups: 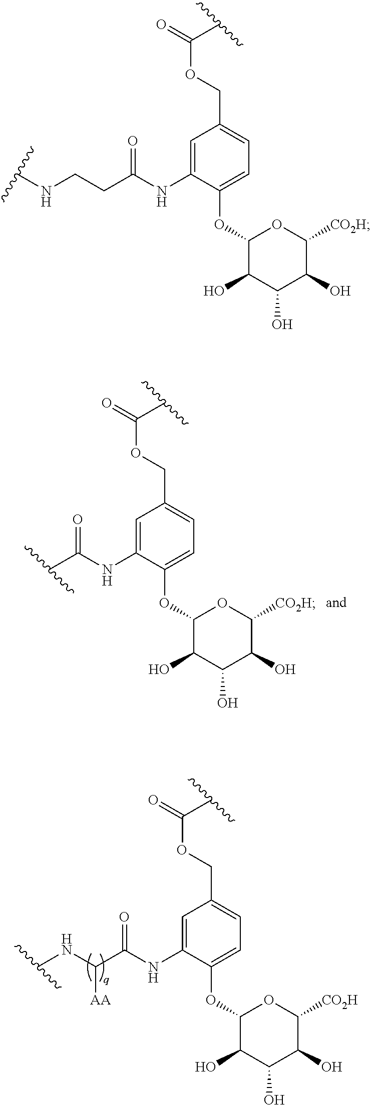
where q is 1 to 8, and AA is an amino acid side chain;
where alkyl, alkyldiyl, alkenyl, alkenyldiyl, alkynyl, alkynyldiyl, aryl, aryldiyl carbocyclyl, carbocyclyldiyl, heterocyclyl, heterocyclyldiyl, heteroaryl, and heteroaryldiyl are optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OP(O)(OH)₂, —S(O)₂N(CH₃)₂, —SCH₃, —S(O)₂CH₃, and —S(O)₃H.

2. The immunoconjugate of claim 1 wherein the antibody is an antibody construct that has an antigen binding domain that binds PD-L1.

3. The immunoconjugate of claim 2 wherein the antibody is selected from the group consisting of atezolizumab, durvalumab, and avelumab, or a biosimilar or a biobetter thereof.

4. The immunoconjugate of claim 1 wherein the antibody is an antibody construct that has an antigen binding domain that binds HER2.

5. The immunoconjugate of claim 4 wherein the antibody is selected from the group consisting of trastuzumab and pertuzumab, or a biosimilar or a biobetter thereof.

6. The immunoconjugate of claim 1 wherein the antibody is an antibody construct that has an antigen binding domain that binds CEA.

7. The immunoconjugate of claim 6 wherein the antibody is labetuzumab, or a biosimilar or a biobetter thereof.

8. The immunoconjugate of claim 1 wherein y is 0.

9. The immunoconjugate of claim 1 wherein y is 1.

10. The immunoconjugate of claim 1 wherein PEP has the formula: 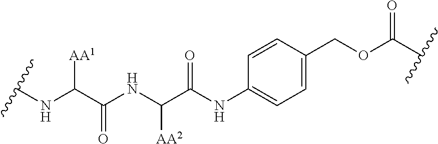
wherein AA₁ and AA₂ are independently selected from a side chain of a naturally-occurring amino acid.

11. The immunoconjugate of claim 10 wherein AA₁ or AA₂ with an adjacent nitrogen atom form a 5-membered ring proline amino acid.

12. The immunoconjugate of claim 11 wherein PEP has the formula:

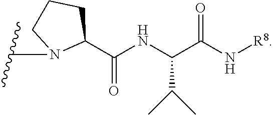

13. The immunoconjugate of claim 1 wherein MCgluc has the formula:

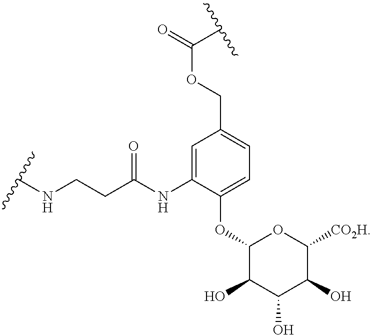 [SMILES: *NCCC(=O)Nc1cc(COC(*)=O)ccc1O[C@@H]1O[C@H](C(=O)O)[C@@H](O)[C@H](O)[C@H]1O]

14. The immunoconjugate of claim 10 wherein AA₁ and AA₂ are independently selected from H, —CH₃, —CH(CH₃)₂, —CH₂(C₆H₅), —CH₂CH₂CH₂CH₂NH₂, —CH₂CH₂CH₂NHC(NH)NH₂, —CHCH(CH₃)CH₃, —CH₂SO₃H, and —CH₂CH₂CH₂NHC(O)NH₂.

15. The immunoconjugate of claim 10 wherein AA₁ is —CH(CH₃)₂, and AA₂ is —CH₂CH₂CH₂NHC(O)NH₂.

16. The immunoconjugate of claim 1 wherein X¹ is a bond, and R¹ is H.

17. The immunoconjugate of claim 1 wherein X² is a bond, and R² is C₁-C₈ alkyl.

18. The immunoconjugate of claim 1 wherein X² and X³ are each a bond, and R² and R³ are independently selected from C₁-C₈ alkyl, —O—(C₁-C₁₂ alkyl), —(C₁-C₁₂ alkyldiyl)-OR⁵, —(C₁-C₈ alkyldiyl)-N(R⁵)CO₂R⁵, and —O—(C₁-C₁₂ alkyl)-N(R⁵)CO₂R⁵.

19. The immunoconjugate of claim 18 wherein R² and R³ are each independently selected from —CH₂CH₂CH₃, —OCH₂CH₃, —CH₂CH₂CF₃, and —CH₂CH₂CH₂OH.

20. The immunoconjugate of claim 18 wherein R² is C₁-C₈ alkyl and R³ is —(C₁-C₈ alkyldiyl)-N(R⁵)CO₂R⁴.

21. The immunoconjugate of claim 20 wherein R² is —CH₂CH₂CH₃ and R³ is —CH₂CH₂CH₂NHCO₂(t-Bu).

22. The immunoconjugate of claim 23 wherein R² and R³ are each —CH₂CH₂CH₃.

23. The immunoconjugate of claim 17 wherein X³—R³ is selected from the group consisting of:

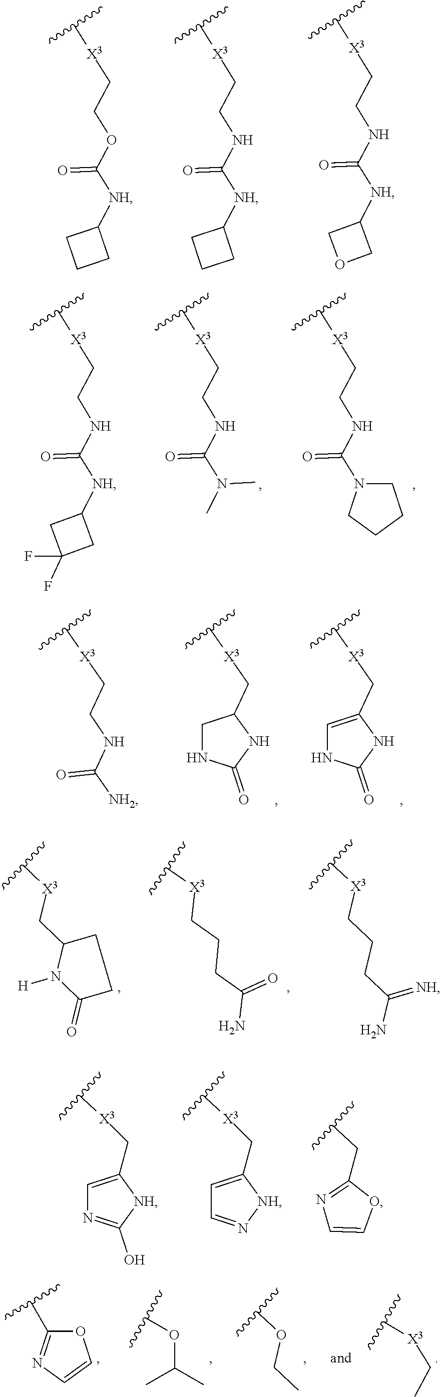

24. The immunoconjugate of claim 1 wherein Het is a 5- or 6-membered monocyclic heteroaryldiyl selected from the group consisting of pyridyldiyl, imidazolyldiyl, pyrimidinyldiyl, pyrazolyldiyl, triazolyldiyl, pyrazinyldiyl, tetrazolyldiyl, furyldiyl, thienyldiyl, isoxazolyldiyldiyl, thiazolyldiyl, oxadiazolyldiyl, oxazolyldiyl, isothiazolyldiyl, and pyrrolyldiyl.

25. The immunoconjugate of claim 1 wherein Het is a 5- or 6-membered monocyclic heterocyclyldiyl selected from the group consisting of morpholinyldiyl, piperidinyldiyl, piperazinyldiyl, pyrrolidinyldiyl, dioxanyldiyl, thiomorpholinyldiyl, and S-dioxothiomorpholinyldiyl.

26. The immunoconjugate of claim 1 wherein Het is 1,6-naphthyridyl or 1,6-naphthyridiyl.

27. The immunoconjugate of claim 1 wherein L is selected from the group consisting of:
—C(═O)—CH₂CH₂OCH₂CH₂—(C₁-C₂₀ heteroaryldiyl)-CH₂O—(PEG)-C(═O)-(MCgluc)-NR⁵(C₁-C₁₂ alkyldiyl)NR⁵C(═O)—(C₂-C₅ monoheterocyclyldiyl)-;
—C(═O)-(PEG)-C(═O)—NR⁵(C₁-C₁₂ alkyldiyl)-;
—C(═O)-(PEG)-C(═O)—NR⁵(C₁-C₁₂ alkyldiyl)NR⁵C(═O)—(C₂-C₅ monoheterocyclyldiyl)-;
—C(═O)-(PEG)-C(═O)-(PEP)-NR⁵(C₁-C₁₂ alkyldiyl)-;
—C(═O)-(PEG)-C(═O)-(PEP)-NR⁵(C₁-C₁₂ alkyldiyl)NR⁵C(═O)—(C₂-C₅ monoheterocyclyldiyl)-; and
-(succinimidyl)-(CH₂)ₘ—C(═O)-(PEP)-NR⁵(C₁-C₁₂ alkyldiyl)NR⁵C(═O)—(C₂-C₅ monoheterocyclyldiyl)-.

28. The immunoconjugate of claim 1 selected from Formulae Ia-d:

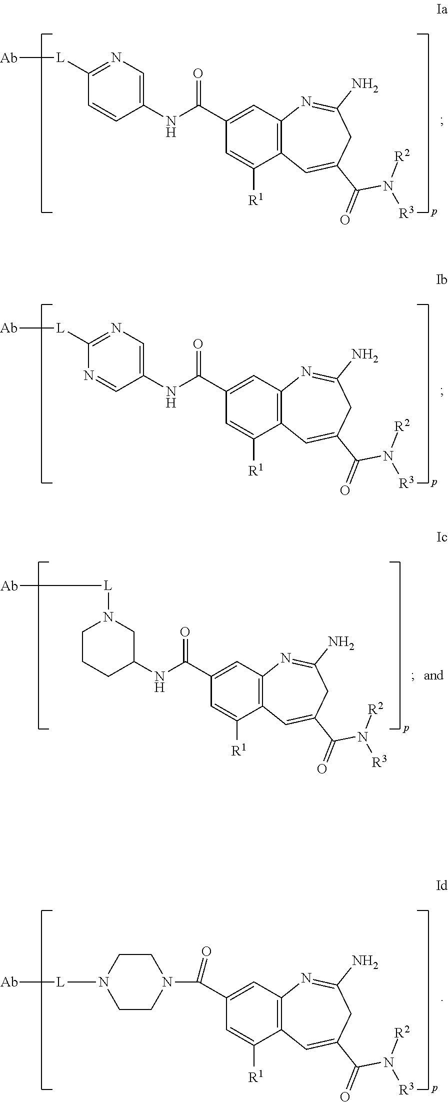

29. An 8-amido-2-aminobenzazepine-linker compound of Formula II: 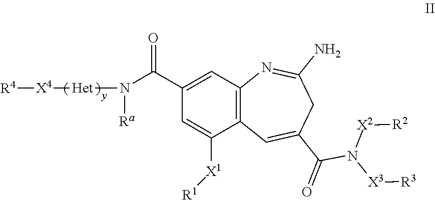
wherein
y is 0 or 1;
Het is selected from the group consisting of heterocyclyl, heterocyclyldiyl, heteroaryl, and heteroaryldiyl;
R^{a} is H or forms Het with the nitrogen atom it is bound to;
R¹, R², R³, and R⁴ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
—(C₁-C₁₂ alkyldiyl)-N(R⁵)—*;
—(C₁-C₁₂ alkyldiyl)-N(R⁵)₂;
—(C₁-C₁₂ alkyldiyl)-OR⁵;
—(C₃-C₁₂ carbocyclyl);
—(C₃-C₁₂ carbocyclyl)-*;
—(C₃-C₁₂ carbocyclyl)-(C₁-C₁₂ alkyldiyl)-NR⁵—*;
—(C₃-C₁₂ carbocyclyl)-(C₁-C₁₂ alkyldiyl)-N(R⁵)₂;
—(C₃-C₁₂ carbocyclyl)-NR⁵—C(═NR⁵)NR⁵—*;
—(C₆-C₂₀ aryl);
—(C₆-C₂₀ aryl)-*;
—(C₆-C₂₀ aryldiyl)-N(R⁵)—*;
—(C₆-C₂₀ aryldiyl)-(C₁-C₁₂ alkyldiyl)-N(R⁵)—*;
—(C₆-C₂₀ aryldiyl)-(C₁-C₁₂ alkyldiyl)-(C₂-C₂₀ heterocyclyldiyl)-*;
—(C₆-C₂₀ aryldiyl)-(C₁-C₁₂ alkyldiyl)-N(R⁵)₂;
—(C₆-C₂₀ aryldiyl)-(C₁-C₁₂ alkyldiyl)-NR⁵—C(═NR^{5a})N(R⁵)—*;
—(C₂-C₂₀ heterocyclyl);
—(C₂-C₂₀ heterocyclyl)-*;
—(C₂-C₉ heterocyclyl)-(C₁-C₁₂ alkyldiyl)-NR⁵—*;
—(C₂-C₉ heterocyclyl)-(C₁-C₁₂ alkyldiyl)-N(R⁵)₂;
—(C₂-C₉ heterocyclyl)-NR⁵—C(═NR^{5a})NR⁵—*;
—(C₁-C₂₀ heteroaryl);
—(C₁-C₂₀ heteroaryl)-*;
—(C₁-C₂₀ heteroaryl)-(C₁-C₁₂ alkyldiyl)-N(R⁵)—*;
—(C₁-C₂₀ heteroaryl)-(C₁-C₁₂ alkyldiyl)-N(R⁵)₂;
—(C₁-C₂₀ heteroaryl)-NR⁵—C(═NR^{5a})N(R⁵)—*;
—C(═O)—*;
—C(═O)—(C₁-C₁₂ alkyldiyl)-N(R⁵)—*;
—C(═O)—(C₂-C₂₀ heterocyclyldiyl)-*;
—C(═O)N(R⁵)₂;
—C(═O)N(R⁵)—*;
—C(═O)N(R⁵)—(C₁-C₁₂ alkyldiyl)-N(R⁵)C(═O)R⁵;
—C(═O)N(R⁵)—(C₁-C₁₂ alkyldiyl)-N(R⁵)C(═O)N(R⁵)₂;
—C(═O)NR⁵—(C₁-C₁₂ alkyldiyl)-N(R⁵)CO₂R⁵;
—C(═O)NR⁵—(C₁-C₁₂ alkyldiyl)-N(R⁵)C(═NR^{5a})N(R⁵)₂;
—C(═O)NR⁵—(C₁-C₁₂ alkyldiyl)-NR⁵C(═NR^{5a})R⁵;
—C(═O)NR⁵—(C₁-C₈ alkyldiyl)-NR⁵(C₂-C₅ heteroaryl);
—C(═O)NR⁵—(C₁-C₂₀ heteroaryldiyl)-N(R⁵)—*;
—C(═O)NR⁵—(C₁-C₂₀ heteroaryldiyl)-*;
—C(═O)NR⁵—(C₁-C₂₀ heteroaryldiyl)-(C₁-C₁₂ alkyldiyl)-N(R⁵)₂;
—C(═O)NR⁵—(C₁-C₂₀ heteroaryldiyl)-(C₂-C₂₀ heterocyclyldiyl)-C(═O)NR⁵—(C₁-C₁₂ alkyldiyl)-NR⁵—*;
—N(R⁵)₂;
—N(R⁵)—*;
—N(R⁵)C(═O)R⁵;
—N(R⁵)C(═O)—*;
—N(R⁵)C(═O)N(R⁵)₂;
—N(R⁵)C(═O)N(R⁵)—*;
—N(R⁵)CO₂R⁵;
—NR⁵C(═NR^{5a})N(R⁵)₂;
—NR⁵C(═NR^{5a})N(R⁵)—*;
—NR⁵C(═NR^{5a})R⁵;
—N(R⁵)—(C₂-C₅ heteroaryl);
—O—(C₁-C₁₂ alkyl);
—O—(C₁-C₁₂ alkyldiyl)-N(R⁵)₂;
—O—(C₁-C₁₂ alkyldiyl)-N(R⁵)—*;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁵)₂;
—S(═O)₂—(C₂-C₂₀ heterocyclyldiyl)-(C₁-C₁₂ alkyldiyl)-NR⁵—*; and
—S(═O)₂—(C₂-C₂₀ heterocyclyldiyl)-(C₁-C₁₂ alkyldiyl)-OH;
or R² and R³ together form a 5- or 6-membered heterocyclyl ring;
X¹, X², X³, and X⁴ are independently selected from the group consisting of a bond, C(═O), C(═O)N(R⁵), O, N(R⁵), S, S(O)₂, and S(O)₂N(R⁵);
R⁵ is selected from the group consisting of H, C₆-C₂₀ aryl, C₆-C₂₀ aryldiyl, C₁-C₁₂ alkyl, and C₁-C₁₂ alkyldiyl, or two R⁵ groups together form a 5- or 6-membered heterocyclyl ring;
R^{5a} is selected from the group consisting of C₆-C₂₀ aryl and C₁-C₂₀ heteroaryl;
where the asterisk * indicates the attachment site of L, and where one of R¹, R², R³ and R⁴ is attached to L;
L is the linker selected from the group consisting of:
Q-C(═O)-(PEG)-C(═O)-(PEP)-;
Q-C(═O)-(PEG)-NR⁵—;
Q-C(═O)-(PEG)-NR⁵-(PEG)-C(═O)-(PEP)-;
Q-C(═O)-(PEG)-N⁺(R⁵)₂-(PEG)-C(═O)-(PEP)-;
Q-C(═O)-(PEG)-C(═O)—;
Q-C(═O)-(PEG)-NR⁵CH(AA₁)C(═O)-(PEG)-C(═O)-(PEP)-;
Q-C(═O)-(PEG)-SS—(C₁-C₁₂ alkyldiyl)-OC(═O)—;
Q-C(═O)-(PEG)-SS—(C₁-C₁₂ alkyldiyl)-C(═O)—;
Q-C(═O)-(PEG)-;
Q-C(═O)-(PEG)-C(═O)NR(C₁-C₁₂ alkyldiyl)NR⁵C(═O)—(C₂-C₅ monoheterocyclyldiyl)-;
Q-C(═O)-(PEG)-C(═O)NR⁵(C₁-C₁₂ alkyldiyl)-;
Q-C(═O)—(C₁-C₁₂ alkyldiyl)-C(═O)-(PEP)-;
Q-C(═O)—(C₁-C₁₂ alkyldiyl)-C(═O)-(PEP)-NR⁵(C₁-C₁₂ alkyldiyl)-;
Q-C(═O)—(C₁-C₁₂ alkyldiyl)-C(═O)-(PEP)-NR⁵(C₁-C₁₂ alkyldiyl)NR⁵—C(═O);
Q-C(═O)—(C₁-C₁₂ alkyldiyl)-C(═O)-(PEP)-NR⁵(C₁-C₁₂ alkyldiyl)NR⁵C(═O)—(C₂-C₅ monoheterocyclyldiyl)-;
Q-C(═O)—CH₂CH₂OCH₂CH₂—(C₁-C₂₀ heteroaryldiyl)-CH₂O—(PEG)-C(═O)-(MCgluc)-;
Q-C(═O)—CH₂CH₂OCH₂CH₂—(C₁-C₂₀ heteroaryldiyl)-CH₂O—(PEG)-C(═O)-(MCgluc)-NR⁵(C₁-C₁₂ alkyldiyl)NR⁵C(═O)—(C₂-C₅ monoheterocyclyldiyl)-;
Q-C(═O)-(PEG)-C(═O)—NR⁵(C₁-C₁₂ alkyldiyl)-;
Q-C(═O)-(PEG)-C(═O)—NR⁵(C₁-C₁₂ alkyldiyl)NR⁵C(═O)—(C₂-C₅ monoheterocyclyldiyl)-;
Q-C(═O)-(PEG)-C(═O)-(PEP)-NR⁵(C₁-C₁₂ alkyldiyl)-;
Q-C(═O)-(PEG)-C(═O)-(PEP)-NR⁵(C₁-C₁₂ alkyldiyl)NR⁵C(═O)—(C₂-C₅ monoheterocyclyldiyl)-; and
Q-(CH₂)ₘC(═O)-(PEP)-NR⁵(C₁-C₁₂ alkyldiyl)NR⁵C(═O)—(C₂-C₅ monoheterocyclyldiyl)-;
where PEG has the formula: —(CH₂CH₂O)ₙ—(CH₂)ₘ—; m is an integer from 1 to 5, and n is an integer from 2 to 50;
PEP has the formula: 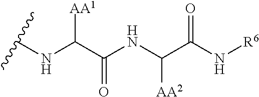
where AA₁ and AA₂ are independently selected from an amino acid side chain, or AA₁ or AA₂ and an adjacent nitrogen atom form a 5-membered ring proline amino acid, and the wavy line indicates a point of attachment and;
R⁶ is selected from the group consisting of C₆-C₂₀ aryldiyl and C₁-C₂₀ heteroaryldiyl, substituted with —CH₂O—C(═O)— and optionally with: 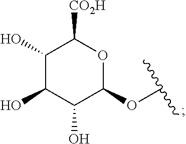 [SMILES: *O[C@@H]1O[C@H](C(=O)O)[C@@H](O)[C@H](O)[C@H]1O]
and
MCgluc is selected from the groups: 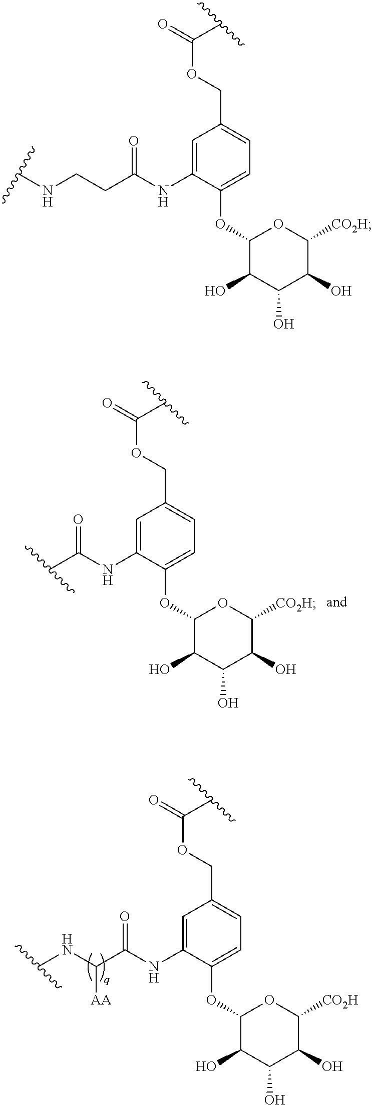
where q is 1 to 8, and AA is an amino acid side chain; and
Q is selected from the group consisting of N-hydroxysuccinimidyl, N-hydroxysulfosuccinimidyl, maleimide, and phenoxy substituted with one or more groups independently selected from F, Cl, NO₂, and SO₃—;
where alkyl, alkyldiyl, alkenyl, alkenyldiyl, alkynyl, alkynyldiyl, aryl, aryldiyl carbocyclyl, carbocyclyldiyl, heterocyclyl, heterocyclyldiyl, heteroaryl, and heteroaryldiyl are optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OP(O)(OH)₂, —S(O)₂N(CH₃)₂, —SCH₃, —S(O)₂CH₃, and —S(O)₃H.

30. The 8-amido-2-aminobenzazepine-linker compound of claim 29 wherein y is 0.

31. The 8-amido-2-aminobenzazepine-linker compound of claim 29 wherein y is 1.

32. The 8-amido-2-aminobenzazepine-linker compound of claim 29 wherein PEP has the formula: 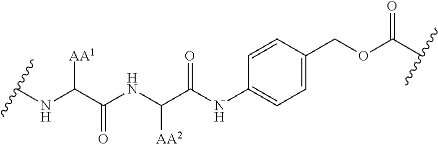
wherein AA₁ and AA₂ are independently selected from a side chain of a naturally-occurring amino acid.

33. The 8-amido-2-aminobenzazepine-linker compound of claim 32 wherein AA₁ or AA₂ with an adjacent nitrogen atom form a 5-membered ring to form a proline amino acid.

34. The 8-amido-2-aminobenzazepine-linker compound of claim 33 wherein PEP has the formula:

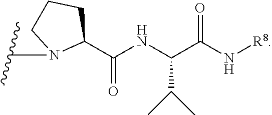

35. The 8-amido-2-aminobenzazepine-linker compound of claim 29 wherein MCgluc has the formula:

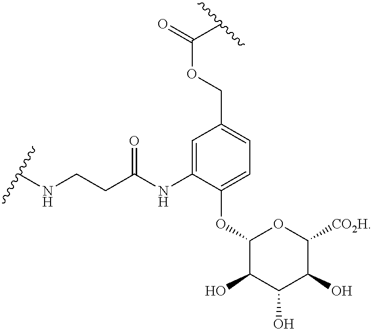 [SMILES: *NCCC(=O)Nc1cc(COC(*)=O)ccc1O[C@@H]1O[C@H](C(=O)O)[C@@H](O)[C@H](O)[C@H]1O]

36. The 8-amido-2-aminobenzazepine-linker compound of claim 32 wherein AA₁ and AA₂ are independently selected from H, —CH₃, —CH(CH₃)₂, —CH₂(C₆H₅), —CH₂CH₂CH₂CH₂NH₂, —CH₂CH₂CH₂NHC(NH)NH₂, —CHCH(CH₃)CH₃, —CH₂SO₃H, and —CH₂CH₂CH₂NHC(O)NH₂.

37. The 8-amido-2-aminobenzazepine-linker compound of claim 32 wherein AA₁ is —CH(CH₃)₂, and AA₂ is —CH₂CH₂CH₂NHC(O)NH₂.

38. The 8-amido-2-aminobenzazepine-linker compound of claim 29 wherein X¹ is a bond, and R¹ is H.

39. The 8-amido-2-aminobenzazepine-linker compound of claim 29 wherein X² is a bond, and R² is C₁-C₈ alkyl.

40. The 8-amido-2-aminobenzazepine-linker compound of claim 29 wherein X² and X³ are each a bond, and R² and R³ are independently selected from C₁-C₈ alkyl, —O—(C₁-C₁₂ alkyl), —(C₁-C₁₂ alkyldiyl)-OR⁵, —(C₁-C₈ alkyldiyl)-N(R⁵)CO₂R⁵, and —O—(C₁-C₁₂ alkyl)-N(R⁵)CO₂R⁵.

41. The 8-amido-2-aminobenzazepine-linker compound of claim 40 wherein R² and R³ are each independently selected from —CH₂CH₂CH₃, —OCH₂CH₃, —CH₂CH₂CF₃, and —CH₂CH₂CH₂OH.

42. The 8-amido-2-aminobenzazepine-linker compound of claim 40 wherein R² is C₁-C₈ alkyl and R³ is —(C₁-C₈ alkyldiyl)-N(R⁵)CO₂R⁴.

43. The 8-amido-2-aminobenzazepine-linker compound of claim 42 wherein R² is —CH₂CH₂CH₃ and R³ is —CH₂CH₂CH₂NHCO₂(t-Bu).

44. The 8-amido-2-aminobenzazepine-linker compound of claim 40 wherein R² and R³ are each —CH₂CH₂CH₃.

45. The 5-amino-pyrazoloazepine-linker compound of claim 39 wherein X³—R³ is selected from the group of:

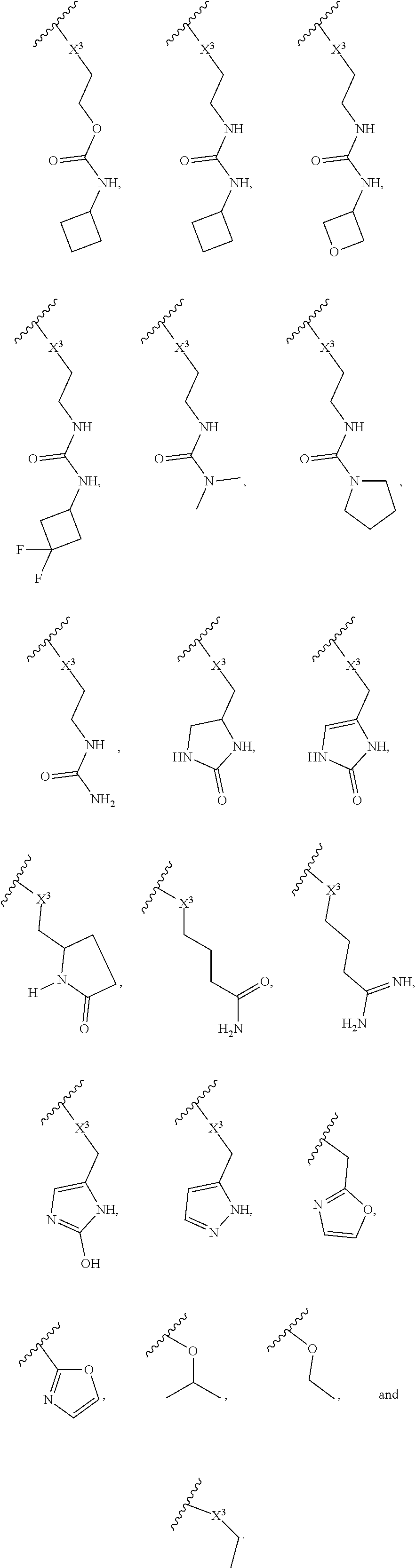

46. The 8-amido-2-aminobenzazepine-linker compound of claim 29 wherein Het is a 5- or 6-membered monocyclic heteroaryldiyl selected from the group consisting of pyridyldiyl, imidazolyldiyl, pyrimidinyldiyl, pyrazolyldiyl, triazolyldiyl, pyrazinyldiyl, tetrazolyldiyl, furyldiyl, thienyldiyl, isoxazolyldiyldiyl, thiazolyldiyl, oxadiazolyldiyl, oxazolyldiyl, isothiazolyldiyl, and pyrrolyldiyl.

47. The 8-amido-2-aminobenzazepine-linker compound of claim 29 wherein Het is a 5- or 6-membered monocyclic heterocyclyldiyl selected from the group consisting of morpholinyldiyl, piperidinyldiyl, piperazinyldiyl, pyrrolidinyldiyl, dioxanyldiyl, thiomorpholinyldiyl, and S-dioxothiomorpholinyldiyl.

48. The 8-amido-2-aminobenzazepine-linker compound of claim 29 wherein Het is 1,6-naphthyridyl or 1,6-naphthyridiyl.

49. The 8-amido-2-aminobenzazepine-linker compound of claim 29 wherein L is selected from the group consisting of:
Q-C(═O)—CH₂CH₂OCH₂CH₂—(C₁-C₂₀ heteroaryldiyl)-CH₂O—(PEG)-C(═O)-(MCgluc)-NR⁵(C₁-C₁₂ alkyldiyl)NR⁵C(═O)—(C₂-C₅ monoheterocyclyldiyl)-;
Q-C(═O)-(PEG)-C(═O)—NR⁵(C₁-C₁₂ alkyldiyl)-;
Q-C(═O)-(PEG)-C(═O)—NR⁵(C₁-C₁₂ alkyldiyl)NR⁵C(═O)—(C₂-C₅ monoheterocyclyldiyl)-;
Q-C(═O)-(PEG)-C(═O)-(PEP)-NR⁵(C₁-C₁₂ alkyldiyl)-;
Q-C(═O)-(PEG)-C(═O)-(PEP)-NR⁵(C₁-C₁₂ alkyldiyl)NR⁵C(═O)—(C₂-C₅ monoheterocyclyldiyl)-; and
Q-(CH₂)ₘ—C(═O)-(PEP)-NR⁵(C₁-C₁₂ alkyldiyl)NR⁵C(═O)—(C₂-C₅ monoheterocyclyldiyl)-.

50. The 8-amido-2-aminobenzazepine-linker compound of claim 29 wherein Q is selected from:

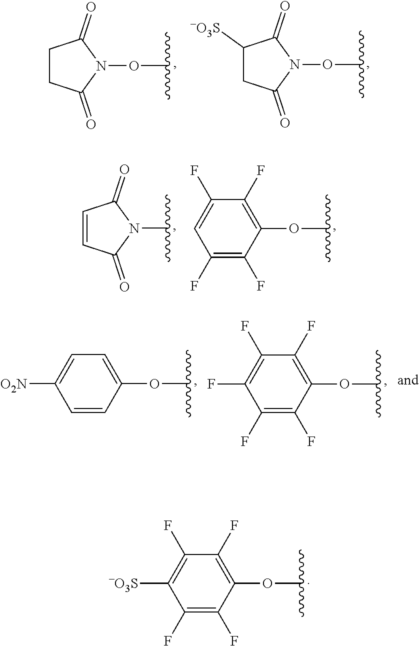

51. The aminoquinoline-linker compound of claim 29 wherein Q is phenoxy substituted with one or more F.

52. The aminoquinoline-linker compound of claim 51 wherein Q is 2,3,5,6-tetrafluorophenoxy.

53. The 8-amido-2-aminobenzazepine-linker compound of claim 29 selected from Formulae IIa-d:

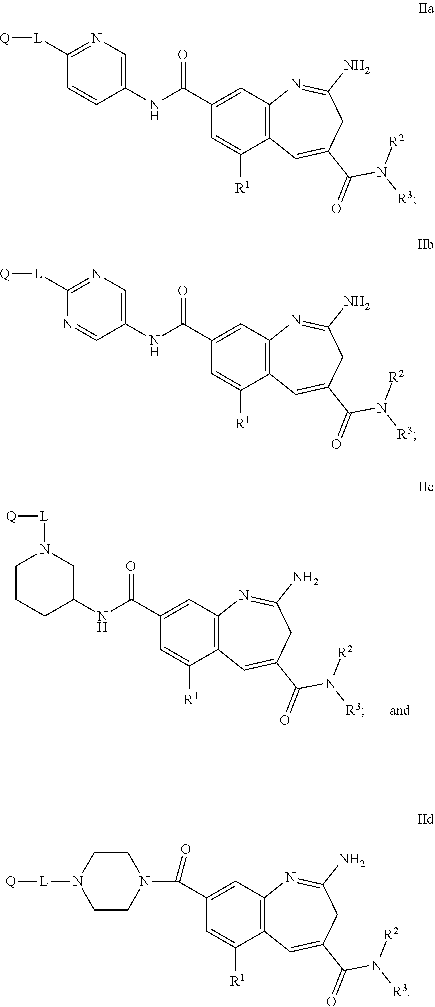

54. The 8-amido-2-aminobenzazepine-linker compound of claim 29 selected from the group consisting of:

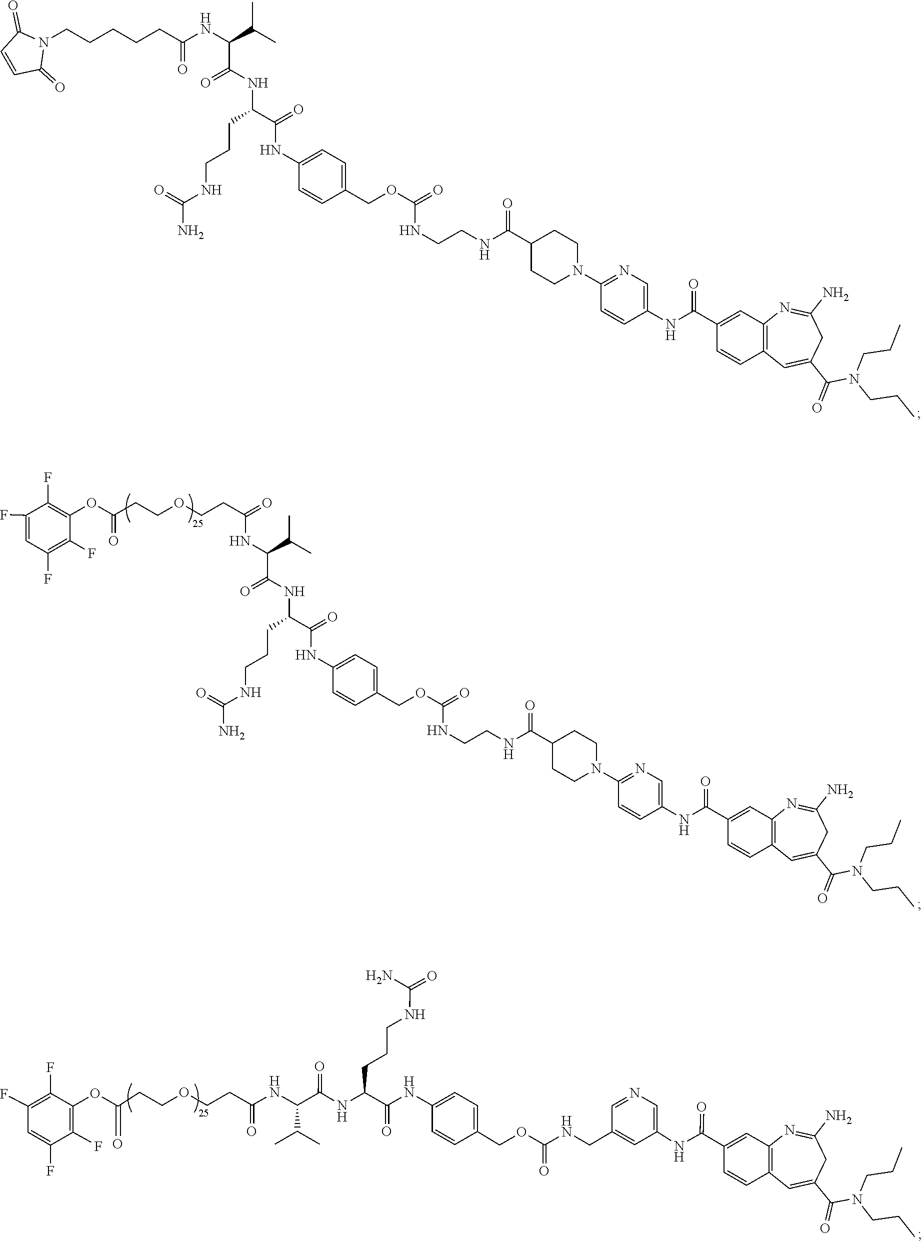

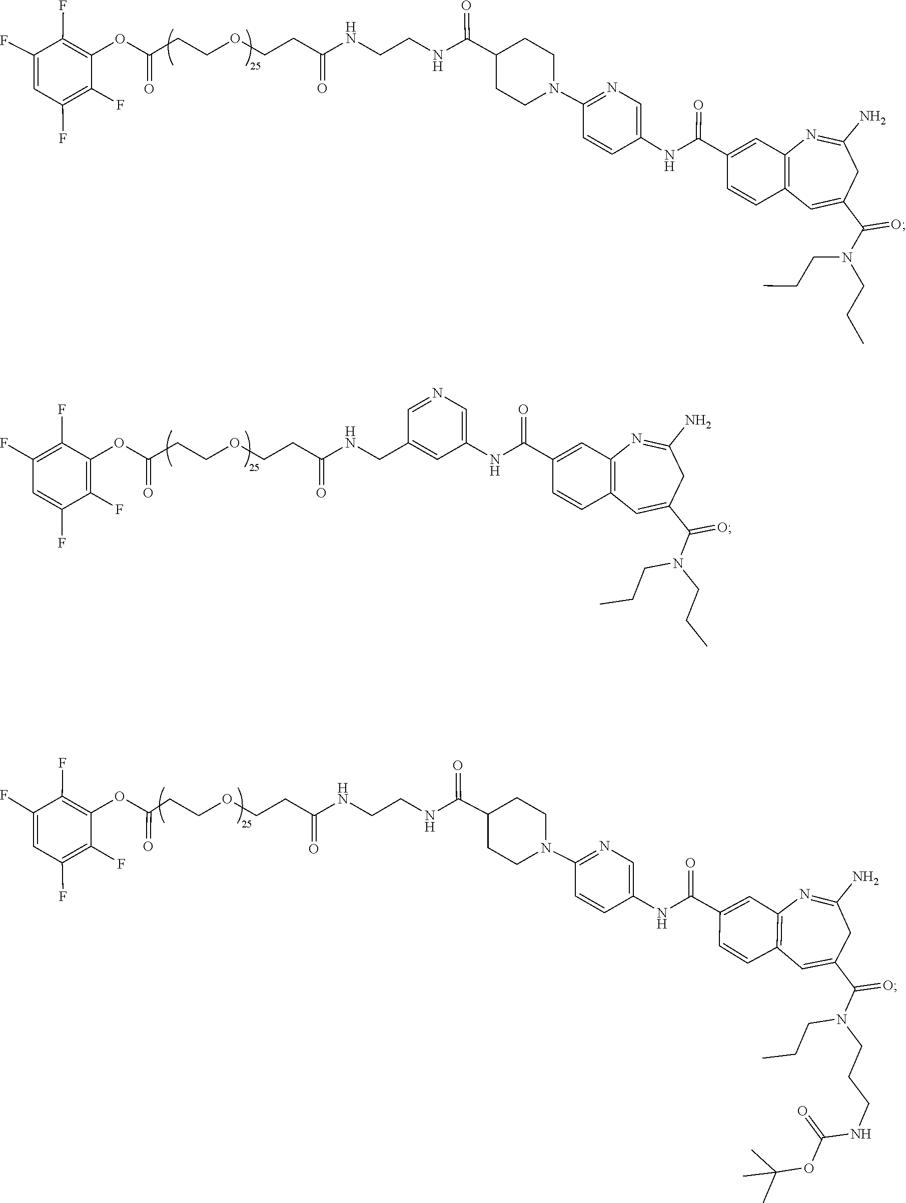

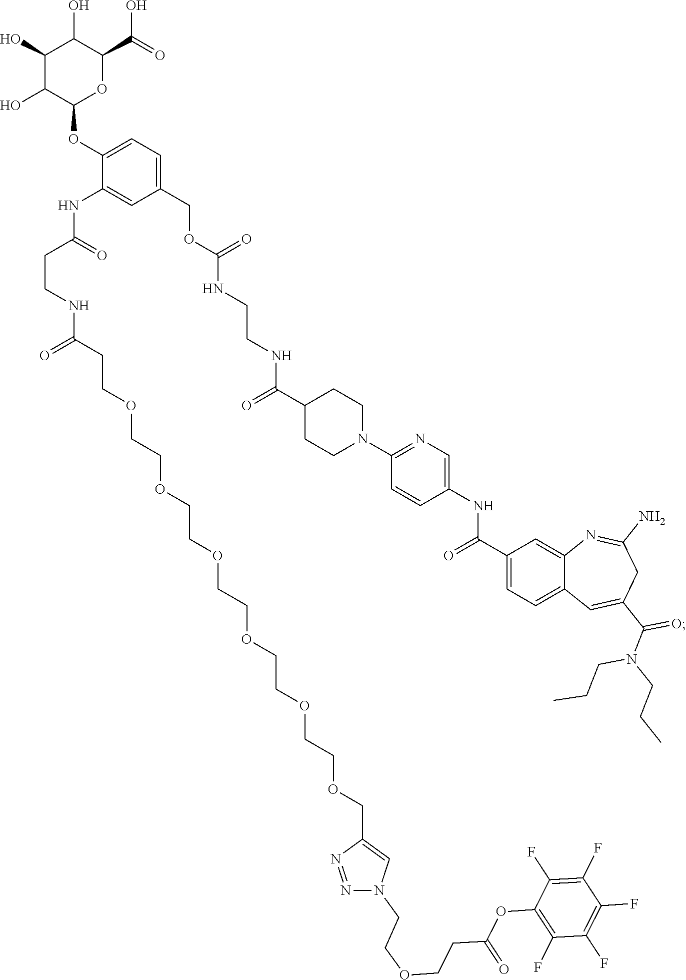 [SMILES: CCCN(CCC)C(=O)C1=Cc2ccc(C(=O)Nc3ccc(N4CCC(C(=O)NCCNC(=O)OCc5ccc(O[C@@H]6O[C@H](C(=O)O)C(O)[C@H](O)C6O)c(NC(=O)CCNC(=O)CCOCCOCCOCCOCCOCCOCc6cn(CCOCCC(=O)Oc7c(F)c(F)c(F)c(F)c7F)nn6)c5)CC4)nc3)cc2N=C(N)C1]

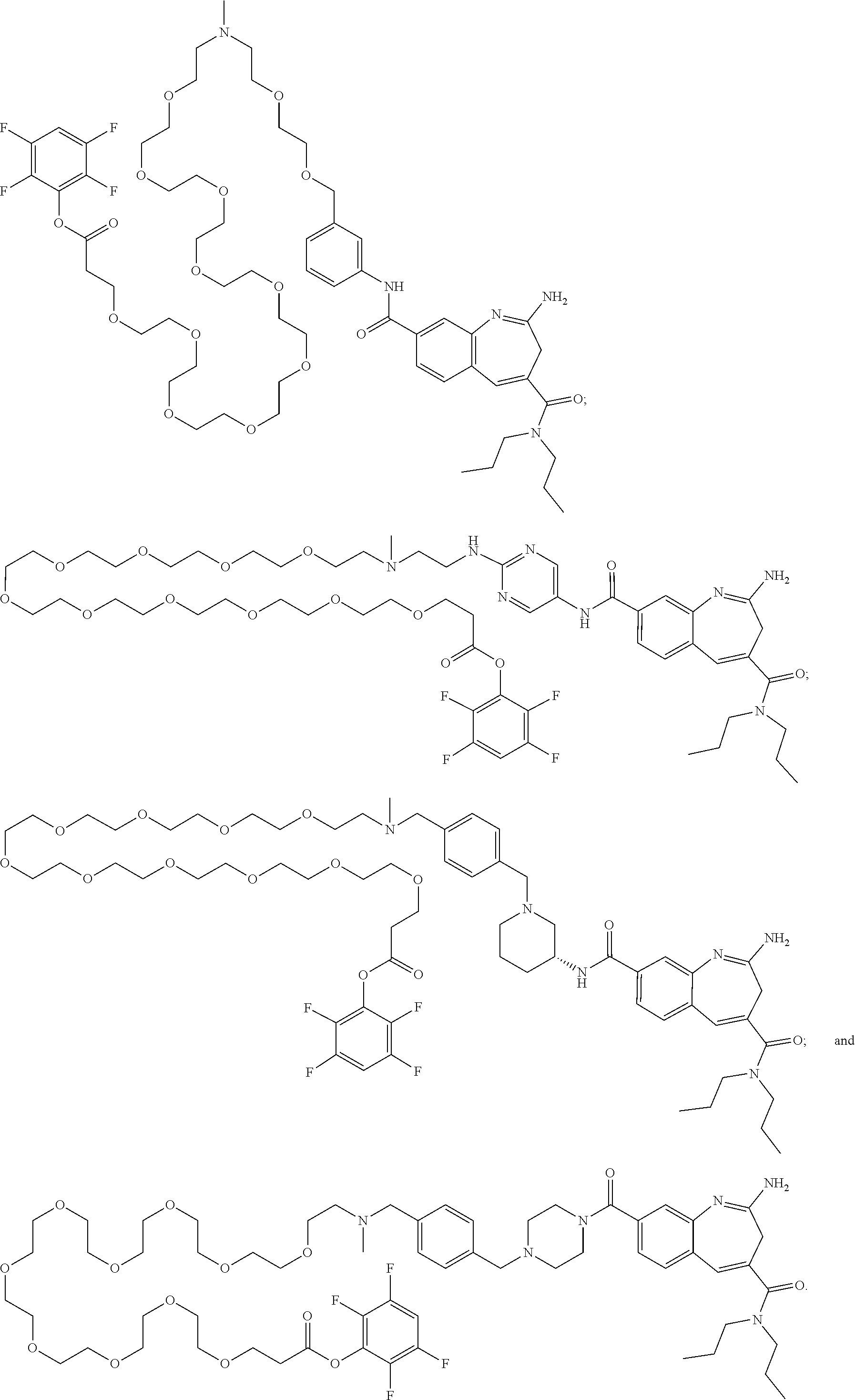

55. The 8-amido-2-aminobenzazepine-linker compound of claim 29 selected from the group consisting of:

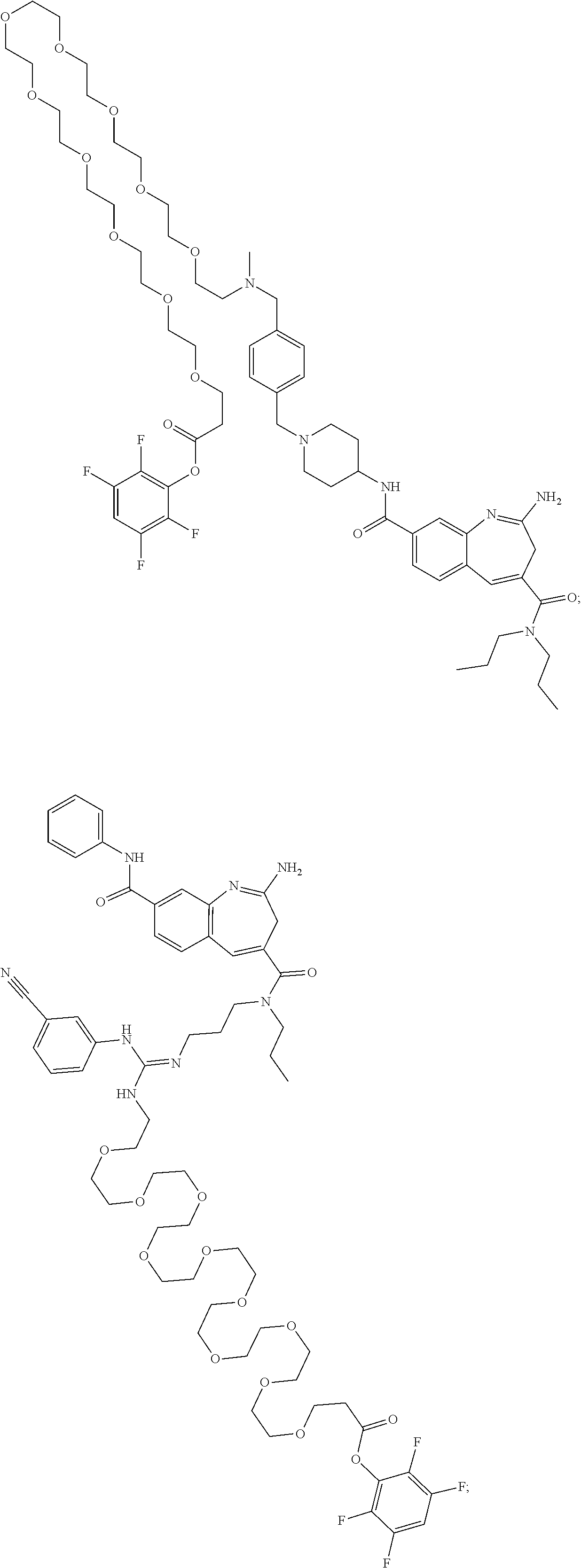 [SMILES: CCCN(CCC)C(=O)C1=Cc2ccc(C(=O)NC3CCN(Cc4ccc(CN(C)CCOCCOCCOCCOCCOCCOCCOCCOCCOCCOCCC(=O)Oc5c(F)c(F)cc(F)c5F)cc4)CC3)cc2N=C(N)C1 ; CCCN(CCC/N=C(/NCCOCCOCCOCCOCCOCCOCCOCCOCCOCCOCCC(=O)Oc1c(F)c(F)cc(F)c1F)Nc1cccc(C#N)c1)C(=O)C1=Cc2ccc(C(=O)Nc3ccccc3)cc2N=C(N)C1]

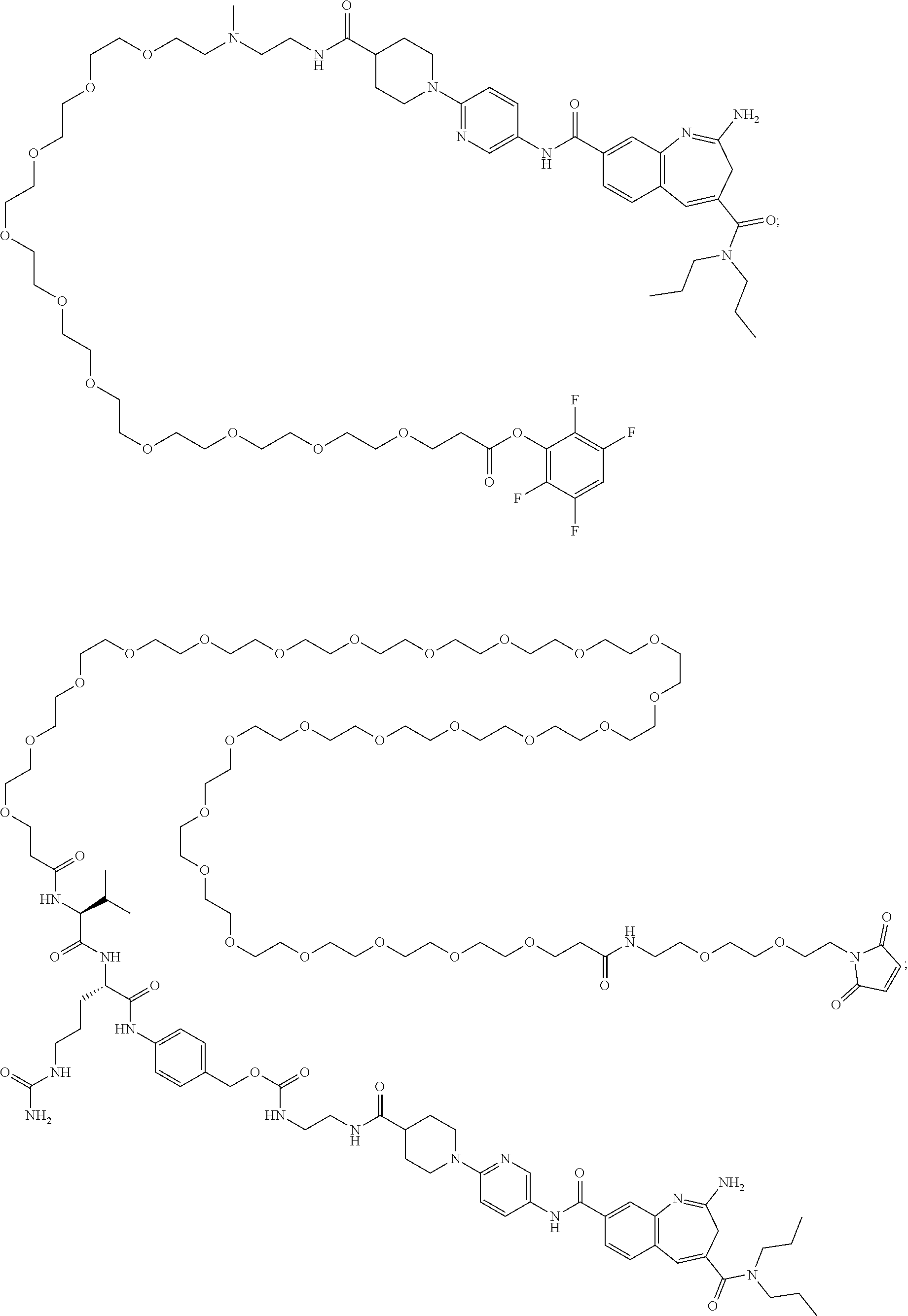 [SMILES: CCCN(CCC)C(=O)C1=Cc2ccc(C(=O)Nc3ccc(N4CCC(C(=O)NCCN(C)CCOCCOCCOCCOCCOCCOCCOCCOCCOCCOCCC(=O)Oc5c(F)c(F)cc(F)c5F)CC4)nc3)cc2N=C(N)C1 ; CCCN(CCC)C(=O)C1=Cc2ccc(C(=O)Nc3ccc(N4CCC(C(=O)NCCNC(=O)OCc5ccc(NC(=O)[C@H](CCCNC(N)=O)NC(=O)[C@@H](NC(=O)CCOCCOCCOCCOCCOCCOCCOCCOCCOCCOCCOCCOCCOCCOCCOCCOCCOCCOCCOCCOCCOCCOCCOCCOCCOCCC(=O)NCCOCCOCCN6C(=O)C=CC6=O)C(C)C)cc5)CC4)nc3)cc2N=C(N)C1]

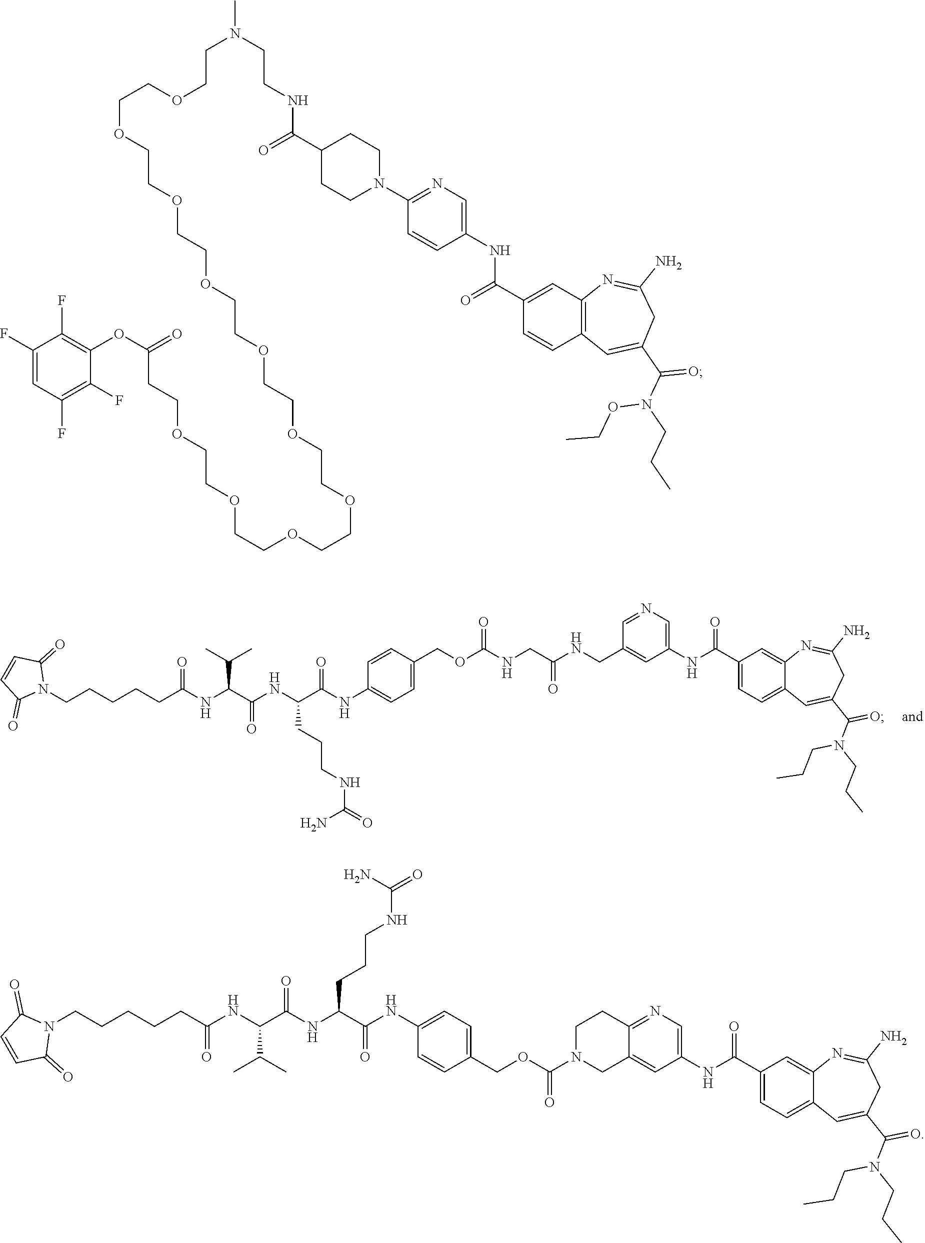 [SMILES: CCCN(OCC)C(=O)C1=Cc2ccc(C(=O)Nc3ccc(N4CCC(C(=O)NCCN(C)CCOCCOCCOCCOCCOCCOCCOCCOCCOCCOCCC(=O)Oc5c(F)c(F)cc(F)c5F)CC4)nc3)cc2N=C(N)C1 ; CCCN(CCC)C(=O)C1=Cc2ccc(C(=O)Nc3cncc(CNC(=O)CNC(=O)OCc4ccc(NC(=O)[C@H](CCCNC(N)=O)NC(=O)[C@@H](NC(=O)CCCCCN5C(=O)C=CC5=O)C(C)C)cc4)c3)cc2N=C(N)C1 ; "and" ; CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1]

56. A method for treating cancer comprising administering a therapeutically effective amount of an immunoconjugate according to claim 1, to a patient in need thereof, wherein the cancer is selected from bladder cancer, urinary tract cancer, urothelial carcinoma, lung cancer, non-small cell lung cancer, Merkel cell carcinoma, colon cancer, colorectal cancer, gastric cancer, and breast cancer.

57. The method of claim 56, wherein the cancer is susceptible to a pro-inflammatory response induced by TLR7 and/or TLR8 agonism.

58. The method of claim 56, wherein the cancer is selected from a PD-L1-expressing cancer, a HER2-expressing cancer, and a CEA-expressing cancer.

59-62. (canceled)

63. The method of claim 56, wherein the cancer is selected from triple-negative breast cancer, metastatic Merkel cell carcinoma, HER2 overexpressing gastric cancer, and gastroesophageal junction adenocarcinoma.

64-67. (canceled)

68. A method of preparing an immunoconjugate of Formula I of claim 1 wherein an 8-amido-2-aminobenzazepine-linker compound of Formula II of claim 29 is conjugated with the antibody.

69. The method of claim 68 wherein the 8-amido-2-aminobenzazepine-linker compound is selected from the group consisting of: 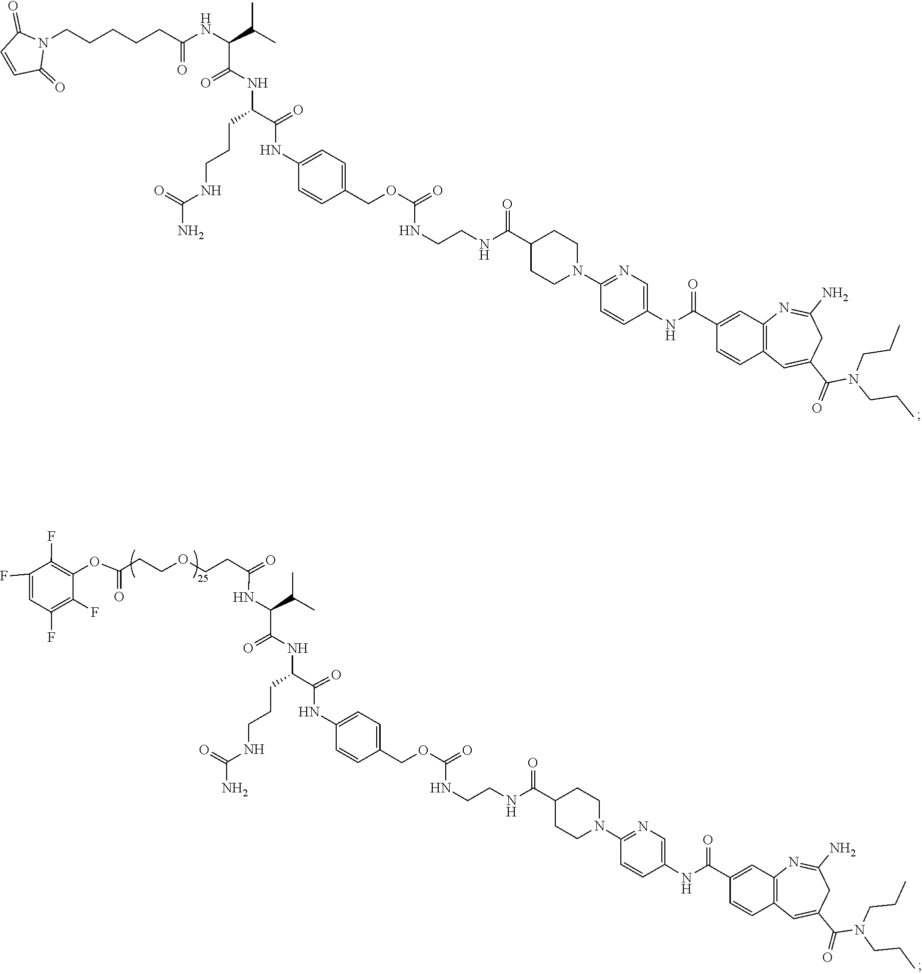 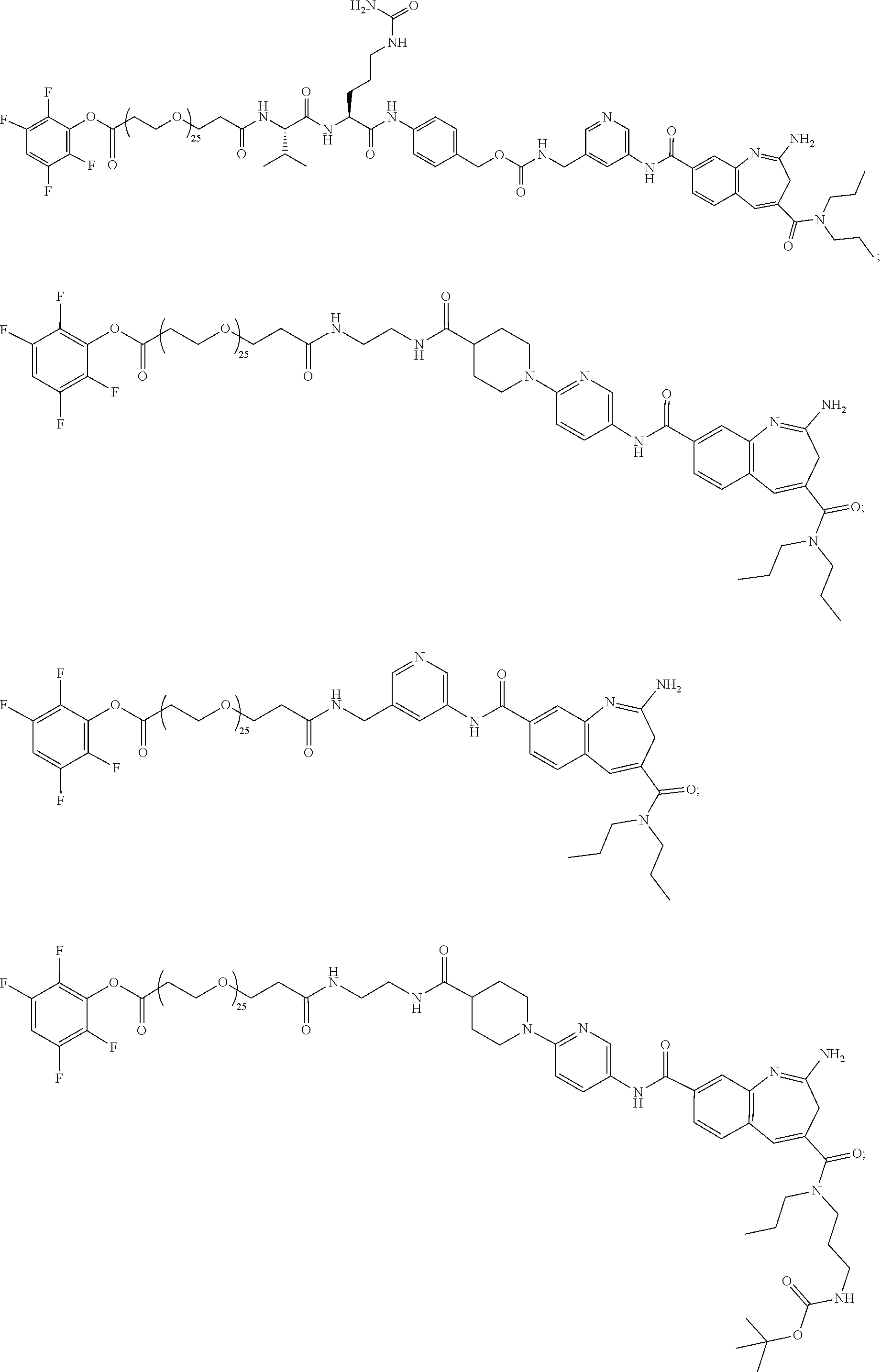 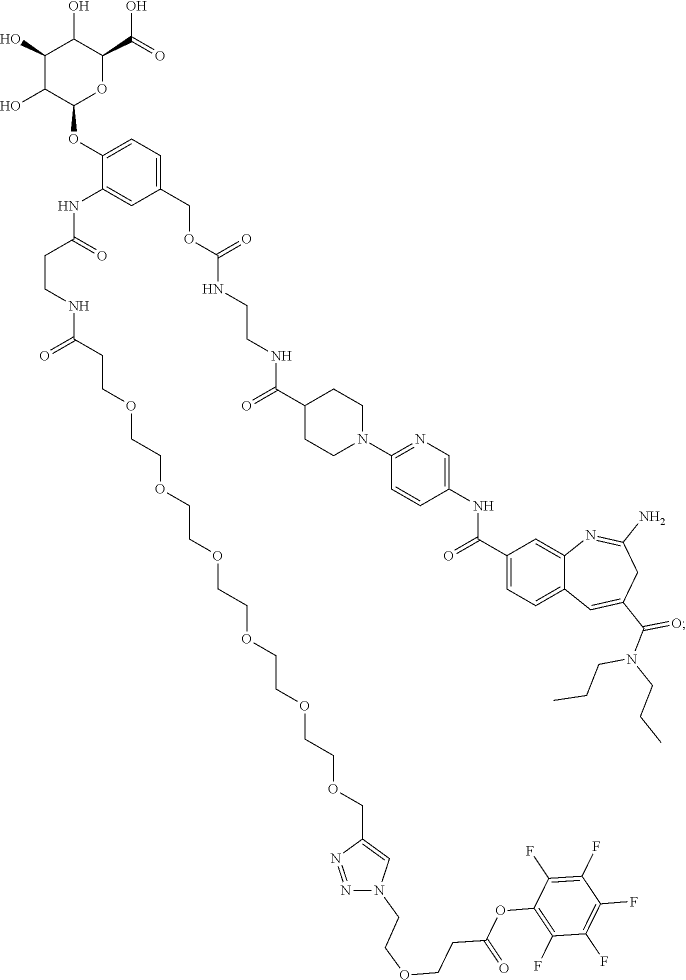 [SMILES: CCCN(CCC)C(=O)C1=Cc2ccc(C(=O)Nc3ccc(N4CCC(C(=O)NCCNC(=O)OCc5ccc(O[C@@H]6O[C@H](C(=O)O)C(O)[C@H](O)C6O)c(NC(=O)CCNC(=O)CCOCCOCCOCCOCCOCCOCc6cn(CCOCCC(=O)Oc7c(F)c(F)c(F)c(F)c7F)nn6)c5)CC4)nc3)cc2N=C(N)C1] 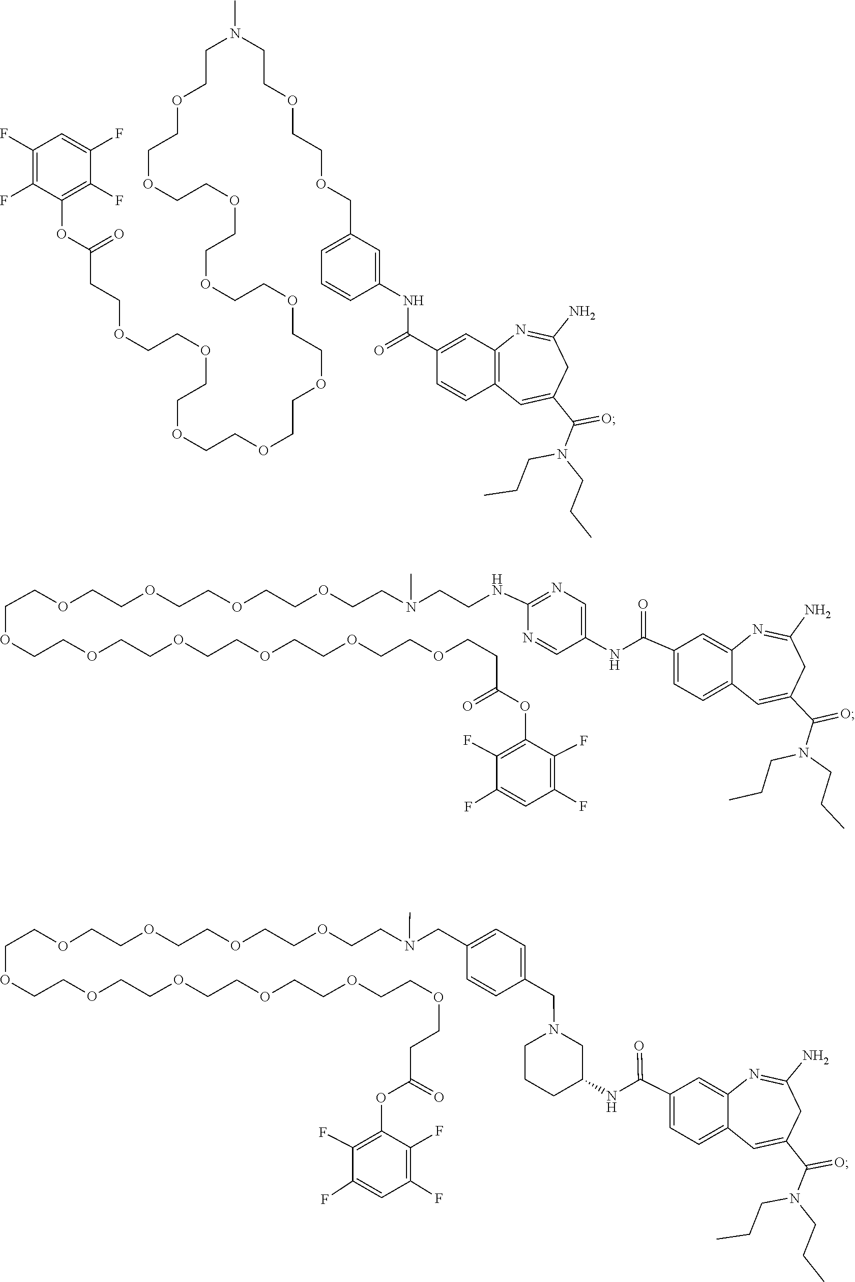 [SMILES: CCCN(CCC)C(=O)C1=Cc2ccc(C(=O)Nc3cccc(COCCOCCN(C)CCOCCOCCOCCOCCOCCOCCOCCOCCOCCOCCC(=O)Oc4c(F)c(F)cc(F)c4F)c3)cc2N=C(N)C1 ; CCCN(CCC)C(=O)C1=Cc2ccc(C(=O)Nc3cnc(NCCN(C)CCOCCOCCOCCOCCOCCOCCOCCOCCOCCOCCC(=O)Oc4c(F)c(F)cc(F)c4F)nc3)cc2N=C(N)C1 ; CCCN(CCC)C(=O)C1=Cc2ccc(C(=O)N[C@@H]3CCCN(Cc4ccc(CN(C)CCOCCOCCOCCOCCOCCOCCOCCOCCOCCOCCC(=O)Oc5c(F)c(F)cc(F)c5F)cc4)C3)cc2N=C(N)C1] 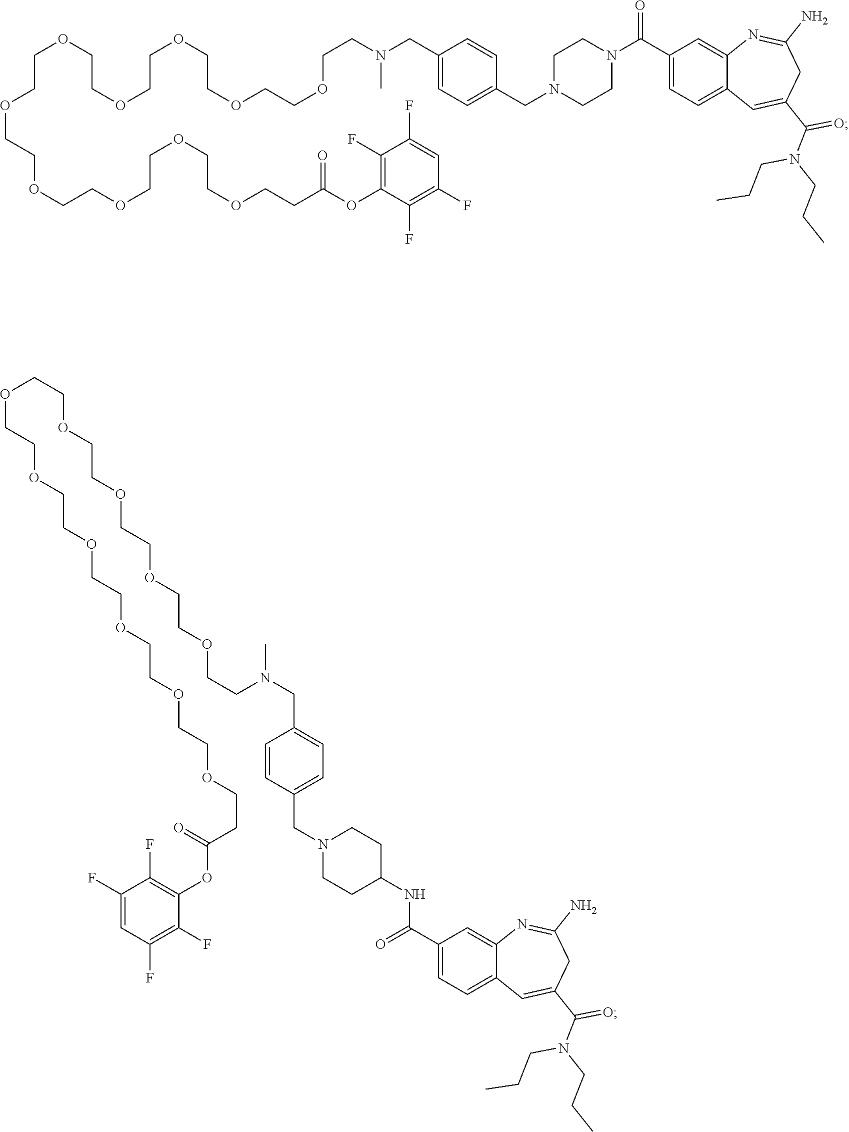 [SMILES: CCCN(CCC)C(=O)C1=Cc2ccc(C(=O)N3CCN(Cc4ccc(CN(C)CCOCCOCCOCCOCCOCCOCCOCCOCCOCCOCCC(=O)Oc5c(F)c(F)cc(F)c5F)cc4)CC3)cc2N=C(N)C1 ; CCCN(CCC)C(=O)C1=Cc2ccc(C(=O)NC3CCN(Cc4ccc(CN(C)CCOCCOCCOCCOCCOCCOCCOCCOCCOCCOCCC(=O)Oc5c(F)c(F)cc(F)c5F)cc4)CC3)cc2N=C(N)C1] 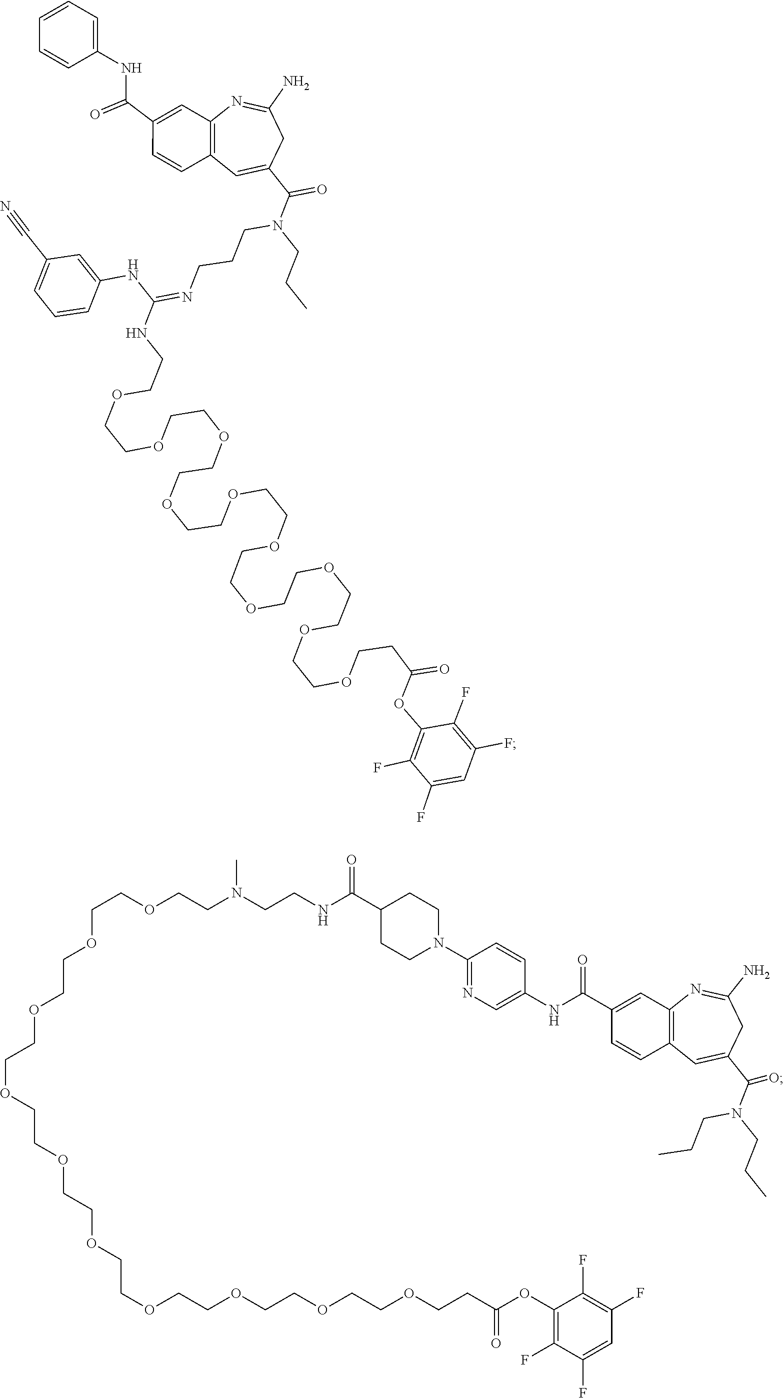 [SMILES: CCCN(CCC/N=C(/NCCOCCOCCOCCOCCOCCOCCOCCOCCOCCOCCC(=O)Oc1c(F)c(F)cc(F)c1F)Nc1cccc(C#N)c1)C(=O)C1=Cc2ccc(C(=O)Nc3ccccc3)cc2N=C(N)C1 ; CCCN(CCC)C(=O)C1=Cc2ccc(C(=O)Nc3ccc(N4CCC(C(=O)NCCN(C)CCOCCOCCOCCOCCOCCOCCOCCOCCOCCOCCC(=O)Oc5c(F)c(F)cc(F)c5F)CC4)nc3)cc2N=C(N)C1] 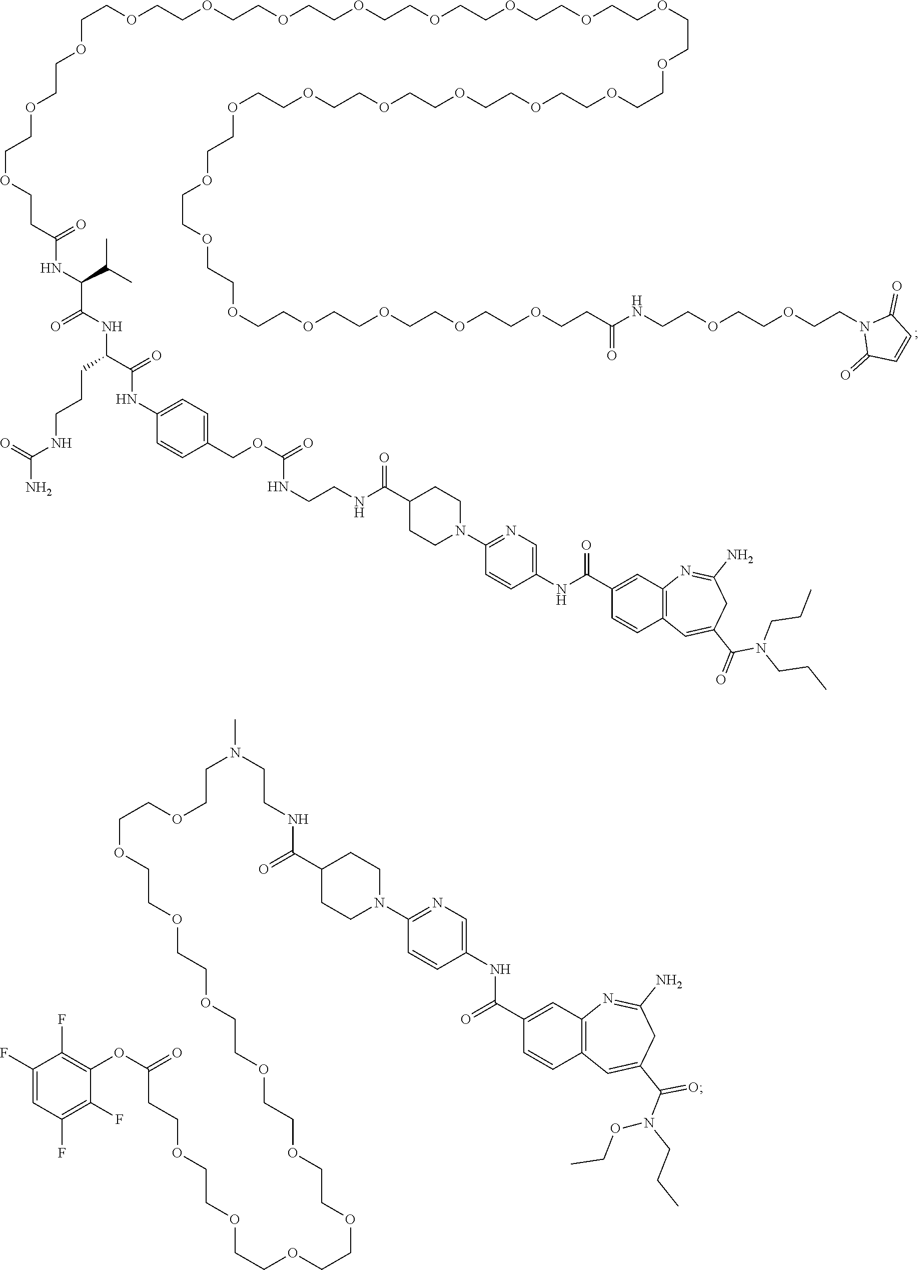 [SMILES: CCCN(CCC)C(=O)C1=Cc2ccc(C(=O)Nc3ccc(N4CCC(C(=O)NCCNC(=O)OCc5ccc(NC(=O)[C@H](CCCNC(N)=O)NC(=O)[C@@H](NC(=O)CCOCCOCCOCCOCCOCCOCCOCCOCCOCCOCCOCCOCCOCCOCCOCCOCCOCCOCCOCCOCCOCCOCCOCCOCCOCCC(=O)NCCOCCOCCN6C(=O)C=CC6=O)C(C)C)cc5)CC4)nc3)cc2N=C(N)C1 ; CCCN(OCC)C(=O)C1=Cc2ccc(C(=O)Nc3ccc(N4CCC(C(=O)NCCN(C)CCOCCOCCOCCOCCOCCOCCOCCOCCOCCOCCC(=O)Oc5c(F)c(F)cc(F)c5F)CC4)nc3)cc2N=C(N)C1] 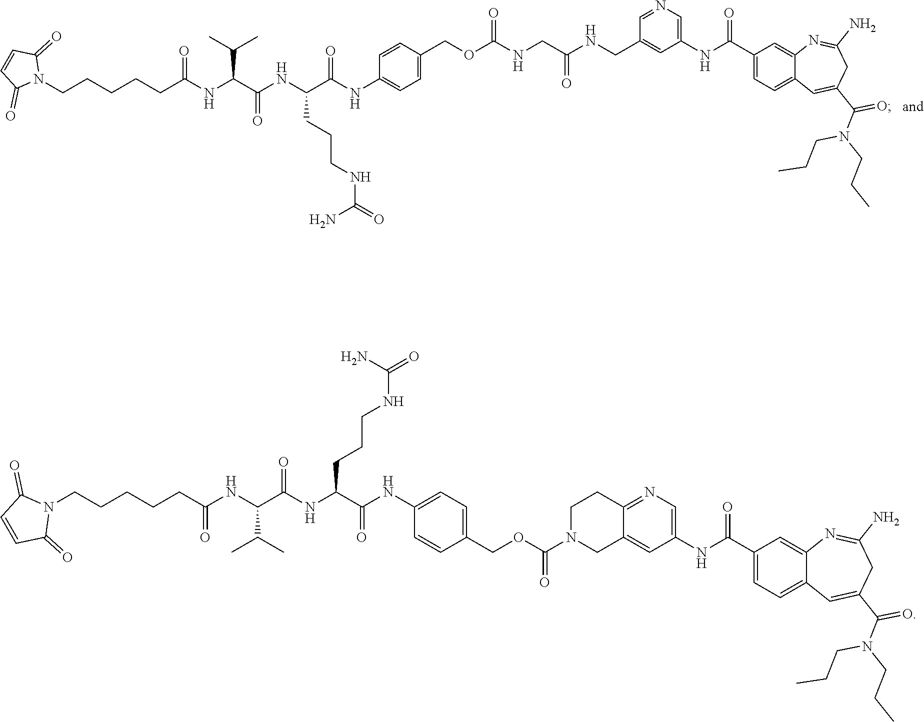 [SMILES: CCCN(CCC)C(=O)C1=Cc2ccc(C(=O)Nc3cncc(CNC(=O)CNC(=O)OCc4ccc(NC(=O)[C@H](CCCNC(N)=O)NC(=O)[C@@H](NC(=O)CCCCCN5C(=O)C=CC5=O)C(C)C)cc4)c3)cc2N=C(N)C1 ; "and" ; CCCN(CCC)C(=O)C1=Cc2ccc(C(=O)Nc3cnc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)cc2N=C(N)C1]
